SEQUENCE LISTING (1771 sequences)

SEQ ID NO: 1 - protein, 468 aa, Homo sapiens
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVQDSSSVPLPTFLVAGGSLAFGTLLCIAIVLRFKKTWKLRALKEGKTSMHPPYSLGQLVPERPRPTPVLVPLISPPVSPSSLGSDNTSSHNRPDARDPRSPYDISNTDYFFPR

SEQ ID NO: 2 - protein, 8 aa, synthetic construct
GFTFSSYY

SEQ ID NO: 3 - protein, 8 aa, synthetic construct
IYSDGTTH

SEQ ID NO: 4 - protein, 14 aa, synthetic construct
AKGAGPTWWYALDA

SEQ ID NO: 5 - protein, 8 aa, synthetic construct
RFTFDDYA

SEQ ID NO: 6 - protein, 8 aa, synthetic construct
ISWNSGRI

SEQ ID NO: 7 - protein, 9 aa, synthetic construct
AKGRDSFDI

SEQ ID NO: 8 - protein, 9 aa, synthetic construct
GHSISHDHA

SEQ ID NO: 9 - protein, 8 aa, synthetic construct
FISYSGIT

SEQ ID NO: 10 - protein, 12 aa, synthetic construct
ARSLARTTAMDY

SEQ ID NO: 11 - protein, 120 aa, synthetic construct
QVQLVQSGGGLVQPGGSLRLSCAASGFTFSSYYMSWVRQAPGKGLEWVSGIYSDGTTHYGDSVKGRFTISRDNAKNTVYLQLNSLRAEDTAMYYCAKGAGPTWWYALDAWGQGTLVTVSS

SEQ ID NO: 12 - protein, 116 aa, synthetic construct
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSS

SEQ ID NO: 13 - protein, 119 aa, synthetic construct
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSS

SEQ ID NO: 14 - protein, 119 aa, synthetic construct [sequence omitted]

SEQ ID NO: 15 - protein, 10 aa, synthetic construct
QSVLSASNTY

SEQ ID NO: 16 - skipped

SEQ ID NO: 17 - protein, 9 aa, synthetic construct
QQAYRAPVT

SEQ ID NO: 18 - protein, 6 aa, synthetic construct
QGISSW

SEQ ID NO: 19 - skipped

SEQ ID NO: 20 - protein, 9 aa, synthetic construct
QQANSFPYT

SEQ ID NO: 21 - protein, 6 aa, synthetic construct
TDISSH

SEQ ID NO: 22 - skipped

SEQ ID NO: 23 - protein, 9 aa, synthetic construct
GQGNRLPYT

SEQ ID NO: 24 - protein, 111 aa, synthetic construct
DIQLTQSPSSVSVSVGERVTIDCKSSQSVLSASNTYLNWYQQKPGQAPQLLIYYASTRESGVPDRFSGSGSGTDFTLTISSLQAEDAAVYYCQQAYRAPVTFGQGTKLEIK

SEQ ID NO: 25 - protein, 107 aa, synthetic construct
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIK

SEQ ID NO: 26 - protein, 107 aa, synthetic construct
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIE

SEQ ID NO: 27 - protein, 107 aa, synthetic construct [sequence omitted]

SEQ ID NO: 28 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 29 - protein, 180 aa, Homo sapiens
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 30 - protein, 197 aa, Homo sapiens
MTLLPGLLFLTWLHTCLAHHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 31 - protein, 202 aa, Homo sapiens
MLVAGFLLALPPSWAAGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 32 - protein, 177 aa, Homo sapiens
MRERPRLGEDSSLISLFLQVVAFLAMVMGTHTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 33 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 34 - protein, 866 aa, Homo sapiens
MGAARSPPSAVPGPLLGLLLLLLGVLAPGGASLRLLDHRALVCSQPGLNCTVKNSTCLDDSWIHPRNLTPSSPKDLQIQLHFAHTQQGDLFPVAHIEWTLQTDASILYLEGAELSVLQLNTNERLCVRFEFLSKLRHHHRRWRFTFSHFVVDPDQEYEVTVHHLPKPIPDGDPNHQSKNFLVPDCEHARMKVTTPCMSSGSLWDPNITVETLEAHQLRVSFTLWNESTHYQILLTSFPHMENHSCFEHMHHIPAPRPEEFHQRSNVTLTLRNLKGCCRHQVQIQPFFSSCLNDCLRHSATVSCPEMPDTPEPIPDYMPLWVYWFITGISILLVGSVILLIVCMTWRLAGPGSEKYSDDTKYTDGLPAADLIPPPLKPRKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDWFECENLYSADDQDAPSLDEEVFEEPLLPPGTGIVKRAPLVREPGSQACLAIDPLVGEEGGAAVAKLEPHLQPRGQPAPQPLHTLVLAAEEGALVAAVEPGPLADGAAVRLALAGEGEACPLLGSPGAGRNSVLFLPVDPEDSPLGSSTPMASPDLLPEDVREHLEGLMLSLFEQSLSCQAQGGCSRPAMVLTDPHTPYEEEQRQSVQSDQGYISRSSPQPPEGLTEMEEEEEEEQDPGKPALPLSPEDLESLRSLQRQLLFRQLQKNSGWDTMGSESEGPSA

SEQ ID NO: 35 - protein, 502 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 36 - protein, 791 aa, Homo sapiens
MPVPWFLLSLALGRSPVVLSLERLVGPQDATHCSPVSLEPWGDEERLRVQFLAQQSLSLAPVTAATARTALSGLSGADGRREERGRGKSWVCLSLGGSGNTEPQKKGLSCRLWDSDILCLPGDIVPAPGPVLAPTHLQTELVLRCQKETDCDLCLRVAVHLAVHGHWEEPEDEEKFGGAADSGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLQLLTLQSWLLDAPCSLPAEAALCWRAPGGDPCQPLVPPLSWENVTVDKVLEFPLLKGHPNLCVQVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGTPAPGRGVGPGAGPGAGDGT

SEQ ID NO: 37 - protein, 739 aa, Homo sapiens
MAPWLQLCSVFFTVNACLNGSQLAVAAGGSGRARGADTCGWRGVGPASRNSGLYNITFKYDNCTTYLNPVGKHVIADAQNITISQYACHDQVAVTILWSPGALGIEFLKGFRVILEELKSEGRQCQQLILKDPKQLNSSFKRTGMESQPFLNMKFETDYFVKVVPFPSIKNESNYHPFFFRTRACDLLLQPDNLACKPFWKPRNLNISQHGSDMQVSFDHAPHNFGFRFFYLHYKLKHEGPFKRKTCKQEQTTETTSCLLQNVSPGDYIIELVDDTNTTRKVMHYALKPVHSPWAGPIRAVAITVPLVVISAFATLFTVMCRKKQQENIYSHLDEESSESSTYTAALPRERLRPRPKVFLCYSSKDGQNHMNVVQCFAYFLQDFCGCEVALDLWEDFSLCREGQREWVIQKIHESQFIIVVCSKGMKYFVDKKNYKHKGGGRGSGKGELFLVAVSAIAEKLRQAKQSSSAALSKFIAVYFDYSCEGDVPGILDLSTKYRLMDNLPQLCSHLHSRDHGLQEPGQHTRQGSRRNYFRSKSGRSLYVAICNMHQFIDEEPDWFEKQFVPFHPPPLRYREPVLEKFDSGLVLNDVMCKPGPESDFCLKVEAAVLGATGPADSQHESQHGGLDQDGEARPALDGSAALQPLLHTVKAGSPSDMPRDSGIYDSSVPSSELSLPLMEGLSTDQTETSSLTESVSSSSGLGEEEPPALPSKLLSSGSCKADLGCRSYTDELHAVAPL

SEQ ID NO: 38 - protein, 667 aa, Homo sapiens
MGSSRLAALLLPLLLIVIDLSDSAGIGFRHLPHWNTRCPLASHTDDSFTGSSAYIPCRTWWALFSTKPWCVRVWHCSRCLCQHLLSGGSGLQRGLFHLLVQKSKKSSTFKFYRRHKMPAPAQRKLLPRRHLSEKSHHISIPSPDISHKGLRSKRTQPSDPETWESLPRLDSQRHGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKFSFGNSSHVECPHQTGSLTSWNVSMDTQAQQLILHFSSRMHATFSAAWSLPGLGQDTLVPPVYTVSQARGSSPVSLDLIIPFLRPGCCVLVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 39 - protein, 8 aa, synthetic construct
GFTFDDYA

SEQ ID NO: 40 - protein, 7 aa, synthetic construct
NWSSGGI

SEQ ID NO: 41 - protein, 14 aa, synthetic construct
ARDIGGFGEFYWNF

SEQ ID NO: 42 - protein, 8 aa, synthetic construct
GFTFSDYN

SEQ ID NO: 43 - protein, 9 aa, synthetic construct
TITYEGRNT

SEQ ID NO: 44 - protein, 18 aa, synthetic construct
ASPPQYYEGSIYRLWFAH

SEQ ID NO: 45 - protein, 10 aa, synthetic construct
SYSFTSDYAW

SEQ ID NO: 46 - protein, 7 aa, synthetic construct
ITYSGVT

SEQ ID NO: 47 - protein, 13 aa, synthetic construct
ARADYDSYYTMDY

SEQ ID NO: 48 - protein, 9 aa, synthetic construct
GYSFTDYHI

SEQ ID NO: 49 - protein, 10 aa, synthetic construct
VINPMYGTTD

SEQ ID NO: 50 - protein, 12 aa, synthetic construct
ARYDYFTGTGVY

SEQ ID NO: 51 - protein, 8 aa, synthetic construct
GFTFSNYW

SEQ ID NO: 52 - protein, 9 aa, synthetic construct
AINQDGSEK

SEQ ID NO: 53 - protein, 20 aa, synthetic construct [sequence omitted]

SEQ ID NO: 54 - protein, 7 aa, synthetic construct
GYTFTRY

SEQ ID NO: 55 - protein, 8 aa, synthetic construct
ISTYSGNT

SEQ ID NO: 56 - protein, 9 aa, synthetic construct
ARRQLYFDY

SEQ ID NO: 57 - protein, 8 aa, synthetic construct
GGTFATSP

SEQ ID NO: 58 - protein, 7 aa, synthetic construct
ISPSGGD

SEQ ID NO: 59 - protein, 19 aa, synthetic construct
CAVRRRFDGTSYYTGDYDS

SEQ ID NO: 60 - protein, 8 aa, synthetic construct
GFTFSDYT

SEQ ID NO: 61 - protein, 8 aa, synthetic construct
IKSGGSYS

SEQ ID NO: 62 - protein, 15 aa, synthetic construct
ARDGDYGSSYGAMDY

SEQ ID NO: 63 - skipped

SEQ ID NO: 64 - protein, 8 aa, synthetic construct
ITWNSGHI

SEQ ID NO: 65 - protein, 14 aa, synthetic construct
AKVSYLSTASSLDY

SEQ ID NO: 66 - protein, 8 aa, synthetic construct
GGSFGGYG

SEQ ID NO: 67 - protein, 7 aa, synthetic construct
ITPFFGF

SEQ ID NO: 68 - protein, 19 aa, synthetic construct
ARDPNEFWNGYYSTHDFDS

SEQ ID NO: 69 - protein, 8 aa, synthetic construct
GRTFSSYV

SEQ ID NO: 70 - protein, 8 aa, synthetic construct
ISGSGESI

SEQ ID NO: 71 - protein, 16 aa, synthetic construct
TADQEFGYLRFGRSEY

SEQ ID NO: 72 - protein, 6 aa, synthetic construct
GRTYDA

SEQ ID NO: 73 - protein, 8 aa, synthetic construct
ISGSGDDT

SEQ ID NO: 74 - protein, 17 aa, synthetic construct
ATRRGLYYVWDANDYEN

SEQ ID NO: 75 - protein, 8 aa, synthetic construct
GYKFTDYH

SEQ ID NO: 76 - protein, 8 aa, synthetic construct
INPTYGTT

SEQ ID NO: 77 - protein, 12 aa, synthetic construct
ARYDYFTGTGVY

SEQ ID NO: 78-83 - skipped (6 entries)

SEQ ID NO: 84 - protein, 123 aa, synthetic construct
EVQLVESGGGLVQPGRSLRLSCAASGFTFDDYAMHWVRQAPGKGLEWVSGINWSSGGIGYADSVKGRFTISRDNAKNSLYLQMNSLRAEDTALYYCARDIGGFGEFYWNFGLWGRGTLVTVSS

SEQ ID NO: 85 - protein, 125 aa, synthetic construct
EVQLVESGGGLVQPGGSLRLSCAASGFTFSDYNMAWVRQAPGKGLEWVATITYEGRNTYYRDSVKGRFTISRDNAKNSLYLQMNSLRAEDTAVYYCASPPQYYEGSIYRLWFAHWGQGTLVTVSS

SEQ ID NO: 86 - protein, 120 aa, synthetic construct
DVQLQESGPGLVKPSQTLSLTCTVSSYSFTSDYAWSWIRQPPGKGLEWIGYITYSGVTSYNPSLKSRVTISVDTSKNQFSLKLSSVTAADTAVYYCARADYDSYYTMDYWGQGTSVTVSS

SEQ ID NO: 87 - protein, 119 aa, synthetic construct
QVQLVQSGAEVKKPGSSVKVSCKASGYSFTDYHIHWVRQAPGQGLEWMGVINPMYGTTDYNQRFKGRVTITADESTSTAYMELSSLRSEDTAVYYCARYDYFTGTGVYWGQGTLVTVSS

SEQ ID NO: 88 - protein, 127 aa, synthetic construct [sequence omitted]

SEQ ID NO: 89 - protein, 116 aa, synthetic construct
QVQLVQSGAEVKKPGASVKVSCKASGYTFTRYGISWVRQAPGQGLEWMGWISTYSGNTNYAQKLQGRVTMTTDTSTSTAYMELRSLRSDDTAVYYCARRQLYFDYWGQGTLVTVSS

SEQ ID NO: 90 - protein, 125 aa, synthetic construct
QVQLVQSGGGLVQAGGSLRLSCAASGGTFATSPMGWLRQAPGKGTEFVAAISPSGGDRIYADSVKGRFTISRDNAGYFIYLQMNSLKPEDTAVYYCAVRRRFDGTSYYTGDYDSWGQGTLVTVSS

SEQ ID NO: 91 - protein, 122 aa, synthetic construct
EVQLVESGGGLVQPGGSLRLSCAASGFTFSDYTMLWVRQAPGKGLEWVAIIKSGGSYSYYPDSVKGRFTISRDNAKNSLYLQMNSLRAEDTAVYYCARDGDYGSSYGAMDYWGQGTLVTVSS

SEQ ID NO: 92 - protein, 121 aa, synthetic construct [sequence omitted]

SEQ ID NO: 93 - protein, 123 aa, synthetic construct
DVQLVESGGGLVQPGGSLRLSCAASGRTFSSYVVGWFRQAPGKEREFIGAISGSGESIYYAVSEKGRFTISRDNSKNTLYLQMNSLRPEDTAVYYCTADQEFGYLRFGRSEYWGQGTLVTVSS

SEQ ID NO: 94 - protein, 122 aa, synthetic construct
EVQLVESGGGLVQPGGSLRLSCAASGRTYDAMGWLRQAPGKEREFVAAISGSGDDTYYADSVKGRFTISRDNSKNTLYLQMNSLRPEDTAVYYCATRRGLYYVWDANDYENWGQGTLVTVSS

SEQ ID NO: 95 - protein, 119 aa, synthetic construct
QVQLVQSGAEVKKPGSSVKVSCKASGYKFTDYHIHWVRQAPGQCLEWMGVINPTYGTTDYNQRFKGRVTITADESTSTAYMELSSLRSEDTAVYYCARYDYFTGTGVYWGQGTLVTVSS

SEQ ID NO: 96 - protein, 117 aa, synthetic construct
QVQLVQSGAEVKKPGASVKVSCKASGYIFTNYGMNWMRQAPGQGLEWMGWINDHTGEPTYADKFQGRVTFTLDTSISTAYMELSRLRSDDTAVYYCANYGFGYFDYWGQGTLVTVSS

SEQ ID NO: 97 - protein, 123 aa, synthetic construct
EVQLVQSGAEVKKPGASVKVSCKASGYTFTDYEVHWVRQAPGQGLEWMGVIDPGTGGVAYNQKFEGRVTMTADTSTSTAYMELRSLRSDDTAVYYCTRYSLFYGSSPYAMDYWGQGTLVTVSS

SEQ ID NO: 98 - protein, 127 aa, synthetic construct
EVQLVESGGGLVQPGGSLRLSCAASGMSMSDYWMNWVRQAPGKGLEWVAAINQDGDEKYYVGSVKGRFTISRDNAKNSLYLQMNSLRVEDTAVYYCVRDYYDLISDYYIHYWYFDLWGRGTLVTVSS

SEQ ID NO: 99 - protein, 6 aa, synthetic construct
QSVRSY

SEQ ID NO: 100 - skipped

SEQ ID NO: 101 - protein, 10 aa, synthetic construct
QQRSNWPPAT

SEQ ID NO: 102 - protein, 9 aa, synthetic construct
DESVRTLMH

SEQ ID NO: 103 - protein, 5 aa, synthetic construct
LVSNS

SEQ ID NO: 104 - protein, 9 aa, synthetic construct
QQTWSDPWT

SEQ ID NO: 105 - protein, 11 aa, synthetic construct
QSLVHSNGNTY

SEQ ID NO: 106 - skipped

SEQ ID NO: 107 - protein, 8 aa, synthetic construct
SQSTHFWT

SEQ ID NO: 108 - protein, 11 aa, synthetic construct
RSLVHSRGNTY

SEQ ID NO: 109 - protein, 9 aa, synthetic construct
SQSTHLPFT

SEQ ID NO: 110 - protein, 8 aa, synthetic construct
QSVSSSYL

SEQ ID NO: 111 - protein, 9 aa, synthetic construct
QQYGSSPCT

SEQ ID NO: 112 - protein, 6 aa, synthetic construct
QSVSSN

SEQ ID NO: 113 - protein, 9 aa, synthetic construct
QQYDNWPLT

SEQ ID NO: 114 - protein, 8 aa, synthetic construct
SQSVSSSY

SEQ ID NO: 115 - protein, 9 aa, synthetic construct
QQYSYSPVT

SEQ ID NO: 116 - protein, 6 aa, synthetic construct
QDINSY

SEQ ID NO: 117 - skipped

SEQ ID NO: 118 - protein, 11 aa, synthetic construct
CLQYDAFPPYT

SEQ ID NO: 119 - protein, 6 aa, synthetic construct
QGIRNY

SEQ ID NO: 120 - skipped

SEQ ID NO: 121 - protein, 9 aa, synthetic construct
QRYNRAPYT

SEQ ID NO: 122 - protein, 6 aa, synthetic construct
QDIGSE

SEQ ID NO: 123 - protein, 9 aa, synthetic construct
HQTDSLPYT

SEQ ID NO: 124 - protein, 11 aa, synthetic construct
RSLVHSRGETY

SEQ ID NO: 125 - skipped

SEQ ID NO: 126 - protein, 108 aa, synthetic construct
EIVLTQSPATLSLSPGERATLSCRASQSVRSYLAWYQQKPGQAPRLLIYDASNRATGIPARFSGSGSGTDFTLTISSLEPEDFAVYYCQQRSNWPPATFGGGTKVEIK

SEQ ID NO: 127 - protein, 107 aa, synthetic construct
AIQLTQSPSSLSASVGDRVTITCRADESVRTLMHWYQQKPGKAPKLLIYLVSNSEIGVPDRFSGSGSGTDFRLTISSLQPEDFATYYCQQTWSDPWTFGQGTKVEIK

SEQ ID NO: 128 - protein, 111 aa, synthetic construct
DVVMTQTPLSLPVTLGQPASISCRSSQSLVHSNGNTYLHWYLQKPGQSPRLLIYKVSNRFSGVPDRFSGSGSGTDFTLKISRVEAEDLGVYYCSQSTHFWTFGGGTKLEIK

SEQ ID NO: 129 - protein, 112 aa, synthetic construct
DIVMTQTPLSLSVTPGQPASISCRSSRSLVHSRGNTYLHWYLQKPGQSPQLLIYKVSNRFIGVPDRFSGSGSGTDFTLKISRVEAEDVGVYYCSQSTHLPFTFGQGTKLEIK

SEQ ID NO: 130 - protein, 108 aa, synthetic construct [sequence omitted]

SEQ ID NO: 131 - protein, 107 aa, synthetic construct
EIVMTQSPATLSVSPGERATLSCRASQSVSSNLAWFQQKPGQAPRPLIYDASTRATGVPARFSGSGSGTDFTLTISSLQSEDFAVYYCQQYDNWPLTFGGGTKVEIK

SEQ ID NO: 132 - protein, 108 aa, synthetic construct
EIVLTQSPGTLSLSPGERATLSCRASQSVSSSYLAWYQQKPGQAPRLLIYDASSRATGIPDRFSGSGSGTDFTLTISRLEPEDFAVYYCQQYSYSPVTFGQGTKVEIK

SEQ ID NO: 133 - protein, 108 aa, synthetic construct
DIQMTQSPSSLSASVGDRVTITCRASQDINSYLSWFQQKPGKAPKSLIVRANRLVDGVPSRFSGSGSGQDYSLTISSLQPEDFATYYCLQYDAFPPYTFGQGTKLEIK

SEQ ID NO: 134 - protein, 107 aa, synthetic construct [sequence omitted]

SEQ ID NO: 135 - protein, 112 aa, synthetic construct
DIVMTQTPLSLSVTPGQPASISCRSSRSLVHSRGETYLHWYLQKPGQSPQLLIYKVSNRFIGVPDRFSGSGSGTDFTLKISRVEAEDVGVYYCSQSTHLPFTFGCGTKLEIK

SEQ ID NO: 136 - protein, 112 aa, synthetic construct
DVVMTQSPLSLPVTLGQPASISCRSSQSLVHSNGNSYLHWYLQKPGQSPQLLIYKVSNRFSGVPDRFSGSGSGTDFTLKISRVEAEDVGVYYCSQSTHVPYTFGQGTKLEIK

SEQ ID NO: 137 - protein, 106 aa, synthetic construct
EIVLTQSPDFQSVTPKEKVTITCSASSSVNYMHWFQQKPDQSPKLWIYRTSNLASGVPSRFSGSGSGTDYTLTINSLEAEDAATYYCQQRSSYPWTFGQGTKLEIK

SEQ ID NO: 138 - protein, 107 aa, synthetic construct
DIQMTQSPSSLSASVGDRVTITCRASQNVHNRLTWYQQKPGKAPKLLIYGASNLESGVPSRFSGSGSGTDFTLTISSLQPEDFATYYCQQYNGSPTTFGQGTKVEIK

SEQ ID NO: 139 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 140 - protein, 192 aa, Homo sapiens
MQLTKGRLHFSHPLSHTKHISPFVTDAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 141 - protein, 174 aa, Homo sapiens
MRRFLSKVYSFPMRKLILFLVFPVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 142 - protein, 8 aa, synthetic construct
GYTFTSYD

SEQ ID NO: 143 - protein, 8 aa, synthetic construct
LNPNSGYT

SEQ ID NO: 144 - protein, 12 aa, synthetic construct
AREVPETAAFEY

SEQ ID NO: 145 - protein, 8 aa, synthetic construct
GFDIQDTY

SEQ ID NO: 146 - protein, 8 aa, synthetic construct
IDPASGHT

SEQ ID NO: 147 - protein, 9 aa, synthetic construct
ARSGGLPDV

SEQ ID NO: 148 - skipped

SEQ ID NO: 149 - protein, 119 aa, synthetic construct
QVQLVQSGAEVKKPGASVKVSCKASGYTFTSYDINWVRQAPGQGLEWMGWLNPNSGYTGYAQKFQGRVTMTADRSTSTAYMELSSLRSEDTAVYYCAREVPETAAFEYWGQGTLVTVSS

SEQ ID NO: 150 - protein, 116 aa, synthetic construct
QVQLVQSGAEVKKPGASVKVSCKASGFDIQDTYMHWVKQRPGQGLEWMGRIDPASGHTKYDPKFQVRVTITRDTSTSTVYLELSSLRSEDTAVYYCARSGGLPDVWGQGTTVTVSS

SEQ ID NO: 151 - protein, 9 aa, synthetic construct
SSDIGAGLG

SEQ ID NO: 152 - skipped

SEQ ID NO: 153 - protein, 10 aa, synthetic construct
QSWDGTLSAL

SEQ ID NO: 154 - protein, 5 aa, synthetic construct
SSVSY

SEQ ID NO: 155 - skipped

SEQ ID NO: 156 - protein, 9 aa, synthetic construct
QQWEGNPRT

SEQ ID NO: 157 - protein, 110 aa, synthetic construct
QSVLTQPPSVSGAPGQRVTISCTSSSSDIGAGLGVHWYQQLPGTAPKLLIEGYYNRPSGVPDRFSGSKSGTSASLTITGLLPEDEGDYYCQSWDGTLSALFGGGTKLTVL

SEQ ID NO: 158 - protein, 106 aa, synthetic construct
EIVLTQSPGTLSLSPGERATLSCRASSSVSYMYWYQQKPGQAPRPLIYATSNLASGIPDRFSGSGSGTDFTLTISRLEPEDFAVYYCQQWEGNPRTFGGGTKLEIK

SEQ ID NO: 159 - protein, 10 aa, synthetic construct
GFDIQDTYMH

SEQ ID NO: 160 - protein, 17 aa, synthetic construct
RIDPASGHTKYDPKFQV

SEQ ID NO: 161 - protein, 17 aa, synthetic construct
RIEPASGHIKYDPKFQG

SEQ ID NO: 162 - protein, 17 aa, synthetic construct
RIEPASGHIKYSPKFQG

SEQ ID NO: 163 - protein, 17 aa, synthetic construct
RIEPASGHVKYSPKFQV

SEQ ID NO: 164 - protein, 17 aa, synthetic construct
RIEPASGHVKYDPKFQT

SEQ ID NO: 165 - protein, 17 aa, synthetic construct
RIDPASGHIKYDPKFQK

SEQ ID NO: 166 - protein, 17 aa, synthetic construct
RIDPASGHVKIDPKFQV

SEQ ID NO: 167 - protein, 17 aa, synthetic construct
RIDPASGHLKYDPKFQV

SEQ ID NO: 168 - protein, 17 aa, synthetic construct
RIDPASGHLKYDPKFQR

SEQ ID NO: 169 - protein, 17 aa, synthetic construct
RIDPASGHLKYDPKFQN

SEQ ID NO: 170 - protein, 17 aa, synthetic construct
RIEPASGHLKYDPKFQE

SEQ ID NO: 171 - protein, 17 aa, synthetic construct
RIDPASGHLKYDPKFQG

SEQ ID NO: 172 - protein, 17 aa, synthetic construct
RIDPASGHTKYDPKFQG

SEQ ID NO: 173 - protein, 17 aa, synthetic construct
RIDPASGHSKYDPKFQV

SEQ ID NO: 174 - skipped

SEQ ID NO: 175 - protein, 9 aa, synthetic construct
SRSGGLPDV

SEQ ID NO: 176 - protein, 9 aa, synthetic construct
ARSGGLPDW

SEQ ID NO: 177 - protein, 9 aa, synthetic construct
ARSGGLPDM

SEQ ID NO: 178 - protein, 9 aa, synthetic construct
ARSGGLPDK

SEQ ID NO: 179 - protein, 9 aa, synthetic construct
ARSGGLPDF

SEQ ID NO: 180 - protein, 9 aa, synthetic construct
ARSGGLPDL

SEQ ID NO: 181 - protein, 5 aa, synthetic construct
DTYMH

SEQ ID NO: 182 - protein, 5 aa, synthetic construct
PASGH

SEQ ID NO: 183 - protein, 6 aa, synthetic construct
SGGLPD

SEQ ID NO: 184 - protein, 116 aa, synthetic construct
QVQLVQSGAEVKKPGASVKVSCKASGFDIQDTYMHWVRQAPGQGLEWMGRIDPASGHTKYDPKFQVRVTMTRDTSTSTVYMELSSLRSEDTAVYYCARSGGLPDVWGQGTTVTVSS

SEQ ID NO: 185 - protein, 116 aa, synthetic construct
QVQLVQSGAEVKKPGASVKVSCKASGFDIQDTYMHWVRQAPGQGLEWMGRIEPASGHIKYDPKFQGRVTMTRDTSTSTVYMELSSLRSEDTAVYYCARSGGLPDWWGQGTTVTVSS

SEQ ID NO: 186 - protein, 116 aa, synthetic construct
QVQLVQSGAEVKKPGASVKVSCKASGFDIQDTYMHWVRQAPGQGLEWMGRIEPASGHIKYSPKFQGRVTMTRDTSTSTVYMELSSLRSEDTAVYYCARSGGLPDWWGQGTTVTVSS

SEQ ID NO: 187 - protein, 116 aa, synthetic construct
QVQLVQSGAEVKKPGASVKVSCKASGFDIQDTYMHWVRQAPGQGLEWMGRIEPASGHVKYSPKFQVRVTMTRDTSTSTVYMELSSLRSEDTAVYYCARSGGLPDWWGQGTTVTVSS

SEQ ID NO: 188 - protein, 116 aa, synthetic construct
QVQLVQSGAEVKKPGASVKVSCKASGFDIQDTYMHWVRQAPGQGLEWMGRIEPASGHVKYDPKFQTRVTMTRDTSTSTVYMELSSLRSEDTAVYYCARSGGLPDWWGQGTTVTVSS

SEQ ID NO: 189 - protein, 116 aa, synthetic construct
QVQLVQSGAEVKKPGASVKVSCKASGFDIQDTYMHWVRQAPGQGLEWMGRIDPASGHIKYDPKFQKRVTMTRDTSTSTVYMELSSLRSEDTAVYYCARSGGLPDMWGQGTTVTVSS

SEQ ID NO: 190 - protein, 116 aa, synthetic construct
QVQLVQSGAEVKKPGASVKVSCKASGFDIQDTYMHWVRQAPGQGLEWMGRIDPASGHVKIDPKFQVRVTMTRDTSTSTVYMELSSLRSEDTAVYYCARSGGLPDMWGQGTTVTVSS

SEQ ID NO: 191 - protein, 116 aa, synthetic construct
QVQLVQSGAEVKKPGASVKVSCKASGFDIQDTYMHWVRQAPGQGLEWMGRIDPASGHLKYDPKFQVRVTMTRDTSTSTVYMELSSLRSEDTAVYYCARSGGLPDMWGQGTTVTVSS

SEQ ID NO: 192 - protein, 116 aa, synthetic construct
QVQLVQSGAEVKKPGASVKVSCKASGFDIQDTYMHWVRQAPGQGLEWMGRIDPASGHLKYDPKFQRRVTMTRDTSTSTVYMELSSLRSEDTAVYYCARSGGLPDMWGQGTTVTVSS

SEQ ID NO: 193 - protein, 116 aa, synthetic construct
QVQLVQSGAEVKKPGASVKVSCKASGFDIQDTYMHWVRQAPGQGLEWMGRIDPASGHLKYDPKFQNRVTMTRDTSTSTVYMELSSLRSEDTAVYYCARSGGLPDKWGQGTTVTVSS

SEQ ID NO: 194 - protein, 116 aa, synthetic construct
QVQLVQSGAEVKKPGASVKVSCKASGFDIQDTYMHWVRQAPGQGLEWMGRIEPASGHLKYDPKFQERVTMTRDTSTSTVYMELSSLRSEDTAVYYCARSGGLPDKWGQGTTVTVSS

SEQ ID NO: 195 - protein, 116 aa, synthetic construct
QVQLVQSGAEVKKPGASVKVSCKASGFDIQDTYMHWVRQAPGQGLEWMGRIDPASGHLKYDPKFQGRVTITRDTSASTAYMELSSLRSEDTAVYYCARSGGLPDMWGQGTTVTVSS

SEQ ID NO: 196 - protein, 116 aa, synthetic construct
QVQLVQSGAEVKKPGASVKVSCKASGFDIQDTYMHWVRQAPGQGLEWMGRIDPASGHLKYDPKFQGRVTITRDTSASTVYMELSSLRSEDTAVYYCARSGGLPDMWGQGTTVTVSS

SEQ ID NO: 197 - protein, 116 aa, synthetic construct
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDVWGQGTTVTVSS

SEQ ID NO: 198 - protein, 116 aa, synthetic construct
QVQLVQSGAEVKKPGASVKVSCKASGFDIQDTYMHWVRQAPGQGLEWMGRIDPASGHTKYDPKFQVRATITTDTSASTAYLQLSSLRSEDTAVYYCARSGGLPDFWGQGTTVTVSS

SEQ ID NO: 199 - protein, 116 aa, synthetic construct
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDFWGQGTTVTVSS

SEQ ID NO: 200 - protein, 116 aa, synthetic construct
QVQLVQSGAEVKKPGASVKVSCKASGFDIQDTYMHWVRQAPGQGLEWMGRIDPASGHTKYDPKFQGRATITTDTSASTAYLQLSSLRSEDTAVYYCARSGGLPDLWGQGTTVTVSS

SEQ ID NO: 201 - protein, 116 aa, synthetic construct
QVQLVQSGAEVKKPGASVKVSCKASGFDIQDTYMHWVRQAPGQGLEWMGRIDPASGHSKYDPKFQVRATITTDTSASTAYLQLSSLRSEDTAVYYCARSGGLPDFWGQGTTVTVSS

SEQ ID NO: 202 - protein, 116 aa, synthetic construct
QVQLVQSGAEVKKPGASVKVSCKASGFDIQDTYMHWVRQAPGQGLEWMGRIDPASGHYKYDPKFQVRATITTDTSASTAYLQLSSLRSEDTAVYYCARSGGLPDFWGQGTTVTVSS

SEQ ID NO: 203 - protein, 10 aa, synthetic construct
RASSSVSYMY

SEQ ID NO: 204 - protein, 10 aa, synthetic construct
GASSSVSYMY

SEQ ID NO: 205 - protein, 7 aa, synthetic construct
ATSNLAS

SEQ ID NO: 206 - protein, 9 aa, synthetic construct
QQWSGNPRT

SEQ ID NO: 207 - protein, 9 aa, synthetic construct
QQWQGNPRT

SEQ ID NO: 208 - protein, 9 aa, synthetic construct
SQWSGNPRT

SEQ ID NO: 209 - protein, 9 aa, synthetic construct
QQWSGNPRS

SEQ ID NO: 210 - protein, 9 aa, synthetic construct
QQWSRNPRT

SEQ ID NO: 211 - protein, 9 aa, synthetic construct
QQWKGNPRT

SEQ ID NO: 212 - protein, 9 aa, synthetic construct
ASSSVSYMY

SEQ ID NO: 213 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRTFGGGTKLEIK

SEQ ID NO: 214 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWEGNPRTFGGGTKLEIK

SEQ ID NO: 215 - protein, 106 aa, synthetic construct
EIVLTQSPGTLSLSPGERATLSCGASSSVSYMYWYQQKPGQAPRLLIYATSNLASGIPDRFSGSGSGTDFTLTISRLEPEDFAVYYCQQWEGNPRTFGGGTKLEIK

SEQ ID NO: 216 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWQGNPRTFGGGTKLEIK

SEQ ID NO: 217 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCSQWSGNPRTFGGGTKLEIK

SEQ ID NO: 218 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWSGNPRSFGGGTKLEIK

SEQ ID NO: 219 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWSRNPRTFGGGTKLEIK

SEQ ID NO: 220 - protein, 106 aa, synthetic construct
EIVLTQSPGTLSLSPGERATLSCRASSSVSYMYWYQQKPGQAPRLLIYATSNLASGIPDRFSGSGSGTDFTLTISRLEPEDFAVYYCQQWKGNPRTFGGGTKLEIK

SEQ ID NO: 221 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 222 - protein, 8 aa, synthetic construct
GFSLSNYY

SEQ ID NO: 223 - protein, 7 aa, synthetic construct
IYGSDET

SEQ ID NO: 224 - protein, 14 aa, synthetic construct
ARDDSSDWDAKFNL

SEQ ID NO: 225 - protein, 8 aa, synthetic construct
GFNFNDYF

SEQ ID NO: 226 - protein, 10 aa, synthetic construct
MRNKNYQYGT

SEQ ID NO: 227 - protein, 11 aa, synthetic construct
ARESYYGFTSY

SEQ ID NO: 228 - protein, 8 aa, synthetic construct
GFTFSSFA

SEQ ID NO: 229 - protein, 8 aa, synthetic construct
ISSGGSYT

SEQ ID NO: 230 - protein, 12 aa, synthetic construct
ARGLWGYYALDY

SEQ ID NO: 231 - protein, 8 aa, synthetic construct
GFTFSPFA

SEQ ID NO: 232 - protein, 8 aa, synthetic construct
ISPGGSWT

SEQ ID NO: 233 - protein, 12 aa, synthetic construct
ARQLWGYYALDI

SEQ ID NO: 234 - protein, 8 aa, synthetic construct
GYVLPNYL

SEQ ID NO: 235 - protein, 8 aa, synthetic construct
TTPGGGTI

SEQ ID NO: 236 - protein, 15 aa, synthetic construct
ARSRWDPLYYYALEY

SEQ ID NO: 237 - protein, 8 aa, synthetic construct
GFTISSNY

SEQ ID NO: 238 - protein, 8 aa, synthetic construct
LYYYAGDT

SEQ ID NO: 239 - protein, 13 aa, synthetic construct
ARWADDHPPWIDL

SEQ ID NO: 240 - protein, 120 aa, synthetic construct
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSS

SEQ ID NO: 241 - protein, 120 aa, synthetic construct [sequence omitted]

SEQ ID NO: 242 - protein, 119 aa, synthetic construct [sequence omitted]

SEQ ID NO: 243 - protein, 119 aa, synthetic construct
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSS

SEQ ID NO: 244 - protein, 122 aa, synthetic construct
QVQLVQSGAEVKKPGSSVKVSCKASGYVLPNYLIEWVRQAPGQGLEWMGVTTPGGGTINYAQKFQGRVTITADESTSTAYMELSSLRSEDTAVYYCARSRWDPLYYYALEYWGQGTTVTVSS

SEQ ID NO: 245 - protein, 120 aa, synthetic construct
EVQLVESGGGLVQPGGSLRLSCAASGFTISSNYMIWVRQAPGKGLEWVSDLYYYAGDTYYADSVKGRFTMSRDISKNTVYLQMNSLRAEDTAVYYCARWADDHPPWIDLWGRGTLVTVSS

SEQ ID NO: 246 - protein, 6 aa, synthetic construct
QSINNE

SEQ ID NO: 247 - skipped

SEQ ID NO: 248 - protein, 12 aa, synthetic construct
QQGYSLRNIDNA

SEQ ID NO: 249 - protein, 6 aa, synthetic construct
QDIGIS

SEQ ID NO: 250 - skipped

SEQ ID NO: 251 - protein, 9 aa, synthetic construct
LQHNSAPYT

SEQ ID NO: 252 - protein, 5 aa, synthetic construct
SSVSY

SEQ ID NO: 253 - skipped

SEQ ID NO: 254 - protein, 9 aa, synthetic construct
QQWSGYPYT

SEQ ID NO: 255 - protein, 5 aa, synthetic construct
ISVSY

SEQ ID NO: 256 - skipped

SEQ ID NO: 257 - protein, 9 aa, synthetic construct
MQWSGYPYT

SEQ ID NO: 258 - protein, 10 aa, synthetic construct
ESVDNYGIPF

SEQ ID NO: 259 - skipped

SEQ ID NO: 260 - protein, 9 aa, synthetic construct
QQSEEVPLT

SEQ ID NO: 261 - protein, 6 aa, synthetic construct
QGISSW

SEQ ID NO: 262 - skipped

SEQ ID NO: 263 - protein, 8 aa, synthetic construct
QQSWLGGS

SEQ ID NO: 264 - protein, 109 aa, synthetic construct
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIK

SEQ ID NO: 265 - protein, 107 aa, synthetic construct [sequence omitted]

SEQ ID NO: 266 - protein, 106 aa, synthetic construct [sequence omitted]

SEQ ID NO: 267 - protein, 106 aa, synthetic construct
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIK

SEQ ID NO: 268 - protein, 111 aa, synthetic construct
DIVMTQSPDSLAVSLGERATINCRASESVDNYGIPFMNWYQQKPGQPPKLLIYAASNRGSGVPDRFSGSGSGTDFTLTISSLQAEDVAVYYCQQSEEVPLTFGQGTKLEIK

SEQ ID NO: 269 - protein, 106 aa, synthetic construct
DIQMTQSPSTLSASVGDRVTITCRASQGISSWLAWYQQKPGKAPKVLIYKASTLESGVPSRFSGSGSGTEFTLTISSLQPDDFATYYCQQSWLGGSFGQGTKLEIK

SEQ ID NO: 270 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 271 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 272 - protein, 8 aa, synthetic construct
GFTFSSYG

SEQ ID NO: 273 - protein, 8 aa, synthetic construct
IRYDGSNK

SEQ ID NO: 274 - protein, 8 aa, synthetic construct
KTHGSHDN

SEQ ID NO: 275 - protein, 8 aa, synthetic construct
GYTFTSYW

SEQ ID NO: 276 - protein, 8 aa, synthetic construct
MSPVDSDI

SEQ ID NO: 277 - protein, 12 aa, synthetic construct
ARRRPGQGYFDF

SEQ ID NO: 278 - protein, 8 aa, synthetic construct
GYSFTTYW

SEQ ID NO: 279 - protein, 8 aa, synthetic construct
MSPVDSDI

SEQ ID NO: 280 - protein, 12 aa, synthetic construct
ARRRPGQGYFDF

SEQ ID NO: 281 - protein, 115 aa, Homo sapiens [sequence omitted]

SEQ ID NO: 282 - protein, 119 aa, Homo sapiens
EVQLVQSGAEVKKPGESLKISCQSSGYTFTSYWIGWVRQMPGQGLEWIGIMSPVDSDIRYNPMFRGQVTMSVDKSSSTAYLQWSSLKASDTAMYYCARRRPGQGYFDFWGQGTMVTVSS

SEQ ID NO: 283 - protein, 119 aa, Homo sapiens
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 284 - protein, 8 aa, synthetic construct
RSNIGSNT

SEQ ID NO: 285-286 - skipped (2 entries)

SEQ ID NO: 287 - protein, 6 aa, synthetic construct
QSVGTW

SEQ ID NO: 288-289 - skipped (2 entries)

SEQ ID NO: 290 - protein, 6 aa, synthetic construct
QGISSW

SEQ ID NO: 291-293 - skipped (3 entries)

SEQ ID NO: 294 - protein, 12 aa, synthetic construct
QSYDRYTHPALL

SEQ ID NO: 295-296 - skipped (2 entries)

SEQ ID NO: 297 - protein, 9 aa, synthetic construct
QQYNIYPYT

SEQ ID NO: 298-299 - skipped (2 entries)

SEQ ID NO: 300 - protein, 9 aa, synthetic construct
QQYNIYPYT

SEQ ID NO: 301 - protein, 111 aa, synthetic construct [sequence omitted]

SEQ ID NO: 302 - protein, 107 aa, synthetic construct
EIVLTQSPATLSASPGERATISCRASQSVGTWVAWYQQKPGQAPRSLIYAASNLQSGIPARFSGSGSGTDFTLTISSLEPEDFAVYYCQQYNIYPYTFGQGTRLEIK

SEQ ID NO: 303 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 304 - protein, 189 aa, Homo sapiens
MLGSRAVMLLLLLPWTAQGRAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 305 - protein, 8 aa, synthetic construct
GFTFSSYG

SEQ ID NO: 306 - protein, 8 aa, synthetic construct
IWYDGSNE

SEQ ID NO: 307 - protein, 17 aa, synthetic construct
ARDRGYTSSWYPDAFDI

SEQ ID NO: 308 - protein, 8 aa, synthetic construct
GYSFSNYW

SEQ ID NO: 309 - protein, 8 aa, synthetic construct
IDPSNSYT

SEQ ID NO: 310 - protein, 10 aa, synthetic construct
ARWYYKPFDV

SEQ ID NO: 311 - protein, 8 aa, synthetic construct
GYKFTRYV

SEQ ID NO: 312 - protein, 8 aa, synthetic construct
INPYNDGT

SEQ ID NO: 313 - protein, 8 aa, synthetic construct
ARNWDTGL

SEQ ID NO: 314 - protein, 8 aa, synthetic construct
GYTFTSYL

SEQ ID NO: 315 - protein, 8 aa, synthetic construct
INPYNEGT

SEQ ID NO: 316 - protein, 8 aa, synthetic construct
ARNWDLPY

SEQ ID NO: 317 - protein, 8 aa, synthetic construct
GYTFTDQT

SEQ ID NO: 318 - protein, 8 aa, synthetic construct
IYPRDDSP

SEQ ID NO: 319 - protein, 13 aa, synthetic construct
AIPDRSGYAWFIY

SEQ ID NO: 320 - protein, 8 aa, synthetic construct
GYIFITYW

SEQ ID NO: 321 - protein, 8 aa, synthetic construct
IFPASGSA

SEQ ID NO: 322 - protein, 9 aa, synthetic construct
ARGGGGFAY

SEQ ID NO: 323 - protein, 5 aa, synthetic construct
SYGMH

SEQ ID NO: 324 - protein, 17 aa, synthetic construct
VIWYDGSNEYYADSVKG

SEQ ID NO: 325 - protein, 12 aa, synthetic construct
DRGYTSSWYPDA

SEQ ID NO: 326 - skipped

SEQ ID NO: 327 - protein, 17 aa, synthetic construct
VIWYDGSNKYYADSVKG

SEQ ID NO: 328 - protein, 14 aa, synthetic construct
DRGYSSSWYPDAFD

SEQ ID NO: 329 - skipped

SEQ ID NO: 330 - protein, 17 aa, synthetic construct
VISFDGSLKYYADSVKG

SEQ ID NO: 331 - protein, 12 aa, synthetic construct
ERTTLSGSYFDY

SEQ ID NO: 332 - protein, 5 aa, synthetic construct
SYAMH

SEQ ID NO: 333 - protein, 17 aa, synthetic construct
VISHDGSIKYYADSVKG

SEQ ID NO: 334 - skipped

SEQ ID NO: 335 - protein, 5 aa, synthetic construct
SYSMN

SEQ ID NO: 336 - protein, 17 aa, synthetic construct
YISSRSSTIYIADSVKG

SEQ ID NO: 337 - protein, 16 aa, synthetic construct
RIAAAGGFHYYYALDV

SEQ ID NO: 338 - protein, 5 aa, synthetic construct
TYSMN

SEQ ID NO: 339 - protein, 17 aa, synthetic construct
YISSSSSTRYHADSVKG

SEQ ID NO: 340 - protein, 16 aa, synthetic construct
RIAAAGPWGYYYAMDV

SEQ ID NO: 341 - protein, 5 aa, synthetic construct
SFSMN

SEQ ID NO: 342 - protein, 17 aa, synthetic construct
YISSRSSTIYYADSVKG

SEQ ID NO: 343 - skipped

SEQ ID NO: 344 - protein, 5 aa, synthetic construct
TYYWS

SEQ ID NO: 345 - protein, 16 aa, synthetic construct
LIYTSGSTNYNPSLKS

SEQ ID NO: 346 - protein, 10 aa, synthetic construct
DRGYYYGVDV

SEQ ID NO: 347 - protein, 7 aa, synthetic construct
SGGYYWS

SEQ ID NO: 348 - protein, 16 aa, synthetic construct
HIHYSGNTYYNPSLKS

SEQ ID NO: 349 - protein, 10 aa, synthetic construct
NRGFYYGMDV

SEQ ID NO: 350 - skipped

SEQ ID NO: 351 - protein, 16 aa, synthetic construct
YIYYSGSSYYNPSLKS

SEQ ID NO: 352 - protein, 10 aa, synthetic construct
DRGHYYGMDV

SEQ ID NO: 353 - skipped

SEQ ID NO: 354 - protein, 16 aa, synthetic construct
YIYYSGSTYYNPSLKS

SEQ ID NO: 355 - skipped

SEQ ID NO: 356 - protein, 5 aa, synthetic construct
SYFWS

SEQ ID NO: 357 - protein, 16 aa, synthetic construct
YIYYSGSTNYNPSLKS

SEQ ID NO: 358 - protein, 10 aa, synthetic construct
DRGSYYGSDY

SEQ ID NO: 359 - protein, 7 aa, synthetic construct
SGGYYWT

SEQ ID NO: 360 - protein, 16 aa, synthetic construct
YIYYSGNTYYNPSLKS

SEQ ID NO: 361 - protein, 10 aa, synthetic construct
NRGYYYGMDV

SEQ ID NO: 362-368 - skipped (7 entries)

SEQ ID NO: 369 - protein, 17 aa, synthetic construct
LIWYDGSNKYYADSVKG

SEQ ID NO: 370 - protein, 14 aa, synthetic construct
ENTVTIYYNYGMDV

SEQ ID NO: 371 - protein, 124 aa, synthetic construct
QVQLVESGGGVVQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSS

SEQ ID NO: 372 - protein, 117 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 373 - protein, 115 aa, synthetic construct
QVQLVQSGAEVKKPGSSVKVSCKASGYKFTRYVMHWVRQAPGQGLEWMGYINPYNDGTNYNEKFKGRVTITADKSTSTAYMELSSLRSEDTAVYYCARNWDTGLWGQGTTVTVSS

SEQ ID NO: 374 - protein, 115 aa, synthetic construct
QVQLVQSGAEVKKPGASVKVSCKASGYTFTSYLMHWVRQAPGQGLEWMGYINPYNEGTNYAQKFQGRVTMTRDTSISTAYMELSRLRSDDTAVYYCARNWDLPYWGQGTLVTVSS

SEQ ID NO: 375 - protein, 120 aa, synthetic construct
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSS

SEQ ID NO: 376 - protein, 116 aa, synthetic construct [sequence omitted]

SEQ ID NO: 377 - protein, 123 aa, synthetic construct
QVQLVESGGGWQPGRSLRLSCAASGFTFSSYGMHWVRQAPGKGLEWVAVIWYDGSNEYYADSVKGRFTISRDNSKNTLYLQMNSLRAEDTAVYYCARDRGYTSSWYPDAFDIWGQGTMVTVSS

SEQ ID NO: 378 - protein, 123 aa, synthetic construct
QVQLVESGGGWQPGRSLRLSCAASGFTFSSYGMHWVRQAPGKGLEWVAVIWYDGSNKYYADSVKGRFTISRDNSKNTLYLQMNSLRAEDTAVYYCARDRGYSSSWYPDAFDIWGQGTMVTVSS

SEQ ID NO: 379 - protein, 120 aa, synthetic construct
QVQLVESGGGWQPGRSLRLSCAASGFTFSSYGMHWVRQAPGKGLEWVAVISFDGSLKYYADSVKGRFTISRDNSKNTLYLQMNSLRAEDTAVYYCARERTTLSGSYFDYWGQGTLVTVSS

SEQ ID NO: 380 - protein, 120 aa, synthetic construct
QVQLVESGGGWQPGRSLRLSCAASGFTFSSYAMHWVRQAPGKGLEWLSVISHDGSIKYYADSVKGRFTISRDNSKNTLYLQMNSLRAEDTAVYYCARERTTLSGSYFDYWGQGTLVTVSS

SEQ ID NO: 381 - protein, 125 aa, synthetic construct
EVQLVESGGGLVQPGGSLRLSCAASGFTFSSYSMNWVRQAPGKGLEWVSYISSRSSTIYIADSVKGRFTISRDNAKNSLYLQMNSLRDEDTAVYYCARRIAAAGGFHYYYALDVWGQGTTVTVSS

SEQ ID NO: 382 - protein, 125 aa, synthetic construct
EVQLVESGGGLVQPGGSLRLSCAASGFTFSTYSMNWVRQAPGKGLEWVSYISSSSSTRYHADSVKGRFTISRDNAKNSLYLQMNSLRDEDTAVYYCARRIAAAGPWGYYYAMDVWGQGTTVTVSS

SEQ ID NO: 383 - protein, 124 aa, synthetic construct
EVQLVESGGGLVQPGGSLRLSCWSGFTFSSFSMNWVRQAPGKGLEWVSYISSRSSTIYYADSVKGRFTISRDNAKNSLYLQMNSLRDEDTAVYYCARRIAAAGPWGYYYAMDVWGQGTTVTVSS

SEQ ID NO: 384 - protein, 118 aa, synthetic construct
QVQLQESGPGLVKPSETLSLTCTVSGGSISTYYWSWIRQPAGKGLEWIGLIYTSGSTNYNPSLKSRVTMSLDTSKNQFSLRLTSVTAADTAVYYCARDRGYYYGVDVWGQGTTVTVSS

SEQ ID NO: 385 - protein, 120 aa, synthetic construct
QVQLQESGPGLVKPSQTLSLTCTVSGGSISSGGYYWSWIRQHPGKGLEWIGHIHYSGNTYYNPSLKSRVTISVDTSKNQFSLKLSSVTAADTAVYYCAKNRGFYYGMDVWGQGTTVTVSS

SEQ ID NO: 386 - protein, 120 aa, synthetic construct
QVQLQESGPGLVKPSQTLSLTCTVSGGSINSGGYYWSWIRQHPGKGLEWIGYIYYSGSSYYNPSLKSRVTISVDTSQNQFSLKLSSVTAADTAVYYCARDRGHYYGMDVWGQGTTVTVSS

SEQ ID NO: 387 - protein, 120 aa, synthetic construct
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARDRGHYYGMDVWGQGTTVTVSS

SEQ ID NO: 388 - protein, 118 aa, synthetic construct
QVQLQESGPRLVKPSETLSLTCTVSGDSISSYFWSWIRQPPGKGLEWLGYIYYSGSTNYNPSLKSRVTISIDTSKNQFSLKLSSVTAADTAVYYCTRDRGSYYGSDYWGQGTLVTVSS

SEQ ID NO: 389 - protein, 120 aa, synthetic construct
QVQLQESGPGLVKPSQTLSLTCTVSGGSISSGGYYWTWIRQHPGKGLEWIGYIYYSGNTYYNPSLKSRITISVDTSKNQFSLSLSSVTAADTAVYYCARNRGYYYGMDVWGQGTTVTVSS

SEQ ID NO: 390 - protein, 120 aa, synthetic construct
QVQLQESGPGLVKPSQTLSLTCTVSGGSISSGGYYWSWIRQHPGKGLEWIGYIYYSGSTYYNPSLKSRVTMSVDTSKNQFSLKLSSVTAADTAVYYCAKNRGFYYGMDVWGQGTTVTVSS

SEQ ID NO: 391 - protein, 120 aa, synthetic construct
QVQLQESGPGLVKPSQTLSLTCTVSGGSINSGGYYWSWIRQHPGKGLEWIGYIYYSGSSYYNPSLKSRVTISVDTSKNQFSLKLSSVTAADTAVYYCARDRGHYYGMDVWGQGTTVTVSS

SEQ ID NO: 392 - protein, 122 aa, synthetic construct
QVQLVESGGGWQPGRSLRLSCAASGFTFSSYGMHWVRQAPGKGLEWVALIWYDGSNKYYADSVKGRFTISRDNSKNTLYLQMNSLRAEDTAVYYCARENTVTIYYNYGMDVWGQGTTVTVSS

SEQ ID NO: 393 - protein, 9 aa, synthetic construct
SSNTGAGYD

SEQ ID NO: 394 - skipped

SEQ ID NO: 395 - protein, 11 aa, synthetic construct
QSYDSSLSGWV

SEQ ID NO: 396 - protein, 9 aa, synthetic construct
SSNIGSGYD

SEQ ID NO: 397 - skipped

SEQ ID NO: 398 - protein, 11 aa, synthetic construct
ASWTDGLSLVV

SEQ ID NO: 399 - protein, 6 aa, synthetic construct
DHILKF

SEQ ID NO: 400 - skipped

SEQ ID NO: 401 - protein, 9 aa, synthetic construct
QMYWSTPFT

SEQ ID NO: 402 - protein, 6 aa, synthetic construct
QSISDY

SEQ ID NO: 403 - skipped

SEQ ID NO: 404 - protein, 9 aa, synthetic construct
QQGHSFPFT

SEQ ID NO: 405 - protein, 6 aa, synthetic construct
RDVAIA

SEQ ID NO: 406 - skipped

SEQ ID NO: 407 - protein, 9 aa, synthetic construct
HQYSSYPFT

SEQ ID NO: 408 - protein, 6 aa, synthetic construct
ENIYSY

SEQ ID NO: 409 - skipped

SEQ ID NO: 410 - protein, 9 aa, synthetic construct
QHHYGIPFT

SEQ ID NO: 411 - protein, 14 aa, synthetic construct
TGSSSNTGAGYDVH

SEQ ID NO: 412 - protein, 7 aa, synthetic construct
GSGNRPS

SEQ ID NO: 413 - skipped

SEQ ID NO: 414 - protein, 14 aa, synthetic construct
TGSSSNIGAGYDVH

SEQ ID NO: 415 - protein, 7 aa, synthetic construct
GSNNRPS

SEQ ID NO: 416 - skipped

SEQ ID NO: 417 - protein, 14 aa, synthetic construct
TLRSGINVGTYRIY

SEQ ID NO: 418 - protein, 11 aa, synthetic construct
YKSDSDKQQGS

SEQ ID NO: 419 - protein, 9 aa, synthetic construct
MIWHSSASV

SEQ ID NO: 420-422 - skipped (3 entries)

SEQ ID NO: 423 - protein, 12 aa, synthetic construct
TLNSGYSDYKVD

SEQ ID NO: 424 - protein, 12 aa, synthetic construct
VGTGGIVGSKGD

SEQ ID NO: 425 - protein, 13 aa, synthetic construct
GADHGSGSNFVYV

SEQ ID NO: 426 - protein, 12 aa, synthetic construct
TLSSGYSDYKVD

SEQ ID NO: 427 - protein, 12 aa, synthetic construct
VGTGGIVGSKGE

SEQ ID NO: 428-429 - skipped (2 entries)

SEQ ID NO: 430 - protein, 12 aa, synthetic construct
VGTGGTVGSKGE

SEQ ID NO: 431 - skipped

SEQ ID NO: 432 - protein, 11 aa, synthetic construct
RASQGIAGWLA

SEQ ID NO: 433 - protein, 7 aa, synthetic construct
AASSLQS

SEQ ID NO: 434 - protein, 9 aa, synthetic construct
QQADSFPPT

SEQ ID NO: 435 - protein, 11 aa, synthetic construct
RASQVISSWLA

SEQ ID NO: 436 - skipped

SEQ ID NO: 437 - protein, 9 aa, synthetic construct
QQANSFPFT

SEQ ID NO: 438 - protein, 11 aa, synthetic construct
RASQGSSSWFA

SEQ ID NO: 439-440 - skipped (2 entries)

SEQ ID NO: 441 - protein, 11 aa, synthetic construct
RASQGISSWFA

SEQ ID NO: 442-443 - skipped (2 entries)

SEQ ID NO: 444 - protein, 11 aa, synthetic construct
RAGQVISSWLA

SEQ ID NO: 445 - skipped

SEQ ID NO: 446 - protein, 9 aa, synthetic construct
QQATSFPLT

SEQ ID NO: 447 - protein, 11 aa, synthetic construct
RASQGFSGWLA

SEQ ID NO: 448-449 - skipped (2 entries)

SEQ ID NO: 450 - protein, 11 aa, synthetic construct
RASQVISSWFA

SEQ ID NO: 451-455 - skipped (5 entries)

SEQ ID NO: 456 - protein, 11 aa, synthetic construct
RASQGIRNDLG

SEQ ID NO: 457 - skipped

SEQ ID NO: 458 - protein, 9 aa, synthetic construct
LQHNSYPPT

SEQ ID NO: 459 - protein, 111 aa, synthetic construct
QSVLTQPPSVSGAPGQRVTISCTGSSSNTGAGYDVHWYQQVPGTAPKLLIYGSGNRPSGVPDRFSGSKSGTSASLAITGLQAEDEADYYCQSYDSSLSGWVFGGGTRLTVL

SEQ ID NO: 460 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 461 - protein, 107 aa, synthetic construct
DIQMTQSPSSLSASVGDRVTITCKASDHILKFLTWYQQKPGKAPKLLIYGATSLETGVPSRFSGSGSGTDFTLTISSLQPEDFATYYCQMYWSTPFTFGGGTKVEIK

SEQ ID NO: 462 - protein, 107 aa, synthetic construct
DIQMTQSPSSLSASVGDRVTITCRASQSISDYLHWYQQKPGKAPKLLIKYASQSMSGVPSRFSGSGSGSDFTLTISSLQPEDFATYYCQQGHSFPFTFGQGTKLEIK

SEQ ID NO: 463 - protein, 107 aa, synthetic construct
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIK

SEQ ID NO: 464 - protein, 107 aa, synthetic construct [sequence omitted]

SEQ ID NO: 465 - skipped

SEQ ID NO: 466 - protein, 111 aa, synthetic construct
QSVLTQPPSVSGAPGQRVTISCTGSSSNIGAGYDVHWYQQLPGTAPKLLIYGSNNRPSGVPDRFSGSKSGTSASLAITGLQAEDEADYYCQSYDSSLSGWVFGGGTKLTVL

SEQ ID NO: 467 - protein, 115 aa, synthetic construct
QAVLTQPSSLSASPGASASLTCTLRSGINVGTYRIYWYQQKPGSPPQYLLRYKSDSDKQQGSGVPSRFSGSKDASANAGILLISGLQSEDEADYYCMIWHSSASVFGGGTKLTVL

SEQ ID NO: 468 - skipped

SEQ ID NO: 469 - protein, 116 aa, synthetic construct
QPVLTQPPSASASLGASVTLTCTLNSGYSDYKVDWYQQRPGKGPRFVMRVGTGGIVGSKGDGIPDRFSVLGSGLNRYLTIKNIQEEDESDYHCGADHGSGSNFVYVFGTGTKVTVL

SEQ ID NO: 470 - protein, 116 aa, synthetic construct
QPVLTQPPSASASLGASVTLTCTLSSGYSDYKVDWYQQRPGKGPRFVMRVGTGGIVGSKGEGIPDRFSVLGSGLNRYLTIKNIQEEDESDYHCGADHGSGSNFVYVFGTGTKVTVL

SEQ ID NO: 471 - protein, 116 aa, synthetic construct
QPELTQPPSASASLGASVTLTCTLSSGYSDYKVDWYQLRPGKGPRFVMRVGTGGTVGSKGEGIPDRFSVLGSGLNRSLTIKNIQEEDESDYHCGADHGSGSNFVYVFGTGTKVTVL

SEQ ID NO: 472 - protein, 107 aa, synthetic construct
DIQLTPSPSSVSASVGDRVTITCRASQGIAGWLAWYQQKPGKAPKLLIYAASSLQSGVPSRFSGSGSGTDFTLTISSLQPEDFATYYCQQADSFPPTFGGGTKVEIK

SEQ ID NO: 473 - protein, 107 aa, synthetic construct
DIQMTQSPSSVSASVGDRVTITCRASQVISSWLAWYQQKPGKAPSLLIYAASSLQSGVPSRFSGSVSGTDFTLTISSLQPEDFATYYCQQANSFPFTFGPGTKVDFK

SEQ ID NO: 474 - protein, 107 aa, synthetic construct
DIQMTQSPSSVSASVGDRVTITCRASQGSSSWFAWYQQKPGKAPKLLIYAASSLQSGVPSRFSGSGSGTDFTLTISSLQPEDFATYYCQQANSFPFTFGPGTKVDIK

SEQ ID NO: 475 - protein, 107 aa, synthetic construct
DSQMTQSPSSVSASVGDRVTITCRASQGISSWFAWYQQKPGQAPNLLIYAASSLQSGVPSRFSGSGSGTEFTLTISSLQPEDFATYYCQQANSFPFTFGPGTKVDIK

SEQ ID NO: 476 - protein, 107 aa, synthetic construct
DIQMTQSPSSVSASVGDRVTITCRAGQVISSWLAWYQQKPGKAPKLLIYAASSLQSGVPSRFSGSGSGTDFTLTISSLQPDDFATYYCQQATSFPLTFGGGTKVEIK

SEQ ID NO: 477 - protein, 107 aa, synthetic construct
DIQMTQSPSSVSASVGDRVTITCRASQGFSGWLAWYQQKPGKAPKLLIYAASSLQSGVPSRFSGSGSGTDFTLTISSLQPEDFATYYCQQATSFPLTFGPGTKVDIK

SEQ ID NO: 478 - protein, 107 aa, synthetic construct
DIQLTQSPSSVSASVGDRVTITCRASQVISSWFAWYQQKPGKAPNLLIYAASSLQSGVPSRFSGSGSGTDFTLTISSLQPADFATYFCQQATSFPLTFGPGTKVDVK

SEQ ID NO: 479 - skipped

SEQ ID NO: 480 - protein, 107 aa, synthetic construct
DIQMTQSPSSLSASVGDRVTITCRASQGIRNDLGWYQQKPGKAPKRLIYAASSLQSGVPSRFSGSGSGTEFTLTISSLQPEDFATYYCLQHNSYPPTFGQGTKVEIE

SEQ ID NO: 481 - protein, 399 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPELQLEESCAEAQDGELDGLWTTITIFITLFLLSVCYSATVTFFKVKWIFSSVVDLKQTIIPDYRNMIGQGA

SEQ ID NO: 482 - protein, 395 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPELQLEESCAEAQDGELDGLWTTITIFITLFLLSVCYSATITFFKVKWIFSSVVDLKQTIVPDYRNMIRQGA

SEQ ID NO: 483 - protein, 396 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLELQLEESCAEAQDGELDGLWTTITIFITLFLLSVCYSATVTFFKVKWIFSSVVDLKQTIVPDYRNMIRQGA

SEQ ID NO: 484 - protein, 5 aa, synthetic construct
YYGIS

SEQ ID NO: 485 - protein, 17 aa, synthetic construct
WISTYNGNTHYARMLQG

SEQ ID NO: 486 - protein, 15 aa, synthetic construct
ENYYGSGAYRGGMDV

SEQ ID NO: 487 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 488 - protein, 11 aa, synthetic construct
RASQSVSSYLA

SEQ ID NO: 489 - protein, 7 aa, synthetic construct
DASNRAT

SEQ ID NO: 490 - protein, 9 aa, synthetic construct
QQRSNWPWT

SEQ ID NO: 491 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 492 - protein, 15 aa, synthetic construct
DNDILTGYFDGYFDY

SEQ ID NO: 493 - protein, 15 aa, synthetic construct
DNDILDGYFDGYFDY

SEQ ID NO: 494 - protein, 15 aa, synthetic construct
DNDILTGYDDGYFDY

SEQ ID NO: 495 - protein, 15 aa, synthetic construct
DNDILTGYNDGYFDY

SEQ ID NO: 496-548 - skipped (53 entries)

SEQ ID NO: 549 - protein, 9 aa, synthetic construct
DNWNYVIDH

SEQ ID NO: 550-585 - skipped (36 entries)

SEQ ID NO: 586 - protein, 16 aa, synthetic construct
EIYHSGSTNYNPSLKS

SEQ ID NO: 587-603 - skipped (17 entries)

SEQ ID NO: 604 - protein, 17 aa, synthetic construct
WINTNTGNPTYAQGFTG

SEQ ID NO: 605-732 - skipped (128 entries)

SEQ ID NO: 733 - protein, 6 aa, synthetic construct
SRNWWS

SEQ ID NO: 734-738 - skipped (5 entries)

SEQ ID NO: 739 - protein, 4 aa, synthetic construct
EPEN

SEQ ID NO: 740-741 - skipped (2 entries)

SEQ ID NO: 742 - protein, 4 aa, synthetic construct
EPED

SEQ ID NO: 743-773 - skipped (31 entries)

SEQ ID NO: 774 - protein, 8 aa, synthetic construct
GLTVPFDY

SEQ ID NO: 775-785 - skipped (11 entries)

SEQ ID NO: 786 - protein, 17 aa, synthetic construct
GILPIFGTGHYVQKFQG

SEQ ID NO: 787-856 - skipped (70 entries)

SEQ ID NO: 857 - protein, 17 aa, synthetic construct
WITPNSGGTSYAQRFQG

SEQ ID NO: 858-866 - skipped (9 entries)

SEQ ID NO: 867 - protein, 5 aa, synthetic construct
IYAIS

SEQ ID NO: 868-892 - skipped (25 entries)

SEQ ID NO: 893 - protein, 5 aa, synthetic construct
GDSIH

SEQ ID NO: 894-898 - skipped (5 entries)

SEQ ID NO: 899 - protein, 9 aa, synthetic construct
QQYGSSPLT

SEQ ID NO: 900 - protein, 7 aa, synthetic construct
GASSRAT

SEQ ID NO: 901 - protein, 12 aa, synthetic construct
RASQSVSSSYLA

SEQ ID NO: 902 - protein, 124 aa, synthetic construct
QVQLQESGPGLVKPSGTLSLTCAVSGGSISSRNWWSWVRQPPGKGLEWIGEIYHSGSTNYNPSLKSRVIISVDKSKNQFSLNLNSVTAADTAVYYCARDNDILTGYFDGYFDYWGQGTLVTVSS

SEQ ID NO: 903 - protein, 124 aa, synthetic construct
QVQLQESGPGLVKPSGTLSLTCAVSGGSISSRNWWSWVRQPPGKGLEWIGEIYHSGSTNYNPSLKSRVTISVDKSKNQFSLNLNSVTAADTAVYYCARDNDILDGYFDGYFDYWGQGTLVTVSS

SEQ ID NO: 904 - protein, 124 aa, synthetic construct
QVQLQESGPGLVKPSGTLSLTCAVSGGSISSRNWWSWVRQPPGKGLEWIGEIYHSGSTNYNPSLKSRVTISVDKSKNQFSLNLNSVTAADTAVYYCARDNDILTGYDDGYFDYWGQGTLVTVSS

SEQ ID NO: 905 - protein, 124 aa, synthetic construct
QVQLQESGPGLVKPSGTLSLTCAVSGGSISSRNWWSWVRQPPGKGLEWIGEIYHSGSTNYNPSLKSRVTISVDKSKNQFSLNLNSVTAADTAVYYCARDNDILTGYNDGYFDYWGQGTLVTVSS

SEQ ID NO: 906 - skipped

SEQ ID NO: 907 - protein, 124 aa, synthetic construct
QVQLQESGPGLVKPSGTLSLTCAVSGGSITSRNWWSWVRQPPGKGLEWIGEIYHSGSTNYNPSLKSRVTISVDKSKNQFSLNLNSVTAADTAVYYCARDNDILTGYFDGYFDYWGQGTLVTVSS

SEQ ID NO: 908 - protein, 124 aa, synthetic construct
QVQLQESGPGLVKPSGTLSLTCAVSGGSISSRNWWSWVRQPPGKGLEWIGEIYHSGSTNYNPSLKSRVTISVDKSKNQFSLNVNSVTAADTAVYYCARDNDILTGYFDGYFDYWGQGTLVTVSS

SEQ ID NO: 909 - protein, 124 aa, synthetic construct
QVQLQESGPGLVKPSGTLSLTCAVSGGSISSRNWWSWVRQPPGKGLEWIGEIYHSGSTNYNPSLKSRVTISVDKSKNQFSLNLKSVTAADTAVYYCARDNDILTGYFDGYFDYWGQGTLVTVSS

SEQ ID NO: 910-922 - skipped (13 entries)

SEQ ID NO: 923 - protein, 124 aa, synthetic construct
QVQLQESGPGLVKPSGTLSLTCAVSGGSISSRNWWSWVRQPPGKGLEWIGEIYHSGSTNYNPSLKSRVTISVDKSKNQFSLNLNSVTAADTAVYYCARDNDILTGYFDGYFDYWGQGTLVTVSS

SEQ ID NO: 924-932 - skipped (9 entries)

SEQ ID NO: 933 - protein, 124 aa, synthetic construct
QVQLQESGPGLVEPSGTLSLTCAVSGGSISSRNWWSWVRQPPGKGLEWIGEIYHSGSTNYNPSLKSRVTISVDKSKNQFSLNLNSVTAADTAVYYCARDNDILTGYFDGYFDYWGQGTLVTVSS

SEQ ID NO: 934-1047 - skipped (114 entries)

SEQ ID NO: 1048 - protein, 118 aa, synthetic construct
QVQLVQSGSELKKPGASVKVSCKASGYSFILYGMNWVRQAPGQGLEWMGWINTNTGNPTYAQGFTGRFVFSLDTSVSTAYLQISSLKADDTAVYYCARDNWNYVIDHWGQGTLVTVSS

SEQ ID NO: 1049-1465 - skipped (417 entries)

SEQ ID NO: 1466 - protein, 113 aa, synthetic construct
QVQLVQSGAEVKKPGSSVKVSCKASGGTFTIYAISWVRQAPGQGLEWMGGILPIFGTGHYVQKFQGRVTITADESTSTSYMELSSLRSEDTAVYYCAREPEDWGQGTLVTVSS

SEQ ID NO: 1467-1543 - skipped (77 entries)

SEQ ID NO: 1544 - protein, 113 aa, synthetic construct
QVQLVQSGAEVKKPGSSVKVSCKASGGTFTIYAISWVRQAPGQGLEWMGGILPIFGTGHYVQKFQGRVTITADESTSTSYMELSSLRSEDTAVYYCAREPENWGQGTLVTVSS

SEQ ID NO: 1545-1620 - skipped (76 entries)

SEQ ID NO: 1621 - protein, 117 aa, synthetic construct
QVQLVQSGAEVKKPGASVKVSCTASGDAFTGDSIHWVRQAPGQGLEWMGWITPNSGGTSYAQRFQGRVTLTRDTSISTVYMDLRRLRSDDTAVYFCASGLTVPFDYWGQGTLVTVSS

SEQ ID NO: 1622-1767 - skipped (146 entries)

SEQ ID NO: 1768 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 1769 - protein, 5 aa, synthetic construct
GNPRT

SEQ ID NO: 1770 - protein, 17 aa, synthetic construct
RIDPASGHYKYDPKFQV

SEQ ID NO: 1771 - protein, 5 aa, synthetic construct
LYGMN

(19) United States
(12) Patent Application Publication
(10) Pub. No.: US 2026/0265389 A1
(43) Pub. Date: Sep. 10, 2026

(54) ENGINEERED BISPECIFIC MOLECULES AND METHODS OF USE

(71) Applicant: Cantai Therapeutics Inc., Philadelphia, PA (US)

(72) Inventor: Geeta Vemuri, Bryn Mawr, PA (US)

(21) Appl. No.: 19/686,217

(22) Filed: May 22, 2026

Related U.S. Application Data
(63) Continuation of application No. PCT/US24/57167, filed on Nov. 22, 2024.
(60) Provisional application No. 63/602,120, filed on Nov. 22, 2023.
(60) Provisional application No. 63/551,580, filed on Feb. 9, 2024.
(60) Provisional application No. 63/677,245, filed on Jul. 30, 2024.
(60) Provisional application No. 63/696,844, filed on Sep. 19, 2024.

(51) Int. Cl. C07K 16/28; C07K 16/24; C07K 16/46

(52) U.S. Cl. CPC C07K 16/2875; C07K 16/244; C07K 16/248; C07K 16/2866; C07K 16/468; C07K 2317/31; C07K 2317/52; C07K 2317/76; C07K 2317/92

(57) ABSTRACT

Provided herein are bispecific molecules and methods of treating using the bispecific molecules, wherein the bispecific molecules comprise a first binding domain and a second binding domain, wherein the first binding domain binds TL1A, a variant thereof or a functional fragment thereof, and wherein the second binding domain binds any one of IL-6R, IL-6, IL-12, IL-23, an IL-17 family cytokine, IL-17R, a variant thereof, and a functional fragment thereof.

### CROSS-REFERENCED APPLICATIONS

This application is a continuation of International Application No. PCT/US2024/57167, filed Nov. 22, 2024, which claims the benefit of priority to U.S. Provisional Application No. 63/602,120, filed on Nov. 22, 2023, U.S. Provisional Application No. 63/551,580, filed on Feb. 9, 2024, U.S. Provisional Application No. 63/677,245, filed on Jul. 30, 2024, and U.S. Provisional Application No. 63/696,844, filed on Sep. 19, 2024, the entire contents of each of which are incorporated herein by reference.

### INCORPORATION BY REFERENCE OF SEQUENCE LISTING

The instant application contains a Sequence Listing, which has been submitted via Patent Center. The Sequence Listing titled 220710-703601_PCT_SL.xml, which was created on Nov. 22, 2024, and is 673,981 bytes in size, is hereby incorporated by reference in its entirety.

### FIELD

The disclosure generally relates to bispecific molecules is capable of binding to TL1A, and a protein selected from IL-6, IL-23, IL-12, IL-6R, an IL-17 family cytokine, and IL-17R.

### BACKGROUND OF THE DISCLOSURE

Bispecific antibodies (BsAbs) are antibodies with two binding sites each independently directed at two different targets, or alternatively, two different epitopes on the same target. The therapeutic utility of BsAbs has shown to result in the potential for enhanced activity in comparison to that of monospecific antibodies. BsAbs are understood to have broader applications for immunotherapy in treatment of various diseases.

### SUMMARY OF THE DISCLOSURE

Provided herein are IgG antibody constructs comprising: a TL1A binding region that binds an epitope on TL1A, a variant thereof or a functional fragment thereof, and an IL-23 binding region that binds an epitope on IL-23, a variant thereof or a functional fragment thereof, wherein said TL1A binding region comprises a TL1A binding heavy chain variable domain and a TL1A binding light chain variable domain, wherein at least one amino acid modification in said TL1A binding heavy chain variable domain and/or said TL1A binding light chain variable domain, and wherein said amino acid modification changes an isoelectric point (pI) of said IgG antibody construct relative to a pI of a corresponding IgG antibody construct prior to said amino acid modification, thereby resulting in pH-dependent binding activity for said TL1A in said IgG antibody construct, as measured by surface plasmon resonance spectroscopy. In some embodiments, said IL-23 binding region comprises an IL-23 binding heavy chain variable domain and an IL-23 binding light chain variable domain, and at least one amino acid modification in said IL-23 binding heavy chain variable domain and/or said IL-23 binding light chain variable domain, wherein said amino acid modification changes an isoelectric point (pI) of said IgG antibody construct relative to a pI of a corresponding IgG antibody construct prior to said amino acid modification, thereby resulting in pH-dependent binding activity for said IL-23 in said IgG antibody construct, as measured by surface plasmon resonance spectroscopy. In some embodiments, said at least one amino acid modification is within a CDR-H1, a CDR-H2, and/or a CDR-H3 of said TL1A binding heavy chain variable domain. In some embodiments, said at least one amino acid modification is within a CDR-H3 of said TL1A binding heavy chain variable domain. In some embodiments, said at least one amino acid modification comprises a substitution of at least one uncharged amino acid with a charged amino acid. In some embodiments, said at least one amino acid modification is within at least one framework region of said TL1A binding heavy chain variable domain. In some embodiments, said at least one amino acid modification is within a CDR-L1, a CDR-L2, and/or a CDR-L3 of said TL1A binding light chain variable domain. In some embodiments, said at least one amino acid modification is within at least one framework region of said TL1A binding light chain variable domain. In some embodiments, said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to said binding affinity of corresponding TL1A binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said at least one amino acid modification is within a CDR-H1, a CDR-H2, and/or a CDR-H3 of said IL-23 binding heavy chain variable domain. In some embodiments, said at least one amino acid modification is within at least one framework region of said IL-23 binding heavy chain variable domain. In some embodiments, said at least one amino acid modification is within a CDR-L1, a CDR-L2, and/or a CDR-L3 of said IL-23 binding light chain variable domain. In some embodiments, said at least one amino acid modification is within at least one framework region of said TL1A binding light chain variable domain. In some embodiments, said at least one amino acid modification increases binding affinity of said IL-23 binding region for said IL-23 in neutral pH condition by at least 2%, at least 5%, at least 10%, at least 15%, at least 20%, at least 30%, at least 40%, at least 50% or more relative to said binding affinity of corresponding IL-23 binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said IL-23 binding heavy chain variable region further binds an epitope that is present on IL-12p40, a variant thereof or a functional fragment thereof. In some embodiments, said IgG antibody construct has higher binding affinity for trimeric TL1A relative to monomeric TL1A under neutral pH condition. In some embodiments, said IgG antibody construct has higher binding affinity for monomeric TL1A relative to trimeric TL1A under neutral pH condition. In some embodiments, said IgG antibody construct has higher binding affinity for monomeric IL-23 relative to heterodimeric IL-23 under neutral pH condition. In some embodiments, said IgG antibody construct has higher binding affinity for heterodimeric IL-23 relative to monomeric IL-23 under neutral pH condition. In some embodiments, said TL1A binding light chain variable domain and said IL-23 binding light chain variable domain are at least 90%, at least 95%, at least 98% or 100% identical relative to each other. In some embodiments, said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

Also provided herein are IgG antibody constructs comprising a TL1A binding region that binds an epitope on TL1A, a variant thereof or a functional fragment thereof, and an IL-23 binding region that binds an epitope on IL-23, a variant thereof or a functional fragment thereof, wherein said TL1A binding region comprises a TL1A binding heavy chain variable domain, wherein said IL-23 binding region comprises an IL-23 binding heavy chain variable domain, and wherein a binding affinity of said TL1A binding region for said TL1A is at least 10% higher at pH 7.4 relative to said binding affinity at pH 5.8, as measured by surface plasmon resonance spectroscopy. In some embodiments, a binding affinity of said IL-23 binding region for said IL-23 is at least 10% higher at pH 7.4 relative to said binding affinity at pH 5.8, as measured by surface plasmon resonance spectroscopy. In some embodiments, said IgG antibody construct comprises a TL1A binding light chain variable domain that interact with said TL1A binding heavy chain variable domain, thereby forming said TL1A binding region, said TL1A binding heavy chain variable domain and/or said TL1A binding light chain variable domain comprises at least one amino acid modification that (a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or (b) decreases binding affinity of said TL1A binding region for said TL1A relative to said binding affinity of corresponding TL1A binding region prior to said at least one amino acid modification under acidic pH condition, as measured by surface plasmon resonance spectroscopy. In some embodiments, said IgG antibody construct comprises an IL-23 binding light chain variable domain that interact with said IL-23 binding heavy chain variable domain, thereby forming said IL-23 binding region, said IL-23 binding heavy chain variable domain and/or said IL-23 binding light chain variable domain comprises at least one amino acid modification that (a) increases binding affinity of said IL-23 binding region for said IL-23 relative to said binding affinity of corresponding IL-23 binding region prior to said at least one amino acid modification under neutral pH condition, as measured by surface plasmon resonance spectroscopy, and/or (b) decreases binding affinity of said IL-23 binding region for said IL-23 relative to said binding affinity of corresponding IL-23 binding region prior to said at least one amino acid modification under acidic pH condition, as measured by surface plasmon resonance spectroscopy. In some embodiments, said at least one amino acid modification is within a CDR-H1, a CDR-H2, and/or a CDR-H3 of said TL1A binding heavy chain variable domain. In some embodiments, said at least one amino acid modification is within a CDR-H3 of said TL1A binding heavy chain variable domain. In some embodiments, said at least one amino acid modification comprises a substitution of at least one uncharged amino acid with a charged amino acid. In some embodiments, said at least one amino acid modification is within at least one framework region of said TL1A binding heavy chain variable domain. In some embodiments, said at least one amino acid modification is within a CDR-L1, a CDR-L2, and/or a CDR-L3 of said TL1A binding light chain variable domain. In some embodiments, said at least one amino acid modification is within at least one framework region of said TL1A binding light chain variable domain. In some embodiments, said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to said binding affinity of corresponding TL1A binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said at least one amino acid modification is within a CDR-H1, a CDR-H2, and/or a CDR-H3 of said IL-23 binding heavy chain variable domain. In some embodiments, said at least one amino acid modification is within at least one framework region of said IL-23 binding heavy chain variable domain. In some embodiments, said at least one amino acid modification is within a CDR-L1, a CDR-L2, and/or a CDR-L3 of said IL-23 binding light chain variable domain. In some embodiments, said at least one amino acid modification is within at least one framework region of said TL1A binding light chain variable domain. In some embodiments, said at least one amino acid modification increases binding affinity of said IL-23 binding region for said IL-23 in neutral pH condition by at least 2%, at least 5%, at least 10%, at least 15%, at least 20%, at least 30%, at least 40%, at least 50% or more relative to said binding affinity of corresponding IL-23 binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said IL-23 binding heavy chain variable region further binds an epitope that is present on IL-12p40, a variant thereof or a functional fragment thereof. In some embodiments, said IgG antibody construct has higher binding affinity for trimeric TL1A relative to monomeric TL1A under neutral pH condition. In some embodiments, said IgG antibody construct has higher binding affinity for monomeric TL1A relative to trimeric TL1A under neutral pH condition. In some embodiments, said IgG antibody construct has higher binding affinity for monomeric IL-23 relative to heterodimeric IL-23 under neutral pH condition. In some embodiments, said IgG antibody construct has higher binding affinity for heterodimeric IL-23 relative to monomeric IL-23 under neutral pH condition. In some embodiments, said TL1A binding light chain variable domain and said IL-23 binding light chain variable domain are at least 90%, at least 95%, at least 98% or 100% identical relative to each other. In some embodiments, said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

Also provided herein are IgG antibody constructs comprising a TL1A binding region that binds an epitope on TL1A, a variant thereof or a functional fragment thereof, and an IL-23 binding region that binds an epitope on IL-23, a variant thereof or a functional fragment thereof, wherein said TL1A binding region comprises a TL1A binding heavy chain variable domain, and wherein said IL-23 binding region comprises an IL-23 binding heavy chain variable domain. In some embodiments, a binding affinity of said TL1A binding region for said TL1A is higher than a binding affinity of said IL-23 binding region for said IL-23 under neutral pH condition, as measured by surface plasmon resonance spectroscopy. In some embodiments, said binding affinity of said TL1A binding region for said TL1A is at least two times higher than said binding affinity of said IL-23 binding region for said IL-23. In some embodiments, a binding affinity of said IL-23 binding region for said IL-23 is higher than a binding affinity of said TL1A binding region for said TL1A under neutral pH condition, as measured by surface plasmon resonance spectroscopy. In some embodiments, wherein a binding affinity of said IL-23 binding region for said IL-23 is at least two times higher than a binding affinity of said TL1A binding region for said TL1A. In some embodiments, said IgG antibody construct comprises a TL1A binding light chain variable domain that interact with said TL A binding heavy chain variable domain, thereby forming said TL1A binding region, said TL1A binding heavy chain variable domain and/or said TL1A binding light chain variable domain comprises at least one amino acid modification that (a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or (b) decreases binding affinity of said TL1A binding region for said TL1A relative to said binding affinity of corresponding TL1A binding region prior to said at least one amino acid modification under acidic pH condition, as measured by surface plasmon resonance spectroscopy. In some embodiments, said IgG antibody construct comprises an IL-23 binding light chain variable domain that interact with said IL-23 binding heavy chain variable domain, thereby forming said IL-23 binding region, said IL-23 binding heavy chain variable domain and/or said IL-23 binding light chain variable domain comprises at least one amino acid modification that (a) increases binding affinity of said IL-23 binding region for said IL-23 relative to said binding affinity of corresponding IL-23 binding region prior to said at least one amino acid modification under neutral pH condition, as measured by surface plasmon resonance spectroscopy, and/or (b) decreases binding affinity of said IL-23 binding region for said IL-23 relative to said binding affinity of corresponding IL-23 binding region prior to said at least one amino acid modification under acidic pH condition, as measured by surface plasmon resonance spectroscopy. In some embodiments, said at least one amino acid modification is within a CDR-H1, a CDR-H2, and/or a CDR-H3 of said TL1A binding heavy chain variable domain. In some embodiments, said at least one amino acid modification is within a CDR-H3 of said TL1A binding heavy chain variable domain. In some embodiments, said at least one amino acid modification comprises a substitution of at least one uncharged amino acid with a charged amino acid. In some embodiments, said at least one amino acid modification is within at least one framework region of said TL1A binding heavy chain variable domain. In some embodiments, said at least one amino acid modification is within a CDR-L1, a CDR-L2, and/or a CDR-L3 of said TL1A binding light chain variable domain. In some embodiments, said at least one amino acid modification is within at least one framework region of said TL1A binding light chain variable domain. In some embodiments, said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to said binding affinity of corresponding TL1A binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said at least one amino acid modification is within a CDR-H1, a CDR-H2, and/or a CDR-H3 of said IL-23 binding heavy chain variable domain. In some embodiments, said at least one amino acid modification is within at least one framework region of said IL-23 binding heavy chain variable domain. In some embodiments, said at least one amino acid modification is within a CDR-L1, a CDR-L2, and/or a CDR-L3 of said IL-23 binding light chain variable domain. In some embodiments, said at least one amino acid modification is within at least one framework region of said TL1A binding light chain variable domain. In some embodiments, said at least one amino acid modification increases binding affinity of said IL-23 binding region for said IL-23 in neutral pH condition by at least 2%, at least 5%, at least 10%, at least 15%, at least 20%, at least 30%, at least 40%, at least 50% or more relative to said binding affinity of corresponding IL-23 binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said IL-23 binding heavy chain variable region further binds an epitope that is present on IL-12p40, a variant thereof or a functional fragment thereof. In some embodiments, said IgG antibody construct has higher binding affinity for trimeric TL1A relative to monomeric TL1A under neutral pH condition. In some embodiments, said IgG antibody construct has higher binding affinity for monomeric TL1A relative to trimeric TL1A under neutral pH condition. In some embodiments, said IgG antibody construct has higher binding affinity for monomeric IL-23 relative to heterodimeric IL-23 under neutral pH condition. In some embodiments, said IgG antibody construct has higher binding affinity for heterodimeric IL-23 relative to monomeric IL-23 under neutral pH condition. In some embodiments, said TL1A binding light chain variable domain and said IL-23 binding light chain variable domain are at least 90%, at least 95%, at least 98% or 100% identical relative to each other. In some embodiments, said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

Also provided herein are IgG antibody constructs comprising a TL1A binding region that binds an epitope on TL1A, a variant thereof or a functional fragment thereof, and an IL-12 binding region that binds an epitope on IL-12p40, IL-12p35, a variant thereof or a functional fragment thereof, wherein said TL1A binding region comprises a TL1A binding heavy chain variable domain, wherein said IL-12 binding region comprises an IL-12 binding heavy chain variable domain, and wherein a binding affinity of said TL1A binding region for said TL1A is at least 10% higher at pH 7.4 relative to said binding affinity at pH 5.8, as measured by surface plasmon resonance spectroscopy. In some embodiments, a binding affinity of said IL-12 binding region for said IL-12 is at least 10% higher at pH 7.4 relative to said binding affinity at pH 5.8, as measured by surface plasmon resonance spectroscopy. In some embodiments, said at least one amino acid modification is within a CDR-H1, a CDR-H2, and/or a CDR-H3 of said TL1A binding heavy chain variable domain. In some embodiments, said at least one amino acid modification is within a CDR-H3 of said TL1A binding heavy chain variable domain. In some embodiments, said at least one amino acid modification comprises a substitution of at least one uncharged amino acid with a charged amino acid. In some embodiments, said at least one amino acid modification is within at least one framework region of said TL1A binding heavy chain variable domain. In some embodiments, said at least one amino acid modification is within a CDR-L1, a CDR-L2, and/or a CDR-L3 of said TL1A binding light chain variable domain. In some embodiments, said at least one amino acid modification is within at least one framework region of said TL1A binding light chain variable domain. In some embodiments, said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said at least one amino acid modification is within a CDR-H1, a CDR-H2, and/or a CDR-H3 of said IL-12 binding heavy chain variable domain. In some embodiments, said at least one amino acid modification is within at least one framework region of said IL-12 binding heavy chain variable domain. In some embodiments, said at least one amino acid modification is within a CDR-L1, a CDR-L2, and/or a CDR-L3 of said IL-12 binding light chain variable domain. In some embodiments, said at least one amino acid modification increases binding affinity of said IL-12 binding region for said IL-12 in neutral pH condition by at least 2%, at least 5%, at least 10%, at least 15%, at least 20%, at least 30%, at least 40%, at least 50% or more relative to binding affinity of corresponding IL-12 binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said IL-12 binding heavy chain variable domain further binds an epitope that is present on IL-23, a variant thereof or a functional fragment thereof. In some embodiments, said IgG antibody construct has higher binding affinity for trimeric TL1A relative to monomeric TL1A under neutral pH condition. In some embodiments, said IgG antibody construct has higher binding affinity for monomeric TL1A relative to trimeric TL1A under neutral pH condition. In some embodiments, said TL1A binding light chain variable domain and said IL-12 binding light chain variable domain are at least 90%, at least 95%, at least 98% or 100% identical relative to each other. In some embodiments, said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

Also provided herein are IgG antibody constructs comprising a TL1A binding region that binds an epitope on TL1A, a variant thereof or a functional fragment thereof, and an IL-12 binding region that binds an epitope on IL-12p40, IL-12p35, a variant thereof or a functional fragment thereof, wherein said TL1A binding region comprises a TL1A binding heavy chain variable domain, wherein said IL-12 binding region comprises an IL-12 binding heavy chain variable domain, and wherein a binding affinity of the TL1A binding region for said TL1A is more than four times a binding affinity of the IL-12 binding region for said IL-12 under neutral pH condition, as measured by surface plasmon resonance spectroscopy. In some embodiments, a binding affinity of the IL-12 binding region for said IL-12 is at least two times higher than a binding affinity of the TL1A binding region for said TL1A. In some embodiments, said IgG antibody construct comprises a TL1A binding light chain variable domain that interact with said TL1A binding heavy chain variable domain, thereby forming said TL1A binding region, said TL1A binding heavy chain variable domain and/or said TL1A binding light chain variable domain comprises at least one amino acid modification that (a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or (b) decreases binding affinity of said TL1A binding region for said TL1A relative to said binding affinity of corresponding TL1A binding region prior to said at least one amino acid modification under acidic pH condition, as measured by surface plasmon resonance spectroscopy. In some embodiments, said IgG antibody construct comprises an IL-12 binding light chain variable domain that interact with said IL-12 binding heavy chain variable domain, thereby forming said IL-12 binding region, said IL-12 binding heavy chain variable domain and/or said IL-12 binding light chain variable domain comprises at least one amino acid modification that (a) increases binding affinity of said IL-12 binding region for said IL-12 relative to said binding affinity of corresponding IL-12 binding region prior to said at least one amino acid modification under neutral pH condition, as measured by surface plasmon resonance spectroscopy, and/or (b) decreases binding affinity of said IL-12 binding region for said IL-12 relative to said binding affinity of corresponding IL-12 binding region prior to said at least one amino acid modification under acidic pH condition, as measured by surface plasmon resonance spectroscopy. In some embodiments, said at least one amino acid modification is within a CDR-H1, a CDR-H2, and/or a CDR-H3 of said TL1A binding heavy chain variable domain. In some embodiments, said at least one amino acid modification is within a CDR-H3 of said TL1A binding heavy chain variable domain. In some embodiments, said at least one amino acid modification comprises a substitution of at least one uncharged amino acid with a charged amino acid. In some embodiments, said at least one amino acid modification is within at least one framework region of said TL1A binding heavy chain variable domain. In some embodiments, said at least one amino acid modification is within a CDR-L1, a CDR-L2, and/or a CDR-L3 of said TL1A binding light chain variable domain. In some embodiments, said at least one amino acid modification is within at least one framework region of said TL1A binding light chain variable domain. In some embodiments, said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said at least one amino acid modification is within a CDR-H1, a CDR-H2, and/or a CDR-H3 of said IL-12 binding heavy chain variable domain. In some embodiments, said at least one amino acid modification is within at least one framework region of said IL-12 binding heavy chain variable domain. In some embodiments, said at least one amino acid modification is within a CDR-L1, a CDR-L2, and/or a CDR-L3 of said IL-12 binding light chain variable domain. In some embodiments, said at least one amino acid modification increases binding affinity of said IL-12 binding region for said IL-12 in neutral pH condition by at least 2%, at least 5%, at least 10%, at least 15%, at least 20%, at least 30%, at least 40%, at least 50% or more relative to binding affinity of corresponding IL-12 binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said IL-12 binding heavy chain variable domain further binds an epitope that is present on IL-23, a variant thereof or a functional fragment thereof. In some embodiments, said IgG antibody construct has higher binding affinity for trimeric TL1A relative to monomeric TL1A under neutral pH condition. In some embodiments, said IgG antibody construct has higher binding affinity for monomeric TL1A relative to trimeric TL1A under neutral pH condition. In some embodiments, said TL1A binding light chain variable domain and said IL-12 binding light chain variable domain are at least 90%, at least 95%, at least 98% or 100% identical relative to each other. In some embodiments, said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

Also provided herein are IgG antibody constructs comprising a TL1A binding region that binds an epitope on TL1A, a variant thereof or a functional fragment thereof, and an IL-12 binding region that binds an epitope on IL-12p40, IL-12p35, a variant thereof or a functional fragment thereof, wherein said TL1A binding region comprises a TL1A binding heavy chain variable domain, wherein said IL-12 binding region comprises an IL-12 binding heavy chain variable domain, and wherein a binding affinity of the IL-12 binding region for said IL-12 is higher than a binding affinity of the TL A binding region for said TL1A under neutral pH condition, as measured by surface plasmon resonance spectroscopy. In some embodiments, a binding affinity of the IL-12 binding region for said IL-12 is at least two times higher than a binding affinity of the TL1A binding region for said TL1A. In some embodiments, said IgG antibody construct comprises a TL1A binding light chain variable domain that interact with said TL1A binding heavy chain variable domain, thereby forming said TL1A binding region, said TL1A binding heavy chain variable domain and/or said TL1A binding light chain variable domain comprises at least one amino acid modification that (a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or (b) decreases binding affinity of said TL1A binding region for said TL1A relative to said binding affinity of corresponding TL1A binding region prior to said at least one amino acid modification under acidic pH condition, as measured by surface plasmon resonance spectroscopy. In some embodiments, said IgG antibody construct comprises an IL-12 binding light chain variable domain that interact with said IL-12 binding heavy chain variable domain, thereby forming said IL-12 binding region, said IL-12 binding heavy chain variable domain and/or said IL-12 binding light chain variable domain comprises at least one amino acid modification that (a) increases binding affinity of said IL-12 binding region for said IL-12 relative to said binding affinity of corresponding IL-12 binding region prior to said at least one amino acid modification under neutral pH condition, as measured by surface plasmon resonance spectroscopy, and/or (b) decreases binding affinity of said IL-12 binding region for said IL-12 relative to said binding affinity of corresponding IL-12 binding region prior to said at least one amino acid modification under acidic pH condition, as measured by surface plasmon resonance spectroscopy. In some embodiments, said at least one amino acid modification is within a CDR-H1, a CDR-H2, and/or a CDR-H3 of said TL1A binding heavy chain variable domain. In some embodiments, said at least one amino acid modification is within a CDR-H3 of said TL1A binding heavy chain variable domain. In some embodiments, said at least one amino acid modification comprises a substitution of at least one uncharged amino acid with a charged amino acid. In some embodiments, said at least one amino acid modification is within at least one framework region of said TL1A binding heavy chain variable domain. In some embodiments, said at least one amino acid modification is within a CDR-L1, a CDR-L2, and/or a CDR-L3 of said TL1A binding light chain variable domain. In some embodiments, said at least one amino acid modification is within at least one framework region of said TL1A binding light chain variable domain. In some embodiments, said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said at least one amino acid modification is within a CDR-H1, a CDR-H2, and/or a CDR-H3 of said IL-12 binding heavy chain variable domain. In some embodiments, said at least one amino acid modification is within at least one framework region of said IL-12 binding heavy chain variable domain. In some embodiments, said at least one amino acid modification is within a CDR-L1, a CDR-L2, and/or a CDR-L3 of said IL-12 binding light chain variable domain. In some embodiments, said at least one amino acid modification increases binding affinity of said IL-12 binding region for said IL-12 in neutral pH condition by at least 2%, at least 5%, at least 10%, at least 15%, at least 20%, at least 30%, at least 40%, at least 50% or more relative to binding affinity of corresponding IL-12 binding region prior to said at least one amino acid modification, as measured by surface plasmon resonance spectroscopy. In some embodiments, said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy. In some embodiments, said IL-12 binding heavy chain variable domain further binds an epitope that is present on IL-23, a variant thereof or a functional fragment thereof. In some embodiments, said IgG antibody construct has higher binding affinity for trimeric TL1A relative to monomeric TL1A under neutral pH condition. In some embodiments, said IgG antibody construct has higher binding affinity for monomeric TL1A relative to trimeric TL1A under neutral pH condition. In some embodiments, said TL1A binding light chain variable domain and said IL-12 binding light chain variable domain are at least 90%, at least 95%, at least 98% or 100% identical relative to each other. In some embodiments, said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

Also provided herein are IgG antibody constructs comprising a TL1A binding region that binds an epitope on TL1A, a variant thereof or a functional fragment thereof, and a second binding region that binds a second epitope on IL-6R, IL-6, IL-17 family cytokine, IL-17R, a variant thereof or a functional fragment thereof, wherein said TL1A binding region comprises a TL1A binding heavy chain variable domain and a TL1A binding light chain variable domain, wherein at least one amino acid modification in said TL1A binding heavy chain variable domain and/or said TL1A binding light chain variable domain, and wherein said amino acid modification changes an isoelectric point (pI) of said IgG antibody construct relative to a pI of a corresponding IgG antibody construct prior to said amino acid modification, thereby resulting in pH-dependent binding activity for said TL1A in said IgG antibody construct, as measured by surface plasmon resonance spectroscopy. In some embodiments, said second binding region comprises a second binding heavy chain variable domain and a second binding light chain variable domain, and at least one amino acid modification in said second binding heavy chain variable domain and/or said second binding light chain variable domain, wherein said amino acid modification changes an isoelectric point (pI) of said IgG antibody construct relative to a pI of a corresponding IgG antibody construct prior to said amino acid modification, thereby resulting in pH-dependent binding activity for said second epitope in said IgG antibody construct, as measured by surface plasmon resonance spectroscopy. In some embodiments, said at least one amino acid modification is within a CDR-H1, a CDR-H2, and/or a CDR-H3 of said TL1A binding heavy chain variable domain. In some embodiments, said at least one amino acid modification is within a CDR-H3 of said TL1A binding heavy chain variable domain. In some embodiments, said at least one amino acid modification comprises a substitution of at least one uncharged amino acid with a charged amino acid. In some embodiments, said at least one amino acid modification is within at least one framework region of said second binding heavy chain variable domain. In some embodiments, said at least one amino acid modification is within a CDR-L1, a CDR-L2, and/or a CDR-L3 of said second binding light chain variable domain. In some embodiments, said at least one amino acid modification is within at least one framework region of said second binding light chain variable domain. In some embodiments, said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy. In some embodiments, said at least one amino acid modification reduces binding affinity of said TL1A binding region for said TL1A in acidic pH condition by at least 10% relative to binding affinity of corresponding TL1A binding region prior to said at least one amino acid modification, as measured by surface plasmon resonance spectroscopy. In some embodiments, said at least one amino acid modification is within a CDR-H1, a CDR-H2, and/or a CDR-H3 of said second binding heavy chain variable domain. In some embodiments, said at least one amino acid modification is within at least one framework region of said second binding heavy chain variable domain. In some embodiments, said IgG antibody construct has higher binding affinity for trimeric TL1A relative to monomeric TL1A under neutral pH condition. In some embodiments, said IgG antibody construct has higher binding affinity for monomeric TL1A relative to trimeric TL1A under neutral pH condition. In some embodiments, said TL1A binding light chain variable domain and said second binding light chain variable domain are at least 90%, at least 95%, at least 98% or 100% identical relative to each other. In some embodiments, said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

Also provided herein are IgG antibody constructs comprising a TL1A binding region that binds an epitope on TL1A, a variant thereof or a functional fragment thereof, and a second binding region that binds a second epitope on IL-6R, IL-6, IL-17 family cytokine, IL-17R, a variant thereof or a functional fragment thereof, wherein said TL1A binding region comprises a TL1A binding heavy chain variable domain, wherein said second binding region comprises a second binding heavy chain variable domain, and wherein a binding affinity of said TL1A binding region for said TL1A is at least 10% higher at pH 7.4 relative to said binding affinity at pH 5.8, as measured by surface plasmon resonance spectroscopy. In some embodiments, a binding affinity of said second binding region for said second epitope is at least 10% higher at pH 7.4 relative to said binding affinity at pH 5.8, as measured by surface plasmon resonance spectroscopy. In some embodiments, said IgG antibody construct comprises a TL1A binding light chain variable domain that interact with said TL1A binding heavy chain variable domain, thereby forming said TL1A binding region, said TL1A binding heavy chain variable domain and/or said TL1A binding light chain variable domain comprises at least one amino acid modification that (a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or (b) decreases binding affinity of said TL1A binding region for said TL1A relative to said binding affinity of corresponding TL1A binding region prior to said at least one amino acid modification under acidic pH condition, as measured by surface plasmon resonance spectroscopy. In some embodiments, said IgG antibody construct comprises a second binding light chain variable domain that interact with said second binding heavy chain variable domain, thereby forming said second binding region, said second binding heavy chain variable domain and/or said second binding light chain variable domain comprises at least one amino acid modification that (a) increases binding affinity of said second binding region second epitope relative to said binding affinity of corresponding second binding region prior to said at least one amino acid modification under neutral pH condition, as measured by surface plasmon resonance spectroscopy, and/or (b) decreases binding affinity of said second binding region second epitope relative to said binding affinity of corresponding second binding region prior to said at least one amino acid modification under acidic pH condition, as measured by surface plasmon resonance spectroscopy. In some embodiments, said at least one amino acid modification is within a CDR-H1, a CDR-H2, and/or a CDR-H3 of said TL1A binding heavy chain variable domain. In some embodiments, said at least one amino acid modification is within a CDR-H3 of said TL1A binding heavy chain variable domain. In some embodiments, said at least one amino acid modification comprises a substitution of at least one uncharged amino acid with a charged amino acid. In some embodiments, said at least one amino acid modification is within at least one framework region of said second binding heavy chain variable domain. In some embodiments, said at least one amino acid modification is within a CDR-L1, a CDR-L2, and/or a CDR-L3 of said second binding light chain variable domain. In some embodiments, said at least one amino acid modification is within at least one framework region of said second binding light chain variable domain. In some embodiments, said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy. In some embodiments, said at least one amino acid modification reduces binding affinity of said TL1A binding region for said TL1A in acidic pH condition by at least 10% relative to binding affinity of corresponding TL1A binding region prior to said at least one amino acid modification, as measured by surface plasmon resonance spectroscopy. In some embodiments, said at least one amino acid modification is within a CDR-H1, a CDR-H2, and/or a CDR-H3 of said second binding heavy chain variable domain. In some embodiments, said at least one amino acid modification is within at least one framework region of said second binding heavy chain variable domain. In some embodiments, said IgG antibody construct has higher binding affinity for trimeric TL1A relative to monomeric TL1A under neutral pH condition. In some embodiments, said IgG antibody construct has higher binding affinity for monomeric TL1A relative to trimeric TL1A under neutral pH condition. In some embodiments, said TL1A binding light chain variable domain and said second binding light chain variable domain are at least 90%, at least 95%, at least 98% or 100% identical relative to each other. In some embodiments, said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

Also provided herein are IgG antibody constructs comprising a TL1A binding region that binds an epitope on TL1A, a variant thereof or a functional fragment thereof, and an second binding region that binds a second epitope on IL-6R, IL-6, IL-17 family cytokine, IL-17R, a variant thereof or a functional fragment thereof, wherein said TL1A binding region comprises a TL1A binding heavy chain variable domain, and wherein said second binding region comprises a second binding heavy chain variable domain. In some embodiments, a binding affinity of said TL1A binding region for said TL1A is higher than a binding affinity of said second binding region for said second epitope under neutral pH condition, as measured by surface plasmon resonance spectroscopy. In some embodiments, said binding affinity of said TL1A binding region for said TL A is at least two times higher than said binding affinity of said second binding region for said second epitope. In some embodiments, a binding affinity of said second binding region for said second epitope is higher than a binding affinity of said TL1A binding region for said TL1A under neutral pH condition, as measured by surface plasmon resonance spectroscopy. In some embodiments, a binding affinity of said second binding region for said second epitope is at least two times higher than a binding affinity of said TL1A binding region for said TL1A. In some embodiments, said IgG antibody construct comprises a TL1A binding light chain variable domain that interact with said TL1A binding heavy chain variable domain, thereby forming said TL1A binding region, said TL1A binding heavy chain variable domain and/or said TL1A binding light chain variable domain comprises at least one amino acid modification that (a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or (b) decreases binding affinity of said TL1A binding region for said TL1A relative to said binding affinity of corresponding TL1A binding region prior to said at least one amino acid modification under acidic pH condition, as measured by surface plasmon resonance spectroscopy. In some embodiments, said IgG antibody construct comprises a second binding light chain variable domain that interact with said second binding heavy chain variable domain, thereby forming said second binding region, said second binding heavy chain variable domain and/or said second binding light chain variable domain comprises at least one amino acid modification that (a) increases binding affinity of said second binding region second epitope relative to said binding affinity of corresponding second binding region prior to said at least one amino acid modification under neutral pH condition, as measured by surface plasmon resonance spectroscopy, and/or (b) decreases binding affinity of said second binding region second epitope relative to said binding affinity of corresponding second binding region prior to said at least one amino acid modification under acidic pH condition, as measured by surface plasmon resonance spectroscopy. In some embodiments, said at least one amino acid modification is within a CDR-H1, a CDR-H2, and/or a CDR-H3 of said TL1A binding heavy chain variable domain. In some embodiments, said at least one amino acid modification is within a CDR-H3 of said TL1A binding heavy chain variable domain. In some embodiments, said at least one amino acid modification comprises a substitution of at least one uncharged amino acid with a charged amino acid. In some embodiments, said at least one amino acid modification is within at least one framework region of said second binding heavy chain variable domain. In some embodiments, said at least one amino acid modification is within a CDR-L1, a CDR-L2, and/or a CDR-L3 of said second binding light chain variable domain. In some embodiments, said at least one amino acid modification is within at least one framework region of said second binding light chain variable domain. In some embodiments, said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy. In some embodiments, said at least one amino acid modification reduces binding affinity of said TL1A binding region for said TL1A in acidic pH condition by at least 10% relative to binding affinity of corresponding TL1A binding region prior to said at least one amino acid modification, as measured by surface plasmon resonance spectroscopy. In some embodiments, said at least one amino acid modification is within a CDR-H1, a CDR-H2, and/or a CDR-H3 of said second binding heavy chain variable domain. In some embodiments, said at least one amino acid modification is within at least one framework region of said second binding heavy chain variable domain. In some embodiments, said IgG antibody construct has higher binding affinity for trimeric TL1A relative to monomeric TL1A under neutral pH condition. In some embodiments, said IgG antibody construct has higher binding affinity for monomeric TL1A relative to trimeric TL1A under neutral pH condition. In some embodiments, said TL1A binding light chain variable domain and said second binding light chain variable domain are at least 90%, at least 95%, at least 98% or 100% identical relative to each other. In some embodiments, said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region. In some embodiments, the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the IgG antibody construct is an antibody, a variant thereof, or a functional fragment thereof. In some embodiments, the IgG antibody constructs further comprise a constant region, wherein said constant region comprises a first Fc domain and/or a second Fc domain. In some embodiments, said first Fc domain and said second Fc domain are derived from IgG1, IgG2, IgG3 or IgG4. In some embodiments, said first Fc domain and said second Fc domain are derived from IgG1 constant domain. In some embodiments, said first Fc domain and said second Fc domain independently comprise M252Y, S254T, and T256E substitutions. In some embodiments, said first Fc domain and said second Fc domain independently comprise L234A and L235A substitutions. In some embodiments, said first Fc domain and said second Fc domain independently comprise P329G substitution. In some embodiments, said first Fc domain comprises a knob modification and said second Fc domain comprises a hole modification, wherein: said knob modification comprises replacing an original amino acid residue from an interface of the Fc region with an amino acid residue selected from tyrosine and tryptophan, and wherein said hole modification comprises replacing an original amino acid residue from the interface of the Fc region with an amino acid residue selected from serine, threonine, valine and alanine. In some embodiments, the first Fc domain comprises at least two modifications at positions selected from L351, F405 and Y407, per EU numbering, and the second Fc domain comprises at least one modification at positions selected from T366, K392 and T394, per EU numbering. In some embodiments, the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering. In some embodiments, the first Fc domain comprises at least one substitution at positions selected from S354 and T366, per EU numbering, and the second Fc domain comprises at least one modification at positions selected from T366, L368 and Y407, per EU numbering. In some embodiments, the first Fc domain comprises at least one substitution selected from S354 and T366W, per EU numbering, and the second Fc domain comprises at least one substitution selected from T366S. L368A, Y407T, and Y407V, per EU numbering. In some embodiments, said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification. In some embodiments, said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, said at least one modification decreases plasma clearance (CL), increases plasma retention time, or increases plasma half-life (t½) relative to a corresponding IgG antibody construct prior to said at least one modification in said constant region. In some embodiments, a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification. In some embodiments, said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification. In some embodiments, the at least one modification increases plasma clearance of TL1A by the IgG antibody construct, compared to a corresponding plasma clearance by an IgG antibody construct prior to the at least one modification in the constant region.

Also provided herein are antibody constructs comprising a TL1A binding region that binds an epitope on TL1A, a variant thereof or a functional fragment thereof, wherein the TL1A binding region comprises a heavy chain variable region, wherein the heavy chain variable region comprises at least one modification at positions selected from S30, T69, L83, N84, T104 and F107, relative to the amino acid position numbering of SEQ ID NO: 923. In some embodiments, the heavy chain variable region comprises at least one modification comprises a substitution selected from the group consisting of S30T, T69I, L83V, N84K, T104D, F107N and F107D, relative to the amino acid position numbering of SEQ ID NO: 923. In some embodiments, the at least one modification improves binding interaction with H109, H118 and/or H121 of TL1A comprising an amino acid sequencer of SEQ ID NO: 139.

Also provided herein are antibody constructs comprising an IL-23 binding region that binds an epitope on IL-23, a variant thereof or a functional fragment thereof, wherein the IL-23 binding region comprises a heavy chain variable region, wherein the heavy chain variable region comprises at least one modification at positions selected from T28, T30, I31, A33, 134, G56, G58, H59, Q62, Q65, R98, E101, N102 and L108 relative to the amino acid position numbering of SEQ ID NO: 1544. In some embodiments, heavy chain variable region comprises at least one modification comprises a substitution selected from the group consisting of T28V, T28A, T28L, T28P, T30S, 131Q, A33T, I34M, 134V, G56K, G56A, G56N, G58A, H59Y, H59V, Q62S, Q62K, Q65R, Q65K, Q65A, R981, E101S, E101Y, E101T, N102F, N102Y, N102S, N102D, N102K, N102R, L108T and L108M relative to the amino acid position numbering of SEQ ID NO: 1544.

Also provided herein are pharmaceutical compositions comprising the IgG antibody construct described herein, or the antibody construct described herein, and a pharmaceutically acceptable carrier.

Also described herein are methods of treating a disease or a condition, wherein the method comprises administering an effective amount of the IgG antibody construct described herein, the antibody construct described herein, or the pharmaceutical composition of claim 109 to a subject in need thereof results in treatment of a disease or condition. In some embodiments, the disease or condition is related to impaired mitochondrial dysfunction. In some embodiments, the disease or condition is selected from the group consisting of: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-1F depict a bispecific antibody comprising two binding domains. Specifically, FIG. 1A depicts a bispecific antibody having TL1A binding domain and IL-6R binding domain. FIG. 1B depicts a bispecific antibody having a Fab domain and an scFv domain, wherein the Fab domain is a TL1A binding domain and the scFv domain is an IL-17 family cytokine binding domain. FIG. 1C depicts a bispecific antibody is Fab2 having two Fab domains, wherein the first Fab domain is a TL1A binding domain and the second Fab domain is an IL-17R binding domain. FIG. 1D depicts a bispecific antibody having TL1A binding domain and IL-12 binding domain. FIG. 1E depicts a bispecific antibody having a Fab domain and an scFv domain, wherein the Fab domain is a TL1A binding domain and the scFv domain is an IL-23 binding domain. FIG. 1F depicts a bispecific antibody is Fab2 having two Fab domains, wherein the first Fab domain is a TL1A binding domain, and the second Fab domain is an IL-6 binding domain.

FIGS. 2A-2B show representative results of competitive binding assay for TL1A binding antibody candidates. FIG. 2A shows the competitive binding assay result for TL1A binding antibody candidate 1 having a VH amino acid sequence of SEQ ID NO: 923, and a VL amino acid sequence of SEQ ID NO: 1768. FIG. 2B shows the competitive binding assay result for TL1A binding antibody candidate 2 having a VH amino acid sequence of SEQ ID NO: 1048, and a VL amino acid sequence of SEQ ID NO: 1768.

FIGS. 3A-3B show representative results of competitive binding assay for IL-23 binding antibody candidates. FIG. 3A shows the competitive binding assay result for IL-23 binding antibody candidate 1 having a VH amino acid sequence of SEQ ID NO: 1544, and a VL amino acid sequence of SEQ ID NO: 1768. FIG. 3B shows the competitive binding assay result for IL-23 binding antibody candidate 2 having a VH amino acid sequence of SEQ ID NO: 1621, and a VL amino acid sequence of SEQ ID NO: 1768.

FIG. 4 summarizes results blocking effect of TL1A binding region of multispecific antibodies prepared by fab-arm exchange method using luciferase assay. Luciferase assay was conducted at 130.0 ng/mL concentration of TL1A with each of five antibodies at a concentration ranging from 0.1, 1.0 or g/mL. The five antibodies included (1) monomeric IL-23 binding antibody comprising a VH amino acid sequence of SEQ ID NO: 375 and a VL amino acid sequence of SEQ ID NO: 463, (2) heterodimeric IL-23/IL-12 binding antibody comprising a VH amino acid sequence of SEQ ID NO: 283 and a VL amino acid sequence of SEQ ID NO: 303, (3) TL1A binding antibody comprising a VH amino acid sequence of SEQ ID NO: 487 and a VL sequence comprising an amino acid sequence of SEQ ID NO: 491, (4) bispecific antibody-1 comprising a first VH amino acid sequence of SEQ ID NO: 375, a first VL amino acid sequence of SEQ ID NO: 463, a second VH amino acid sequence of SEQ ID NO: 487 and a second VL sequence comprising an amino acid sequence of SEQ ID NO: 491, and (5) bispecific antibody-2 comprising a first VH amino acid sequence of SEQ ID NO: 283, a first VL amino acid sequence of SEQ ID NO:303, a second VH amino acid sequence of SEQ ID NO: 487 and a second VL sequence comprising an amino acid sequence of SEQ ID NO: 491. Each antibody candidate comprised hIgG1 constant region with L234A, L235A, M252Y, S254T, T256E and P329G modifications. For negative and positive control, only cells and cells in combination with TL1A were used, respectively. Results of the assay are showed for the monomeric IL-23 binding antibody, heterodimeric IL-23/IL-12 binding antibody, TL1A binding antibody, bispecific antibody-1 and bispecific antibody-2 (from left to right).

FIG. 5 summarizes results blocking effect of TL1A binding region of multispecific antibodies prepared by key-in-hole method using luciferase assay. Luciferase assay was conducted at 130.0 ng/mL concentration of TL1A with each of four antibodies at a concentration ranging from 0.1, 1.0 or 10 g/mL. The four antibodies are described in TABLE 45 as bispecific antibody candidate II-1 to II-4. For negative and positive control, only cells and cells in combination with TL1A were used, respectively. Results of the assay are showed for bispecific antibody candidate 11-1, bispecific antibody candidate 11-2, bispecific antibody candidate 11-3, and bispecific antibody candidate 11-4 (from left to right).

FIGS. 6A-6C show representative binding curves for three knob-in-hole antibody constructions prepared in Example 10. FIGS. 6A, 6B and 6C show bispecific antibody candidate II-1, bispecific antibody candidate 11-2, and bispecific antibody candidate 11-3, respectively.

FIG. 7 summarizes results blocking effect of IL-23 binding region of multispecific antibodies prepared by fab-arm exchange method using luciferase assay. Luciferase assay was conducted on cells treated with IL-23 and each of five antibodies at a concentration ranging from 0.1, 1.0 or 10 μg/mL. The five antibodies included (1) monomeric IL-23 binding antibody comprising a VH amino acid sequence of SEQ ID NO: 375 and a VL amino acid sequence of SEQ ID NO: 463, (2) heterodimeric IL-23/IL-12 binding antibody comprising a VH amino acid sequence of SEQ ID NO: 283 and a VL amino acid sequence of SEQ ID NO: 303, (3) TL1A binding antibody comprising a VH amino acid sequence of SEQ ID NO: 487 and a VL sequence comprising an amino acid sequence of SEQ ID NO: 491, (4) bispecific antibody-1 comprising a first VH amino acid sequence of SEQ ID NO: 375, a first VL amino acid sequence of SEQ ID NO: 463, a second VH amino acid sequence of SEQ ID NO: 487 and a second VL sequence comprising an amino acid sequence of SEQ ID NO: 491, and (5) bispecific antibody-2 comprising a first VH amino acid sequence of SEQ ID NO: 283, a first VL amino acid sequence of SEQ ID NO:303, a second VH amino acid sequence of SEQ ID NO: 487 and a second VL sequence comprising an amino acid sequence of SEQ ID NO: 491. Each antibody candidate comprised hIgG1 constant region with L234A, L235A, M252Y, S254T, T256E and P329G modifications. For negative and positive control, only cells and cells in combination with IL-23 were used, respectively. Results of the assay are showed for the monomeric IL-23 binding antibody, heterodimeric IL-23/IL-12 binding antibody, TL1A binding antibody, bispecific antibody-1 and bispecific antibody-2 (from left to right).

FIG. 8 summarizes results blocking effect of IL-23 binding region of multispecific antibodies prepared by key-in-hole method using luciferase assay. Luciferase assay was conducted for IL-23 with each of four antibodies at a concentration ranging from 0.1, 1.0 or 10 μg/mL. The four antibodies are described in TABLE 45 as bispecific antibody candidate II-1 to II-4. For negative and positive control, only cells and cells in combination with IL-23 were used, respectively. Results of the assay are showed for bispecific antibody candidate II-1, bispecific antibody candidate II-2, bispecific antibody candidate II-3, and bispecific antibody candidate II-4 (from left to right).

### DETAILED DESCRIPTION OF EMBODIMENTS

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features can also be provided separately or in any suitable combination. Conversely, although the present disclosure may be described herein in the context of separate embodiments for clarity, the present disclosure can also be implemented in a single embodiment.

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the terms, “disease”, “disorder”, and “condition,” which are used interchangeably herein, refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, or affectation.

As used herein, the term, “in need thereof,” when used in the context of a therapeutic or prophylactic treatment, means having a disease, being diagnosed with a disease, or being in need of preventing a disease, e.g., for one at risk of developing the disease. Thus, a subject in need thereof can be a subject in need of treating or preventing a disease.

As used herein, the term, “administering,” refers to the placement of a compound (e.g., an antibody or fragment thereof as disclosed herein) into a subject by a method or route that results in at least partial delivery of the agent at a desired site. Pharmaceutical compositions comprising an antibody or fragment thereof, disclosed herein can be administered by any appropriate route which results in an effective treatment in the subject, including but not limited to intravenous, intraarterial, subcutaneous injection or infusion directly into a tissue parenchyma, etc. Where necessary or desired, administration can include, for example, intracerebroventricular (“icv”) administration, intranasal administration, intracranial administration, intracelial administration, intracerebellar administration, subcutaneous administration, or intrathecal administration.

As used herein, the term, “subject”, “patient”, “individual” and like terms, which are used interchangeably, refer to a vertebrate, a mammal, a primate, or a human. Mammals include, without limitation, humans, primates, rodents, wild or domesticated animals, including feral animals, farm animals, sport animals, and pets. Primates include, for example, chimpanzees, cynomolgus monkeys, spider monkeys, and macaques (e.g., Rhesus). Rodents include, for example, mice, rats, woodchucks, ferrets, rabbits and hamsters. Domestic and game animals include, for example, cows, horses, pigs, deer, bison, buffalo, feline species (e.g., domestic cat), and canine species (e.g., dog, fox and wolf,) avian species (e.g., chicken, emu and ostrich), and fish (e.g., trout, catfish and salmon). The terms, “individual,” “patient” and “subject” are used interchangeably herein. A subject can be male or female. In some embodiments, the subject is a mammal. The mammal can be a human, non-human primate, mouse, rat, dog, cat, horse, or cow, but is not limited to these examples. Mammals other than humans can be advantageously used as subjects that represent animal models of conditions or disorders. Non-limiting examples include murine models. In addition, the compositions and methods described herein can be used to treat domesticated animals and/or pets. A subject can be one who is diagnosed and currently being treated for, or seeking treatment, monitoring, adjustment or modification of an existing therapeutic treatment, or is at a risk of developing a given disorder.

As used herein, the terms, “protein”, “peptide” and “polypeptide,” which are used interchangeably, refer to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric. A polypeptide can have the amino acid sequence of naturally occurring polypeptide from any mammal. Such native sequence polypeptide can be isolated from nature or can be produced by recombinant or synthetic means. In some embodiments, the polypeptide is a “variant”. “Variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide. A “derivative” of a polypeptide is a polypeptide (e.g., an antibody) that has been chemically modified, e.g., via conjugation to another chemical moiety (such as, for example, polyethylene glycol or albumin, e.g., human serum albumin), phosphorylation, and glycosylation.

As used herein, the term, “percent identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., using publicly available computer software such as BLAST, BLASTP, BLASTN, BLAST-2, ALIGN, MEGALIGN (DNASTAR), CLUSTALW, CLUSTAL OMEGA, or MUSCLE software or other algorithms available to persons of skill) or by visual inspection. Software for performing BLAST analyses is publicly available through the National Center for Biotechnology Information (ncbi.nlm.nih.gov). Those skilled in the art can determine appropriate parameters for aligning sequences, including any algorithms needed to achieve maximal alignment over the full length of the sequences being compared. Depending on the application, the percent “identity” can exist over a region of the sequence being compared, e.g., over a functional domain, or, alternatively, exist over the full length of the two sequences to be compared. For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

As used herein, the terms, “increased”, “increase”, and “enhance,” refer to an increase by a statistically significant amount; for the avoidance of doubt, the terms “increased”, “increase”, or “enhance”, mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “antibody” refers to an immunoglobulin molecule that specifically binds to, or is immunologically reactive toward, a specific target which in the current instance can be, for example, TL1A, IL-6R, IL-6, IL-12, IL-23, IL-12p35, IL-12p40, IL-23p19, an IL-17 family cytokine, IL-17R, a variant thereof, or fragments thereof. Antibody can include, for example, polyclonal, monoclonal, genetically engineered, and fragments thereof. An antibody can be, for example, murine, chimeric, humanized, heteroconjugate, bispecific, diabody, triabody, or tetrabody. The fragment can include, for example, Fab′, F(ab′)2, Fab, Fv, rlgG, scFv, hcAbs (heavy chain antibodies), a single domain antibody, V_{HH}, V_{NAR}, sdAbs, or nanobody. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same target. A whole antibody may comprise four polypeptides: two identical copies of a heavy (H) chain polypeptide and two identical copies of a light (L) chain polypeptide. Each of the heavy chains may contain one N-terminal variable (VH) region and three C-terminal constant (CH1, CH2 and CH3) regions, and each light chain may contain one N-terminal variable (VL) region and one C-terminal constant (CL) region. The variable regions of each pair of light and heavy chains may form a binding site of an antibody. In exemplary embodiments of bispecific antibodies, multiple distinct binding sites may be present. The VH and VL regions may have a similar general structure, with each region comprising four framework regions, whose sequences are relatively conserved. In some embodiments, the framework regions may be connected by three complementarity determining regions (CDRs). In some embodiments, the three CDRs, known as CDR1, CDR2, and CDR3, form the “hypervariable region” of an antibody, which is responsible for binding.

As used herein, the term, “chimeric antibody,” refers to an antibody in which a portion of the heavy and/or light chain is derived from a particular source or species, while the remainder of the heavy and/or light chain is derived from a different source or species.

As used herein, the term, “human antibody,” refers to an antibody comprising an amino acid sequence corresponding to that of an antibody produced by a human or a human cell, or derived from a non-human source that utilizes a human antibody repertoire or human antibody-encoding sequences (e.g., obtained from human sources or designed de novo).

As used herein, the term, “humanized antibody,” refers to an amino acid sequence that differs from the amino acid sequence of an antibody derived from a non-human species by one or more amino acid substitutions, deletions, and/or additions, such that the humanized antibody is less likely to induce an immune response, and/or induces a less severe immune response, as compared to the non-human species antibody, when it is administered to a human subject. In some embodiments, certain amino acids in the framework and constant domains of the heavy and/or light chains of the non-human species antibody are mutated to produce the humanized antibody. In some embodiments, the constant domain(s) from a human antibody are fused to the variable domain(s) of a non-human species. In another embodiment, one or more amino acid residues in one or more CDR sequences of a non-human antibody are changed to reduce the likely immunogenicity of the non-human antibody when it is administered to a human subject, wherein the changed amino acid residues either are not critical for immunospecific binding of the antibody to its target, or the changes to the amino acid sequence that are made are conservative changes, such that the binding of the humanized antibody to the target is not significantly worse than the binding of the non-human antibody to the target. Examples of how to make humanized antibodies can be found in U.S. Pat. Nos. 6,054,297, 5,886,152 and 5,877,293. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

As used herein, the term, “epitope,” means a portion of a target that specifically binds to an antibody. Epitopes frequently consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to TL1A, a variant thereof or a fragment thereof; any one of IL-6, IL-6R, IL-12, IL-23, IL-12p35, IL-12p40, IL-23p19, an IL-17 family cytokine, IL-17R, a variant thereof or a fragment thereof; or a combination thereof.

As used herein, the term, “Complementarity Determining Regions” (CDRs, i.e., CDR1, CDR2, and CDR3), refers to the amino acid residues of an antibody variable domain the presence of which are necessary for binding. Each variable domain typically has three CDR regions identified as CDR1, CDR2 and CDR3. The CDRs of variable heavy chain can be CDR-H1, CDR-H2 and CDR-H3. The CDRs of variable light chain can be CDR-L1, CDR-L2 and CDR-L3. Exemplary hypervariable loops occur at amino acid residues 26-32 (L1), 50-52 (L2), 91-96 (L3), 26-32 (H1), 53-55 (H2), and 96-101 (H3). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. Thus, the HVs may be comprised within the corresponding CDRs and references herein to the “hypervariable loops” of VH and VL domains should be interpreted as also encompassing the corresponding CDRs, and vice versa, unless otherwise indicated. The more highly conserved regions of variable domains are called the framework region (FR), as defined below. The variable domains of native heavy and light chains each comprise four FRs (FR1, FR2, FR3 and FR4, respectively), largely adopting a [beta]-sheet configuration, connected by the three hypervariable loops. The hypervariable loops in each chain are held together in close proximity by the FRs and, with the hypervariable loops from the other chain, contribute to the formation of the binding site of antibodies. Structural analysis of antibodies revealed the relationship between the sequence and the shape of the binding site formed by the complementarity determining regions. Despite their high sequence variability, five of the six loops adopt just a small repertoire of main-chain conformations, called “canonical structures”. These conformations are first of all determined by the length of the loops and secondly by the presence of key residues at certain positions in the loops and in the framework regions that determine the conformation through their packing, hydrogen bonding or the ability to assume unusual main-chain conformations. The antibodies or fragment thereof of the present disclosure can comprise a CDR3 region that is a length of at least about 10, 11, 12, 13, 14, 15, 16, 17, 18, 19, 20, 21, 22, 23, 24, 25, 26, 27, 28, 29, or 30 amino acids in length. The antibodies or fragment thereof of the present disclosure can comprise a CDR3 region that is at least about 18 amino acids in length.

As used herein, the term, “variable region,” when used in reference to an antibody, refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the target-binding site of antibodies. There are at least two techniques for determining CDRs: (1) an approach based on cross-species sequence variability; and (2) an approach based on crystallographic studies of target-antibody complexes. A CDR may refer to CDRs defined by either approach or by a combination of both approaches. Six hypervariable loops (three loops each from the Heavy and Light chain) contribute the amino acid residues for target-binding and confer target-binding specificity to the antibody. However, even a single variable domain (or half of an Fv comprising only three CDRs specific for a target) has the ability to recognize and bind target, although at a lower affinity than the entire binding site.

As used herein, the term, “constant region,” when used in reference to an antibody, refers to the constant region of the antibody light chain (i.e., a light chain constant region) or the constant region of the antibody heavy chain (i.e., a heavy chain constant region) either alone or in combination. The constant region does not vary with respect to target specificity.

As used herein, the terms, “heavy chain region,” includes amino acid sequences derived from the constant domains of an immunoglobulin heavy chain. A polypeptide comprising a heavy chain region comprises at least one of: a CH1 domain, a hinge (e.g., upper, middle, and/or lower hinge region) domain, a CH2 domain, a CH3 domain, or a variant or fragment thereof. In an embodiment, an antibody or a fragment thereof may comprise the Fc region of an immunoglobulin heavy chain (e.g., a hinge portion, a CH2 domain, and a CH3 domain). In another embodiment, an antibody or a fragment thereof lacks at least a region of a constant domain (e.g., all or part of a CH2 domain). In some embodiments, at least one, and preferably all, of the constant domains are derived from a human immunoglobulin heavy chain. For example, in one preferred embodiment, the heavy chain region comprises a fully human hinge domain. In other preferred embodiments, the heavy chain region comprising a fully human Fc region (e.g., hinge, CH2 and CH3 domain sequences from a human immunoglobulin). In some embodiments, the constituent constant domains of the heavy chain region are from different immunoglobulin molecules.

As used herein, the term, “hinge region,” includes the region of a heavy chain molecule that joins the CH1 domain to the CH2 domain. The hinge region can comprise approximately 25 residues and is flexible, thus allowing the two N-terminal binding regions to move independently. Hinge regions can be subdivided into three distinct domains: upper, middle, and lower hinge domains.

As used herein, the term “Fv” is the minimum antibody fragment that contains a complete target-recognition and -binding site. This fragment consists of a dimer of one heavy- and one light-chain variable region domain in tight, non-covalent association. From the folding of these two domains emanate six hypervariable loops (three loops each from the H and L chain) that contribute the amino acid residues for binding and confer binding specificity to the antibody. However, even a single variable domain (or half of an Fv comprising only three CDRs specific for a target) has the ability to recognize and bind target, although at a lower affinity than the entire binding site.

As used herein, the term, “heavy chain variable region” or “VH,” when used in reference to an antibody, refers to the fragment of the heavy chain that contains three CDRs interposed between flanking stretches known as framework regions, these framework regions are generally more highly conserved than the CDRs and form a scaffold to support the CDRs.

As used herein, the term, “light chain variable region” or “VL,” when used in reference to an antibody, refers to the fragment of the light heavy chain that contains three CDRs interposed between flanking stretches known as framework regions, these framework regions are generally more highly conserved than the CDRs and form a scaffold to support the CDRs.

As used herein, the term, “framework residues” or “FR,” are those variable domain amino acid residues other than the hypervariable region amino acid residues.

As used herein, the term, “antibody heavy chain,” refers to the larger of the two types of polypeptide chains present in antibody molecules in their naturally occurring conformations, and which normally determines the class to which the antibody belongs.

As used herein, the term, “antibody light chain,” refers to the smaller of the two types of polypeptide chains present in antibody molecules in their naturally occurring conformations. Kappa (“κ”) and lambda (“λ”) light chains refer to the two major antibody light chain isotypes.

As used herein, the phrase, “specifically binds” or “preferentially binds,” refers to an antibody or fragment thereof that binds to a target with greater affinity and/or avidity than it binds to epitopes on unrelated polypeptides. The specificity of an antibody or fragment thereof can be determined based on affinity and/or avidity. Methods to determine such specific binding are also well known in the art.

As used herein, the term, “binding affinity,” refers to the strength of the sum total of noncovalent interactions between a first molecule and a second molecule. Unless indicated otherwise, the binding affinity refers to intrinsic binding affinity reflecting interaction between the first molecule and the second molecule at a ratio of 1:1.

As used herein, the term, “multispecific antibody,” is an antibody that comprises two or more different target-binding domains that collectively specifically bind two or more different epitopes. The two or more different epitopes may be epitopes on the same cell or on different cells. In some embodiments, a multi-specific antibody binds two different epitopes (i.e., a “bispecific antibody”). In some embodiments, a multi-specific antibody binds three different epitopes (i.e., a “trispecific antibody”).

As used herein a “recombinant antibody” is an antibody that comprises an amino acid sequence derived from two different species, or two different sources, and includes synthetic and/or non-naturally-occurring molecules. By way of non-limiting example, a recombinant antibody may comprise a non-human CDR and a human variable region framework or constant or Fc region, an antibody with binding domains from two different monoclonal antibodies, or an antibody comprising a mutation of one or more amino acid residues to increase or decrease biological activity or binding of a part of the antibody. In certain embodiments, recombinant antibodies are produced from a recombinant DNA molecule or synthesized. In certain embodiments, the antibodies described herein are a polypeptide(s) encoded by one or more polynucleotides.

As used herein, “recognize” or “bind” or “selective for” refers to the association or binding between a binding domain and a target domain.

As used herein, an “antibody construct” refers to a construct that may contain a binding domain and an Fc domain.

As used herein, a “binding domain” refers to an antibody or non-antibody domain.

As used herein, a “binding domain” refers to a domain from an antibody or from a non-antibody that can bind to a target domain. Binding domains can be numbered when there is more than one binding domains in a given conjugate or antibody construct (e.g., first binding domain, second binding domain, third binding domain, etc.). Different binding domains in the same conjugate or construct can target the same target or different targets.

As used herein, an “Fc domain” refers to an Fc domain from an antibody or from a non-antibody that can bind to an Fc receptor. As used herein, an “Fc domain” and an “Fc comprising domain” can be used interchangeably.

As used herein, a “target binding domain” refers to a construct that contains a binding domain from an antibody or from a non-antibody that can bind to a target (e.g. TL1A, IL-6, IL-6R, IL-12, IL-23, IL-12p35, IL-12p40, IL-23p19, an IL-17 family cytokine, IL-17R).

As used herein, the abbreviations for the natural 1-enantiomeric amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gin); glycine (G, Gly); histidine (H, His); isoleucine (I, He); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Unless otherwise specified, X can indicate any amino acid.

The phrase “pharmaceutically acceptable” is employed herein to refer to those compounds, materials, compositions, and/or dosage forms which are, within the scope of sound medical judgment, suitable for use in contact with the tissues of subjects for instance, human beings and animals, without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) tale; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The terms “fragment of an antibody,” “antibody fragment,” “functional fragment of an antibody,” “binding domain” or their grammatical equivalents are used interchangeably herein to mean one or more fragments or portions of an antibody that retain the ability to specifically bind to a target. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment that may comprise VL, VH, CL, and CH1 domains; (ii) a F(ab′)2 fragment, which is a bivalent fragment comprising two Fab fragments linked by a disulfide bridge at the stalk region; (iii) a Fv fragment consisting of the VL and VH domains of a single arm of an antibody; (iv) a single chain Fv (scFv), which is a monovalent molecule consisting of the two domains of the Fv fragment (i.e., VL and VH) joined by a synthetic linker which enables the two domains to be synthesized as a single polypeptide chain; and (v) a diabody, which is a dimer of polypeptide chains, wherein each polypeptide chain may comprise a VH connected to a VL by a peptide linker that is too short to allow pairing between the VH and VL on the same polypeptide chain, thereby driving the pairing between the complementary domains on different VH-VL polypeptide chains to generate a dimeric molecule having two functional target binding sites. Antibody fragments are known in the art. Other antibody fragments can include variable fragments of heavy chain antibodies (VHH).

As used herein, the term, “Fab,” refers to a region of an antibody composed of one constant and one variable domain of each of the heavy and the light chains (monovalent target-binding fragment), but wherein the heavy chain is truncated such that it lacks the CH2 and CH3 domain (i.e., VH, CH1, VL, and CL), and may also lack some or all of the hinge region. It can be produced by digestion of a whole antibody with the enzyme papain. Fab may refer to this region in isolation, or this region in the context of a full-length antibody, immunoglobulin construct or Fab fusion protein. Fab can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner.

As used herein, the term, “scFv,” refers to an antibody fragment comprising the VH and VL domains of an antibody, wherein these domains are present in a single polypeptide chain. Generally, the Fv polypeptide further comprises a polypeptide linker between the VH and VL domains that enables the scFv to form the desired structure for target-binding.

The term, “conservative amino acid substitution” or “conservative mutation,” refers to the replacement of one amino acid by another amino acid with a common property. A functional way to define common properties between individual amino acids is to analyze the normalized frequencies of amino acid changes between corresponding proteins of homologous organisms. According to such analyses, groups of amino acids may be defined where amino acids within a group exchange preferentially with each other, and therefore resemble each other most in their impact on the overall protein structure. Examples of conservative mutations include amino acid substitutions of amino acids within the sub-groups above, for example, lysine for arginine and vice versa such that a positive charge may be maintained; glutamic acid for aspartic acid and vice versa such that a negative charge may be maintained; serine for threonine such that a free —OH can be maintained; and glutamine for asparagine such that a free —NH₂ can be maintained. Alternatively or additionally, the therapeutic agents can comprise the amino acid sequence of the reference protein with at least one non-conservative amino acid substitution.

The terms “non-conservative mutation” or “non-conservative amino acid substitution” involve amino acid substitutions between different groups, for example, lysine for tryptophan, or phenylalanine for serine, etc. In this case, it is preferable for the non-conservative amino acid substitution to not interfere with or inhibit the biological activity of the therapeutic agent. The non-conservative amino acid substitution may enhance the biological activity of the therapeutic agent, such that the biological activity of the therapeutic agent is increased as compared to the wild-type therapeutic agent.

A “multispecific antibody” is an antibody that can bind simultaneously to at least two targets that are of different structure, e.g., two different target, two different epitopes on the same target, or a hapten and/or an epitope. A “multivalent antibody” is an antibody that can bind simultaneously to at least two targets that are of the same or different structure. Valency indicates how many binding arms or sites the antibody has to a single target or epitope (i.e., monovalent, bivalent, trivalent or multivalent). The multivalency of the antibody means that it can take advantage of multiple interactions in binding to a target, thus increasing the avidity of binding to the target. Specificity indicates how many targets or epitopes an antibody is able to bind (i.e., monospecific, bispecific, trispecific, multispecific). Using these definitions, a natural antibody is bivalent because it has two binding arms but is monospecific because it binds to one epitope. Multispecific, multivalent antibodies are constructs that have more than one binding region of different specificity. For example, the bispecific antibody constructs disclosed herein have a first binding region and a second binding region, wherein the first and second binding regions are distinct.

A “bispecific antibody” is an antibody that can bind simultaneously to two targets which are of different structure. Bispecific antibodies (BsAbs) and bispecific antibody fragments (bsFab) may have at least one arm (or binding domain) that specifically binds to, for example, a first target, and at least one other arm (or binding domain) that specifically binds to a second target. At least one of the first and the second targets may be a target produced by or associated with a diseased cell, tissue, organ or pathogen. A variety of bispecific antibodies can be produced using molecular engineering.

As used herein, the term, “vector,” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the terms, “host cell,” “host cell line” and “host cell culture,” are interchangeable and refer to cells into which an exogenous nucleic acid has been introduced, and the progeny of such cells. Host cells include “transformants” (or “transformed cells”) and “transfectants” (or “transfected cells”), which each include the primary transformed or transfected cell and progeny derived therefrom. Such progeny may not be completely identical in nucleic acid content to a parent cell, and may contain mutations.

A bispecific antibody construct, or a composition described herein, is said to be administered in a “therapeutically effective amount” if the amount administered is physiologically significant. An agent is physiologically significant if its presence results in a detectable change in the physiology of a recipient subject. In particular embodiments, a bispecific antibody construct disclosed herein is physiologically significant if its presence invokes a response or mitigates the signs and symptoms of an infectious or autoimmune disease state. A physiologically significant effect could also be the evocation of a humoral and/or cellular immune response in the recipient subject.

The term “linker” is used to denote polypeptides comprising two or more amino acid residues joined by peptide bonds and are used to link one or more binding portions or variable domains.

An “Fv” or “Fv fragment” may consist of only the light chain variable domain (VL) and heavy chain variable domain (VH) of a “single arm” of an immunoglobulin. Thus an “Fv” is the minimum antibody fragment which contains a complete target-recognition and binding site. A “two-chain” Fv fragment consists of a dimer of one heavy- and one light-chain variable domain in tight, non-covalent association. A single-chain Fv species (scFv) may include a VH and a VL domain of an immunoglobulin, with these domains being present in a single polypeptide chain in which they are covalently linked to each other by a linker peptide. Typically, in a scFv fragment the variable domains of the light and heavy chain associate in a dimeric structure analogous to that in a two-chain Fv species. In single chain Fv fragments, it is possible to either have the variable domain of the light chain arranged at the N-terminus of the single polypeptide chain, followed by the linker and the variable domain of the heavy chain arranged at the C-terminus of the polypeptide chain or vice versa, having the variable domain of the heavy chain arranged on the N-terminus and the variable domain of the light chain at the C-terminus with the linker peptide arranged in between. The linker peptide can be any flexible linker known in the art, for example, made from glycine and serine residues. It is also possible to additionally stabilize the domain association between the VH and the VL domain by introducing disulfide bonds into conserved framework regions. Such scFv fragments are also known as disulfide-stabilized scFv fragments (ds-scFv).

As used herein, the term, “treating” (and variations thereof such as “treat” or “treatment”), refers to clinical intervention in an attempt to alter the natural course of a disease or condition in a subject in need thereof. Treatment can be performed during the course of clinical pathology. Desirable effects of treatment include cure (if applicable), delay the onset of, reduce the severity of, alleviate, ameliorate one or more symptoms of the disease, improve the disease, reduce or improve any associated symptoms of the disease or the predisposition toward the development of the disease.

As used herein, the term, “sufficient amount,” means an amount sufficient to produce a desired effect, e.g., an amount sufficient to modulate an immune response in a subject.

As used herein, the terms, “modulate” and “modulation,” refer to reducing or inhibiting or, alternatively, activating or increasing, a recited variable.

### Antibody Fragments

Antibody fragments which recognize specific epitopes can be generated by known techniques. Antibody fragments are binding portions of an antibody, such as, for example, F(ab′)2, Fab′, F(ab)2, Fab, Fv, scFv and the like. F(ab′)2 fragments can be produced by pepsin digestion of the antibody molecule and Fab′ fragments can be generated by reducing disulfide bridges of the F(ab′)2 fragments. Alternatively, Fab′ expression libraries can be constructed to allow rapid and easy identification of monoclonal Fab′ fragments with the desired specificity. F(ab)2 fragments may be generated by papain digestion of an antibody.

A single chain Fv molecule (scFv) may comprise a VL domain and a VH domain. The VL and VH domains associate to form a target binding site. These two domains may be further covalently linked by a peptide linker (L).

Techniques for producing single domain antibodies (DABs or VHH) are also known in the art, as disclosed for example in Cossins et al. (2006, Prot Express Purif 51:253-259), incorporated herein by reference. Single domain antibodies may be obtained, for example, from camels, alpacas or llamas by standard immunization techniques. The VHH may have potent target binding capacity and can interact with novel epitopes that are inaccessible to conventional VH-VL pairs. Alpaca serum IgG contains about 50% camelid heavy chain only IgG antibodies (HCAbs). Alpacas may be immunized with known targets, such as TNF-α, and VHHs can be isolated that bind to and neutralize the target. PCR primers that amplify virtually all alpaca VHH coding sequences have been identified and may be used to construct alpaca VHH phage display libraries, which can be used for antibody fragment isolation by standard biopanning techniques well known in the art. In certain embodiments, VHH antibody fragments may be utilized in the claimed compositions and methods.

An antibody fragment can be prepared by proteolytic hydrolysis of the full-length antibody or by expression in E. coli or another host of the DNA coding for the fragment. An antibody fragment can be obtained by pepsin or papain digestion of full-length antibodies by conventional methods.

### Multispecific Molecules

Multispecific molecules are antibodies that are capable of binding at least two different targets. In some embodiments, the at least two different targets comprise two different epitopes. In some embodiments, the two different epitopes are TL1A, a variant thereof or a functional fragment thereof, and a protein from any one of IL-6R, IL-6, IL-12, IL-23, IL-12p35, IL-12p40, IL-23p19, an IL-17 family cytokine, IL-17R, a variant thereof, and a functional fragment thereof. Multispecific molecules described herein may be considered multispecific antibodies.

Methods for making multispecific antibodies are known in the art. Traditionally, the recombinant production of multispecific antibodies is based on the co-expression of two immunoglobulin heavy-chain/light-chain pairs, where the two heavy chains have different specificities. The purification of the correct molecule is usually accomplished by affinity chromatography steps.

There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively. Accordingly, multispecific antibodies described herein comprise kappa constant region, lambda constant region, alpha constant region, gamma constant region, delta constant region, epsilon constant region, mu constant region, a functional fragment thereof, or a combination thereof.

A class of antibody or immunoglobulin refers to the type of constant domain or constant region possessed by its heavy chain. In some embodiment, the heavy chain is an IgA. In some embodiment, the heavy chain is an IgD. In some embodiment, the heavy chain is an IgE. In some embodiment, the heavy chain is an IgG. In some embodiment, the heavy chain is an IgM. In some embodiment, the heavy chain is an IgG1. In some embodiment, the heavy chain is an IgG2. In some embodiment, the heavy chain is an IgG3. In some embodiment, the heavy chain is an IgG4. In some embodiment, the heavy chain is an IgA1. In some embodiment, the heavy chain is an IgA2.

In some embodiments, an antibody is an IgG1 antibody. In some embodiments, an antibody is an IgG3 antibody. In some embodiments, an antibody is an IgG2 antibody. In some embodiments, an antibody is an IgG4 antibody. In some embodiments, an antibody is an IgA1 antibody. In some embodiments, an antibody is an IgA2 antibody.

In some embodiments, a multispecific antibody described herein comprise two heavy chains, wherein each heavy chain binds nonidentical epitopes. Each heavy chain can have at one end a variable domain (VH) followed by a number of constant domains (three or four constant domains, CH1, CH2, CH3 and CH4, depending on the antibody class). In some embodiments, multispecific antibodies described herein comprise one or more light chains. Each light chain can have a variable domain (VL) at one end and a constant domain (CL) at its other end; the constant domain of the light chain is aligned with the first constant domain (CH1) of the heavy chain, and the light chain variable domain is aligned with the variable domain of the heavy chain. In some embodiments, the light chains comprise kappa light chain or lambda light chain. Multispecific antibodies such as kappa or lambda antibodies can be made using any of a variety of art-recognized techniques, including those disclosed in WO 2012/023053, the contents of which are hereby incorporated by reference in their entirety.

In some embodiments, antibody variable domains with the desired binding specificities can be linked to immunoglobulin constant domain sequences to form multispecific antibodies. In some embodiments, the fusion preferably is with an immunoglobulin heavy-chain constant domain, comprising at least part of the hinge, CH2, and CH3 regions. In some embodiments, it is preferred to have the first heavy-chain constant region (CH1) containing the site necessary for light-chain binding present in at least one of the fusions. DNAs encoding the immunoglobulin heavy-chain fusions and, if desired, the immunoglobulin light chain, may be inserted into separate expression vectors, and may be co-transfected into a suitable host organism.

In some embodiments, the interface between a pair of antibody molecules in constructs herein is engineered to maximize the percentage of heterodimers which are recovered from recombinant cell culture. In this method, one or more small amino acid side chains from the interface of the first antibody molecule are replaced with larger side chains to form a protuberance or knob (e.g., tyrosine or tryptophan). Compensatory cavities or holes of identical or similar size to the large side chain(s) are created on the interface of the second antibody molecule by replacing large amino acid side chains with smaller ones (e.g., serine, threonine, valine or alanine). This provides a mechanism for increasing the yield of the heterodimer over other unwanted end-products such as homodimers.

Techniques for generating bispecific antibodies from antibody functional fragments have been described in the literature. For example, bispecific antibodies can be prepared using chemical linkage. The bispecific antibodies can be used as agents for the selective immobilization of enzymes.

Various techniques for making and isolating bispecific antibody functional fragments directly from recombinant cell culture have also been described. For example, bispecific antibodies have been produced using leucine zippers. The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology provides an alternative mechanism for making bispecific antibody functional fragments. The functional fragments comprise a heavy-chain variable domain (VH) connected to a light-chain variable domain (VL) by a linker which is too short to allow pairing between the two domains on the same chain. Accordingly, the VH and VL domains of one functional fragment are forced to pair with the complementary VL and VH domains of another functional fragment, thereby forming two target-binding sites. Another strategy for making bispecific antibody functional fragments includes use of single-chain Fv (sFv) dimers.

Antibodies with more than two valences are contemplated. For example, trispecific antibodies can be prepared. Exemplary bispecific antibodies can bind to two different epitopes, at least one of which originates in the target described herein. Alternatively, an anti-target arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular protein. Bispecific antibodies can also be used to direct cytotoxic agents to cells which express a particular protein. These antibodies may possess a target-binding arm and an arm which binds a cytotoxic agent or a radionuclide chelator, such as EOTUBE, DPTA, DOTA, or TETA. Another bispecific antibody of interest binds the target described herein and further binds tissue factor (TF).

Several strategies have been used to generate such multispecific molecules (e.g., bispecific molecules, trispecific molecules) such as chemical cross-linking of antibody functional fragments, forced heterodimerization, quadroma technology, fusion of antibody functional fragments via polypeptide linkers and use of single domain antibodies. The availability of recombinant DNA technologies has led to the generation of a multitude of bispecific antibody formats. Linkers and mutations have frequently been introduced into different regions of the antibody to force heterodimer formation or to connect different binding moieties into a single molecule.

In some embodiments, a multispecific antibody (e.g., bispecific antibody, trispecific antibody) comprises a first binding domain and a second binding domain, wherein the first binding domain binds a first target of TL1A, a variant thereof or a functional fragment thereof, and wherein a second binding domain binds a second target selected from IL-6R, IL-6, IL-12, IL-23, IL-23p19, IL-12p40, IL-12p35, an IL-17 family cytokine, IL-17R, a variant thereof and a functional fragment thereof. In some embodiments, a first binding domain comprises a first heavy chain variable region. In some embodiments, multispecific antibodies described herein comprise a first binding region, wherein the first binding region comprises a first heavy chain variable region and, optionally, a first light chain variable region. In some embodiments, a second binding domain comprises a second heavy chain variable region. In some embodiments, multispecific antibodies described herein comprise a second binding region, wherein the second binding region comprises a second heavy chain variable region and, optionally, a second light chain variable region. In some embodiments, the first heavy chain variable region and the first light chain variable region can bind the first target (TL1A, a variant thereof or a functional fragment thereof). In some embodiments, the second heavy chain variable region and the second light chain variable region can bind the second target (IL-6R, IL-6, IL-12, IL-23, IL-23p19, IL-12p40, IL-12p35, an IL-17 family cytokine, IL-17R, a variant thereof or a functional fragment thereof). In some embodiments, each of the first and second heavy chain variable regions comprises CDR-H1, CDR-H2, and CDR-H3. In some embodiments, each of the first and second light chain variable regions comprise CDR-L1, CDR-L2, and CDR-L3. In some embodiments, each of the first heavy chain variable region, the first light chain variable region, the second heavy chain variable region and the second light chain variable region comprise four framework regions. In some embodiments, at least one of the two heavy chain variable regions comprises a constant region (Fc). In some embodiments, at least one binding domain/binding region of a multispecific antibody comprises at least one modification that changes isoelectric point (pI) of the multispecific antibody. In some embodiments, the at least one modification in the variable region is within CDR-H1, CDR-H2, CDR-H3, at least one of framework regions of a heavy chain variable region, CDR-L1, CDR-L2, CDR-L3, at least one of framework regions of a light chain variable region, or combinations thereof. In some embodiments, the at least one modification in the variable region increases binding affinity of the multispecific antibody for at least one of the first and second target in neutral pH relative to binding affinity of a corresponding multispecific antibody prior to the at least one modification. In some embodiments, the at least one modification increases binding affinity of the multispecific antibody for at least one of the first and second target in neutral pH by at least 2%, at least 5%, at least 10%, at least 15%, at least 20%, at least 30%, at least 40%, at least 50% or more relative to binding affinity of a corresponding multispecific antibody prior to the at least one modification. Alternatively, in some embodiments, the at least one modification in CDR-H1, CDR-H2, CDR-H3, at least one of framework regions of a heavy chain variable region, CDR-L1, CDR-L2, CDR-L3, at least one of framework regions of a light chain variable region, or a combination thereof increases binding affinity of the multispecific antibody for at least one of the first and second target in neutral pH by at least 2%, at least 5%, at least 10%, at least 15%, at least 20%, at least 30%, at least 40%, at least 50% or more relative to binding affinity of a corresponding multispecific antibody prior to the at least one modification. In some embodiments, a binding affinity of the multispecific antibody comprising the at least one modification in the variable region for at least one of the first and second target in acidic pH remains within 5%, 10%, 15%, 20%, 25%, 30%, or 50% relative to binding affinity of a corresponding multispecific antibody prior to the at least one modification. Alternatively, in some embodiments, a binding affinity of the multispecific antibody comprising the at least one modification in CDR-H1, CDR-H2, CDR-H3, at least one of framework regions of a heavy chain variable region, CDR-L1, CDR-L2, CDR-L3, at least one of framework regions of a light chain variable region, or a combination thereof for at least one of the first and second target in acidic pH remains within 5%, 10%, 15%, 20%, 25%, 30%, or 50% relative to binding affinity of a corresponding multispecific antibody prior to the at least one modification. In some embodiments, the at least one modification in the variable region decreases binding affinity of the multispecific antibody for at least one of the first and second target in acidic pH by at least 2%, at least 5%, at least 10%, at least 15%, at least 20%, at least 30%, at least 40%, at least 50% or more relative to binding affinity of a corresponding multispecific antibody prior to the at least one modification. Alternatively, in some embodiments, the at least one modification in CDR-H1, CDR-H2, CDR-H3, at least one of framework regions of a heavy chain variable region, CDR-L1, CDR-L2, CDR-L3, at least one of framework regions of a light chain variable region, or a combination thereof decreases binding affinity of the multispecific antibody for at least one of the first and second target in acidic pH by at least 2%, at least 5%, at least 10%, at least 15%, at least 20%, at least 30%, at least 40%, at least 50% or more relative to binding affinity of a corresponding multispecific antibody prior to the at least one modification. In some embodiments, the at least one modification in a first heavy chain variable region and/or a first light chain variable region increases binding interaction with histidine rich binding pocket of TL1A, a variant thereof, or a functional fragment thereof. Likewise, in some embodiments, the at least one modification in a second heavy chain variable region and/or a second light chain variable region increases binding interaction with histidine rich binding pocket of IL-6R, IL-6, IL-12, IL-23, IL-23p19, IL-12p40, IL-12p35, an IL-17 family cytokine, IL-17R, a variant thereof or a functional fragment thereof.

In some embodiments, multispecific antibody described herein comprise at least one constant region. In some embodiments, a constant region comprises at least one modification. In some embodiments, the at least one modification increases binding affinity of the constant region for a neonatal fragment crystallizable receptor (FcRn) in acidic pH relative to binding affinity of a corresponding constant region prior to the at least one modification. In some embodiments, a binding affinity of the constant region comprising the at least one modification for FcRn is increased in acidic pH by at least 5%, at least 10%, at least 15%, at least 20%, at least 25%, at least 30%, at least 50%, at least 70% or more relative to binding affinity of a corresponding constant region prior to the at least one modification. In some embodiments, a binding affinity of the constant region comprising at least one modification for FcRn remains within remains within 5%, 10%, 15%, 20%, 25%, 30%, or 50% relative to binding affinity of a corresponding constant region prior to the at least one modification.

Alternatively, in some embodiments, at least one modification in a constant region increases binding affinity of the constant region for a neonatal fragment crystallizable receptor (FcRn) in neutral pH relative to binding affinity of a corresponding constant region prior to the at least one modification. Accordingly, in some embodiments, a binding affinity of the constant region comprising at least one modification is increased in neutral pH for FcRn by at least 5%, at least 10%, at least 15%, at least 20%, at least 25%, at least 30%, at least 50%, at least 70% or more relative to binding affinity of a corresponding constant region prior to the at least one modification. In some embodiments, a binding affinity of the constant region comprising at least one modification for FcRn in acidic pH remains within 5%, 10%, 15%, 20%, 25%, 30%, or 50% relative to binding affinity of a corresponding constant region prior to the at least one modification.

In some embodiments, the multispecific molecule (e.g., bispecific molecules, trispecific molecules) comprises a mutant Fc domain. In some embodiments, the mutant Fc domain comprises one or more mutations. In some embodiments, the multispecific molecule comprising a mutant Fc domain has a longer half-life relative to the multispecific molecule comprising unmodified Fc domain. In some embodiments, the mutant Fc domain comprises one or more of M252Y, S254T, and T256E mutations relative to corresponding wildtype Fc domain, wherein the mutations extend half-life of the multispecific molecule. In some embodiments, the mutant Fc domain comprises one or more of M428L and N434S mutations relative to corresponding wildtype Fc domain, wherein the mutations extend half-life of the multispecific molecule. In some embodiments, the mutant Fc domain comprises one or more of T307A, E380A, and N434A mutations relative to corresponding wildtype Fc domain, wherein the mutations extend half-life of the multispecific molecule. In some embodiments, the mutant Fc domain comprises one or more of T250Q and M428L mutations relative to corresponding wildtype Fc domain, wherein the mutations extend half-life of the multispecific molecule. In some embodiments, the mutant Fc domain comprises one or more of T307Q, Q311V, and A378V mutations relative to corresponding wildtype Fc domain, wherein the mutations extend half-life of the multispecific molecule. In some embodiments, the mutant Fc domain comprises one or more of T256D, H286D, T307R, Q311V, and A378V mutations relative to corresponding wildtype Fc domain, wherein the mutations extend half-life of the multispecific molecule. In some embodiments, the mutant Fc domain comprises one or more of H285D, T307Q, and A378V mutations relative to corresponding wildtype Fc domain, wherein the mutations extend half-life of the multispecific molecule. In some embodiments, the mutant Fc domain comprises one or more of T256D, Q311V, and A378V mutations relative to corresponding wildtype Fc domain, wherein the mutations extend half-life of the multispecific molecule. In some embodiments, the mutant Fc domain comprises one or more of H285N, T307Q, and N315D mutations relative to corresponding wildtype Fc domain, wherein the mutations extend half-life of the multispecific molecule. In some embodiments, the mutant Fc domain comprises one or more of L235A and G237A mutations relative to corresponding wildtype Fc domain, wherein the mutations extend half-life of the multispecific molecule.

In some embodiments, the multispecific molecule is used for subcutaneous administration. In some embodiments, a mutant Fc domain of the multispecific molecule comprises a deletion of a c-terminal lysine relative to corresponding wild-type Fc domain. In some embodiments, the deletion of the c-terminal lysine of the Fc domain improves subcutaneous bioavailability of the multispecific molecule. Accordingly, in some embodiments, subcutaneous administration of an effective amount of a composition comprising multispecific molecule to a subject in need thereof results in treatment of a disease or condition, wherein the multispecific molecule comprises a mutant Fc domain, wherein the mutant Fc domain comprises a deletion of a c-terminal lysine relative to corresponding wild-type Fc domain.

### IL-6R

IL-6 family proteins bind to Interleukin-6 receptor (IL-6R). The IL-6 family, as described herein, include Interleukin-6 cytokine (IL-6), Interleukin-11 cytokine (IL-11), a functional fragment thereof, a variant thereof, a multimeric form thereof or a combination thereof. In some embodiments, antibodies targeting IL-6R are used for treating rheumatoid arthritis, giant cell arthritis, systemic sclerosis-interstitial lung disease (SSc-ILD), juvenile idiopathic arthritis (JIA), cytokine release syndrome (CRS), corona virus disease (COVID-2019), polymyalgia rheumatica, neuromyelitis optica spectrum disorder (NMOSD), scleroderma-associated interstitial lung disease (SSc-ILD), or combinations thereof. Amino acid sequences of IL-6R is recited in TABLE 1.

TABLE 1
Amino Acid Sequences of IL-6R

| | SEQ | |
|---|---|---|
| | ID | |
| Name | NO: | Amino Acid Sequences |
| IL-6R | 1 | MLAVGCALLAALLAAPGAALAPRRCPAQEVARGVLTSLPGDSVTLTCPGVEPEDNA |
| | | TVHWVLRKPAAGSHPSRWAGMGRRLLLRSVQLHDSGNYSCYRAGRPAGTVHLLVDV |
| | | PPEEPQLSCFRKSPLSNVVCEWGPRSTPSLTTKAVLLVRKFQNSPAEDFQEPCQYS |
| | | QESQKFSCQLAVPEGDSSFYIVSMCVASSVGSKFSKTQTFQGCGILQPDPPANITV |
| | | TAVARNPRWLSVTWQDPHSWNSSFYRLRFELRYRAERSKTFTTWMVKDLQHHCVIH |
| | | DAWSGLRHVVQLRAQEEFGQGEWSEWSPEAMGTPWTESRSPPAENEVSTPMQALTT |
| | | NKDDDNILFRDSANATSLPVQDSSSVPLPTFLVAGGSLAFGTLLCIAIVLRFKKTW |
| | | KLRALKEGKTSMHPPYSLGQLVPERPRPTPVLVPLISPPVSPSSLGSDNTSSHNRP |
| | | DARDPRSPYDISNTDYFFPR |

In some embodiments, multispecific molecules described herein bind to an amino acid sequence that is at least 60%, at least 70%, at least 75%, at least 80%, at least 85%, at least 90%, at least 95%, at least 99% or 100% identical to the sequences recited in TABLE 1. In some embodiments, multispecific molecules described herein bind to an amino acid sequence that is at least 60%, 70%, 75%, 80%, 85%, 90%, 95%, 99% or 100% identical to SEQ ID NO: 1.

In some embodiments, multispecific molecules described herein comprise at least one of CDR-Hs described in TABLE 2 or a variant thereof, wherein the multispecific molecule is capable of binding an amino acid sequence that is at least 60%, 70%, 75%, 80%, 85%, 90%, 95%, 99% or 100% identical to any one of SEQ ID NOs: 2-10. In some embodiments, multispecific molecules described herein comprise any one of CDR-H1 described in TABLE 2 or a variant thereof, any one of CDR-H2 described in TABLE 2 or a variant thereof, and any one of CDR-H3 described in TABLE 2 or a variant thereof, wherein the multispecific molecule is capable of binding an amino acid sequence that is at least 60%, 70%, 75%, 80%, 85%, 90%, 95%, 99% or 100% identical to SEQ ID NO: 1. In some embodiments, multispecific molecules described herein comprise any one of combinations of CDR-Hs or variants thereof described in TABLE 2, wherein the multispecific molecule is capable of binding an amino acid sequence that is at least 60%, 70%, 75%, 80%, 85%, 90%, 95%, 99% or 100% identical to SEQ ID NO: 1. In some embodiments, the CDR-H variant comprises at least one, at least two or at least three substitutions, deletions, additions or combination thereof relative to a corresponding parent CDR-H sequence described in TABLE 2. In some embodiments, the CDR-H or the variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 2.

TABLE 2
Exemplary CDR-H sequences of antibodies for
binding to IL-6R

| | SEQ | | SEQ | | SEQ | |
|---|---|---|---|---|---|---|
| | ID | | ID | | ID | |
| Target | NO: | CDR-H1 | NO: | CDR-H2 | NO: | CDR-H3 |
| IL-6R | 2 | GFTFSSYY | 3 | IYSDGTTH | 4 | AKGAGPTWWYALDA |
| IL-6R | 5 | RFTFDDYA | 6 | ISWNSGRI | 7 | AKGRDSFDI |
| IL-6R | 8 | GHSISHDHA | 9 | FISYSGIT | 10 | ARSLARTTAMDY |

In some embodiments, multispecific antibodies described herein comprise at least one modification within any one of the CDR-Hs described in TABLE 2, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-6R, a variant thereof or a functional fragment thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 3, wherein the multispecific molecule is capable of binding an amino acid sequence that is at least 60%, 70%, 75%, 80%, 85%, 90%, 95%, 99% or 100% identical to SEQ ID NO: 1.

TABLE 3
Exemplary VH sequence of antibodies for binding to IL-6R

| SEQ | | |
|---|---|---|
| ID | | |
| NO: | Target | VH Sequences |
| 11 | IL-6R | QVQLVQSGGGLVQPGGSLRLSCAASGFTFSSYYMSWVRQAPGKGLEWVS |
| | | GIYSDGTTHYGDSVKGRFTISRDNAKNTVYLQLNSLRAEDTAMYYCAKG |
| | | AGPTWWYALDAWGQGTLVTVSS |
| 12 | IL-6R | EVQLVESGGGLVQPGRSLRLSCAASRFTFDDYAMHWVRQAPGKGLEWVS |
| | | GISWNSGRIGYADSVKGRFTISRDNAENSLFLQMNGLRAEDTALYYCAK |
| | | GRDSFDIWGQGTMVTVSS |
| 13 | IL-6R | QVQLQESGPGLVKPSETLSLTCAVSGHSISHDHAWSWVRQPPGEGLEWI |
| | | GFISYSGITNYNPSLQGRVTISRDNSKNTLYLQMNSLRAEDTAVYYCAR |
| | | SLARTTAMDYWGEGTLVTVSS |
| 14 | IL-6R | QVQLQESGPGLVRPSQTLSLTCTVSGYSITSDHAWSWVRQPPGRGLEWI |
| | | GYISYSGITTYNPSLKSRVTMLRDTSKNQFSLRLSSVTAADTAVYYCAR |
| | | SLARTTAMDYWGQGSLVTVSS |

In some embodiments, multispecific antibodies described herein comprise at least one modification within any one of the VH sequences described in TABLE 3, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-6R, a variant thereof or a functional fragment thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise at least one of CDR-Ls described in TABLE 4 or a variant thereof, wherein the multispecific molecule is capable of binding an amino acid sequence that is at least 60%, 70%, 75%, 80%, 85%, 90%, 95%, 99% or 100% identical to SEQ ID NO: 1. In some embodiments, multispecific molecules described herein comprise any one of CDR-L1 described in TABLE 4 or a variant thereof, any one of CDR-L2 described in TABLE 4 or a variant thereof, and any one of CDR-L3 described in TABLE 4 or a variant thereof, wherein the multispecific molecule is capable of binding an amino acid sequence that is at least 60%, 70%, 75%, 80%, 85%, 90%, 95%, 99% or 100% identical to SEQ ID NO: 1. In some embodiments, multispecific molecules described herein comprise any one of combinations of CDR-Ls or variants thereof described in TABLE 4, wherein the multispecific molecule is capable of binding an amino acid sequence that is at least 60%, 70%, 75%, 80%, 85%, 90%, 95%, 99% or 100% identical to SEQ ID NO: 1. In some embodiments, the CDR-L variant comprises at least one, at least two or at least three substitutions, deletions, additions or combination thereof relative to a corresponding parent CDR-L sequence described in TABLE 4. In some embodiments, the CDR-L or the variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 4.

TABLE 4
Exemplary CDR-L sequences of antibodies for binding to IL-6R

| | SEQ | | SEQ | | SEQ | |
|---|---|---|---|---|---|---|
| | ID | | ID | | ID | |
| Target | NO: | CDR-L1 | NO: | CDR-L2 | NO: | CDR-L3 |
| IL-6R | 15 | QSVLSASNTY | 16 | YAS | 17 | QQAYRAPVT |
| IL-6R | 18 | QGISSW | 19 | GAS | 20 | QQANSFPYT |
| IL-6R | 21 | TDISSH | 22 | YGS | 23 | GQGNRLPYT |

In some embodiments, multispecific antibodies described herein comprise at least one modification within any one of the CDR-Ls described in TABLE 4, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-6R, a variant thereof or a functional fragment thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of the combinations provided in TABLE 5.

TABLE 5
Exemplary CDR sequences of antibodies for binding to IL-6R

| Target | CDR-H1 | CDR-H2 | CDR-H3 | CDR-L1 | CDR-L2 | CDR-L3 |
|---|---|---|---|---|---|---|
| IL-6R | 2 | 3 | 4 | 15 | 16 | 17 |
| IL-6R | 5 | 6 | 7 | 18 | 19 | 20 |
| IL-6R | 8 | 9 | 10 | 21 | 22 | 23 |

In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 6, wherein the multispecific molecule is capable of binding an amino acid sequence that is at least 60%, 70%, 75%, 80%, 85%, 90%, 95%, 99% or 100% identical to SEQ ID NO: 1.

TABLE 6
Exemplary VL sequence of antibodies for binding to IL-6R

| SEQ ID NO: | Target | VL Sequences |
|---|---|---|
| 24 | IL-6R | DIQLTQSPSSVSVSVGERVTIDCKSSQSVLSASNTYLNWYQQKPGQ |
| | | APQLLIYYASTRESGVPDRFSGSGSGTDFTLTISSLQAEDAAVYYC |
| | | QQAYRAPVTFGQGTKLEIK |
| 25 | IL-6R | DIQMTQSPSSVSASVGDRVTITCRASQGISSWLAWYQQKPGKAPKL |
| | | LIYGASSLESGVPSRFSGSGSGTDFTLTISSLQPEDFASYYCQQAN |
| | | SFPYTFGQGTKLEIK |
| 26 | IL-6R | DIQMTQSPSSLSASVGDSVTITCQASTDISSHLNWYQQKPGKAPEL |
| | | LIYYGSHLLSGVPSRFSGSGSGTDFTFTISSLEAEDAATYYCGQGN |
| | | RLPYTFGQGTKVEIE |
| 27 | IL-6R | DIQMTQSPSSLSASVGDRVTITCRASQDISSYLNWYQQKPGKAPKL |
| | | LIYYTSRLHSGVPSRFSGSGSGTDFTFTISSLQPEDIATYYCQQGN |
| | | TLPYTFGQGTKVEIK |

In some embodiments, multispecific antibodies described herein comprise at least one modification within any one of the VL sequences described in TABLE 6, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-6R, a variant thereof or a functional fragment thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification. In some embodiments, multispecific antibodies described herein comprise two light chain variable region that are at least 90%, at least 95%, at least 98% or 100% identical relative to each other.

In some embodiments, multispecific antibodies comprise an IL-6R binding region, wherein the IL-6R binding region comprises an IL-6R binding heavy chain variable domain and, optionally, an IL-6R binding light chain variable domain. In some embodiments, multispecific antibodies described herein comprise a binding affinity for IL-6R, a variant thereof or a functional fragment thereof that is at least 10%, at least 20%, at least 30%, at least 50%, at least 80%, at least 100%, at least 200% or more higher under neutral pH condition relative to the binding affinity under acidic pH condition. In some embodiments, multispecific antibodies described herein comprise a ratio of binding affinities for IL-6R, a variant thereof or a functional fragment under neutral pH condition and acidic pH condition is at least 1.1, 1.5, 2, 3, 5, 8, 10, 100 or more. In some embodiments, multispecific antibodies described herein comprise a binding affinity for IL-6R, a variant thereof or a functional fragment thereof that is at least 10%, at least 20%, at least 30%, at least 50%, at least 80%, at least 100%, at least 200% or more higher at about pH 7.4 relative to the binding affinity at about pH 5.8. In some embodiments, multispecific antibodies described herein comprise a ratio of binding affinities for IL-6R, a variant thereof or a functional fragment at about pH 7.4 and about pH 5.8 is at least 1.1, 1.5, 2, 3, 5, 8, 10, 100 or more.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 3; and (b) a VL sequence that is at least 60%, at least 65%, at least 70%, at least 75%, at least 80%, at least 85%, at least 90%, at least 95% or 100% identical to any one of amino acid sequences described in TABLE 6, wherein the multispecific molecule comprises the VH sequence and the VL sequence according to the combination described in TABLE 7.

TABLE 7
Exemplary VH and VL sequences of antibodies for binding IL-6R

| | VH | VL |
|---|---|---|
| Target | (SEQ ID NO:) | (SEQ ID NO:) |
| IL-6R | 11 | 24 |
| IL-6R | 12 | 25 |
| IL-6R | 13 | 26 |
| IL-6R | 14 | 27 |

### IL-6

The IL-6 family, as described herein, include Interleukin-6 cytokine (IL-6), a functional fragment thereof, a variant thereof, a multimeric form thereof or a combination thereof. In some embodiments, antibodies targeting IL-6 are used for treating rheumatoid arthritis, giant cell arthritis, systemic sclerosis-interstitial lung disease (SSc-ILD), juvenile idiopathic arthritis (JIA), cytokine release syndrome (CRS), corona virus disease (COVID-2019), polymyalgia rheumatica, neuromyelitis optica spectrum disorder (NMOSD), scleroderma-associated interstitial lung disease (SSc-ILD), or combinations thereof. Amino acid sequences of IL-6 is recited in TABLE 8.

TABLE 8
Amino Acid Sequences of IL-6

| | SEQ | |
|---|---|---|
| | ID | |
| Name | NO: | Amino Acid Sequences |
| IL-6 | 221 | MNSFSTSAFGPVAFSLGLLLVLPAAFPAPVPPGEDSKDVAAPHRQPLTSSERI |
| | | DKQIRYILDGISALRKETCNKSNMCESSKEALAENNLNLPKMAEKDGCFQSGF |
| | | NEETCLVKIITGLLEFEVYLEYLQNRFESSEEQARAVQMSTKVLIQFLQKKAK |
| | | NLDAITTPDPTTNASLLTKLQAQNQWLQDMTTHLILRSFKEFLQSSLRALRQM |

In some embodiments, multispecific molecules described herein bind to an amino acid sequence that is at least 60%, at least 70%, at least 75%, at least 80%, at least 85%, at least 90%, at least 95%, at least 99% or 100% identical to the sequences recited in TABLE 8. In some embodiments, multispecific molecules described herein bind to an amino acid sequence that is at least 60%, 70%, 75%, 80%, 85%, 90%, 95%, 99% or 100% identical to SEQ ID NO: 221.

In some embodiments, multispecific molecules described herein comprise at least one of CDR-Hs described in TABLE 9 or a variant thereof, wherein the multispecific molecule is capable of binding an amino acid sequence that is at least 60%, 70%, 75%, 80%, 85%, 90%, 95%, 99% or 100% identical to any one of SEQ ID NOs: 222-239. In some embodiments, multispecific molecules described herein comprise any one of CDR-H1 described in TABLE 9 or a variant thereof, any one of CDR-H2 described in TABLE 9 or a variant thereof, and any one of CDR-H3 described in TABLE 9 or a variant thereof, wherein the multispecific molecule is capable of binding an amino acid sequence that is at least 60%, 70%, 75%, 80%, 85%, 90%, 95%, 99% or 100% identical to SEQ ID NO: 221. In some embodiments, multispecific molecules described herein comprise any one of combinations of CDR-Hs or variants thereof described in TABLE 9, wherein the multispecific molecule is capable of binding an amino acid sequence that is at least 60%, 70%, 75%, 80%, 85%, 90%, 95%, 99% or 100% identical to SEQ ID NO: 221. In some embodiments, the CDR-H variant comprises at least one, at least two or at least three substitutions, deletions, additions or combination thereof relative to a corresponding parent CDR-H sequence described in TABLE 9. In some embodiments, the CDR-H or the variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 9.

TABLE 9
Exemplary CDR-H sequences of antibodies for binding to IL-6

| | SEQ | | SEQ | | SEQ | |
|---|---|---|---|---|---|---|
| | ID | | ID | | ID | |
| Target | NO: | CDR-H1 | NO: | CDR-H2 | NO: | CDR-H3 |
| IL-6 | 222 | GFSLSNYY | 223 | IYGSDET | 224 | ARDDSSDWDAKFNL |
| IL-6 | 225 | GFNFNDYF | 226 | MRNKNYQYGT | 227 | ARESYYGFTSY |
| IL-6 | 228 | GFTFSSFA | 229 | ISSGGSYT | 230 | ARGLWGYYALDY |
| IL-6 | 231 | GFTFSPFA | 232 | ISPGGSWT | 233 | ARQLWGYYALDI |
| IL-6 | 234 | GYVLPNYL | 235 | TTPGGGTI | 236 | ARSRWDPLYYYALEY |
| IL-6 | 237 | GFTISSNY | 238 | LYYYAGDT | 239 | ARWADDHPPWIDL |

In some embodiments, multispecific antibodies described herein comprise at least one modification within any one of the CDR-Hs described in TABLE 9, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-6, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 10, wherein the multispecific molecule is capable of binding an amino acid sequence that is at least 60%, 70%, 75%, 80%, 85%, 90%, 95%, 99% or 100% identical to SEQ ID NO: 221.

TABLE 10
Exemplary VH sequence of antibodies for binding to IL-6

| SEQ ID NO: | Target | VH Sequences |
|---|---|---|
| 240 | IL-6 | EVQLVESGGGLVQPGGSLRLSCAASGFSLSNYYVTWVRQAPGK |
| | | GLEWVGIIYGSDETAYATSAIGRFTISRDNSKNTLYLQMNSLR |
| | | AEDTAVYYCARDDSSDWDAKFNLWGQGTLVTVSS |
| 241 | IL-6 | EVQLVESGGGLVQPGGSLRLSCAASGFNFNDYFMNWVRQAPGK |
| | | GLEWVAQMRNKNYQYGTYYAESLEGRFTISRDDSKNSLYLQMN |
| | | SLKTEDTAVYYCARESYYGFTSYWGQGTLVTVSS |
| 242 | IL-6 | EVQLVESGGKLLKPGGSLKLSCAASGFTFSSFAMSWFRQSPEK |
| | | RLEWVAEISSGGSYTYYPDTVTGRFTISRDNAKNTLYLEMSSL |
| | | RSEDTAMYYCARGLWGYYALDYWGQGTSVTVSS |
| 243 | IL-6 | EVQLVESGGGLVQPGGSLRLSCAASGFTFSPFAMSWVRQAPGK |
| | | GLEWVAKISPGGSWTYYSDTVTGRFTISRDNAKNSLYLQMNSL |
| | | RAEDTAVYYCARQLWGYYALDIWGQGTTVTVSS |
| 244 | IL-6 | QVQLVQSGAEVKKPGSSVKVSCKASGYVLPNYLIEWVRQAPGQ |
| | | GLEWMGVTTPGGGTINYAQKFQGRVTITADESTSTAYMELSSL |
| | | RSEDTAVYYCARSRWDPLYYYALEYWGQGTTVTVSS |
| 245 | IL-6 | EVQLVESGGGLVQPGGSLRLSCAASGFTISSNYMIWVRQAPGK |
| | | GLEWVSDLYYYAGDTYYADSVKGRFTMSRDISKNTVYLQMNSL |
| | | RAEDTAVYYCARWADDHPPWIDLWGRGTLVTVSS |

In some embodiments, multispecific antibodies described herein comprise at least one modification within any one of the VH sequences described in TABLE 10, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-6, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise at least one of CDR-Ls described in TABLE 11 or a variant thereof, wherein the multispecific molecule is capable of binding an amino acid sequence that is at least 60%, 70%, 75%, 80%, 85%, 90%, 95%, 99% or 100% identical to SEQ ID NO: 221. In some embodiments, multispecific molecules described herein comprise any one of CDR-L1 described in TABLE 11 or a variant thereof, any one of CDR-L2 described in TABLE 11 or a variant thereof, and any one of CDR-L3 described in TABLE 11 or a variant thereof, wherein the multispecific molecule is capable of binding an amino acid sequence that is at least 60%, 70%, 75%, 80%, 85%, 90%, 95%, 99% or 100% identical to SEQ ID NO: 221. In some embodiments, multispecific molecules described herein comprise any one of combinations of CDR-Ls or variants thereof described in TABLE 11, wherein the multispecific molecule is capable of binding an amino acid sequence that is at least 60%, 70%, 75%, 80%, 85%, 90%, 95%, 99% or 100% identical to SEQ ID NO: 221. In some embodiments, the CDR-L variant comprises at least one, at least two or at least three substitutions, deletions, additions or combination thereof relative to a corresponding parent CDR-L sequence described in TABLE 11. In some embodiments, the CDR-L or the variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 11.

TABLE 11
Exemplary CDR-L sequences of antibodies for binding to IL-6

| | SEQ | | SEQ | | SEQ | |
|---|---|---|---|---|---|---|
| | ID | | ID | | ID | |
| Target | NO: | CDR-L1 | NO: | CDR-L2 | NO: | CDR-L3 |
| IL-6 | 246 | QSINNE | 247 | RAS | 248 | QQGYSLRNIDNA |
| IL-6 | 249 | QDIGIS | 250 | NAN | 251 | LQHNSAPYT |
| IL-6 | 252 | SSVSY | 253 | DTS | 254 | QQWSGYPYT |
| IL-6 | 255 | ISVSY | 256 | DMS | 257 | MQWSGYPYT |
| IL-6 | 258 | ESVDNYGIPF | 259 | AAS | 260 | QQSEEVPLT |
| IL-6 | 261 | QGISSW | 262 | KAS | 263 | QQSWLGGS |

In some embodiments, multispecific antibodies described herein comprise at least one modification within any one of the CDR-Ls described in TABLE 11, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-6, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of the combinations provided in TABLE 12.

TABLE 12
Exemplary CDR sequences of antibodies for binding to IL-6

| Target | CDR-H1 | CDR-H2 | CDR-H3 | CDR-L1 | CDR-L2 | CDR-L3 |
|---|---|---|---|---|---|---|
| IL-6 | 222 | 223 | 224 | 246 | 247 | 248 |
| IL-6 | 225 | 226 | 227 | 249 | 250 | 251 |
| IL-6 | 228 | 229 | 230 | 252 | 253 | 254 |
| IL-6 | 231 | 232 | 233 | 255 | 256 | 257 |
| IL-6 | 234 | 235 | 236 | 258 | 259 | 260 |
| IL-6 | 237 | 238 | 239 | 261 | 262 | 263 |

In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 13, wherein the multispecific molecule is capable of binding an amino acid sequence that is at least 60%, 70%, 75%, 80%, 85%, 90%, 95%, 99% or 100% identical to SEQ ID NO: 221.

TABLE 13
Exemplary VL sequence of antibodies for binding to IL-6

| SEQ ID NO: | Target | VL Sequences |
|---|---|---|
| 264 | IL-6 | IQMTQSPSSLSASVGDRVTITCQASQSINNELSWYQQKPGKAPKLL |
| | | IYRASTLASGVPSRFSGSGSGTDFTLTISSLQPDDFATYYCQQGYS |
| | | LRNIDNAFGGGTKVEIK |
| 265 | IL-6 | DIQMTQSPSSLSASVGDRVTITCQASQDIGISLSWYQQKPGKAPKL |
| | | LIYNANNLADGVPSRFSGSGSGTDFTLTISSLQPEDFATYYCLQHN |
| | | SAPYTFGQGTKLEIK |
| 266 | IL-6 | QIVLIQSPAIMSASPGEKVTMTCSASSSVSYMYWYQQKPGSSPRLL |
| | | IYDTSNLASGVPVRFSGSGSGTSYSLTISRMEAEDAATYYCQQWSG |
| | | YPYTFGGGTKLEIK |
| 267 | IL-6 | EIVLTQSPATLSLSPGERATLSCSASISVSYMYWYQQKPGQAPRLL |
| | | IYDMSNLASGIPARFSGSGSGTDFTLTISSLEPEDFAVYYCMQWSG |
| | | YPYTFGGGTKVEIK |
| 268 | IL-6 | DIVMTQSPDSLAVSLGERATINCRASESVDNYGIPFMNWYQQKPGQ |
| | | PPKLLIYAASNRGSGVPDRFSGSGSGTDFTLTISSLQAEDVAVYYC |
| | | QQSEEVPLTFGQGTKLEIK |
| 269 | IL-6 | DIQMTQSPSTLSASVGDRVTITCRASQGISSWLAWYQQKPGKAPKV |
| | | LIYKASTLESGVPSRFSGSGSGTEFTLTISSLQPDDFATYYCQQSW |
| | | LGGSFGQGTKLEIK |

In some embodiments, multispecific antibodies described herein comprise at least one modification within any one of the VL sequences described in TABLE 13, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-6, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification. In some embodiments, multispecific antibodies described herein comprise two light chain variable region that are at least 90%, at least 95%, at least 98% or 100% identical relative to each other.

In some embodiments, multispecific antibodies comprise an IL-6 binding region, wherein the IL-6 binding region comprises an IL-6 binding heavy chain variable domain and, optionally, an IL-6 binding light chain variable domain. In some embodiments, multispecific antibodies described herein comprise a binding affinity for IL-6, a variant thereof or a functional fragment thereof that is at least 10%, at least 20%, at least 30%, at least 50%, at least 80%, at least 100%, at least 200% or more higher under neutral pH condition relative to the binding affinity under acidic pH condition. In some embodiments, multispecific antibodies described herein comprise a ratio of binding affinities for IL-6, a variant thereof or a functional fragment under neutral pH condition and acidic pH condition is at least 1.1, 1.5, 2, 3, 5, 8, 10, 100 or more. In some embodiments, multispecific antibodies described herein comprise a binding affinity for IL-6, a variant thereof or a functional fragment thereof that is at least 10%, at least 20%, at least 30%, at least 50%, at least 80%, at least 100%, at least 200% or more higher at about pH 7.4 relative to the binding affinity at about pH 5.8. In some embodiments, multispecific antibodies described herein comprise a ratio of binding affinities for IL-6, a variant thereof or a functional fragment at about pH 7.4 and about pH 5.8 is at least 1.1, 1.5, 2, 3, 5, 8, 10, 100 or more.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 10; and (b) a VL sequence that is at least 60%, at least 65%, at least 70%, at least 75%, at least 80%, at least 85%, at least 90%, at least 95% or 100% identical to any one of amino acid sequences described in TABLE 13, wherein the multispecific molecule comprises the VH sequence and the VL sequence according to the combination described in TABLE 14.

TABLE 14
Exemplary VH and VL sequences of antibodies for binding IL-6

| | VH | VL |
|---|---|---|
| Target | (SEQ ID NO:) | (SEQ ID NO:) |
| IL-6 | 240 | 264 |
| IL-6 | 241 | 265 |
| IL-6 | 242 | 266 |
| IL-6 | 243 | 267 |
| IL-6 | 244 | 268 |
| IL-6 | 245 | 269 |

### IL-12

Interleukin-12 (IL-12) is a heterodimeric cytokine comprising p40 subunit of IL-12 (IL-12p40) and p35 subunit of IL-12 (IL-12p35). Accordingly, the IL-12 family, as described herein, include IL12, IL-12p40, IL-12p35, a functional fragment thereof, a variant thereof, a multimeric form thereof or a combination thereof. Amino acid sequences of IL-12 family proteins are recited in TABLE 15.

TABLE 15
Amino Acid Sequences of IL-12

| | SEQ | |
|---|---|---|
| | ID | |
| Name | NO: | Amino Acid Sequences |
| IL- | 270 | MCPARSLLLVATLVLLDHLSLARNLPVATPDPGMFPCLHHSQNLLRAVSNM |
| 12p35 | | LQKARQTLEFYPCTSEEIDHEDITKDKTSTVEACLPLELTKNESCLNSRET |
| | | SFITNGSCLASRKTSFMMALCLSSIYEDLKMYQVEFKTMNAKLLMDPKRQI |
| | | FLDQNMLAVIDELMQALNFNSETVPQKSSLEEPDFYKTKIKLCILLHAFRI |
| | | RAVTIDRVMSYLNAS |
| IL- | 271 | MCHQQLVISWFSLVFLASPLVAIWELKKDVYVVELDWYPDAPGEMVVLTCD |
| 12p40 | | TPEEDGITWTLDQSSEVLGSGKTLTIQVKEFGDAGQYTCHKGGEVLSHSLL |
| | | LLHKKEDGIWSTDILKDQKEPKNKTFLRCEAKNYSGRFTCWWLTTISTDLT |
| | | FSVKSSRGSSDPQGVTCGAATLSAERVRGDNKEYEYSVECQEDSACPAAEE |
| | | SLPIEVMVDAVHKLKYENYTSSFFIRDIIKPDPPKNLQLKPLKNSRQVEVS |
| | | WEYPDTWSTPHSYFSLTFCVQVQGKSKREKKDRVFTDKTSATVICRKNASI |
| | | SVRAQDRYYSSSWSEWASVPCS |

In some embodiments, multispecific molecules described herein bind to IL-12, a subunit thereof, a functional fragment thereof, a variant thereof, a multimeric form thereof or a combination thereof. In some embodiments, multispecific molecules bind to an amino acid sequence that is at least 60%, at least 70%, at least 75%, at least 80%, at least 85%, at least 90%, at least 95%, at least 99% or 100% identical to the sequences recited in TABLE 15. In some embodiments, multispecific molecules bind to an amino acid sequence that is at least 60%, 70%, 75%, 80%, 85%, 90%, 95%, 99% or 100% identical to SEQ ID NO: 270. In some embodiments, multispecific molecules bind to an amino acid sequence that is at least 60%, 70%, 75%, 80%, 85%, 90%, 95%, 99% or 100% identical to SEQ ID NO: 271.

In some embodiments, multispecific molecules described herein comprise at least one of CDR-Hs described in TABLE 16 or a variant thereof, wherein the multispecific molecule is capable of binding an amino acid sequence that is at least 60%, 70%, 75%, 80%, 85%, 90%, 95%, 99% or 100% identical to any one of SEQ ID NOs: 272-280. In some embodiments, multispecific molecules described herein comprise any one of CDR-H1 described in TABLE 16 or a variant thereof, any one of CDR-H2 described in TABLE 16 or a variant thereof, and any one of CDR-H3 described in TABLE 16 or a variant thereof, wherein the multispecific molecule is capable of binding an amino acid sequence that is at least 60%, 70%, 75%, 80%, 85%, 90%, 95%, 99% or 100% identical to SEQ ID NO: 271. In some embodiments, multispecific molecules described herein comprise any one of combinations of CDR-Hs or variants thereof described in TABLE 16, wherein the multispecific molecule is capable of binding an amino acid sequence that is at least 60%, 70%, 75%, 80%, 85%, 90%, 95%, 99% or 100% identical to SEQ ID NO: 271. In some embodiments, the CDR-H variant comprises at least one, at least two or at least three substitutions, deletions, additions or combination thereof relative to a corresponding parent CDR-H sequence described in TABLE 16. In some embodiments, the CDR-H or the variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 16.

TABLE 16
Exemplary CDR-H sequences of antibodies for binding to IL-12

| | SEQ | | SEQ | | SEQ | |
|---|---|---|---|---|---|---|
| | ID | | ID | | ID | |
| Target | NO: | CDR-H1 | NO: | CDR-H2 | NO: | CDR-H3 |
| IL-12p40 | 272 | GFTFSSYG | 273 | IRYDGSNK | 274 | KTHGSHDN |
| IL-12p40 | 275 | GYTFTSYW | 276 | MSPVDSDI | 277 | ARRRPGQGYFDF |
| IL-12p40 | 278 | GYSFTTYW | 279 | MSPVDSDI | 280 | ARRRPGQGYFDF |

In some embodiments, multispecific antibodies described herein comprise at least one modification within any one of the CDR-Hs described in TABLE 16, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-12, a subunit thereof, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 17, wherein the multispecific molecule is capable of binding an amino acid sequence that is at least 60%, 70%, 75%, 80%, 85%, 90%, 95%, 99% or 100% identical to SEQ ID NO: 271.

TABLE 17
Exemplary VH sequence of antibodies for binding to IL-12

| SEQ ID | | |
|---|---|---|
| NO: | Target | VH Sequences |
| 281 | IL-12p40 | QVQLVESGGGVVQPGRSLRLSCAASGFTFSSYGMHWVRQAPGKGLEWVA |
| | | FIRYDGSNKYYADSVKGRFTISRDNSKNTLYLQMNSLRAEDTAVYYCKT |
| | | HGSHDNWGQGTMVTVSS |
| 282 | IL-12p40 | EVQLVQSGAEVKKPGESLKISCQSSGYTFTSYWIGWVRQMPGQGLEWIG |
| | | IMSPVDSDIRYNPMFRGQVTMSVDKSSSTAYLQWSSLKASDTAMYYCAR |
| | | RRPGQGYFDFWGQGTMVTVSS |
| 283 | IL-12p40 | EVQLVQSGAEVKKPGESLKISCKGSGYSFTTYWLGWVRQMPGKGLDWIG |
| | | IMSPVDSDIRYSPSFQGQVTMSVDKSITTAYLQWNSLKASDTAMYYCAR |
| | | RRPGQGYFDFWGQGTLVTVSS |

In some embodiments, multispecific antibodies described herein comprise at least one modification within any one of the VH sequences described in TABLE 17, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-12, a subunit thereof, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise at least one of CDR-Ls described in TABLE 18 or a variant thereof, wherein the multispecific molecule is capable of binding an amino acid sequence that is at least 60%, 70%, 75%, 80%, 85%, 90%, 95%, 99% or 100% identical to SEQ ID NO: 271. In some embodiments, multispecific molecules described herein comprise any one of CDR-L1 described in TABLE 18 or a variant thereof, any one of CDR-L2 described in TABLE 18 or a variant thereof, and any one of CDR-L3 described in TABLE 18 or a variant thereof, wherein the multispecific molecule is capable of binding an amino acid sequence that is at least 60%, 70%, 75%, 80%, 85%, 90%, 95%, 99% or 100% identical to SEQ ID NO: 271. In some embodiments, multispecific molecules described herein comprise any one of combinations of CDR-Ls or variants thereof described in TABLE 18, wherein the multispecific molecule is capable of binding an amino acid sequence that is at least 60%, 70%, 75%, 80%, 85%, 90%, 95%, 99% or 100% identical to SEQ ID NO: 271. In some embodiments, the CDR-L variant comprises at least one, at least two or at least three substitutions, deletions, additions or combination thereof relative to a corresponding parent CDR-L sequence described in TABLE 18. In some embodiments, the CDR-L or the variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 18.

TABLE 18
Exemplary CDR-L sequences of antibodies for binding to IL-12

| | SEQ | | SEQ | | SEQ | |
|---|---|---|---|---|---|---|
| | ID | | ID | | ID | |
| Target | NO: | CDR-L1 | NO: | CDR-L2 | NO: | CDR-L3 |
| IL-12p40 | 284 | RSNIGSNT | 293 | YND | 294 | QSYDRYTHPALL |
| IL-12p40 | 287 | QSVGTW | 296 | AAS | 297 | QQYNIYPYT |
| IL-12p40 | 290 | QGISSW | 299 | AAS | 300 | QQYNIYPYT |

In some embodiments, multispecific antibodies described herein comprise at least one modification within any one of the CDR-Ls described in TABLE 18, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-12, a subunit thereof, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of the combinations provided in TABLE 19.

TABLE 19
Exemplary CDR sequences of antibodies for binding to IL-12

| Target | CDR-H1 | CDR-H2 | CDR-H3 | CDR-L1 | CDR-L2 | CDR-L3 |
|---|---|---|---|---|---|---|
| IL-12p40 | 272 | 273 | 274 | 284 | 293 | 294 |
| IL-12p40 | 275 | 276 | 277 | 287 | 296 | 297 |
| IL-12p40 | 278 | 279 | 280 | 290 | 299 | 300 |

In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 20, wherein the multispecific molecule is capable of binding an amino acid sequence that is at least 60%, 70%, 75%, 80%, 85%, 90%, 95%, 99% or 100% identical to SEQ ID NO: 271.

TABLE 20
Exemplary VL sequence of antibodies for binding to IL-12

| SEQ ID NO: | Target | VL Sequences |
|---|---|---|
| 301 | IL- | QSVLTQPPSVSGAPGQRVTISCSGSRSNIGSNTVKWYQQLPGTAPK |
| | 12p40 | LLIYYNDQRPSGVPDRFSGSKSGTSASLAITGLQAEDEADYYCQSY |
| | | DRYTHPALLFGTGTKVTVL |
| 302 | IL- | EIVLTQSPATLSASPGERATISCRASQSVGTWVAWYQQKPGQAPRS |
| | 12p40 | LIYAASNLQSGIPARFSGSGSGTDFTLTISSLEPEDFAVYYCQQYN |
| | | IYPYTFGQGTRLEIK |
| 303 | IL- | DIQMTQSPSSLSASVGDRVTITCRASQGISSWLAWYQQKPEKAPKS |
| | 12p40 | LIYAASSLQSGVPSRFSGSGSGTDFTLTISSLQPEDFATYYCQQYN |
| | | IYPYTFGQGTKLEIK |

In some embodiments, multispecific antibodies described herein comprise at least one modification within any one of the VL sequences described in TABLE 20, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-12, a subunit thereof, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification. In some embodiments, multispecific antibodies described herein comprise two light chain variable region that are at least 90%, at least 95%, at least 98% or 100% identical relative to each other.

In some embodiments, multispecific antibodies comprise an IL-12 binding region, wherein the IL-12 binding region comprises an IL-12 binding heavy chain variable domain and, optionally, an IL-12 binding light chain variable domain. In some embodiments, multispecific antibodies described herein comprise a binding affinity for IL-12, a variant thereof or a functional fragment thereof that is at least 10%, at least 20%, at least 30%, at least 50%, at least 80%, at least 100%, at least 200% or more higher under neutral pH condition relative to the binding affinity under acidic pH condition. In some embodiments, multispecific antibodies described herein comprise a ratio of binding affinities for IL-12, a variant thereof or a functional fragment under neutral pH condition and acidic pH condition is at least 1.1, 1.5, 2, 3, 5, 8, 10, 100 or more. In some embodiments, multispecific antibodies described herein comprise a binding affinity for IL-12, a variant thereof or a functional fragment thereof that is at least 10%, at least 20%, at least 30%, at least 50%, at least 80%, at least 100%, at least 200% or more higher at about pH 7.4 relative to the binding affinity at about pH 5.8. In some embodiments, multispecific antibodies described herein comprise a ratio of binding affinities for IL-12, a variant thereof or a functional fragment at about pH 7.4 and about pH 5.8 is at least 1.1, 1.5, 2, 3, 5, 8, 10, 100 or more.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 17; and (b) a VL sequence that is at least 60%, at least 65%, at least 70%, at least 75%, at least 80%, at least 85%, at least 90%, at least 95% or 100% identical to any one of amino acid sequences described in TABLE 20, wherein the multispecific molecule comprises the VH sequence and the VL sequence according to the combination described in TABLE 21.

TABLE 21
Exemplary VH and VL sequences of antibodies for binding IL-12

| | VH | VL |
|---|---|---|
| Target | (SEQ ID NO:) | (SEQ ID NO:) |
| IL-12p40 | 281 | 301 |
| IL-12p40 | 282 | 302 |
| IL-12p40 | 283 | 303 |

### IL-23

Interleukin-23 (IL-23) is a heterodimeric cytokine comprising p19 subunit of IL-23 (IL-23p19) and IL-12p40. Accordingly, the IL-23 family, as described herein, includes IL-23, IL-23p19, IL-12p40, a functional fragment thereof, a variant thereof, a multimeric form thereof or a combination thereof. Amino acid sequences of IL-23p19 and IL-12p40 are recited in TABLE 22.

TABLE 22
Amino Acid Sequences of IL-23

| | SEQ | |
|---|---|---|
| | ID | |
| Name | NO: | Amino Acid Sequences |
| IL- | 271 | MCHQQLVISWFSLVFLASPLVAIWELKKDVYVVELDWYPDAPGEMVVLTCDT |
| 12p40 | | PEEDGITWTLDQSSEVLGSGKTLTIQVKEFGDAGQYTCHKGGEVLSHSLLLL |
| | | HKKEDGIWSTDILKDQKEPKNKTFLRCEAKNYSGRFTCWWLTTISTDLTFSV |
| | | KSSRGSSDPQGVTCGAATLSAERVRGDNKEYEYSVECQEDSACPAAEESLPI |
| | | EVMVDAVHKLKYENYTSSFFIRDIIKPDPPKNLQLKPLKNSRQVEVSWEYPD |
| | | TWSTPHSYFSLTFCVQVQGKSKREKKDRVFTDKTSATVICRKNASISVRAQD |
| | | RYYSSSWSEWASVPCS |
| IL- | 304 | MLGSRAVMLLLLLPWTAQGRAVPGGSSPAWTQCQQLSQKLCTLAWSAHPLVG |
| 23p19 | | HMDLREEGDEETTNDVPHIQCGDGCDPQGLRDNSQFCLQRIHQGLIFYEKLL |
| | | GSDIFTGEPSLLPDSPVGQLHASLLGLSQLLQPEGHHWETQQIPSLSPSQPW |
| | | QRLLLRFKILRSLQAFVAVAARVFAHGAATLSP |

In some embodiments, multispecific molecules described herein bind to IL-23, a subunit thereof, a functional fragment thereof, a variant thereof, a multimeric form thereof or a combination thereof. In some embodiments, the multispecific molecules bind to an amino acid sequence that is at least 60%, at least 70%, at least 75%, at least 80%, at least 85%, at least 90%, at least 95%, at least 99% or 100% identical to the sequences recited in TABLE 22. In some embodiments, multispecific molecules bind to an amino acid sequence that is at least 60%, 70%, 75%, 80%, 85%, 90%, 95%, 99% or 100% identical to SEQ ID NO: 304. In some embodiments, multispecific molecules bind to an amino acid sequence that is at least 60%, 70%, 75%, 80%, 85%, 90%, 95%, 99% or 100% identical to SEQ ID NO: 271.

In some embodiments, multispecific molecules described herein comprise at least one of CDR-Hs described in TABLE 23 or a variant thereof, wherein the multispecific molecule is capable of binding an amino acid sequence that is at least 60%, 70%, 75%, 80%, 85%, 90%, 95%, 99% or 100% identical to any one of SEQ ID NOs: 305-370. In some embodiments, multispecific molecules described herein comprise any one of CDR-H1 described in TABLE 23 or a variant thereof, any one of CDR-H2 described in TABLE 23 or a variant thereof and any one of CDR-H3 described in TABLE 23 or a variant thereof, wherein the multispecific molecule is capable of binding an amino acid sequence that is at least 60%, 70%, 75%, 80%, 85%, 90%, 95%, 99% or 100% identical to SEQ ID NO: 304. In some embodiments, multispecific molecules described herein comprise any one of combinations of CDR-Hs or variants thereof described in TABLE 23, wherein the multispecific molecule is capable of binding an amino acid sequence that is at least 60%, 70%, 75%, 80%, 85%, 90%, 95%, 99% or 100% identical to SEQ ID NO: 304. In some embodiments, the CDR-H variant comprises at least one, at least two or at least three substitutions, deletions, additions or combination thereof relative to a corresponding parent CDR-H sequence described in TABLE 23. In some embodiments, the CDR-H or the variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 23.

TABLE 23
Exemplary CDR-H sequences of antibodies for binding to IL-23

| | SEQ | | SEQ | | SEQ | |
|---|---|---|---|---|---|---|
| | ID | | ID | | ID | |
| Target | NO: | CDR-H1 | NO: | CDR-H2 | NO: | CDR-H3 |
| IL- | 305 | GFTFSSYG | 306 | IWYDGSNE | 307 | ARDRGYTSSWYPDAFDI |
| 23p19 | | | | | | |
| IL- | 308 | GYSFSNYW | 309 | IDPSNSYT | 310 | ARWYYKPFDV |
| 23p19 | | | | | | |
| IL- | 311 | GYKFTRYV | 312 | INPYNDGT | 313 | ARNWDTGL |
| 23p19 | | | | | | |
| IL- | 314 | GYTFTSYL | 315 | INPYNEGT | 316 | ARNWDLPY |
| 23p19 | | | | | | |
| IL- | 317 | GYTFTDQT | 318 | IYPRDDSP | 319 | AIPDRSGYAWFIY |
| 23p19 | | | | | | |
| IL- | 320 | GYIFITYW | 321 | IFPASGSA | 322 | ARGGGGFAY |
| 23p19 | | | | | | |
| IL-23 | 323 | SYGMH | 324 | VIWYDGSNEYYADSVKG | 325 | DRGYTSSWYPDA |
| IL-23 | 323 | SYGMH | 327 | VIWYDGSNKYYADSVKG | 328 | DRGYSSSWYPDAFD |
| IL-23 | 323 | SYGMH | 330 | VISFDGSLKYYADSVKG | 331 | ERTTLSGSYFDY |
| IL-23 | 332 | SYAMH | 333 | VISHDGSIKYYADSVKG | 331 | ERTTLSGSYFDY |
| IL-23 | 335 | SYSMN | 336 | YISSRSSTIYIADSVKG | 337 | RIAAAGGFHYYYALDV |
| IL-23 | 338 | TYSMN | 339 | YISSSSSTRYHADSVKG | 340 | RIAAAGPWGYYYAMDV |
| IL-23 | 341 | SFSMN | 342 | YISSRSSTIYYADSVKG | 340 | RIAAAGPWGYYYAMDV |
| IL-23 | 344 | TYYWS | 345 | LIYTSGSTNYNPSLKS | 346 | DRGYYYGVDV |
| IL-23 | 347 | SGGYYWS | 348 | HIHYSGNTYYNPSLKS | 349 | NRGFYYGMDV |
| IL-23 | 347 | SGGYYWS | 351 | YIYYSGSSYYNPSLKS | 352 | DRGHYYGMDV |
| IL-23 | 347 | SGGYYWS | 354 | YIYYSGSTYYNPSLKS | 352 | DRGHYYGMDV |
| IL-23 | 356 | SYFWS | 357 | YIYYSGSTNYNPSLKS | 358 | DRGSYYGSDY |
| IL-23 | 359 | SGGYYWT | 360 | YIYYSGNTYYNPSLKS | 361 | NRGYYYGMDV |
| IL-23 | 347 | SGGYYWS | 354 | YIYYSGSTYYNPSLKS | 349 | NRGFYYGMDV |
| IL-23 | 347 | SGGYYWS | 351 | YIYYSGSSYYNPSLKS | 352 | DRGHYYGMDV |
| IL-23 | 323 | SYGMH | 369 | LIWYDGSNKYYADSVKG | 370 | ENTVTIYYNYGMDV |
| IL-23 | 867 | IYAIS | 786 | GILPIFGTGHYVQKFQG | 739 | EPEN |
| IL-23 | 893 | GDSIH | 857 | WITPNSGGTSYAQRFQG | 774 | GLTVPFDY |
| IL-23 | 867 | IYAIS | 786 | GILPIFGTGHYVQKFQG | 742 | EPED |

In some embodiments, multispecific antibodies described herein comprise at least one modification within any one of the CDR-Hs described in TABLE 23, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-23, a subunit thereof, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 24, wherein the multispecific molecule is capable of binding an amino acid sequence that is at least 60%, 70%, 75%, 80%, 85%, 90%, 95%, 99% or 100% identical to SEQ ID NO: 304.

TABLE 24
Exemplary VH sequence of antibodies for binding to IL-23

| SEQ ID | | |
|---|---|---|
| NO: | Target | VH Sequences |
| 371 | IL- | QVQLVESGGGVVQPGRSLRLSCAASGFTFSSYGMHWVRQAPGKGLE |
| | 23p19 | WVAVIWYDGSNEYYADSVKGRFTISRDNSKNTLYLQMNSLRAEDTA |
| | | VYYCARDRGYTSSWYPDAFDIWGQGTMVTVSS |
| 372 | IL- | EVQLVQSGAEVKKPGESLKISCKGSGYSFSNYWIGWVRQMPGKGLE |
| | 23p19 | WMGIIDPSNSYTRYSPSFQGQVTISADKSISTAYLQWSSLKASDTA |
| | | MYYCARWYYKPFDVWGQGTLVTVSS |
| 373 | IL- | QVQLVQSGAEVKKPGSSVKVSCKASGYKFTRYVMHWVRQAPGQGLE |
| | 23p19 | WMGYINPYNDGTNYNEKFKGRVTITADKSTSTAYMELSSLRSEDTA |
| | | VYYCARNWDTGLWGQGTTVTVSS |
| 374 | IL- | QVQLVQSGAEVKKPGASVKVSCKASGYTFTSYLMHWVRQAPGQGLE |
| | 23p19 | WMGYINPYNEGTNYAQKFQGRVTMTRDTSISTAYMELSRLRSDDTA |
| | | VYYCARNWDLPYWGQGTLVTVSS |
| 375 | IL- | QVQLVQSGAEVKKPGSSVKVSCKASGYTFTDQTIHWMRQAPGQGLE |
| | 23p19 | WIGYIYPRDDSPKYNENFKGKVTITADKSTSTAYMELSSLRSEDTA |
| | | VYYCAIPDRSGYAWFIYWGQGTLVTVSS |
| 376 | IL- | QVQLVQSGAEVKKPGASVKVSCKASGYIFITYWMTWVRQAPGQGLE |
| | 23p19 | WMGQIFPASGSADYNEKFEGRVTMTTDTSTSTAYMELRSLRSDDTA |
| | | VYYCARGGGGFAYWGQGTLVTVSS |
| 283 | IL-23 | EVQLVQSGAEVKKPGESLKISCKGSGYSFTTYWLGWVRQMPGKGLD |
| | | WIGIMSPVDSDIRYSPSFQGQVTMSVDKSITTAYLQWNSLKASDTA |
| | | MYYCARRRPGQGYFDFWGQGTLVTVSS |
| 377 | IL-23 | QVQLVESGGGWQPGRSLRLSCAASGFTFSSYGMHWVRQAPGKGLEW |
| | | VAVIWYDGSNEYYADSVKGRFTISRDNSKNTLYLQMNSLRAEDTAV |
| | | YYCARDRGYTSSWYPDAFDIWGQGTMVTVSS |
| 378 | IL-23 | QVQLVESGGGWQPGRSLRLSCAASGFTFSSYGMHWVRQAPGKGLEW |
| | | VAVIWYDGSNKYYADSVKGRFTISRDNSKNTLYLQMNSLRAEDTAV |
| | | YYCARDRGYSSSWYPDAFDIWGQGTMVTVSS |
| 379 | IL-23 | QVQLVESGGGWQPGRSLRLSCAASGFTFSSYGMHWVRQAPGKGLEW |
| | | VAVISFDGSLKYYADSVKGRFTISRDNSKNTLYLQMNSLRAEDTAV |
| | | YYCARERTTLSGSYFDYWGQGTLVTVSS |
| 380 | IL-23 | QVQLVESGGGWQPGRSLRLSCAASGFTFSSYAMHWVRQAPGKGLEW |
| | | LSVISHDGSIKYYADSVKGRFTISRDNSKNTLYLQMNSLRAEDTAV |
| | | YYCARERTTLSGSYFDYWGQGTLVTVSS |
| 381 | IL-23 | EVQLVESGGGLVQPGGSLRLSCAASGFTFSSYSMNWVRQAPGKGLE |
| | | WVSYISSRSSTIYIADSVKGRFTISRDNAKNSLYLQMNSLRDEDTA |
| | | VYYCARRIAAAGGFHYYYALDVWGQGTTVTVSS |
| 382 | IL-23 | EVQLVESGGGLVQPGGSLRLSCAASGFTFSTYSMNWVRQAPGKGLE |
| | | WVSYISSSSSTRYHADSVKGRFTISRDNAKNSLYLQMNSLRDEDTA |
| | | VYYCARRIAAAGPWGYYYAMDVWGQGTTVTVSS |
| 383 | IL-23 | EVQLVESGGGLVQPGGSLRLSCWSGFTFSSFSMNWVRQAPGKGLEW |
| | | VSYISSRSSTIYYADSVKGRFTISRDNAKNSLYLQMNSLRDEDTAV |
| | | YYCARRIAAAGPWGYYYAMDVWGQGTTVTVSS |
| 384 | IL-23 | QVQLQESGPGLVKPSETLSLTCTVSGGSISTYYWSWIRQPAGKGLE |
| | | WIGLIYTSGSTNYNPSLKSRVTMSLDTSKNQFSLRLTSVTAADTAV |
| | | YYCARDRGYYYGVDVWGQGTTVTVSS |
| 385 | IL-23 | QVQLQESGPGLVKPSQTLSLTCTVSGGSISSGGYYWSWIRQHPGKG |
| | | LEWIGHIHYSGNTYYNPSLKSRVTISVDTSKNQFSLKLSSVTAADT |
| | | AVYYCAKNRGFYYGMDVWGQGTTVTVSS |
| 386 | IL-23 | QVQLQESGPGLVKPSQTLSLTCTVSGGSINSGGYYWSWIRQHPGKG |
| | | LEWIGYIYYSGSSYYNPSLKSRVTISVDTSQNQFSLKLSSVTAADT |
| | | AVYYCARDRGHYYGMDVWGQGTTVTVSS |
| 387 | IL-23 | QVQLQESGPGLVKPSQTLSLTCTVSGGSISSGGYYWSWIRQHPGKG |
| | | LEWIGYIYYSGSTYYNPSLKSRVTISVDTSKNQFSLKLSSVTAADT |
| | | AVYYCARDRGHYYGMDVWGQGTTVTVSS |
| 388 | IL-23 | QVQLQESGPRLVKPSETLSLTCTVSGDSISSYFWSWIRQPPGKGLE |
| | | WLGYIYYSGSTNYNPSLKSRVTISIDTSKNQFSLKLSSVTAADTAV |
| | | YYCTRDRGSYYGSDYWGQGTLVTVSS |
| 389 | IL-23 | QVQLQESGPGLVKPSQTLSLTCTVSGGSISSGGYYWTWIRQHPGKG |
| | | LEWIGYIYYSGNTYYNPSLKSRITISVDTSKNQFSLSLSSVTAADT |
| | | AVYYCARNRGYYYGMDVWGQGTTVTVSS |
| 390 | IL-23 | QVQLQESGPGLVKPSQTLSLTCTVSGGSISSGGYYWSWIRQHPGKG |
| | | LEWIGYIYYSGSTYYNPSLKSRVTMSVDTSKNQFSLKLSSVTAADT |
| | | AVYYCAKNRGFYYGMDVWGQGTTVTVSS |
| 391 | IL-23 | QVQLQESGPGLVKPSQTLSLTCTVSGGSINSGGYYWSWIRQHPGKG |
| | | LEWIGYIYYSGSSYYNPSLKSRVTISVDTSKNQFSLKLSSVTAADT |
| | | AVYYCARDRGHYYGMDVWGQGTTVTVSS |
| 392 | IL-23 | QVQLVESGGGWQPGRSLRLSCAASGFTFSSYGMHWVRQAPGKGLEW |
| | | VALIWYDGSNKYYADSVKGRFTISRDNSKNTLYLQMNSLRAEDTAV |
| | | YYCARENTVTIYYNYGMDVWGQGTTVTVSS |
| 1466 | IL-23 | QVQLVQSGAEVKKPGSSVKVSCKASGGTFTIYAISWVRQAPGQGLE |
| | | WMGGILPIFGTGHYVQKFQGRVTITADESTSTSYMELSSLRSEDTA |
| | | VYYCAREPEDWGQGTLVTVSS |
| 1544 | IL-23 | QVQLVQSGAEVKKPGSSVKVSCKASGGTFTIYAISWVRQAPGQGLE |
| | | WMGGILPIFGTGHYVQKFQGRVTITADESTSTSYMELSSLRSEDTA |
| | | VYYCAREPENWGQGTLVTVSS |
| 1621 | IL-23 | QVQLVQSGAEVKKPGASVKVSCTASGDAFTGDSIHWVRQAPGQGLE |
| | | WMGWITPNSGGTSYAQRFQGRVTLTRDTSISTVYMDLRRLRSDDTA |
| | | VYFCASGLTVPFDYWGQGTLVTVSS |

In some embodiments, multispecific antibodies described herein comprise at least one modification within any one of the VH sequences described in TABLE 24, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-23, a subunit thereof, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise at least one of CDR-Ls described in TABLE 25 or a variant thereof, wherein the multispecific molecule is capable of binding an amino acid sequence that is at least 60%, 70%, 75%, 80%, 85%, 90%, 95%, 99% or 100% identical to SEQ ID NO: 304. In some embodiments, multispecific molecules described herein comprise any one of CDR-L1 described in TABLE 25 or a variant thereof, any one of CDR-L2 described in TABLE 25 or a variant thereof, and any one of CDR-L3 described in TABLE 25 or a variant thereof, wherein the multispecific molecule is capable of binding an amino acid sequence that is at least 60%, 70%, 75%, 80%, 85%, 90%, 95%, 99% or 100% identical to SEQ ID NO: 304. In some embodiments, multispecific molecules described herein comprise any one of combinations of CDR-Ls or variants thereof described in TABLE 25, wherein the multispecific molecule is capable of binding an amino acid sequence that is at least 60%, 70%, 75%, 80%, 85%, 90%, 95%, 99% or 100% identical to SEQ ID NO: 304. In some embodiments, the CDR-L variant comprises at least one, at least two or at least three substitutions, deletions, additions or combination thereof relative to a corresponding parent CDR-L sequence described in TABLE 25. In some embodiments, the CDR-L or the variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 25.

TABLE 25
Exemplary CDR-L sequences of antibodies for binding to IL-23

| | SEQ | | SEQ | | SEQ | |
|---|---|---|---|---|---|---|
| | ID | | ID | | ID | |
| Target | NO: | CDR-L1 | NO: | CDR-L2 | NO: | CDR-L3 |
| IL- | 393 | SSNTGAGYD | 394 | GSG | 395 | QSYDSSLSGWV |
| 23p19 | | | | | | |
| IL- | 396 | SSNIGSGYD | 397 | GNS | 398 | ASWTDGLSLVV |
| 23p19 | | | | | | |
| IL- | 399 | DHILKF | 400 | GAT | 401 | QMYWSTPFT |
| 23p19 | | | | | | |
| IL- | 402 | QSISDY | 403 | YAS | 404 | QQGHSFPFT |
| 23p19 | | | | | | |
| IL- | 405 | RDVAIA | 406 | WAS | 407 | HQYSSYPFT |
| 23p19 | | | | | | |
| IL- | 408 | ENIYSY | 409 | NAK | 410 | QHHYGIPFT |
| 23p19 | | | | | | |
| IL-23 | 411 | TGSSSNTGAGYDVH | 412 | GSGNRPS | 395 | QSYDSSLSGWV |
| IL-23 | 414 | TGSSSNIGAGYDVH | 415 | GSNNRPS | 395 | QSYDSSLSGWV |
| IL-23 | 417 | TLRSGINVGTYRIY | 418 | YKSDSDKQQGS | 419 | MIWHSSASV |
| IL-23 | 417 | TLRSGINVGTYRIY | 418 | YKSDSDKQQGS | 419 | MIWHSSASV |
| IL-23 | 423 | TLNSGYSDYKVD | 424 | VGTGGIVGSKGD | 425 | GADHGSGSNFVYV |
| IL-23 | 426 | TLSSGYSDYKVD | 427 | VGTGGIVGSKGE | 425 | GADHGSGSNFVYV |
| IL-23 | 426 | TLSSGYSDYKVD | 430 | VGTGGTVGSKGE | 425 | GADHGSGSNFVYV |
| IL-23 | 432 | RASQGIAGWLA | 433 | AASSLQS | 434 | QQADSFPPT |
| IL-23 | 435 | RASQVISSWLA | 433 | AASSLQS | 437 | QQANSFPFT |
| IL-23 | 438 | RASQGSSSWFA | 433 | AASSLQS | 437 | QQANSFPFT |
| IL-23 | 441 | RASQGISSWFA | 433 | AASSLQS | 437 | QQANSFPFT |
| IL-23 | 444 | RAGQVISSWLA | 433 | AASSLQS | 446 | QQATSFPLT |
| IL-23 | 447 | RASQGFSGWLA | 433 | AASSLQS | 446 | QQATSFPLT |
| IL-23 | 450 | RASQVISSWFA | 433 | AASSLQS | 446 | QQATSFPLT |
| IL-23 | 438 | RASQGSSSWFA | 433 | AASSLQS | 437 | QQANSFPFT |
| IL-23 | 456 | RASQGIRNDLG | 433 | AASSLQS | 458 | LQHNSYPPT |
| IL-23 | 901 | RASQSVSSSYLA | 900 | GASSRAT | 899 | QQYGSSPLT |

In some embodiments, multispecific antibodies described herein comprise at least one modification within any one of the CDR-Ls described in TABLE 25, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-23, a subunit thereof, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L 3 or a variant thereof, and wherein the combination is according to any one of the combinations provided in TABLE 26.

TABLE 26
Exemplary CDR sequences of antibodies for binding to IL-23

| Target | CDR-H1 | CDR-H2 | CDR-H3 | CDR-L1 | CDR-L2 | CDR-L3 |
|---|---|---|---|---|---|---|
| IL-23p19 | 305 | 306 | 307 | 393 | 394 | 395 |
| IL-23p19 | 308 | 309 | 310 | 396 | 397 | 398 |
| IL-23p19 | 311 | 312 | 313 | 399 | 400 | 401 |
| IL-23p19 | 314 | 315 | 316 | 402 | 403 | 404 |
| IL-23p19 | 317 | 318 | 319 | 405 | 406 | 407 |
| IL-23p19 | 320 | 321 | 322 | 408 | 409 | 410 |
| IL-23 | 323 | 324 | 325 | 411 | 412 | 395 |
| IL-23 | 323 | 327 | 328 | 414 | 415 | 395 |
| IL-23 | 323 | 330 | 331 | 417 | 418 | 419 |
| IL-23 | 332 | 333 | 331 | 417 | 418 | 419 |
| IL-23 | 335 | 336 | 337 | 423 | 424 | 425 |
| IL-23 | 338 | 339 | 340 | 426 | 427 | 425 |
| IL-23 | 341 | 342 | 340 | 426 | 430 | 425 |
| IL-23 | 344 | 345 | 346 | 432 | 433 | 434 |
| IL-23 | 347 | 348 | 349 | 435 | 433 | 437 |
| IL-23 | 347 | 351 | 352 | 438 | 433 | 437 |
| IL-23 | 347 | 354 | 352 | 441 | 433 | 437 |
| IL-23 | 356 | 357 | 358 | 444 | 433 | 446 |
| IL-23 | 359 | 360 | 361 | 447 | 433 | 446 |
| IL-23 | 347 | 354 | 349 | 450 | 433 | 446 |
| IL-23 | 347 | 351 | 352 | 438 | 433 | 437 |
| IL-23 | 323 | 369 | 370 | 456 | 433 | 458 |
| IL-23 | 867 | 786 | 739 | 901 | 900 | 899 |
| IL-23 | 867 | 786 | 742 | 901 | 900 | 899 |
| IL-23 | 893 | 857 | 774 | 901 | 900 | 899 |

In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 27, wherein the multispecific molecule is capable of binding an amino acid sequence that is at least 60%, 70%, 75%, 80%, 85%, 90%, 95%, 99% or 100% identical to SEQ ID NO: 304.

TABLE 27
Exemplary VL sequence of antibodies for binding to IL-23

| SEQ ID | | |
|---|---|---|
| NO: | Target | VL Sequences |
| 459 | IL- | QSVLTQPPSVSGAPGQRVTISCTGSSSNTGAGYDVHWYQQVPGTAP |
| | 23p19 | KLLIYGSGNRPSGVPDRFSGSKSGTSASLAITGLQAEDEADYYCQS |
| | | YDSSLSGWVFGGGTRLTVL |
| 460 | IL- | QSVLTQPPSVSGAPGQRVTISCTGSSSNIGSGYDVHWYQQLPGTAP |
| | 23p19 | KLLIYGNSKRPSGVPDRFSGSKSGTSASLAITGLQSEDEADYYCAS |
| | | WTDGLSLVVFGGGTKLTVL |
| 461 | IL- | DIQMTQSPSSLSASVGDRVTITCKASDHILKFLTWYQQKPGKAPKL |
| | 23p19 | LIYGATSLETGVPSRFSGSGSGTDFTLTISSLQPEDFATYYCQMYW |
| | | STPFTFGGGTKVEIK |
| 462 | IL- | DIQMTQSPSSLSASVGDRVTITCRASQSISDYLHWYQQKPGKAPKL |
| | 23p19 | LIKYASQSMSGVPSRFSGSGSGSDFTLTISSLQPEDFATYYCQQGH |
| | | SFPFTFGQGTKLEIK |
| 463 | IL- | DIQMTQSPSSLSASVGDRVTITCKASRDVAIAVAWYQQKPGKVPKL |
| | 23p19 | LIYWASTRHTGVPSRFSGSGSRTDFTLTISSLQPEDVADYFCHQYS |
| | | SYPFTFGSGTKLEIK |
| 464 | IL- | DIQMTQSPSSLSASVGDRVTITCRTSENIYSYLAWYQQKPGKAPKL |
| | 23p19 | LIYNAKTLAEGVPSRFSGSGSGTDFTLTISSLQPEDFATYYCQHHY |
| | | GIPFTFGQGTKVEIK |
| 466 | IL-23 | QSVLTQPPSVSGAPGQRVTISCTGSSSNIGAGYDVHWYQQLPGTAP |
| | | KLLIYGSNNRPSGVPDRFSGSKSGTSASLAITGLQAEDEADYYCQS |
| | | YDSSLSGWVFGGGTKLTVL |
| 467 | IL-23 | QAVLTQPSSLSASPGASASLTCTLRSGINVGTYRIYWYQQKPGSPP |
| | | QYLLRYKSDSDKQQGSGVPSRFSGSKDASANAGILLISGLQSEDEA |
| | | DYYCMIWHSSASVFGGGTKLTVL |
| 469 | IL-23 | QPVLTQPPSASASLGASVTLTCTLNSGYSDYKVDWYQQRPGKGPRF |
| | | VMRVGTGGIVGSKGDGIPDRFSVLGSGLNRYLTIKNIQEEDESDYH |
| | | CGADHGSGSNFVYVFGTGTKVTVL |
| 470 | IL-23 | QPVLTQPPSASASLGASVTLTCTLSSGYSDYKVDWYQQRPGKGPRF |
| | | VMRVGTGGIVGSKGEGIPDRFSVLGSGLNRYLTIKNIQEEDESDYH |
| | | CGADHGSGSNFVYVFGTGTKVTVL |
| 471 | IL-23 | QPELTQPPSASASLGASVTLTCTLSSGYSDYKVDWYQLRPGKGPRF |
| | | VMRVGTGGTVGSKGEGIPDRFSVLGSGLNRSLTIKNIQEEDESDYH |
| | | CGADHGSGSNFVYVFGTGTKVTVL |
| 472 | IL-23 | DIQLTPSPSSVSASVGDRVTITCRASQGIAGWLAWYQQKPGKAPKL |
| | | LIYAASSLQSGVPSRFSGSGSGTDFTLTISSLQPEDFATYYCQQAD |
| | | SFPPTFGGGTKVEIK |
| 473 | IL-23 | DIQMTQSPSSVSASVGDRVTITCRASQVISSWLAWYQQKPGKAPSL |
| | | LIYAASSLQSGVPSRFSGSVSGTDFTLTISSLQPEDFATYYCQQAN |
| | | SFPFTFGPGTKVDFK |
| 474 | IL-23 | DIQMTQSPSSVSASVGDRVTITCRASQGSSSWFAWYQQKPGKAPKL |
| | | LIYAASSLQSGVPSRFSGSGSGTDFTLTISSLQPEDFATYYCQQAN |
| | | SFPFTFGPGTKVDIK |
| 475 | IL-23 | DSQMTQSPSSVSASVGDRVTITCRASQGISSWFAWYQQKPGQAPNL |
| | | LIYAASSLQSGVPSRFSGSGSGTEFTLTISSLQPEDFATYYCQQAN |
| | | SFPFTFGPGTKVDIK |
| 476 | IL-23 | DIQMTQSPSSVSASVGDRVTITCRAGQVISSWLAWYQQKPGKAPKL |
| | | LIYAASSLQSGVPSRFSGSGSGTDFTLTISSLQPDDFATYYCQQAT |
| | | SFPLTFGGGTKVEIK |
| 477 | IL-23 | DIQMTQSPSSVSASVGDRVTITCRASQGFSGWLAWYQQKPGKAPKL |
| | | LIYAASSLQSGVPSRFSGSGSGTDFTLTISSLQPEDFATYYCQQAT |
| | | SFPLTFGPGTKVDIK |
| 478 | IL-23 | DIQLTQSPSSVSASVGDRVTITCRASQVISSWFAWYQQKPGKAPNL |
| | | LIYAASSLQSGVPSRFSGSGSGTDFTLTISSLQPADFATYFCQQAT |
| | | SFPLTFGPGTKVDVK |
| 480 | IL-23 | DIQMTQSPSSLSASVGDRVTITCRASQGIRNDLGWYQQKPGKAPKR |
| | | LIYAASSLQSGVPSRFSGSGSGTEFTLTISSLQPEDFATYYCLQHN |
| | | SYPPTFGQGTKVEIE |
| 303 | IL-23 | DIQMTQSPSSLSASVGDRVTITCRASQGISSWLAWYQQKPEKAPKS |
| | | LIYAASSLQSGVPSRFSGSGSGTDFTLTISSLQPEDFATYYCQQYN |
| | | TYPYTFGQGTKLEIK |
| 1768 | IL-23 | EIVLTQSPGTLSLSPGERATLSCRASQSVSSSYLAWYQQKPGQAPR |
| | | LLIYGASSRATGIPDRFSGSGSGTDFTLTISRLEPEDFAVYYCQQY |
| | | GSSPLTFGQGTKVEIK |

In some embodiments, multispecific antibodies described herein comprise at least one modification within any one of the VL sequences described in TABLE 27, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is IL-23, a subunit thereof, a functional fragment thereof, a variant thereof, a multimeric form thereof or a combination thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification. In some embodiments, multispecific antibodies described herein comprise two light chain variable region that are at least 90%, at least 95%, at least 98% or 100% identical relative to each other.

In some embodiments, the at least one modification in CDR-H1, CDR-H2d and/or CDR-H3. In some embodiments, the at least one modification is a substitution in CDR-H3. In some embodiments, the at least one modification comprises a substitution of an uncharged amino acid with a charged amino acid in CDR-H1, CDR-H2 and/or CDR-H3. In some embodiments, the at least one modification comprises a substitution of a charged amino acid with an uncharged amino acid in CDR-H1, CDR-H2 and/or CDR-H3. The uncharged amino acid can be an amino acid with a hydrophobic side chain (e.g., glycine, alanine, valine, cysteine, proline, leucine, isoleucine, methionine, tryptophan, phenylalanine). Alternatively, the uncharged amino acid can be a polar uncharged amino acid (e.g., serine, threonine, tyrosine, asparagine, glutamine). The charged amino acid can be a negatively charged amino acid (e.g., aspartic acid, glutamic acid). Alternatively, the charged amino acid can be a positively charged amino acid (e.g., lysine, arginine, histidine). In some embodiments, the at least one modification is a substitution of a positively charged amino acid residue with a negatively charged amino acid residue in CDR-H1, CDR-H2 and/or CDR-H3. In some embodiments, the at least one modification is a substitution of a negatively charged amino acid residue with a positively charged amino acid residue in CDR-H1, CDR-H2 and/or CDR-H3. In some embodiments, a ratio of the binding affinities of target binding domains, variants thereof or functional fragments thereof, as described herein, for an IL-23, a variant thereof, or a fragment thereof at neutral pH and acidic pH, respective, is at least 1.2, at least 10, at least 100, at least 200, or more. In some embodiments, the VH sequence comprises an amino acid sequence of SEQ ID NO: 1544. In some embodiments, the VH sequence comprises at least one modification at positions selected from 28, 30, 31, 33, 34, 56, 58, 59, 62, 65, 98, 101, 102 and 108, relative to the amino acid position numbering of SEQ ID NO: 1544. In some embodiments, the VH sequence comprises at least one modification at positions selected from T28, T30, I31, A33, 134, G56, G58, H59, Q62, Q65, R98, E101, N102 and L108, relative to the amino acid position numbering of SEQ ID NO: 1544. In some embodiments, the VH sequence comprises at least one substitution selected from a group consisting of T28V, T28A, T28L, T28P, T30S, 131Q, A33T, 134M, 134V, G56K, G56A, G56N, G58A, H59Y, H59V, Q62S, Q62K, Q65R, Q65K, Q65A, R981, E101S, E101Y, E101T, N102F, N102Y, N102S, N102D, N102K, N102R, L108T and L108M relative to the amino acid position numbering of SEQ ID NO: 1544.

In some embodiments, multispecific antibodies comprise an IL-23 binding region, wherein the IL-23 binding region comprises an IL-23 binding heavy chain variable domain and, optionally, an IL-23 binding light chain variable domain. In some embodiments, multispecific antibodies described herein comprise a binding affinity for IL-23, a variant thereof or a functional fragment thereof that is at least 10%, at least 20%, at least 30%, at least 50%, at least 80%, at least 100%, at least 200% or more higher under neutral pH condition relative to the binding affinity under acidic pH condition. In some embodiments, multispecific antibodies described herein comprise a ratio of binding affinities for IL-23, a variant thereof or a functional fragment under neutral pH condition and acidic pH condition is at least 1.1, 1.5, 2, 3, 5, 8, 10, 100 or more. In some embodiments, multispecific antibodies described herein comprise a binding affinity for IL-23, a variant thereof or a functional fragment thereof that is at least 10%, at least 20%, at least 30%, at least 50%, at least 80%, at least 100%, at least 200% or more higher at about pH 7.4 relative to the binding affinity at about pH 5.8. In some embodiments, multispecific antibodies described herein comprise a ratio of binding affinities for IL-23, a variant thereof or a functional fragment at about pH 7.4 and about pH 5.8 is at least 1.1, 1.5, 2, 3, 5, 8, 10, 100 or more.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 24; and (b) a VL sequence that is at least 60%, at least 65%, at least 70%, at least 75%, at least 80%, at least 85%, at least 90%, at least 95% or 100% identical to any one of amino acid sequences described in TABLE 27, wherein the multispecific molecule comprises the VH sequence and the VL sequence according to the combination described in TABLE 28.

TABLE 28
Exemplary VH and VL sequences of antibodies for binding IL-23

| | VH | VL |
|---|---|---|
| Target | (SEQ ID NO:) | (SEQ ID NO:) |
| IL-23p19 | 371 | 459 |
| IL-23p19 | 372 | 460 |
| IL-23p19 | 373 | 461 |
| IL-23p19 | 374 | 462 |
| IL-23p19 | 375 | 463 |
| IL-23p19 | 376 | 464 |
| IL-23 | 377 | 459 |
| IL-23 | 378 | 466 |
| IL-23 | 379 | 467 |
| IL-23 | 380 | 467 |
| IL-23 | 381 | 469 |
| IL-23 | 382 | 470 |
| IL-23 | 383 | 471 |
| IL-23 | 384 | 472 |
| IL-23 | 385 | 473 |
| IL-23 | 386 | 474 |
| IL-23 | 387 | 475 |
| IL-23 | 388 | 476 |
| IL-23 | 389 | 477 |
| IL-23 | 390 | 478 |
| IL-23 | 391 | 474 |
| IL-23 | 392 | 480 |
| IL-23 | 283 | 303 |
| IL-23 | 1466 | 1768 |
| IL-23 | 1544 | 1768 |
| IL-23 | 1621 | 1768 |

### IL-17 and IL-17R Constructs

Interleukin-17 (IL-17) family cytokine in humans comprises IL-17A, IL-17B, IL-17C, IL17-D, IL-17E and IL-17F. These cytokines are involved in proinflammatory responses and can mediate or induce the expression of a variety of other cytokines, factors, and mediators including tissue necrosis factor-alpha (TNF-α), IL-6, IL-8, IL-1β, granulocyte colony-stimulating factor (G-CSF), prostaglandin E2 (PGE2), IL-10, IL-12, IL-1R antagonist, leukemia inhibitory factor, and stromelysin. IL-17 family cytokine also induces nitric oxide in chondrocytes and in human osteoarthritis explants. IL-17 family cytokine can induce the release of cytokines, chemokines, and growth factors and is an important local orchestrator of neutrophil accumulation. IL-17 family cytokine can induce cartilage and bone destruction. IL-17 family cytokine signaling is a target in a variety of autoimmune diseases including rheumatoid arthritis (RA), ankylosing spondylitis, psoriasis, hidradenitis suppurativa, ulcerative colitis, Crohn's disease, multiple sclerosis (MS), psoriatic arthritis, asthma, lupus (SLE), and sepsis.

In some embodiments, multispecific antibodies described herein bind an IL-17 family cytokine amino acid sequence, or portion thereof. In some embodiments, multispecific antibodies described herein bind an IL-17A amino acid sequence, or portion thereof. In some embodiments, multispecific antibodies described herein bind an IL-17A/F amino acid sequence, or portion thereof. In some embodiments, antibodies targeting IL-17A or IL-17A/F are used for treating psoriasis (PsO), psoriatic arthritis (PsA), ankylosing spondylitis (AS), non-radiographic Axial Spondyloarthritis (nr-axSpA), Enthesitis-related arthritis (ERA) or combinations thereof.

In some embodiments, IL-17 family cytokine functions as a heterodimer with an interleukin-17 receptor (IL-17R) family of proteins. In some embodiments, the IL-17R comprises IL-17RA, IL-17RB, IL-17RC, IL-17RD, IL-17RE, IL-17RF, or a combination thereof. Accordingly, in some embodiments, multispecific antibodies described herein are capable of binding any one of IL-17A, IL-17B, IL-17C, IL-17D, IL-17E, IL-17F, IL-17RA, IL-A7RB, IL-17RC, IL-17RD, IL-17RE, or a combination thereof.

Amino acid sequences of IL-17 family are recited in TABLE 29.

TABLE 29
IL-17 and/or IL-17R Amino Acid Sequences

| | SEQ ID | |
|---|---|---|
| Name | NO: | Amino Acid Sequences |
| IL- | 28 | MTPGKTSLVSLLLLLSLEAIVKAGITIPRNPGCPNSEDKNFPRTVMVNLNIHN |
| 17A | | RNTNTNPKRSSDYYNRSTSPWNLHRNEDPERYPSVIWEAKCRHLGCINADGNV |
| | | DYHMNSVPIQQEILVLRREPPHCPNSFRLEKILVSVGCTCVTPIVHHVA |
| IL- | 29 | MDWPHNLLFLLTISIFLGLGQPRSPKSKRKGQGRPGPLAPGPHQVPLDLVSRM |
| 17B | | KPYARMEEYERNIEEMVAQLRNSSELAQRKCEVNLQLWMSNKRSLSPWGYSIN |
| | | HDPSRIPVDLPEARCLCLGCVNPFTMQEDRSMVSVPVFSQVPVRRRLCPPPPR |
| | | TGPCRQRAVMETIAVGCTCIF |
| IL- | 30 | MTLLPGLLFLTWLHTCLAHHDPSLRGHPHSHGTPHCYSAEELPLGQAPPHLLA |
| 17C | | RGAKWGQALPVALVSSLEAASHRGRHERPSATTQCPVLRPEEVLEADTHQRSI |
| | | SPWRYRVDTDEDRYPQKLAFAECLCRGCIDARTGRETAALNSVRLLQSLLVLR |
| | | RRPCSRDGSGLPTPGAFAFHTEFIHVPVGCTCVLPRSV |
| IL- | 31 | MLVAGFLLALPPSWAAGAPRAGRRPARPRGCADRPEELLEQLYGRLAAGVLSA |
| 17D | | FHHTLQLGPREQARNASCPAGGRPADRRFRPPTNLRSVSPWAYRISYDPARYP |
| | | RYLPEAYCLCRGCLTGLFGEEDVRFRSAPVYMPTVVLRRTPACAGGRSVYTEA |
| | | YVTIPVGCTCVPEPEKDADSINSSIDKQGAKLLLGPNDAPAGP |
| IL- | 32 | MRERPRLGEDSSLISLFLQVVAFLAMVMGTHTYSHWPSCCPSKGQDTSEELLR |
| 17E | | WSTVPVPPLEPARPNRHPESCRASEDGPLNSRAISPWRYELDRDLNRLPQDLY |
| | | HARCLCPHCVSLQTGSHMDPRGNSELLYHNQTVFYRRPCHGEKGTHKGYCLER |
| | | RLYRVSLACVCVRPRVMG |
| IL- | 33 | MTVKTLHGPAMVKYLLLSILGLAFLSEAAARKIPKVGHTFFQKPESCPPVPGG |
| 17F | | SMKLDIGIINENQRVSMSRNIESRSTSPWNYTVTWDPNRYPSEVVQAQCRNLG |
| | | CINAQGKEDISMNSVPIQQETLVVRRKHQGCSVSFQLEKVLVTVGCTCVTPVI |
| | | HHVQ |
| IL- | 34 | MGAARSPPSAVPGPLLGLLLLLLGVLAPGGASLRLLDHRALVCSQPGLNCTVK |
| 17RA | | NSTCLDDSWIHPRNLTPSSPKDLQIQLHFAHTQQGDLFPVAHIEWTLQTDASI |
| | | LYLEGAELSVLQLNTNERLCVRFEFLSKLRHHHRRWRFTFSHFVVDPDQEYEV |
| | | TVHHLPKPIPDGDPNHQSKNFLVPDCEHARMKVTTPCMSSGSLWDPNITVETL |
| | | EAHQLRVSFTLWNESTHYQILLTSFPHMENHSCFEHMHHIPAPRPEEFHQRSN |
| | | VTLTLRNLKGCCRHQVQIQPFFSSCLNDCLRHSATVSCPEMPDTPEPIPDYMP |
| | | LWVYWFITGISILLVGSVILLIVCMTWRLAGPGSEKYSDDTKYTDGLPAADLI |
| | | PPPLKPRKVWIIYSADHPLYVDVVLKFAQFLLTACGTEVALDLLEEQAISEAG |
| | | VMTWVGRQKQEMVESNSKIIVLCSRGTRAKWQALLGRGAPVRLRCDHGKPVGD |
| | | LFTAAMNMILPDFKRPACFGTYVVCYFSEVSCDGDVPDLFGAAPRYPLMDRFE |
| | | EVYFRIQDLEMFQPGRMHRVGELSGDNYLRSPGGRQLRAALDRFRDWQVRCPD |
| | | WFECENLYSADDQDAPSLDEEVFEEPLLPPGTGIVKRAPLVREPGSQACLAID |
| | | PLVGEEGGAAVAKLEPHLQPRGQPAPQPLHTLVLAAEEGALVAAVEPGPLADG |
| | | AAVRLALAGEGEACPLLGSPGAGRNSVLFLPVDPEDSPLGSSTPMASPDLLPE |
| | | DVREHLEGLMLSLFEQSLSCQAQGGCSRPAMVLTDPHTPYEEEQRQSVQSDQG |
| | | YISRSSPQPPEGLTEMEEEEEEEQDPGKPALPLSPEDLESLRSLQRQLLFRQL |
| | | QKNSGWDTMGSESEGPSA |
| IL- | 35 | MSLVLLSLAALCRSAVPREPTVQCGSETGPSPEWMLQHDLIPGDLRDLRVEPV |
| 17RB | | TTSVATGDYSILMNVSWVLRADASIRLLKATKICVTGKSNFQSYSCVRCNYTE |
| | | AFQTQTRPSGGKWTFSYIGFPVELNTVYFIGAHNIPNANMNEDGPSMSVNFTS |
| | | PGCLDHIMKYKKKCVKAGSLWDPNITACKKNEETVEVNFTTTPLGNRYMALIQ |
| | | HSTIIGFSQVFEPHQKKQTRASVVIPVTGDSEGATVQLTPYFPTCGSDCIRHK |
| | | GTVVLCPQTGVPFPLDNNKSKPGGWLPLLLLSLLVATWVLVAGIYLMWRHERI |
| | | KKTSFSTTTLLPPIKVLVVYPSEICFHHTICYFTEFLQNHCRSEVILEKWQKK |
| | | KIAEMGPVQWLATQKKAADKVVFLLSNDVNSVCDGTCGKSEGSPSENSQDLFP |
| | | LAFNLFCSDLRSQIHLHKYVVVYFREIDTKDDYNALSVCPKYHLMKDATAFCA |
| | | ELLHVKQQVSAGKRSQACHDGCCSL |
| IL- | 36 | MPVPWFLLSLALGRSPVVLSLERLVGPQDATHCSPVSLEPWGDEERLRVQFLA |
| 17RC | | QQSLSLAPVTAATARTALSGLSGADGRREERGRGKSWVCLSLGGSGNTEPQKK |
| | | GLSCRLWDSDILCLPGDIVPAPGPVLAPTHLQTELVLRCQKETDCDLCLRVAV |
| | | HLAVHGHWEEPEDEEKFGGAADSGVEEPRNASLQAQVVLSFQAYPTARCVLLE |
| | | VQVPAALVQFGQSVGSVVYDCFEAALGSEVRIWSYTQPRYEKELNHTQQLPDC |
| | | RGLEVWNSIPSCWALPWLNVSADGDNVHLVLNVSEEQHFGLSLYWNQVQGPPK |
| | | PRWHKNLTGPQIITLNHTDLVPCLCIQVWPLEPDSVRTNICPFREDPRAHQNL |
| | | WQAARLQLLTLQSWLLDAPCSLPAEAALCWRAPGGDPCQPLVPPLSWENVTVD |
| | | KVLEFPLLKGHPNLCVQVNSSEKLQLQECLWADSLGPLKDDVLLLETRGPQDN |
| | | RSLCALEPSGCTSLPSKASTRAARLGEYLLQDLQSGQCLQLWDDDLGALWACP |
| | | MDKYIHKRWALVWLACLLFAAALSLILLLKKDHAKGWLRLLKQDVRSGAAARG |
| | | RAALLLYSADDSGFERLVGALASALCQLPLRVAVDLWSRRELSAQGPVAWFHA |
| | | QRRQTLQEGGVVVLLFSPGAVALCSEWLQDGVSGPGAHGPHDAFRASLSCVLP |
| | | DFLQGRAPGSYVGACFDRLLHPDAVPALFRTVPVFTLPSQLPDFLGALQQPRA |
| | | PRSGRLQERAEQVSRALQPALDSYFHPPGTPAPGRGVGPGAGPGAGDGT |
| IL- | 37 | MAPWLQLCSVFFTVNACLNGSQLAVAAGGSGRARGADTCGWRGVGPASRNSGL |
| 17RD | | YNITFKYDNCTTYLNPVGKHVIADAQNITISQYACHDQVAVTILWSPGALGIE |
| | | FLKGFRVILEELKSEGRQCQQLILKDPKQLNSSFKRTGMESQPFLNMKFETDY |
| | | FVKVVPFPSIKNESNYHPFFFRTRACDLLLQPDNLACKPFWKPRNLNISQHGS |
| | | DMQVSFDHAPHNFGFRFFYLHYKLKHEGPFKRKTCKQEQTTETTSCLLQNVSP |
| | | GDYIIELVDDTNTTRKVMHYALKPVHSPWAGPIRAVAITVPLVVISAFATLFT |
| | | VMCRKKQQENIYSHLDEESSESSTYTAALPRERLRPRPKVFLCYSSKDGQNHM |
| | | NVVQCFAYFLQDFCGCEVALDLWEDFSLCREGQREWVIQKIHESQFIIVVCSK |
| | | GMKYFVDKKNYKHKGGGRGSGKGELFLVAVSAIAEKLRQAKQSSSAALSKFIA |
| | | VYFDYSCEGDVPGILDLSTKYRLMDNLPQLCSHLHSRDHGLQEPGQHTRQGSR |
| | | RNYFRSKSGRSLYVAICNMHQFIDEEPDWFEKQFVPFHPPPLRYREPVLEKFD |
| | | SGLVLNDVMCKPGPESDFCLKVEAAVLGATGPADSQHESQHGGLDQDGEARPA |
| | | LDGSAALQPLLHTVKAGSPSDMPRDSGIYDSSVPSSELSLPLMEGLSTDQTET |
| | | SSLTESVSSSSGLGEEEPPALPSKLLSSGSCKADLGCRSYTDELHAVAPL |
| IL- | 38 | MGSSRLAALLLPLLLIVIDLSDSAGIGFRHLPHWNTRCPLASHTDDSFTGSSA |
| 17RE | | YIPCRTWWALFSTKPWCVRVWHCSRCLCQHLLSGGSGLQRGLFHLLVQKSKKS |
| | | STFKFYRRHKMPAPAQRKLLPRRHLSEKSHHISIPSPDISHKGLRSKRTQPSD |
| | | PETWESLPRLDSQRHGGPEFSFDLLPEARAIRVTISSGPEVSVRLCHQWALEC |
| | | EELSSPYDVQKIVSGGHTVELPYEFLLPCLCIEASYLQEDTVRRKKCPFQSWP |
| | | EAYGSDFWKSVHFTDYSQHTQMVMALTLRCPLKLEAALCQRHDWHTLCKDLPN |
| | | ATARESDGWYVLEKVDLHPQLCFKFSFGNSSHVECPHQTGSLTSWNVSMDTQA |
| | | QQLILHFSSRMHATFSAAWSLPGLGQDTLVPPVYTVSQARGSSPVSLDLIIPF |
| | | LRPGCCVLVWRSDVQFAWKHLLCPDVSYRHLGLLILALLALLTLLGVVLALTC |
| | | RRPQSGPGPARPVLLLHAADSEAQRRLVGALAELLRAALGGGRDVIVDLWEGR |
| | | HVARVGPLPWLWAARTRVAREQGTVLLLWSGADLRPVSGPDPRAAPLLALLHA |
| | | APRPLLLLAYFSRLCAKGDIPPPLRALPRYRLLRDLPRLLRALDARPFAEATS |
| | | WGRLGARQRRQSRLELCSRLEREAARLADLG |

In some embodiments, multispecific molecules described herein bind to an amino acid sequence that is at least 60%, at least 70%, at least 75%, at least 80%, at least 85%, at least 90%, at least 95%, at least 99% or 100% identical to the sequences recited in TABLE 29. In some embodiments, multispecific molecules described herein bind to an amino acid sequence that is at least 60%, 70%, 75%, 80%, 85%, 90%, 95%, 99% or 100% identical to any one of SEQ ID NOS: 28-38.

In some embodiments, multispecific antibodies described herein comprise any one of combinations of CDR-Hs or variants thereof described in TABLE 30, wherein the multispecific antibody is capable of binding IL-17 family cytokine, IL-17R or a combination thereof. In some embodiments, the CDR-H variant comprises at least one, two, or three substitutions, deletions, additions or combination thereof relative to a corresponding parent CDR-H sequence described in TABLE 30. In some embodiments, the CDR-H or the variant thereof comprises an amino acid sequence that is at least 60%, at least 65%, at least 70%, at least 75%, at least 80%, at least 85%, at least 90%, at least 95% or 100% identical to a corresponding parent corresponding parent CDR-H sequence described in TABLE 30.

TABLE 30
Exemplary CDR-H sequences of antibodies for binding to IL-17 and/or IL-17R

| | SEQ | | SEQ | | SEQ | |
|---|---|---|---|---|---|---|
| | ID | | ID | | ID | |
| Target | NO: | VH-CDR1 | NO: | VH-CDR2 | NO: | VH-CDR3 |
| 17A/F | 39 | GFTFDDYA | 40 | NWSSGGI | 41 | ARDIGGFGEFYWN |
| | | | | | | F |
| 17A/F | 42 | GFTFSDYN | 43 | TITYEGRNT | 44 | ASPPQYYEGSIYR |
| | | | | | | LWFAH |
| 17A | 45 | SYSFTSDYAW | 46 | ITYSGVT | 47 | ARADYDSYYTMDY |
| 17A | 48 | GYSFTDYHI | 49 | VINPMYGTTD | 50 | ARYDYFTGTGVY |
| 17A | 51 | GFTFSNYW | 52 | AINQDGSEK | 53 | VRDYYDILTDYYI |
| | | | | | | HYWYFDL |
| 17RA | 54 | GYTFTRY | 55 | ISTYSGNT | 56 | ARRQLYFDY |
| 17A | 57 | GGTFATSP | 58 | ISPSGGD | 59 | CAVRRRFDGTSYY |
| | | | | | | TGDYDS |
| 17A | 60 | GFTFSDYT | 61 | IKSGGSYS | 62 | ARDGDYGSSYGAM |
| | | | | | | DY |
| 17A | 39 | GFTFDDYA | 64 | ITWNSGHI | 65 | AKVSYLSTASSLD |
| | | | | | | Y |
| 17A | 66 | GGSFGGYG | 67 | ITPFFGF | 68 | ARDPNEFWNGYYS |
| | | | | | | THDFDS |
| 17A | 69 | GRTFSSYV | 70 | ISGSGESI | 71 | TADQEFGYLRFGR |
| | | | | | | SEY |
| 17A/F | 72 | GRTYDA | 73 | ISGSGDDT | 74 | ATRRGLYYVWDAN |
| | | | | | | DYEN |
| 17A | 75 | GYKFTDYH | 76 | INPTYGTT | 77 | ARYDYFTGTGVY |

In some embodiments, multispecific antibodies described herein comprise at least one modification within any one of the CDR-Hs described in TABLE 30, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is an IL-17 family cytokine amino acid sequence, or portion thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific antibodies described herein comprises an amino acid sequence that is at least 60%, at least 65%, at least 70%, at least 75%, at least 80%, at least 85%, at least 90%, at least 95% or 100% identical to any one of VH sequences described in TABLE 31, wherein the multispecific molecule is capable of binding an amino acid sequence that is at least 60%, 70%, 75%, 80%, 85%, 90%, 95%, 99% or 100% identical to any one of SEQ ID NOS: 28-38.

TABLE 31
Exemplary VH sequence of antibodies for binding to IL-17 and/or IL-17R

| | SEQ ID | |
|---|---|---|
| Target | NO: | VH Sequences |
| 17A/F | 84 | EVQLVESGGGLVQPGRSLRLSCAASGFTFDDYAMHWVRQAPGKGLEWV |
| | | SGINWSSGGIGYADSVKGRFTISRDNAKNSLYLQMNSLRAEDTALYYC |
| | | ARDIGGFGEFYWNFGLWGRGTLVTVSS |
| 17A/F | 85 | EVQLVESGGGLVQPGGSLRLSCAASGFTFSDYNMAWVRQAPGKGLEWV |
| | | ATITYEGRNTYYRDSVKGRFTISRDNAKNSLYLQMNSLRAEDTAVYYC |
| | | ASPPQYYEGSIYRLWFAHWGQGTLVTVSS |
| 17A | 86 | DVQLQESGPGLVKPSQTLSLTCTVSSYSFTSDYAWSWIRQPPGKGLEW |
| | | IGYITYSGVTSYNPSLKSRVTISVDTSKNQFSLKLSSVTAADTAVYYC |
| | | ARADYDSYYTMDYWGQGTSVTVSS |
| 17A | 87 | QVQLVQSGAEVKKPGSSVKVSCKASGYSFTDYHIHWVRQAPGQGLEWM |
| | | GVINPMYGTTDYNQRFKGRVTITADESTSTAYMELSSLRSEDTAVYYC |
| | | ARYDYFTGTGVYWGQGTLVTVSS |
| 17A | 88 | EVQLVESGGGLVQPGGSLRLSCAASGFTFSNYWMNWVRQAPGKGLEWV |
| | | AAINQDGSEKYYVGSVKGRFTISRDNAKNSLYLQMNSLRVEDTAVYYC |
| | | VRDYYDILTDYYIHYWYFDLWGRGTLVTVSS |
| 17RA | 89 | QVQLVQSGAEVKKPGASVKVSCKASGYTFTRYGISWVRQAPGQGLEWM |
| | | GWISTYSGNTNYAQKLQGRVTMTTDTSTSTAYMELRSLRSDDTAVYYC |
| | | ARRQLYFDYWGQGTLVTVSS |
| 17A | 90 | QVQLVQSGGGLVQAGGSLRLSCAASGGTFATSPMGWLRQAPGKGTEFV |
| | | AAISPSGGDRIYADSVKGRFTISRDNAGYFIYLQMNSLKPEDTAVYYC |
| | | AVRRRFDGTSYYTGDYDSWGQGTLVTVSS |
| 17A | 91 | EVQLVESGGGLVQPGGSLRLSCAASGFTFSDYTMLWVRQAPGKGLEWV |
| | | AIIKSGGSYSYYPDSVKGRFTISRDNAKNSLYLQMNSLRAEDTAVYYC |
| | | ARDGDYGSSYGAMDYWGQGTLVTVSS |
| 17A | 92 | EVQLVESGGGLVQPGRSLRLSCAASGFTFDDYAMHWVRQAPGKGLEWV |
| | | SAITWNSGHIDYADSVEGRFTISRDNAKNSLYLQMNSLRAEDTAVYYC |
| | | AKVSYLSTASSLDYWGQGTLVTVSS |
| 17A | 93 | DVQLVESGGGLVQPGGSLRLSCAASGRTFSSYVVGWFRQAPGKEREFI |
| | | GAISGSGESIYYAVSEKGRFTISRDNSKNTLYLQMNSLRPEDTAVYYC |
| | | TADQEFGYLRFGRSEYWGQGTLVTVSS |
| 17A/F | 94 | EVQLVESGGGLVQPGGSLRLSCAASGRTYDAMGWLRQAPGKEREFVAA |
| | | ISGSGDDTYYADSVKGRFTISRDNSKNTLYLQMNSLRPEDTAVYYCAT |
| | | RRGLYYVWDANDYENWGQGTLVTVSS |
| 17A | 95 | QVQLVQSGAEVKKPGSSVKVSCKASGYKFTDYHIHWVRQAPGQCLEWM |
| | | GVINPTYGTTDYNQRFKGRVTITADESTSTAYMELSSLRSEDTAVYYC |
| | | ARYDYFTGTGVYWGQGTLVTVSS |
| 17A | 96 | QVQLVQSGAEVKKPGASVKVSCKASGYIFTNYGMNWMRQAPGQGLEWM |
| | | GWINDHTGEPTYADKFQGRVTFTLDTSISTAYMELSRLRSDDTAVYYC |
| | | ANYGFGYFDYWGQGTLVTVSS |
| 17A | 97 | EVQLVQSGAEVKKPGASVKVSCKASGYTFTDYEVHWVRQAPGQGLEWM |
| | | GVIDPGTGGVAYNQKFEGRVTMTADTSTSTAYMELRSLRSDDTAVYYC |
| | | TRYSLFYGSSPYAMDYWGQGTLVTVSS |
| 17A | 98 | EVQLVESGGGLVQPGGSLRLSCAASGMSMSDYWMNWVRQAPGKGLEWV |
| | | AAINQDGDEKYYVGSVKGRFTISRDNAKNSLYLQMNSLRVEDTAVYYC |
| | | VRDYYDLISDYYIHYWYFDLWGRGTLVTVSS |

In some embodiments, multispecific antibodies described herein comprise at least one modification within any one of the VH sequences described in TABLE 31, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is an IL-17 family cytokine amino acid sequence, or portion thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific antibodies described herein comprise any one of combinations of CDR-Ls or variants thereof described in TABLE 32, wherein the multispecific molecule is capable of binding an amino acid sequence that is at least 60%, 70%, 75%, 80%, 85%, 90%, 95%, 99% or 100% identical to any one of SEQ ID NOS: 28-38. In some embodiments, multispecific molecules described herein comprise any one of CDR-L1 described in TABLE 32 or a variant thereof, any one of CDR-L2 described in TABLE 32 or a variant thereof, and any one of CDR-L3 described in TABLE 32 or a variant thereof, wherein the multispecific molecule is capable of binding an amino acid sequence that is at least 60%, 70%, 75%, 80%, 85%, 90%, 95%, 99% or 100% identical to any one of SEQ ID NOS: 28-38. In some embodiments, multispecific molecules described herein comprise any one of combinations of CDR-Ls or variants thereof described in TABLE 32, wherein the multispecific molecule is capable of binding an amino acid sequence that is at least 60%, 70%, 75%, 80%, 85%, 90%, 95%, 99% or 100% identical to any one of SEQ ID NOS: 28-38. In some embodiments, the CDR-L variant comprises at least one, at least two or at least three substitutions, deletions, additions or combination thereof relative to a corresponding parent CDR-L sequence described in TABLE 32. In some embodiments, the CDR-L or the variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 32.

TABLE 32
Exemplary CDR-L sequences of antibodies for binding to IL-17 and/or IL-17R

| | SEQ | | SEQ | | SEQ | VL-CDR3 |
|---|---|---|---|---|---|---|
| | ID | | ID | | ID | |
| Target | NO: | VL-CDR1 | NO: | VL-CDR2 | NO: | |
| 17A/F | 99 | QSVRSY | 100 | DAS | 101 | QQRSNWPPAT |
| 17A/F | 102 | DESVRTLMH | 103 | LVSNS | 104 | QQTWSDPWT |
| 17A | 105 | QSLVHSNGNTY | 106 | KVS | 107 | SQSTHFWT |
| 17A | 108 | RSLVHSRGNTY | 106 | KVS | 109 | SQSTHLPFT |
| 17A | 110 | QSVSSSYL | 19 | GAS | 111 | QQYGSSPCT |
| 17RA | 112 | QSVSSN | 100 | DAS | 113 | QQYDNWPLT |
| 17A | 114 | SQSVSSSY | 100 | DAS | 115 | QQYSYSPVT |
| 17A | 116 | QDINSY | 117 | RAN | 118 | CLQYDAFPPYT |
| 17A | 119 | QGIRNY | 120 | AAS | 121 | QRYNRAPYT |
| 17A | 122 | QDIGSE | 16 | YAS | 123 | HQTDSLPYT |
| 17A | 124 | RSLVHSRGETY | 106 | KVS | 109 | SQSTHLPFT |

In some embodiments, multispecific antibodies described herein comprise at least one modification within any one of the CDR-Ls described in TABLE 32, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is an IL-17 family cytokine amino acid sequence, or portion thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific antibodies described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of the combinations provided in TABLE 33.

TABLE 33
Exemplary CDR sequences of antibodies
for binding to IL-17 and/or IL-17R

| | VH- | | | VL- | VL- | VL- |
|---|---|---|---|---|---|---|
| Target | CDR1 | VH-CDR2 | VH-CDR3 | CDR1 | CDR2 | CDR3 |
| 17A/F | 39 | 40 | 41 | 99 | 100 | 101 |
| 17A/F | 42 | 43 | 44 | 102 | 103 | 104 |
| 17A | 45 | 46 | 47 | 105 | 106 | 107 |
| 17A | 48 | 49 | 50 | 108 | 106 | 109 |
| 17A | 51 | 52 | 53 | 110 | 19 | 111 |
| 17RA | 54 | 55 | 56 | 112 | 100 | 113 |
| 17A | 57 | 58 | 59 | 114 | 100 | 115 |
| 17A | 60 | 61 | 62 | 116 | 117 | 118 |
| 17A | 39 | 64 | 65 | 119 | 120 | 121 |
| 17A/F | 66 | 67 | 68 | 122 | 16 | 123 |
| 17A | 75 | 76 | 77 | 124 | 106 | 109 |

In some embodiments, multispecific antibodies described herein comprise an amino acid sequence that is at least 60%, at least 65%, at least 70%, at least 75%, at least 80%, at least 85%, at least 90%, at least 95% or 100% identical to any one of VL sequences described in TABLE 34, wherein the multispecific molecule is capable of binding an amino acid sequence that is at least 60%, 70%, 75%, 80%, 85%, 90%, 95%, 99% or 100% identical to any one of SEQ ID NOS: 28-38.

TABLE 34
Exemplary VL sequence sequences of antibodies for binding to IL-17
and/or IL-17R

| | SEQ ID | |
|---|---|---|
| Target | NO: | VL Sequences |
| 17A/F | 126 | EIVLTQSPATLSLSPGERATLSCRASQSVRSYLAWYQQKPGQAPR |
| | | LLIYDASNRATGIPARFSGSGSGTDFTLTISSLEPEDFAVYYCQQ |
| | | RSNWPPATFGGGTKVEIK |
| 17A/F | 127 | AIQLTQSPSSLSASVGDRVTITCRADESVRTLMHWYQQKPGKAPK |
| | | LLIYLVSNSEIGVPDRFSGSGSGTDFRLTISSLQPEDFATYYCQQ |
| | | TWSDPWTFGQGTKVEIK |
| 17A | 128 | DVVMTQTPLSLPVTLGQPASISCRSSQSLVHSNGNTYLHWYLQKP |
| | | GQSPRLLIYKVSNRFSGVPDRFSGSGSGTDFTLKISRVEAEDLGV |
| | | YYCSQSTHFWTFGGGTKLEIK |
| 17A | 129 | DIVMTQTPLSLSVTPGQPASISCRSSRSLVHSRGNTYLHWYLQKP |
| | | GQSPQLLIYKVSNRFIGVPDRFSGSGSGTDFTLKISRVEAEDVGV |
| | | YYCSQSTHLPFTFGQGTKLEIK |
| 17A | 130 | EIVLTQSPGTLSLSPGERATLSCRASQSVSSSYLAWYQQKPGQAP |
| | | RLLIYGASSRATGIPDRFSGSGSGTDFTLTISRLEPEDFAVYYCQ |
| | | QYGSSPCTFGQGTRLEIK |
| 17RA | 131 | EIVMTQSPATLSVSPGERATLSCRASQSVSSNLAWFQQKPGQAPR |
| | | PLIYDASTRATGVPARFSGSGSGTDFTLTISSLQSEDFAVYYCQQ |
| | | YDNWPLTFGGGTKVEIK |
| 17A | 132 | EIVLTQSPGTLSLSPGERATLSCRASQSVSSSYLAWYQQKPGQAP |
| | | RLLIYDASSRATGIPDRFSGSGSGTDFTLTISRLEPEDFAVYYCQ |
| | | QYSYSPVTFGQGTKVEIK |
| 17A | 133 | DIQMTQSPSSLSASVGDRVTITCRASQDINSYLSWFQQKPGKAPK |
| | | SLIVRANRLVDGVPSRFSGSGSGQDYSLTISSLQPEDFATYYCLQ |
| | | YDAFPPYTFGQGTKLEIK |
| 17A | 134 | DIQMTQSPSSLSASVGDRVTITCRASQGIRNYLAWYQQKPGKAPK |
| | | LLIYAASTLQSGVPSRFSGSGSGTDFTLTISSLQPEDVATYYCQR |
| | | YNRAPYTFGQGTKVEIK |
| 17A | 135 | DIVMTQTPLSLSVTPGQPASISCRSSRSLVHSRGETYLHWYLQKP |
| | | GQSPQLLIYKVSNRFIGVPDRFSGSGSGTDFTLKISRVEAEDVGV |
| | | YYCSQSTHLPFTFGCGTKLEIK |
| 17A | 136 | DVVMTQSPLSLPVTLGQPASISCRSSQSLVHSNGNSYLHWYLQKP |
| | | GQSPQLLIYKVSNRFSGVPDRFSGSGSGTDFTLKISRVEAEDVGV |
| | | YYCSQSTHVPYTFGQGTKLEIK |
| 17A | 137 | EIVLTQSPDFQSVTPKEKVTITCSASSSVNYMHWFQQKPDQSPKL |
| | | WIYRTSNLASGVPSRFSGSGSGTDYTLTINSLEAEDAATYYCQQR |
| | | SSYPWTFGQGTKLEIK |
| 17A | 138 | DIQMTQSPSSLSASVGDRVTITCRASQNVHNRLTWYQQKPGKAPK |
| | | LLIYGASNLESGVPSRFSGSGSGTDFTLTISSLQPEDFATYYCQQ |
| | | YNGSPTTFGQGTKVEIK |

In some embodiments, multispecific antibodies described herein comprise at least one modification within any one of the VL sequences described in TABLE 34, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is an IL-17 family cytokine amino acid sequence, or portion thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification. In some embodiments, multispecific antibodies described herein comprise two light chain variable region that are at least 90%, at least 95%, at least 98% or 100% identical relative to each other.

In some embodiments, multispecific antibodies comprise n IL-17a binding region, wherein the IL-17 binding region comprises an IL-17 binding heavy chain variable domain and, optionally, an IL-17 binding light chain variable domain. In some embodiments, multispecific antibodies described herein comprise a binding affinity for IL-17, a variant thereof or a functional fragment thereof that is at least 10%, at least 20%, at least 30%, at least 50%, at least 80%, at least 100%, at least 200% or more higher under neutral pH condition relative to the binding affinity under acidic pH condition. In some embodiments, multispecific antibodies described herein comprise a ratio of binding affinities for IL-17, a variant thereof or a functional fragment under neutral pH condition and acidic pH condition is at least 1.1, 1.5, 2, 3, 5, 8, 10, 100 or more. In some embodiments, multispecific antibodies described herein comprise a binding affinity for IL-17, a variant thereof or a functional fragment thereof that is at least 10%, at least 20%, at least 30%, at least 50%, at least 80%, at least 100%, at least 200% or more higher at about pH 7.4 relative to the binding affinity at about pH 5.8. In some embodiments, multispecific antibodies described herein comprise a ratio of binding affinities for IL-17, a variant thereof or a functional fragment at about pH 7.4 and about pH 5.8 is at least 1.1, 1.5, 2, 3, 5, 8, 10, 100 or more.

In some embodiments, multispecific antibodies described herein comprise: (a) a VH sequence that is at least 60%, at least 65%, at least 70%, at least 75%, at least 80%, at least 85%, at least 90%, at least 95% or 100% identical to any one of amino acid sequences described in TABLE 31; and (b) a VL sequence that is at least 60%, at least 65%, at least 70%, at least 75%, at least 80%, at least 85%, at least 90%, at least 95% or 100% identical to any one of amino acid sequences described in TABLE 34, wherein the multispecific molecule comprises the VH sequence and the VL sequence according to the combination described in TABLE 35.

TABLE 35
Exemplary VH and VL sequences of antibodies
for binding to IL-17 and/or IL-17R

| Target | VH Amino Acid Sequence | VL Amino Acid Sequence |
|---|---|---|
| 17A/F | 84 | 126 |
| 17A/F | 85 | 127 |
| 17A | 86 | 128 |
| 17A | 87 | 129 |
| 17A | 88 | 130 |
| 17RA | 89 | 131 |
| 17A | 90 | 132 |
| 17A | 91 | 133 |
| 17A | 92 | 134 |
| 17A/F | 93 and 94 | — |
| 17A | 95 | 135 |
| 17A | 96 | 136 |
| 17A | 97 | 137 |
| 17A | 98 | 138 |

### TL1A

Tumor necrosis factor (TNF)-like cytokine 1A (TL1A; HGNC: 11931, Entrez Gene: 9966, UniProtKB: 095150) is a type 2 transmembrane protein that self-assembles into stable trimers and binds to death receptor 3 (DR3). TL1A can also bind to TNFR2 to produce proinflammatory cytokines like IL-6, ROS, and then impairs mitochondrial dysfunction. TL1A is also known as Tumor Necrosis Factor Ligand Superfamily Member 15 TNFSF15), TL1, VEGI, TNLG1B, or VEGI192A. TL1A is mainly expressed as the membrane-bound form.

TL1A is constitutive expressed in immune cells (e.g., monocyte, macrophage, dendritic cell, and T cells) and non-immune cells (e.g., endothelial cells and synovial fibroblast cells). TL1A expression in immune cells (e.g., macrophages and dendritic cells) increased by TLR4, TLR11, or FcgR. In non-immune cells (e.g., endothelial cells), TL1A constitutively expressed and upregulated in response to TNFα stimulation.

TL1A is also expressed as soluble form (sTL1A) that is produced by alternative splicing or TNFα converting enzyme (TACCE) cleavage. sTL1A can be detected in serum and body fluids of patients with T cell-mediated inflammatory autoimmune diseases, e.g., rheumatoid arthritis, psoriatic arthritis, ankylosing spondylitis. sTL1A/DR3 binding promotes proinflammatory cytokine secretion, lymphocyte proliferation and cell apoptosis. For example, sTL1A can bind to DR3 and initiate one of two downstream pathways to cause: (1) inflammation—TRADD is recruited to the cytoplasmic domain of DR3, and then further recruits TRAF2 and RIP1 to initiate and activate MAPKs, NFkB, and PI3K signaling to regulate expression of pro-inflammatory genes; or (2) apoptosis—TRADD is recruited to the cytoplasmic domain of DR3, and TRADD binds to FADD and RIP3 to activate Caspase-8 to form complexes. It, then, induces apoptotic cell death through caspase pathway (−3 and −7). sTL1A can bind to soluble decoy receptor 3 (DcR3). When DcR3 competitively binds to sTL1A, combination of sTL1A and DR3 may be destroyed and results in less lymphocyte activation, less pro-inflammatory cytokine production, and prevents apoptosis. DcR3 can bind to other ligands like FasL and LIGHT.

TL1A is associated with autoimmune conditions (e.g., rheumatoid arthritis, inflammatory bowel disease, psoriasis, primary biliary cirrhosis, systemic lupus erythematosus, ankylosing spondylitis). The autoimmune conditions and its relationship with the TL1A expression are summarized in TABLE 36.

TABLE 36
TL1A Expression and Autoimmune Conditions

| | | Increased/ |
|---|---|---|
| Disorders | Expression of TL1A | Decreased |
| Rheumatoid arthritis | Serum, synovial fluid | Increased |
| Inflammatory bowel disease | Colonic tissues | Increased |
| Psoriasis | Serum | Increased |
| Primary biliary cirrhosis | Serum | Increased |
| Systemic lupus erythematosus | Serum | Increased |
| Ankylosing spondylitis | Serum | Increased |

In some embodiments, serum level of TL1A is significantly associated with progression of atherosclerotic plaque height in subjects having rheumatoid arthritis. In some embodiments, treatment of collagen-induced arthritis (CIA) mice with anti-TL1A antibody decreases total joint score and clinical inflammation. In some embodiments, TL1A gene knock-out results in improved clinical profiles for CIA mice relative to wildtype mice.

In some embodiments, TL1A affects epithelial to mesenchymal transition (EMT) and increases the barrier permeability (reduces tight function protein), resulting in causing colonic fibrosis and inflammatory responses in subjects having inflammatory bowel disease. In some embodiments, TL1A expression is elevated in subjects having Crohn's disease. In some embodiments, anti-TL1A improves tissue inflammation and inhibited expression of fibrotic pathways.

In some embodiments, TL1A is predominantly expressed in psoriatic lesions, particularly in infiltrating inflammatory cells, keratinocytes, and vascular cells. In some embodiments, TL1A promotes production of IL-17, which ultimately leads to early inflammation. In some embodiments, anti-TL1A antibody treatment alleviates histopathological changes. In some embodiments, TL1A can synergize with IL-23 to stimulate IL-17 secretion in peripheral blood mononuclear cells (PBMCs), thereby aggravating the autoimmune condition.

In some embodiments, serum TL1A levels are higher in subjects having primary biliary cirrhosis (PBS). In some embodiments, TL1A is expressed in biliary epithelial cells, vascular cells and infiltrating mononuclear cells of PBC liver. In some embodiments, the subjects show decrease in serum TL1A level after treatment with ursodeoxycholic acid (UDCA).

In some embodiments, multispecific molecules described herein are capable of binding TL1A protein. In some embodiments, multispecific (e.g., bispecific, trispecific) molecules described herein that bind to a mammalian TL1A sequence. In some embodiments, the TL1A is a human homolog. In some embodiments, the TL1A is a murine homolog.

An amino acid sequence of a human TL1A protein is recited in TABLE 37.

TABLE 37
Amino Acid Sequence of human TL1A protein

| Name | SEQ ID NO: | Amino Acid Sequences |
|---|---|---|
| TL1A | 139 | MAEDLGLSFGETASVEMLPEHGSCRPKARSSSARWALTCCLVLLPFL |
| | | AGLTTYLLVSQLRAQGEACVQFQALKGQEFAPSHQQVYAPLRADGDK |
| | | PRAHLTVVRQTPTQHFKNQFPALHWEHELGLAFTKNRMNYTNKFLLI |
| | | PESGDYFIYSQVTFRGMTSECSEIRQAGRPNKPDSITVVITKVTDSY |
| | | PEPTQLLMGTKSVCEVGSNWFQPIYLGAMFSLQEGDKLMVNVSDISL |
| | | VDYTKEDKTFFGAFLL |
| TL1A | 140 | MQLTKGRLHFSHPLSHTKHISPFVTDAPLRADGDKPRAHLTVVRQTP |
| Isoform | | TQHFKNQFPALHWEHELGLAFTKNRMNYTNKFLLIPESGDYFIYSQV |
| 2 | | TFRGMTSECSEIRQAGRPNKPDSITVVITKVTDSYPEPTQLLMGTKS |
| | | VCEVGSNWFQPIYLGAMFSLQEGDKLMVNVSDISLVDYTKEDKTFFG |
| | | AFLL |
| TL1A | 141 | MRRFLSKVYSFPMRKLILFLVFPVVRQTPTQHFKNQFPALHWEHELG |
| Isoform | | LAFTKNRMNYTNKFLLIPESGDYFIYSQVTFRGMTSECSEIRQAGRP |
| 3 | | NKPDSITVVITKVTDSYPEPTQLLMGTKSVCEVGSNWFQPIYLGAMF |
| | | SLQEGDKLMVNVSDISLVDYTKEDKTFFGAFLL |

In some embodiments, multispecific molecules described herein bind to an amino acid sequence that is at least 60%, at least 70%, at least 75%, at least 80%, at least 85%, at least 90%, at least 95%, at least 99% or 100% identical to any one of the sequences recited in TABLE 37. In some embodiments, multispecific molecules described herein bind to an amino acid sequence that is at least 60%, 70%, 75%, 80%, 85%, 90%, 95%, 99% or 100% identical to SEQ ID NO: 139.

In some embodiments, multispecific molecules described herein comprise at least one of CDR-Hs described in TABLE 38 or a variant thereof, wherein the multispecific molecule is capable of binding an amino acid sequence that is at least 60%, 70%, 75%, 80%, 85%, 90%, 95%, 99% or 100% identical to any one of SEQ ID NOS: 139-141. In some embodiments, multispecific molecules described herein comprise any one of CDR-H1 described in TABLE 38 or a variant thereof, any one of CDR-H2 described in TABLE 38 or a variant thereof, and any one of CDR-H3 described in TABLE 38 or a variant thereof, wherein the multispecific molecule is capable of binding an amino acid sequence that is at least 60%, 70%, 75%, 80%, 85%, 90%, 95%, 99% or 100% identical to any one of SEQ ID NOS: 139-141. In some embodiments, multispecific molecules described herein comprise any one of combinations of CDR-Hs or variants thereof described in TABLE 38, wherein the multispecific molecule is capable of binding an amino acid sequence that is at least 60%, 70%, 75%, 80%, 85%, 90%, 95%, 99% or 100% identical to any one of SEQ ID NOS: 139-141. In some embodiments, the CDR-H variant comprises at least one, at least two or at least three substitutions, deletions, additions or combination thereof relative to a corresponding parent CDR-H sequence described in TABLE 38. In some embodiments, the CDR-H or the variant thereof comprises an amino acid sequence that is at least 60%, at least 65%, at least 70%, at least 75%, at least 80%, at least 85%, at least 90%, at least 95% or 100% identical to a corresponding parent CDR-H sequence described in TABLE 38.

TABLE 38
Exemplary CDR-H sequences of antibodies for binding to TL1A

| SEQ | | SEQ | | SEQ | |
|---|---|---|---|---|---|
| ID | | ID | | ID | |
| NO: | CDR-H1 | NO: | CDR-H2 | NO: | CDR-H3 |
| 142 | GYTFTSYD | 143 | LNPNSGYT | 144 | AREVPETAAFEY |
| 145 | GFDIQDTY | 146 | IDPASGHT | 147 | ARSGGLPDV |
| 159 | GFDIQDTYMH | 160 | RIDPASGHTKYDPKFQV | 175 | SRSGGLPDV |
| | | 161 | RIEPASGHIKYDPKFQG | 147 | ARSGGLPDV |
| | | 162 | RIEPASGHIKYSPKFQG | 176 | ARSGGLPDW |
| | | 163 | RIEPASGHVKYSPKFQV | 177 | ARSGGLPDM |
| | | 164 | RIEPASGHVKYDPKFQT | 178 | ARSGGLPDK |
| | | 165 | RIDPASGHIKYDPKFQK | 179 | ARSGGLPDF |
| | | 166 | RIDPASGHVKIDPKFQV | 180 | ARSGGLPDL |
| | | 167 | RIDPASGHLKYDPKFQV | | |
| | | 168 | RIDPASGHLKYDPKFQR | | |
| | | 169 | RIDPASGHLKYDPKFQN | | |
| | | 170 | RIEPASGHLKYDPKFQE | | |
| | | 171 | RIDPASGHLKYDPKFQG | | |
| | | 172 | RIDPASGHTKYDPKFQG | | |
| | | 173 | RIDPASGHSKYDPKFQV | | |
| | | 1770 | RIDPASGHYKYDPKFQV | | |
| 181 | DTYMH | 182 | PASGH | 183 | SGGLPD |
| 484 | YYGIS | 485 | WISTYNGNTHYARMLQG | 486 | ENYYGSGAYRGGMDV |
| 733 | SRNWWS | 586 | EIYHSGSTNYNPSLKS | 492 | DNDILTGYFDGYFDY |
| | | | | 493 | DNDILDGYFDGYFDY |
| | | | | 494 | DNDILTGYDDGYFDY |
| | | | | 495 | DNDILTGYNDGYFDY |
| 1771 | LYGMN | 604 | WINTNTGNPTYAQGFTG | 549 | DNWNYVIDH |

In some embodiments, multispecific antibodies described herein comprise at least one modification within any one of the CDR-Hs described in TABLE 38, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is TL1A, a variant thereof or a functional fragment thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprises an amino acid sequence that is at least 60%, at least 65%, at least 70%, at least 75%, at least 80%, at least 85%, at least 90%, at least 95% or 100% identical to any one of VH sequences described in TABLE 39, wherein the multispecific molecule is capable of binding an amino acid sequence that is at least 60%, 70%, 75%, 80%, 85%, 90%, 95%, 99% or 100% identical to any one of SEQ ID NOS: 139-141.

TABLE 39
Exemplary VH sequence sequences of antibodies for binding to TL1A

| SEQ ID NO: | VH Sequences |
|---|---|
| 149 | QVQLVQSGAEVKKPGASVKVSCKASGYTFTSYDINWVRQAPGQGLEWMGWLN |
| | PNSGYTGYAQKFQGRVTMTADRSTSTAYMELSSLRSEDTAVYYCAREVPETA |
| | AFEYWGQGTLVTVSS |
| 150 | QVQLVQSGAEVKKPGASVKVSCKASGFDIQDTYMHWVKQRPGQGLEWMGRID |
| | PASGHTKYDPKFQVRVTITRDTSTSTVYLELSSLRSEDTAVYYCARSGGLPD |
| | VWGQGTTVTVSS |
| 184 | QVQLVQSGAEVKKPGASVKVSCKASGFDIQDTYMHWVRQAPGQGLEWMGRID |
| | PASGHTKYDPKFQVRVTMTRDTSTSTVYMELSSLRSEDTAVYYCARSGGLPD |
| | VWGQGTTVTVSS |
| 185 | QVQLVQSGAEVKKPGASVKVSCKASGFDIQDTYMHWVRQAPGQGLEWMGRIE |
| | PASGHIKYDPKFQGRVTMTRDTSTSTVYMELSSLRSEDTAVYYCARSGGLPD |
| | WWGQGTTVTVSS |
| 186 | QVQLVQSGAEVKKPGASVKVSCKASGFDIQDTYMHWVRQAPGQGLEWMGRIE |
| | PASGHIKYSPKFQGRVTMTRDTSTSTVYMELSSLRSEDTAVYYCARSGGLPD |
| | WWGQGTTVTVSS |
| 187 | QVQLVQSGAEVKKPGASVKVSCKASGFDIQDTYMHWVRQAPGQGLEWMGRIE |
| | PASGHVKYSPKFQVRVTMTRDTSTSTVYMELSSLRSEDTAVYYCARSGGLPD |
| | WWGQGTTVTVSS |
| 188 | QVQLVQSGAEVKKPGASVKVSCKASGFDIQDTYMHWVRQAPGQGLEWMGRIE |
| | PASGHVKYDPKFQTRVTMTRDTSTSTVYMELSSLRSEDTAVYYCARSGGLPD |
| | WWGQGTTVTVSS |
| 189 | QVQLVQSGAEVKKPGASVKVSCKASGFDIQDTYMHWVRQAPGQGLEWMGRID |
| | PASGHIKYDPKFQKRVTMTRDTSTSTVYMELSSLRSEDTAVYYCARSGGLPD |
| | MWGQGTTVTVSS |
| 190 | QVQLVQSGAEVKKPGASVKVSCKASGFDIQDTYMHWVRQAPGQGLEWMGRID |
| | PASGHVKIDPKFQVRVTMTRDTSTSTVYMELSSLRSEDTAVYYCARSGGLPD |
| | MWGQGTTVTVSS |
| 191 | QVQLVQSGAEVKKPGASVKVSCKASGFDIQDTYMHWVRQAPGQGLEWMGRID |
| | PASGHLKYDPKFQVRVTMTRDTSTSTVYMELSSLRSEDTAVYYCARSGGLPD |
| | MWGQGTTVTVSS |
| 192 | QVQLVQSGAEVKKPGASVKVSCKASGFDIQDTYMHWVRQAPGQGLEWMGRID |
| | PASGHLKYDPKFQRRVTMTRDTSTSTVYMELSSLRSEDTAVYYCARSGGLPD |
| | MWGQGTTVTVSS |
| 193 | QVQLVQSGAEVKKPGASVKVSCKASGFDIQDTYMHWVRQAPGQGLEWMGRID |
| | PASGHLKYDPKFQNRVTMTRDTSTSTVYMELSSLRSEDTAVYYCARSGGLPD |
| | KWGQGTTVTVSS |
| 194 | QVQLVQSGAEVKKPGASVKVSCKASGFDIQDTYMHWVRQAPGQGLEWMGRIE |
| | PASGHLKYDPKFQERVTMTRDTSTSTVYMELSSLRSEDTAVYYCARSGGLPD |
| | KWGQGTTVTVSS |
| 195 | QVQLVQSGAEVKKPGASVKVSCKASGFDIQDTYMHWVRQAPGQGLEWMGRID |
| | PASGHLKYDPKFQGRVTITRDTSASTAYMELSSLRSEDTAVYYCARSGGLPD |
| | MWGQGTTVTVSS |
| 196 | QVQLVQSGAEVKKPGASVKVSCKASGFDIQDTYMHWVRQAPGQGLEWMGRID |
| | PASGHLKYDPKFQGRVTITRDTSASTVYMELSSLRSEDTAVYYCARSGGLPD |
| | MWGQGTTVTVSS |
| 197 | QVQLVQSGAEVKKPGASVKVSCKASGFDIQDTYMHWVRQAPGQGLEWMGRID |
| | PASGHTKYDPKFQGRATITTDTSASTAYLQLSSLRSEDTAVYYCARSGGLPD |
| | VWGQGTTVTVSS |
| 198 | QVQLVQSGAEVKKPGASVKVSCKASGFDIQDTYMHWVRQAPGQGLEWMGRID |
| | PASGHTKYDPKFQVRATITTDTSASTAYLQLSSLRSEDTAVYYCARSGGLPD |
| | FWGQGTTVTVSS |
| 199 | QVQLVQSGAEVKKPGASVKVSCKASGFDIQDTYMHWVRQAPGQGLEWMGRID |
| | PASGHTKYDPKFQGRATITTDTSASTAYLQLSSLRSEDTAVYYCARSGGLPD |
| | FWGQGTTVTVSS |
| 200 | QVQLVQSGAEVKKPGASVKVSCKASGFDIQDTYMHWVRQAPGQGLEWMGRID |
| | PASGHTKYDPKFQGRATITTDTSASTAYLQLSSLRSEDTAVYYCARSGGLPD |
| | LWGQGTTVTVSS |
| 201 | QVQLVQSGAEVKKPGASVKVSCKASGFDIQDTYMHWVRQAPGQGLEWMGRID |
| | PASGHSKYDPKFQVRATITTDTSASTAYLQLSSLRSEDTAVYYCARSGGLPD |
| | FWGQGTTVTVSS |
| 202 | QVQLVQSGAEVKKPGASVKVSCKASGFDIQDTYMHWVRQAPGQGLEWMGRID |
| | PASGHYKYDPKFQVRATITTDTSASTAYLQLSSLRSEDTAVYYCARSGGLPD |
| | FWGQGTTVTVSS |
| 487 | QVQLVQSGAEVKKPGASVKVSCKASGYDFTYYGISWVRQAPGQGLEWMGWIS |
| | TYNGNTHYARMLQGRVTMTTDTSTRTAYMELRSLRSDDTAVYYCARENYYGS |
| | GAYRGGMDVWGQGTTVTVSS |
| 902 | QVQLQESGPGLVKPSGTLSLTCAVSGGSISSRNWWSWVRQPPGKGLEWIGEI |
| | YHSGSTNYNPSLKSRVIISVDKSKNQFSLNLNSVTAADTAVYYCARDNDILT |
| | GYFDGYFDYWGQGTLVTVSS |
| 903 | QVQLQESGPGLVKPSGTLSLTCAVSGGSISSRNWWSWVRQPPGKGLEWIGEI |
| | YHSGSTNYNPSLKSRVTISVDKSKNQFSLNLNSVTAADTAVYYCARDNDILD |
| | GYFDGYFDYWGQGTLVTVSS |
| 904 | QVQLQESGPGLVKPSGTLSLTCAVSGGSISSRNWWSWVRQPPGKGLEWIGEI |
| | YHSGSTNYNPSLKSRVTISVDKSKNQFSLNLNSVTAADTAVYYCARDNDILT |
| | GYDDGYFDYWGQGTLVTVSS |
| 905 | QVQLQESGPGLVKPSGTLSLTCAVSGGSISSRNWWSWVRQPPGKGLEWIGEI |
| | YHSGSTNYNPSLKSRVTISVDKSKNQFSLNLNSVTAADTAVYYCARDNDILT |
| | GYNDGYFDYWGQGTLVTVSS |
| 907 | QVQLQESGPGLVKPSGTLSLTCAVSGGSITSRNWWSWVRQPPGKGLEWIGEI |
| | YHSGSTNYNPSLKSRVTISVDKSKNQFSLNLNSVTAADTAVYYCARDNDILT |
| | GYFDGYFDYWGQGTLVTVSS |
| 908 | QVQLQESGPGLVKPSGTLSLTCAVSGGSISSRNWWSWVRQPPGKGLEWIGEI |
| | YHSGSTNYNPSLKSRVTISVDKSKNQFSLNVNSVTAADTAVYYCARDNDILT |
| | GYFDGYFDYWGQGTLVTVSS |
| 909 | QVQLQESGPGLVKPSGTLSLTCAVSGGSISSRNWWSWVRQPPGKGLEWIGEI |
| | YHSGSTNYNPSLKSRVTISVDKSKNQFSLNLKSVTAADTAVYYCARDNDILT |
| | GYFDGYFDYWGQGTLVTVSS |
| 923 | QVQLQESGPGLVKPSGTLSLTCAVSGGSISSRNWWSWVRQPPGKGLEWIGEI |
| | YHSGSTNYNPSLKSRVTISVDKSKNQFSLNLNSVTAADTAVYYCARDNDILT |
| | GYFDGYFDYWGQGTLVTVSS |
| 933 | QVQLQESGPGLVEPSGTLSLTCAVSGGSISSRNWWSWVRQPPGKGLEWIGEI |
| | YHSGSTNYNPSLKSRVTISVDKSKNQFSLNLNSVTAADTAVYYCARDNDILT |
| | GYFDGYFDYWGQGTLVTVSS |
| 1048 | QVQLVQSGSELKKPGASVKVSCKASGYSFILYGMNWVRQAPGQGLEWMGWIN |
| | TNTGNPTYAQGFTGRFVFSLDTSVSTAYLQISSLKADDTAVYYCARDNWNYV |
| | IDHWGQGTLVTVSS |

In some embodiments, multispecific antibodies described herein comprise at least one modification within any one of the VH sequences described in TABLE 39, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is TL1A, a variant thereof or a functional fragment thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise at least one of CDR-Ls described in TABLE 40 or a variant thereof, wherein the multispecific molecule is capable of binding an amino acid sequence that is at least 60%, 70%, 75%, 80%, 85%, 90%, 95%, 99% or 100% identical to any one of SEQ ID NOS: 139-141. In some embodiments, multispecific molecules described herein comprise any one of CDR-L1 described in TABLE 40 or a variant thereof, any one of CDR-L2 described in TABLE 40 or a variant thereof, and any one of CDR-L3 described in TABLE 40 or a variant thereof, wherein the multispecific molecule is capable of binding an amino acid sequence that is at least 60%, 70%, 75%, 80%, 85%, 90%, 95%, 99% or 100% identical to any one of SEQ ID NOS: 139-141. In some embodiments, multispecific molecules described herein comprise any one of combinations of CDR-Ls or variants thereof described in TABLE 40, wherein the multispecific molecule is capable of binding an amino acid sequence that is at least 60%, 70%, 75%, 80%, 85%, 90%, 95%, 99% or 100% identical to any one of SEQ ID NOS: 139-141. In some embodiments, the CDR-L variant comprises at least one, at least two or at least three substitutions, deletions, additions or combination thereof relative to a corresponding parent CDR-L sequence described in TABLE 40. In some embodiments, the CDR-L or the variant thereof comprises an amino acid sequence that is at least 60%, at least 65%, at least 70%, at least 75%, at least 80%, at least 85%, at least 90%, at least 95% or 100% identical to a corresponding parent CDR-L sequence described in TABLE 40.

TABLE 40
Exemplary CDR-L sequences of antibodies for binding to TL1A

| SEQ ID | | SEQ ID | | SEQ ID | |
|---|---|---|---|---|---|
| NO: | CDR-L1 | NO: | CDR-L2 | NO: | CDR-L3 |
| 151 | SSDIGAGLG | 152 | GYY | 153 | QSWDGTLSAL |
| 154 | SSVSY | 155 | ATS | 156 | QQWEGNPRT |
| 203 | RASSSVSYMY | 205 | ATSNLAS | 206 | QQWSGNPRT |
| 204 | GASSSVSYMY | | | 156 | QQWEGNPRT |
| | | | | 207 | QQWQGNPRT |
| | | | | 208 | SQWSGNPRT |
| | | | | 209 | QQWSGNPRS |
| | | | | 210 | QQWSRNPRT |
| | | | | 211 | QQWKGNPRT |
| 212 | ASSSVSYMY | 205 | ATSNLAS | 1769 | GNPRT |
| 488 | RASQSVSSYLA | 489 | DASNRAT | 490 | QQRSNWPWT |
| 901 | RASQSVSSSYLA | 900 | GASSRAT | 899 | QQYGSSPLT |

In some embodiments, multispecific antibodies described herein comprise at least one modification within any one of the CDR-Ls described in TABLE 40, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is TL1A, a variant thereof or a functional fragment thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification.

In some embodiments, multispecific molecules described herein comprise a combination of CDRs, wherein the CDRs comprises a CDR-H1 or a variant thereof, a CDR-H2 or a variant thereof, a CDR-H3 or a variant thereof, a CDR-L1 or a variant thereof, a CDR-L2 or a variant thereof, and a CDR-L3 or a variant thereof, and wherein the combination is according to any one of the combinations provided in TABLE 41.

TABLE 41
Exemplary CDR sequences of antibodies for binding to TL1A

| Target | CDR-H1 | CDR-H2 | CDR-H3 | CDR-L1 | CDR-L2 | CDR-L3 |
|---|---|---|---|---|---|---|
| TL1A | 142 | 143 | 144 | 151 | 152 | 153 |
| TL1A | 145 | 146 | 147 | 154 | 155 | 156 |
| TL1A | 159 | 160-172 | 147, | 203-204 | 205 | 156, |
| | | | 175-180 | | | 206-211 |
| TL1A | 181 | 182 | 183 | 212 | 205 | 156 |
| TL1A | 484 | 485 | 486 | 488 | 489 | 490 |
| TL1A | 733 | 586 | 492-495 | 901 | 900 | 899 |

In some embodiments, multispecific molecules described herein comprise an amino acid sequence that is at least 60%, at least 65%, at least 70%, at least 75%, at least 80%, at least 85%, at least 90%, at least 95% or 100% identical to any one of VL sequences described in TABLE 42, wherein the multispecific molecule is capable of binding an amino acid sequence that is at least 60%, 70%, 75%, 80%, 85%, 90%, 95%, 99% or 100% identical to any one of SEQ ID NOS: 139-141.

TABLE 42
Exemplary VL sequence sequences of antibodies for binding to TL1A

| SEQ ID | |
|---|---|
| NO: | VL Sequences |
| 157 | QSVLTQPPSVSGAPGQRVTISCTSSSSDIGAGLGVHWYQQLPGTAPKLLIEGYYNRP |
| | SGVPDRFSGSKSGTSASLTITGLLPEDEGDYYCQSWDGTLSALFGGGTKLTVL |
| 158 | EIVLTQSPGTLSLSPGERATLSCRASSSVSYMYWYQQKPGQAPRPLIYATSNLASGI |
| | PDRFSGSGSGTDFTLTISRLEPEDFAVYYCQQWEGNPRTFGGGTKLEIK |
| 213 | EIVLTQSPGTLSLSPGERATLSCRASSSVSYMYWYQQKPGQAPRLLIYATSNLASGI |
| | PDRFSGSGSGTDFTLTISRLEPEDFAVYYCQQWSGNPRTFGGGTKLEIK |
| 214 | EIVLTQSPGTLSLSPGERATLSCRASSSVSYMYWYQQKPGQAPRLLIYATSNLASGI |
| | PDRFSGSGSGTDFTLTISRLEPEDFAVYYCQQWEGNPRTFGGGTKLEIK |
| 215 | EIVLTQSPGTLSLSPGERATLSCGASSSVSYMYWYQQKPGQAPRLLIYATSNLASGI |
| | PDRFSGSGSGTDFTLTISRLEPEDFAVYYCQQWEGNPRTFGGGTKLEIK |
| 216 | EIVLTQSPGTLSLSPGERATLSCRASSSVSYMYWYQQKPGQAPRLLIYATSNLASGI |
| | PDRFSGSGSGTDFTLTISRLEPEDFAVYYCQQWQGNPRTFGGGTKLEIK |
| 217 | EIVLTQSPGTLSLSPGERATLSCRASSSVSYMYWYQQKPGQAPRLLIYATSNLASGI |
| | PDRFSGSGSGTDFTLTISRLEPEDFAVYYCSQWSGNPRTFGGGTKLEIK |
| 218 | EIVLTQSPGTLSLSPGERATLSCRASSSVSYMYWYQQKPGQAPRLLIYATSNLASGI |
| | PDRFSGSGSGTDFTLTISRLEPEDFAVYYCQQWSGNPRSFGGGTKLEIK |
| 219 | EIVLTQSPGTLSLSPGERATLSCRASSSVSYMYWYQQKPGQAPRLLIYATSNLASGI |
| | PDRFSGSGSGTDFTLTISRLEPEDFAVYYCQQWSRNPRTFGGGTKLEIK |
| 220 | EIVLTQSPGTLSLSPGERATLSCRASSSVSYMYWYQQKPGQAPRLLIYATSNLASGI |
| | PDRFSGSGSGTDFTLTISRLEPEDFAVYYCQQWKGNPRTFGGGTKLEIK |
| 491 | EIVLTQSPATLSLSPGERATLSCRASQSVSSYLAWYQQKPGQAPRLLIYDASNRATG |
| | IPARFSGSGSGTDFTLTISSLEPEDFAVYYCQQRSNWPWTFGQGTKVEIK |
| 1768 | EIVLTQSPGTLSLSPGERATLSCRASQSVSSSYLAWYQQKPGQAPRLLIYGASSRAT |
| | GIPDRFSGSGSGTDFTLTISRLEPEDFAVYYCQQYGSSPLTFGQGTKVEIK |

In some embodiments, multispecific antibodies described herein comprise at least one modification within any one of the VL sequences described in TABLE 42, wherein the at least one modification changes isoelectric point (pI) of the multispecific antibodies relative to a corresponding pI prior to the at least one modification. Alternatively, in some embodiments, multispecific antibodies described herein comprise at least one modification within any one of framework regions, wherein the at least one modification changes isoelectric point (pI) of the multispecific antibodies relative to a corresponding pI prior to the at least one modification. In some embodiments, the at least one modification provides a pH-dependent binding activity for a target peptide to multispecific antibodies, wherein the target peptide is TL1A, a variant thereof or a functional fragment thereof. In some embodiments, the multispecific antibodies comprising the at least one modification have a binding affinity for the target peptide that is at least 60%, at least 75%, at least 80%, at least 85%, at least 90%, at least 95%, 100% or more in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more increase in binding affinity for the target peptide in neutral pH condition relative to a corresponding binding affinity prior to the at least one modification. In some embodiments, the multispecific antibodies comprising the at least one modification have at least 2%, at least 5%, at least 10%, at least 15%, at least 20%, at least 30%, at least 40%, at least 50% or more decrease in binding affinity for the target peptide in acidic pH condition relative to a corresponding binding affinity prior to the at least one modification. In some embodiments, multispecific antibodies described herein comprise two light chain variable region that are at least 90%, at least 95%, at least 98% or 100% identical relative to each other. In some embodiments, at least one amino acid modification in TL1A binding heavy chain variable region increases in vivo half-life of multispecific antibody by at least 2%, at least 5%, at least 10%, at least 15%, at least 20%, at least 30%, at least 40%, at least 50% or more relative to half-life of corresponding multispecific antibody prior to said at least one amino acid modification.

In some embodiments, the at least one modification in CDR-H3. In some embodiments, the at least one modification is a substitution in CDR-H3. In some embodiments, the at least one modification comprises a substitution of an uncharged amino acid with a charged amino acid in CDR-H3. The uncharged amino acid can be an amino acid with a hydrophobic side chain (e.g., glycine, alanine, valine, cysteine, proline, leucine, isoleucine, methionine, tryptophan, phenylalanine). Alternatively, the uncharged amino acid can be a polar uncharged amino acid (e.g., serine, threonine, tyrosine, asparagine, glutamine). The charged amino acid can be a negatively charged amino acid (e.g., aspartic acid, glutamic acid). Alternatively, the charged amino acid can be a positively charged amino acid (e.g., lysine, arginine, histidine). In some embodiments, the at least one modification is a substitution of a positively charged amino acid residue with a negatively charged amino acid residue in CDR-H3. In some embodiments, the at least one modification is a substitution of a negatively charged amino acid residue with a positively charged amino acid residue in CDR-H3. In some embodiments, a ratio of the binding affinities of target binding domains, variants thereof or functional fragments thereof, as described herein, for an epitope present on TL A, a variant thereof, or a fragment thereof at neutral pH and acidic pH, respective, is at least 1.2, at least 10, at least 100, at least 200, or more. In some embodiments, the VH sequence comprises an amino acid sequence of SEQ ID NO: 923. In some embodiments, the VH sequence comprises at least one modification at positions selected from the group consisting of 30, 68, 82, 82A, 100 and 100C, relative to SEQ ID NO: 923, per Kabat numbering. In some embodiments, the VH sequence comprises at least one modification at positions selected from the group consisting of S30, T68, L82, N82A, T100 and F100C, relative to the amino acid position numbering of SEQ ID NO: 923, per Kabat numbering. In some embodiments, the VH sequence comprises at least one modification at positions selected from the group consisting of 30, 69, 83, 84, 104 and 107, relative to the amino acid position numbering of SEQ ID NO: 923. In some embodiments, the VH sequence comprises at least one modification at positions selected from the group consisting of S30, T69, L83, N84, T104 and F107 relative to the amino acid position numbering of SEQ ID NO: 923. In some embodiments, the VH sequence comprises at least one substitution at positions selected from the group consisting of S30T, T69I, L83V, N84K, T104D, F107N and F107D relative to the amino acid position numbering of SEQ ID NO: 923.

In some embodiments, multispecific antibodies comprise a TL1A binding region, wherein the TL1A binding region comprises a TL1A binding heavy chain variable domain and, optionally, a TL1A binding light chain variable domain. In some embodiments, multispecific antibodies described herein comprise a binding affinity for TL1A, a variant thereof or a functional fragment thereof that is at least 10%, at least 20%, at least 30%, at least 50%, at least 80%, at least 100%, at least 200% or more higher under neutral pH condition relative to the binding affinity under acidic pH condition. In some embodiments, multispecific antibodies described herein comprise a ratio of binding affinities for TL1A, a variant thereof or a functional fragment under neutral pH condition and acidic pH condition is at least 1.1, 1.5, 2, 3, 5, 8, 10, 100 or more. In some embodiments, multispecific antibodies described herein comprise a binding affinity for TL A, a variant thereof or a functional fragment thereof is at least 10%, at least 20%, at least 30%, at least 50%, at least 80%, at least 100%, at least 200% or more higher at about pH 7.4 relative to the binding affinity at about pH 5.8. In some embodiments, multispecific antibodies described herein comprise a ratio of binding affinities for TL1A, a variant thereof or a functional fragment at about pH 7.4 and about pH 5.8 is at least 1.1, 1.5, 2, 3, 5, 8, 10, 100 or more. In some embodiments, multispecific antibodies described herein comprise a binding affinity for trimeric TL1A is at least 10%, at least 20%, at least 30%, at least 50%, at least 80%, at least 100%, at least 200% or more higher than the binding affinity for monomeric TL1A under neutral pH condition. In some embodiments, multispecific antibodies described herein comprise a binding affinity for trimeric TL1A is at least 10%, at least 20%, at least 30%, at least 50%, at least 80%, at least 100%, at least 200% or more higher than the binding affinity for monomeric TL1A at about pH 7.4.

In some embodiments, multispecific molecules described herein comprise: (a) a VH sequence that is at least 60%, at least 65%, at least 70%, at least 75%, at least 80%, at least 85%, at least 90%, at least 95% or 100% identical to any one of amino acid sequences described in TABLE 39; and (b) a VL sequence that is at least 60%, at least 65%, at least 70%, at least 75%, at least 80%, at least 85%, at least 90%, at least 95% or 100% identical to any one of amino acid sequences described in TABLE 42, wherein the multispecific molecule comprises the VH sequence and the VL sequence according to the combination described in TABLE 43.

TABLE 43
Exemplary VH and VL sequences of
antibodies for binding to TL1A

| Target | VH Amino Acid Sequence | VL Amino Acid Sequence |
|---|---|---|
| TL1A | 149 | 157 |
| TL1A | 150 | 158 |
| TL1A | 184-202 | 213-220 |
| TL1A | 487 | 491 |
| TL1A | 902-905, 907-909, 923, | 1768 |
| | 933, 1048 | |

### BsAb

Described herein are antibodies that are multispecific. In some embodiments, the multispecific antibodies target two or more different epitopes of the same target. In some embodiments, the multispecific antibodies are bispecific antibodies (BsAb) or trispecific antibodies. In some embodiments, the BsAb comprises a heavy chain region. In some embodiments, the BsAb comprises a light chain region. In some embodiments, the light chain region comprises a kappa light chain constant region. In some embodiments, the BsAb comprises an BsAb light chain variable region. In some embodiments, the BsAb comprises an BsAb heavy chain variable region. In some embodiments, the BsAb comprises an BsAb light chain variable region and an IgG heavy chain variable region. In some embodiments, the BsAb can be a humanized antibody. In some embodiments, the BsAb can be a chimeric antibody. In some embodiments, the BsAb can be a human antibody. In some embodiments, the BsAb comprises a common light chain (L chain). In some embodiments, the L chain acts against a specific target. The use of a common light chain can prioritize heterodimerization in the Fc region. In some embodiments, the L chain comprises a knob mutation. In some embodiments, the L chain comprises a hole mutation. In some embodiments, preferential heterodimer formation is preferred upon binding of the BsAb comprising a common L chain. In some embodiments, the formations of heterodimeric pairs are assembled using glutathione disulfide exchange.

In some embodiments, a BsAb described herein is an engineered BsAb that comprises one or more modifications of amino acids that can result in pH-dependent target binding activity. In some embodiments, an engineered BsAb described herein can exhibit pH-dependent target binding activity for a target peptide selected from TL1A, IL-6R, IL-6, IL-12, IL-23, IL-23p19, IL-12p40, IL-12p35, an IL-17 family cytokine, IL-17R, a variant thereof and a functional fragment thereof. In some embodiments, an engineered BsAb as described herein can readily bind to a target peptide at a neutral pH and dissociates from the target peptide at an acidic pH. Accordingly, upon administration to a subject the engineered BsAb can bind the target peptide in plasma on account of its neutral pH, while remaining dissociated from the target peptide in endosomes which have an acidic pH. Dissociation of the engineered BsAb from the target peptide in endosomes can facilitate recycling of the engineered BsAb into plasma through FcRn, whereas the target peptide can be trafficked to lysosome and degraded. Such characteristics of the engineered BsAb can allow sweeping of a target peptide from the plasma. Accordingly, in some embodiments, the engineered BsAb can comprise a target peptide sweeping activity for a target peptide that is selected from TL1A, IL-6R, IL-6, IL-12, IL-23, IL-23p19, IL-12p40, IL-12p35, an IL-17 family cytokine, IL-17R, a variant thereof and a functional fragment thereof.

In some embodiments, a BsAb described herein is an engineered BsAb that comprises one or more modifications of amino acids that can result in increased FcRn binding at neutral pH. In such embodiments, the engineered BsAb can have increased ability to repeatedly bind to FcRn and remove target peptide from plasma. Alternatively, in some embodiments, a BsAb described herein is an engineered BsAb that comprises one or more modifications of amino acids that can result in increased FcRn binding at acidic pH. In such embodiments, the engineered BsAb can have increased recycling efficiency from endosomes to plasma resulting in improving plasma retention of the engineered BsAb. Accordingly, in some embodiments, a constant domain of an engineered BsAb as described herein can be further modified for increasing FcRn binding activity at neutral pH and/or acidic pH.

In some embodiments, a BsAb described herein is an engineered BsAb that comprises one or more modifications of amino acids that can result in change of isoelectric point of the BsAb. In some embodiments, the one or more modifications of amino acids can result in change of isoelectric point of a VH sequence of the engineered BsAb. In some embodiments, the one or more modifications of amino acids can result in change of isoelectric point of a VL sequence of the engineered BsAb. In some embodiments, the one or more amino acid modifications can increase isoelectric point of the engineered BsAb. In some embodiments, increased isoelectric point can results in increased elimination rate of target peptides from plasma.

In some embodiments, a BsAb described herein is an engineered BsAb that comprises one or more modifications of amino acids that can result in pH-dependent target binding activity and/or increased FcRn binding activity. In some embodiments, the one or more modifications of amino acids comprise at least one, at least two, at least three, at least four, at least five, at least six, at least seven, at least eight, at least nine, at least ten, at least eleven, at least twelve, at least thirteen, at least fourteen, or at least fifteen amino acid modifications. In some embodiments, the one or more modifications of amino acids are in at least one of a Fab region, a scFv region, and a Fc region. In some embodiments, one or more modifications of amino acids are in a VL sequence of a BsAb, a VH sequence of a BsAb, or a combination thereof. In some embodiments, a modification of an amino acid is a deletion, a substitution, or an addition of the amino acid.

In some embodiments, a BsAb described herein comprises two constant domains. In some embodiments, the two constant domains are derived from a human IgG1 heavy constant domain. In some embodiments, the two constant domains are a first constant domain and a second constant domain. In some embodiments, the first constant domain is engineered to comprise a knob, and the second constant domain is engineered to comprise a hole. Accordingly, in some embodiments, the first constant domain comprises a modification at position T366, per EU numbering, and the second constant domain comprises a modification at position Y407, per EU numbering. In some embodiments, the first constant domain comprises a modification at position T366, per EU numbering, and the second constant domain comprises modifications at positions T366 and Y407, per EU numbering. In some embodiments, the first constant domain comprises a modification at position T366, per EU numbering, and the second constant domain comprises modifications at positions T366, L368 and Y407, per EU numbering. In some embodiments, the first constant domain comprises S354C substitution, T366W substitution, or a combination thereof, per EU numbering, and the second constant domain comprises Y349C substitution, T366S substitution, Y407V substitution, or a combination thereof, per EU numbering.

In some embodiments, a BsAb described herein comprises two constant domains that are derived from a human IgG1 heavy chain constant domain. In some embodiments, the two constant domains comprise a first constant domain and a second constant domain, wherein the first constant domain comprises at least two modifications and the second constant domain comprises at least one modification. For example, in some embodiments, the first constant domain comprises at least two modifications selected from modifications at positions L351, F405 and Y407, per EU numbering, and the second constant domain comprises at least one modification selected from modifications at positions T366, K392 and T394, per EU numbering. In some embodiments, the modification at position L351 comprises L351Y and L351A substitutions. In some embodiments, the modification at position F405 comprises F405A, F405S, F405T and F405V substitutions. In some embodiments, the modification at position Y407 comprises Y407A, Y407V, Y407S and Y407I substitutions. In some embodiments, the modification at position T366 comprises T366L, T366M, T366V and T366I substitutions. In some embodiments, the modification at position K392 comprises K392C, K392M, K392L, K392I, K392E, K392D and K392F substitutions. In some embodiments, the modification at position T394 comprises T394D, T394W, T394V and T394S substitutions. In some embodiments, the at least two modifications of the first constant domain further comprises one or more modifications at positions Q347, Y349, T350, K370, G371, D399 and S400, per EU numbering. In some embodiments, the modification at position Q347 comprises Q347R, Q347E and Q347K substitutions. In some embodiments, the modification at position Y349 comprises Y349C substitution. In some embodiments, the modification at position T350 comprises T350V substitution. In some embodiments, the modification at position K370 comprises K370T substitution. In some embodiments, the modification at position G371 comprises G371D and G371S substitutions. In some embodiments, the modification at position D399 comprises D399C, D399R and D399K substitutions. In some embodiments, the modification at position S400 comprises S400D, S400K, S400E and S400R substitutions. In some embodiments, the at least two modifications of the second constant domain further comprises one or more modifications at positions T350, S354, E357, K360, Q362E, S364, N390, K409 and T411, per EU numbering. In some embodiments, the modification at position T350 comprises T350V substitution. In some embodiments, the modification at position S354 comprises S354C substitution. In some embodiments, the modification at position E357 comprises E357Q substitution. In some embodiments, the modification at position K360 comprises K360D and K360E substitutions. In some embodiments, the modification at position Q362 comprises Q362E substitution. In some embodiments, the modification at position S364 comprises S364R substitution. In some embodiments, the modification at position N390 comprises N390K, N390R, N390D and N390E substitutions. In some embodiments, the modification at position K409 comprises K409L, K409M, K409F and K409W substitutions. In some embodiments, the modification at position T411 comprises T411R, T411D, T411I, T411K, T411E, T411N, T411S and T411L substitutions.

Alternatively, in some embodiments, a BsAb described herein comprises two constant domains that are derived from a human IgG1 heavy chain constant domain, wherein the two constant domains comprise a first constant domain and a second constant domain, wherein the first constant domain and the second constant domain are engineered to electrostatically interact with each other. In some embodiments, the first constant domain comprises a substitution at K370, per EU numbering, with a negatively charged amino acid residue (e.g., aspartic acid, glutamic acid), and the second constant domain comprises a substitution at E357, per EU numbering, with a positively charged amino acid residue (e.g., arginine, lysine, histidine). In some embodiments, the first constant domain comprises a substitution at K392 or K409, per EU numbering, with a negatively charged amino acid residue (e.g., aspartic acid, glutamic acid), and the second constant domain comprises a substitution at D399, per EU numbering, with a positively charged amino acid residue (e.g., arginine, lysine, histidine). In some embodiments, the first constant domain comprises a substitution at K439, per EU numbering, with a negatively charged amino acid residue (e.g., aspartic acid, glutamic acid), and the second constant domain comprises a substitution at D356, per EU numbering, with a positively charged amino acid residue (e.g., arginine, lysine, histidine).

### BiTE Antibody

In some embodiments, the molecule described herein comprises a bi-specific T-cell engager (BiTE) antibody construct and may be referred to herein as a “BiTE molecule”. A BiTe antibody construct is a type of fusion protein. In some embodiments, the BiTE molecule activates T-cell activity. In some embodiments, the BiTE molecule comprises two single-chain variable functional fragments. In some embodiments, the BiTE molecule comprises two binding domains. In some embodiments, the first binding domain is a TL1A binding domain. In some embodiments, the second binding domain is selected from any one of IL-6R, IL-6, IL-12, IL-23, IL-23p19, IL-12p40, IL-12p35, an IL-17 family cytokine, IL-17R, a variant thereof and a functional fragment thereof.

### Chemical Cross-Linking.

The use of chemical cross-linking reagents to covalently link two antibodies is known in the art. Antibody functional fragments generated from their respective parent antibodies by enzymatic digestion or generated through recombinant technologies may be conjugated using bifunctional.

### Quadromas

Quadromas and triomas can be generated by fusing either two hybridomas or one hybridoma with a B lymphocyte, respectively. In this case the simultaneous expression of two heavy and two light chains leads to the random assembly of 10 antibody combinations and the desired BsAb represent only a small fraction of the secreted antibodies. The BsAb may be purified using a combination of chromatographic techniques.

### Recombinant Multispecific Antibodies

Recombinant bispecific antibodies described herein comprise two nonidentical binding domains. In some embodiments, one of the two nonidentical binding domains of bispecific antibodies described herein can bind TL1A. In some embodiments, one of the two nonidentical binding domains of bispecific antibodies described herein can bind IL-6, IL-12, IL-23, IL-23p19, IL-12p40, IL-12p35, IL-6R, an IL-17 family cytokine, or IL-17R.

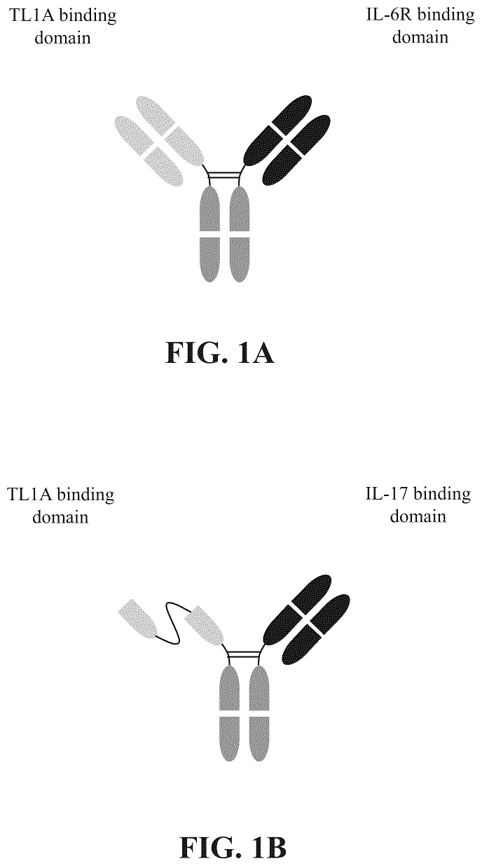

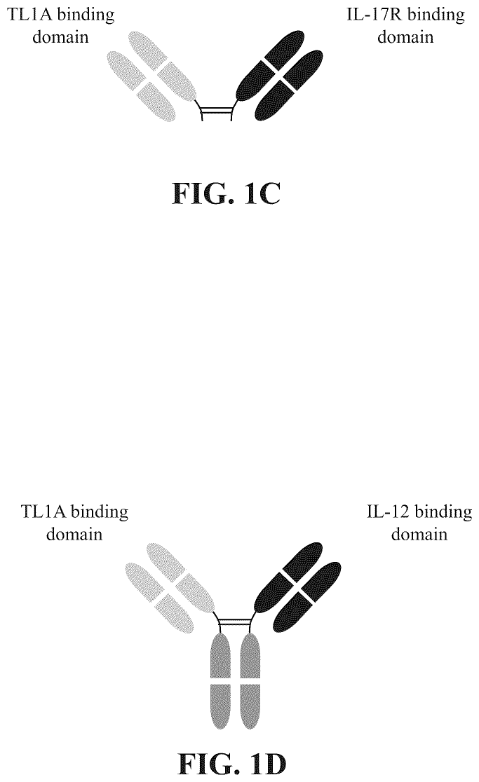

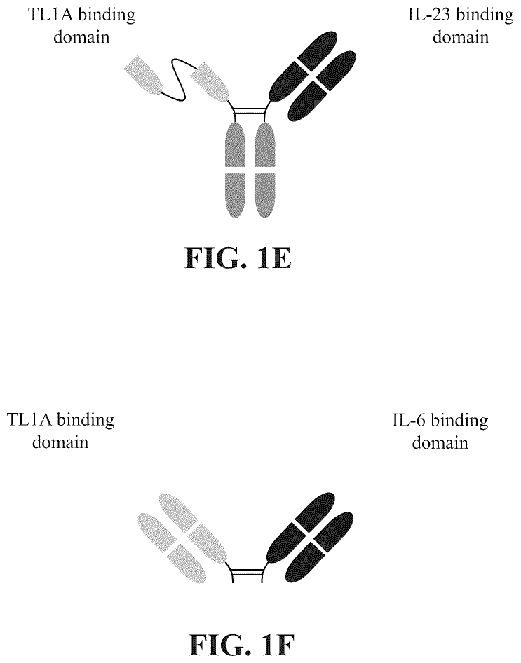

The majority of multispecific antibody formats can be generated by genetic engineering techniques using antibody functional fragment such as scFv or Fab fragments as building blocks connected via polypeptide linkers. Formats based on linked antibody functional fragments include tandem scFv (BiTE), diabodies and tandem-diabodies. These formats include diabody-Fc, tandem diabody-Fc, tandem diabody-CH3, (scFv)4-Fc and DVD-Ig. In some embodiments, the multispecific antibody is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. Additional types of multispecific antibodies and their construction are disclosed in Brinkmann & Kontermann (2017), mAbs, 9:2, 182-212, DOI: 10.1080/19420862.2016.1268307, the contents of which are incorporated by reference. FIGS. 1A-1F depicts exemplary multispecific antibodies as described herein.

Strategies based on forcing the heterodimerization of two heavy chains have been explored. A first approach coined ‘knob into hole’ aims at forcing the pairing of two different IgG heavy chains by introducing mutations into the CH3 domains to modify the contact interface. On one chain amino acids with large side chains were introduced, to create a ‘knob’. Conversely, bulky amino acids were replaced by amino acids with short side chains to create a ‘hole’ into the other CH3 domain. By co-expressing these two heavy chains, more than 90% heterodimer formation was observed (‘knob-hole’) versus homodimers formation (‘hole-hole’ or ‘knob-knob’). A similar concept was developed using strand-exchange engineered domain (SEED) human CH3 domains based on human IgG and human IgA sequences. These engineered domains lead to the formation of heterodimeric molecules that can carry two different specificities.

Recently an improvement over the ‘knob into hole’ approach; “CrossMab” has been described in WO 2009/080253 A1. This method involves the exchange of some of the light chain and heavy chain domains in addition to the ‘knob into hole’ mutations.

In some embodiments, a multispecific antibody described herein comprise two heavy chains, wherein each heavy chain binds nonidentical epitopes. Each heavy chain has at one end a variable domain (VH) followed by a number of constant domains (three or four constant domains, CH1, CH2, CH3 and CH4, depending on the antibody class). Each light chain has a variable domain (VL) at one end and a constant domain (CL) at its other end; the constant domain of the light chain is aligned with the first constant domain of the heavy chain, and the light chain variable domain is aligned with the variable domain of the heavy chain. Accordingly, multispecific antibodies described herein comprises a TL1A binding heavy chain that binds an epitope present on TL1A, variants thereof or functional fragments thereof, and an IL binding heavy chain that binds an epitope present on IL-6, IL-12, IL-23, IL-23p19, IL-12p40, IL-12p35, IL-6R, an IL-17 family cytokine, IL-17R, or a combination thereof, wherein the TL1A binding heavy chain and the IL binding heavy chain interact with each other to form a heterodimeric multispecific antibodies. In some embodiments, the heavy chains described herein comprise a

In some embodiments, a Fc region described herein is derived from an IgG heavy chain constant domain or an IgA heavy constant domain. In some embodiments, the IgG heavy chain constant domain comprises an IgG1 heavy chain constant domain. In some embodiments, the IgG1 heavy chain constant domain comprises a human IgG1 heavy chain constant domain. An amino acid sequence of wildtype human IgG1 heavy chain constant domain is provided in TABLE 44. In some embodiments, the Fc region is engineered to not bind to Fc gamma receptor (FcγR). In some embodiments, the FcγR comprises FcγRI, FcγRII and FcγRIII.

TABLE 44
Amino acid sequence of Fc region of human IgG1

| SEQ ID | |
|---|---|
| NO: | HC Amino Acid Sequences |
| 481 | ASTKGPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCDKTHTCPPCPA |
| | PELLGGPSVFLFPPKPKDTLMISRTPEVTCVVVDVSHEDPEVKFNWYVDGVEVHNAK |
| | TKPREEQYNSTYRVVSVLTVLHQDWLNGKEYKCKVSNKALPAPIEKTISKAKGQPRE |
| | PQVYTLPPSRDELTKNQVSLTCLVKGFYPSDIAVEWESNGQPENNYKTTPPVLDSDG |
| | SFFLYSKLTVDKSRWQQGNVFSCSVMHEALHNHYTQKSLSLSPELQLEESCAEAQDG |
| | ELDGLWTTITIFITLFLLSVCYSATVTFFKVKWIFSSVVDLKQTIIPDYRNMIGQGA |
| 482 | ASTKGPSVFPLAPCSRSTSESTAALGCLVKDYFPEPVTVSWNSGALTSGVHTFPAVL |
| | QSSGLYSLSSVVTVPSSNFGTQTYTCNVDHKPSNTKVDKTVERKCCVECPPCPAPPV |
| | AGPSVFLFPPKPKDTLMISRTPEVTCVVVDVSHEDPEVQFNWYVDGVEVHNAKTKPR |
| | EEQFNSTFRVVSVLTVVHQDWLNGKEYKCKVSNKGLPAPIEKTISKTKGQPREPQVY |
| | TLPPSREEMTKNQVSLTCLVKGFYPSDISVEWESNGQPENNYKTTPPMLDSDGSFFL |
| | YSKLTVDKSRWQQGNVFSCSVMHEALHNHYTQKSLSLSPELQLEESCAEAQDGELDG |
| | LWTTITIFITLFLLSVCYSATITFFKVKWIFSSVVDLKQTIVPDYRNMIRQGA |
| 483 | ASTKGPSVFPLAPCSRSTSESTAALGCLVKDYFPEPVTVSWNSGALTSGVHTFPAVL |
| | QSSGLYSLSSVVTVPSSSLGTKTYTCNVDHKPSNTKVDKRVESKYGPPCPSCPAPEF |
| | LGGPSVFLFPPKPKDTLMISRTPEVTCVVVDVSQEDPEVQFNWYVDGVEVHNAKTKP |
| | REEQFNSTYRVVSVLTVLHQDWLNGKEYKCKVSNKGLPSSIEKTISKAKGQPREPQV |
| | YTLPPSQEEMTKNQVSLTCLVKGFYPSDIAVEWESNGQPENNYKTTPPVLDSDGSFF |
| | LYSRLTVDKSRWQEGNVFSCSVMHEALHNHYTQKSLSLSLELQLEESCAEAQDGELD |
| | GLWTTITIFITLFLLSVCYSATVTFFKVKWIFSSVVDLKQTIVPDYRNMIRQGA |

In some embodiments, a Fc region described herein comprises an amino acid sequence that is at least 60%, at least 65%, at least 70%, at least 75%, at least 80%, at least 85%, at least 90%, at least 95% or 100% identical to a corresponding parent amino acid sequence of the Fc region of IgG1 (SEQ ID NO: 481).

In some embodiments, a Fc region described herein comprises one or more modifications relative to a corresponding parent amino acid sequence of the Fc region of IgG1 (SEQ ID NO: 481). In some embodiments, the Fc region of IgG1 comprises at least one, at least two, at least three, at least four, at least five, at least six, at least seven, at least eight, at least nine, at least ten, at least eleven, at least twelve, at least thirteen, at least fourteen or at least fifteen modifications relative to a corresponding parent amino acid sequence of SEQ ID NO: 481. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen deletions, substitutions, additions or combinations thereof relative to a corresponding parent amino acid sequence of SEQ ID NO: 481. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen substitutions relative to a corresponding parent amino acid sequence of SEQ ID NO: 481.

In some embodiments, a Fc region described herein is derived from a human IgG1 heavy chain constant domain. In some embodiments, the Fc region comprises at least one substitution, at least two substitutions, at least three substitutions, at least four substitutions, at least five substitutions, at least six substitutions or at least seven substitutions relative to the human IgG1 heavy chain constant domain. In some embodiments, the at least one substitution is selected from positions D221, K222, T223, H224, T225, C226, P227, P228, C229, P230, A231, P232, E233, L234, L235, G236, G237, P238, S239, V240, F241, F243, P244, P245, K246, P247, D249, M252, S254, R255, T256, E258, T260, V262, V263, V264, D265, V266, S267, H268, E269, D270, P271, E272, V273, K274, F275, N276, Y278, D280, G281, V282, E283, V284, H285, N286, K288, K290, P291, R292, E293, E294, Q295, Y296, N297, S298, T299, Y300, R301, V302, V303, S304, V305, W313, K317, E318, K320, K322, V323, S324, N325, K326, A327, L328, P329, A330, P331, I332, E333, K334, T335, 1336, S337, and P396, per EU numbering. In some embodiments, the at least two substitutions are selected from positions D221, K222, T223, H224, T225, C226, P227, P228, C229, P230, A231, P232, E233, L234, L235, G236, G237, P238, S239, V240, F241, F243, P244, P245, K246, P247, D249, M252, S254, R255, T256, E258, T260, V262, V263, V264, D265, V266, S267, H268, E269, D270, P271, E272, V273, K274, F275, N276, Y278, D280, G281, V282, E283, V284, H285, N286, K288, K290, P291, R292, E293, E294, Q295, Y296, N297, S298, T299, Y300, R301, V302, V303, S304, V305, W313, K317, E318, K320, K322, V323, S324, N325, K326, A327, L328, P329, A330, P331, I332, E333, K334, T335, 1336, S337, and P396, per EU numbering. In some embodiments, the at least three substitutions are selected from positions D221, K222, T223, H224, T225, C226, P227, P228, C229, P230, A231, P232, E233, L234, L235, G236, G237, P238, S239, V240, F241, F243, P244, P245, K246, P247, D249, M252, S254, R255, T256, E258, T260, V262, V263, V264, D265, V266, S267, H268, E269, D270, P271, E272, V273, K274, F275, N276, Y278, D280, G281, V282, E283, V284, H285, N286, K288, K290, P291, R292, E293, E294, Q295, Y296, N297, S298, T299, Y300, R301, V302, V303, S304, V305, W313, K317, E318, K320, K322, V323, S324, N325, K326, A327, L328, P329, A330, P331, I332, E333, K334, T335, I336, S337, and P396, per EU numbering. In some embodiments, the at least four substitutions are selected from positions D221, K222, T223, H224, T225, C226, P227, P228, C229, P230, A231, P232, E233, L234, L235, G236, G237, P238, S239, V240, F241, F243, P244, P245, K246, P247, D249, M252, S254, R255, T256, E258, T260, V262, V263, V264, D265, V266, S267, H268, E269, D270, P271, E272, V273, K274, F275, N276, Y278, D280, G281, V282, E283, V284, H285, N286, K288, K290, P291, R292, E293, E294, Q295, Y296, N297, S298, T299, Y300, R301, V302, V303, S304, V305, W313, K317, E318, K320, K322, V323, S324, N325, K326, A327, L328, P329, A330, P331, I332, E333, K334, T335, 1336, S337, and P396, per EU numbering. In some embodiments, the at least five substitutions are selected from positions D221, K222, T223, H224, T225, C226, P227, P228, C229, P230, A231, P232, E233, L234, L235, G236, G237, P238, S239, V240, F241, F243, P244, P245, K246, P247, D249, M252, S254, R255, T256, E258, T260, V262, V263, V264, D265, V266, S267, H268, E269, D270, P271, E272, V273, K274, F275, N276, Y278, D280, G281, V282, E283, V284, H285, N286, K288, K290, P291, R292, E293, E294, Q295, Y296, N297, S298, T299, Y300, R301, V302, V303, S304, V305, W313, K317, E318, K320, K322, V323, S324, N325, K326, A327, L328, P329, A330, P331, I332, E333, K334, T335, 1336, S337, and P396, per EU numbering. In some embodiments, the at least six substitutions are selected from positions D221, K222, T223, H224, T225, C226, P227, P228, C229, P230, A231, P232, E233, L234, L235, G236, G237, P238, S239, V240, F241, F243, P244, P245, K246, P247, D249, M252, S254, R255, T256, E258, T260, V262, V263, V264, D265, V266, S267, H268, E269, D270, P271, E272, V273, K274, F275, N276, Y278, D280, G281, V282, E283, V284, H285, N286, K288, K290, P291, R292, E293, E294, Q295, Y296, N297, S298, T299, Y300, R301, V302, V303, S304, V305, W313, K317, E318, K320, K322, V323, S324, N325, K326, A327, L328, P329, A330, P331, I332, E333, K334, T335, I336, S337, and P396, per EU numbering. In some embodiments, the at least seven substitutions are selected from positions D221, K222, T223, H224, T225, C226, P227, P228, C229, P230, A231, P232, E233, L234, L235, G236, G237, P238, S239, V240, F241, F243, P244, P245, K246, P247, D249, M252, S254, R255, T256, E258, T260, V262, V263, V264, D265, V266, S267, H268, E269, D270, P271, E272, V273, K274, F275, N276, Y278, D280, G281, V282, E283, V284, H285, N286, K288, K290, P291, R292, E293, E294, Q295, Y296, N297, S298, T299, Y300, R301, V302, V303, S304, V305, W313, K317, E318, K320, K322, V323, S324, N325, K326, A327, L328, P329, A330, P331, I332, E333, K334, T335, 1336, S337, and P396, per EU numbering. In some embodiments, the substitutions at position D221 comprises D221K, and D221Y substitutions. In some embodiments, the substitutions at position K222 comprises K222E, and K222Y substitutions. In some embodiments, the substitutions at position T223 comprises T223E, and T223K substitutions. In some embodiments, the substitutions at position H224 comprises H224E, and H224Y substitutions. In some embodiments, the substitutions at position T225 comprises T225E, T225K, and T225W substitutions. In some embodiments, the substitutions at position C226 comprises C226S substitution. In some embodiments, the substitutions at position P227 comprises P227E, P227G, P227K, and P227Y substitutions. In some embodiments, the substitutions at position P228 comprises P228E, P228G, P228K, and P228Y substitutions. In some embodiments, the substitutions at position C229 comprises C229S substitution. In some embodiments, the substitutions at position P230 comprises P230A, P230E, P230G, and P230Y substitutions. In some embodiments, the substitutions at position A231 comprises A231E, A231G, A231K, A231P, and A231Y substitutions. In some embodiments, the substitutions at position P232 comprises P232E, P232G, P232K, and P232Y substitutions. In some embodiments, the substitutions at position E233 comprises E233A, E233D, E233F, E233G, E233H, E233I, E233K, E233L, E233M, E233N, E233Q, E233R, E233P, E233S, E233T, E233V, E233W, and E233Y substitutions. In some embodiments, the substitutions at position L234 comprises L234A, L234D, L234E, L234F, L234G, L234H, L234I, L234K, L234M, L234N, L234P, L234Q, L234R, L234S, L234T, L234V, L234W, and L234Y substitutions. In some embodiments, the substitutions at position L235 comprises L235A, L235D, L235E, L235F, L235G, L235H, L235I, L235K, L235M, L235N, L235P, L235Q, L235R, L235S, L235T, L235V, L235W, and L235Y substitutions. In some embodiments, the substitutions at position G236 comprises G236A, G236D, G236E, G236F, G236H, G236I, G236K, G236L, G236M, G236N, G236P, G236Q, G236R, G236S, G236T, G236V, G236W, and G236Y substitutions. In some embodiments, the substitutions at position G237 comprises G237A, G237D, G237E, G237F, G237H, G237I, G237K, G237L, G237M, G237N, G237P, G237Q, G237R, G237S, G237T, G237V, G237W, and G237Y substitutions. In some embodiments, the substitutions at position P238 comprises P238A, P238D, P238E, P238F, P238G, P238H, P238I, P238K, P238L, P238M, P238N, P238Q, P238R, P238S, P238T, P238V, P238W, and P238Y substitutions. In some embodiments, the substitutions at position S239 comprises S239D, S239E, S239F, S239G, S239H, S239I, S239K, S239L, S239M, S239N, S239P, S239Q, S239R, S239T, S239V, S239W, and S239Y substitutions. In some embodiments, the substitutions at position V240 comprises V240A, V240I, V240M, and V240T substitutions. In some embodiments, the substitutions at position F241 comprises F241D, F241E, F241L, F241R, F241S, F241W, and F241Y substitutions. In some embodiments, the substitutions at position F243 comprises F243E, F243H, F243L, F243Q, F243R, F243W, and F243Y substitutions. In some embodiments, the substitutions at position P244 comprises P244H substitution. In some embodiments, the substitutions at position P245 comprises P245A substitution. In some embodiments, the substitutions at position K246 comprises K246D, K246E, K246H, and K246Y substitutions. In some embodiments, the substitutions at position P247 comprises P247G, and P247V substitutions. In some embodiments, the substitutions at position D249 comprises D249H, D249Q, and D249Y substitutions. In some embodiments, the substitutions at position M252 comprises M252Y substitution. In some embodiments, the substitutions at position S254 comprises S254T substitution. In some embodiments, the substitutions at position R255 comprises R255E, and R255Y substitutions. In some embodiments, the substitutions at position T256 comprises T256E substitution. In some embodiments, the substitutions at position E258 comprises E258H, E258S, and E258Y substitutions. In some embodiments, the substitutions at position T260 comprises T260D, T260E, T260H, and T260Y substitutions. In some embodiments, the substitutions at position V262 comprises V262A, V262E, V262F, V262I, and V262T substitutions. In some embodiments, the substitutions at position V263 comprises V263A, V263I, V263M, and V263T substitutions. In some embodiments, the substitutions at position V264 comprises V264A, V264D, V264E, V264F, V264G, V264H, V264I, V264K, V264L, V264M, V264N, V264P, V264Q, V264R, V264S, V264T, V264W, and V264Y substitutions. In some embodiments, the substitutions at position D265 comprises D265A, D265F, D265G, D265H, D265I, D265K, D265L, D265M, D265N, D265P, D265Q, D265R, D265S, D265T, D265V, D265W, and D265Y substitutions. In some embodiments, the substitutions at position V266 comprises V266A, V266I, V266M, and V266T substitutions. In some embodiments, the substitutions at position S267 comprises S267D, S267E, S267F, S267H, S267I, S267K, S267L, S267M, S267N, S267P, S267Q, S267R, S267T, S267V, S267W, and S267Y substitutions. In some embodiments, the substitutions at position H268 comprises H268A, H268D, H268E, H268F, H268G, H268I, H268K, H268L, H268M, H268P, H268Q, H268R, H268T, H268V, and H268W substitutions. In some embodiments, the substitutions at position E269 comprises E269F, E269G, E269H, E269I, E269K, E269L, E269M, E269N, E269P, E269R, E269S, E269T, E269V, E269W, and E269Y substitutions. In some embodiments, the substitutions at position D270 comprises D270A, D270F, D270G, D270H, D270I, D270L, D270M, D270P, D270Q, D270R, D270S, D270T, D270W, and D270Y substitutions. In some embodiments, the substitutions at position P271 comprises P271A, P271D, P271E, P271F, P271G, P271H, P271I, P271K, P271L, P271M, P271N, P271Q, P271R, P271S, P271T, P271V, P271W, and P271Y substitutions. In some embodiments, the substitutions at position E272 comprises E272D, E272F, E272G, E272H, E272I, E272K, E272L, E272M, E272P, E272R, E272S, E272T, E272V, E272W, and E272Y substitutions. In some embodiments, the substitutions at position V273 comprises V273I substitution. In some embodiments, the substitutions at position K274 comprises K274D, K274E, K274F, K274G, K274H, K274I, K274L, K274M, K274N, K274P, K274R, K274T, K274V, K274W, and K274Y substitutions. In some embodiments, the substitutions at position F275 comprises F275L, and F275W substitutions. In some embodiments, the substitutions at position N276 comprises N276D, N276E, N276F, N276G, N276H, N276I, N276L, N276M, N276P, N276R, N276S, N276T, N276V, N276W, and N276Y substitutions. In some embodiments, the substitutions at position Y278 comprises Y278D, Y278E, Y278G, Y278H, Y278I, Y278K, Y278L, Y278M, Y278N, Y278P, Y278Q, Y278R, Y278S, Y278T, Y278V, and Y278W substitutions. In some embodiments, the substitutions at position D280 comprises D280G, D280K, D280L, D280P, and D280W substitutions. In some embodiments, the substitutions at position G281 comprises G281D, G281E, G281K, G281N, G281P, G281Q, and G281Y substitutions. In some embodiments, the substitutions at position V282 comprises V282E, V282G, V282K, V282P, and V282Y substitutions. In some embodiments, the substitutions at position E283 comprises E283G, E283H, E283K, E283L, E283P, E283R, and E283Y substitutions. In some embodiments, the substitutions at position V284 comprises V284D, V284E, V284L, V284N, V284Q, V284T, and V284Y substitutions. In some embodiments, the substitutions at position H285 comprises H285D, H285E, H285K, H285Q, H285W, and H285Y substitutions. In some embodiments, the substitutions at position N286 comprises N286E, N286G, N286P, and N286Y substitutions. In some embodiments, the substitutions at position K288 comprises K288D, K288E, and K288Y substitutions. In some embodiments, the substitutions at position K290 comprises K290D, K290H, K290L, K290N, and K290W substitutions. In some embodiments, the substitutions at position P291 comprises P291D, P291E, P291G, P291H, P291I, P291Q, and P291T substitutions. In some embodiments, the substitutions at position R292 comprises R292D, R292E, R292P, R292T, and R292Y substitutions. In some embodiments, the substitutions at position E293 comprises E293F, E293G, E293H, E293I, E293L, E293M, E293N, E293P, E293R, E293S, E293T, E293V, E293W, and E293Y substitutions. In some embodiments, the substitutions at position E294 comprises E294F, E294G, E294H, E294I, E294K, E294L, E294M, E294P, E294R, E294S, E294T, E294V, E294W, and E294Y substitutions. In some embodiments, the substitutions at position Q295 comprises Q295D, Q295E, Q295F, Q295G, Q295H, Q295I, Q295M, Q295N, Q295P, Q295R, Q295S, Q295T, Q295V, Q295W, and Q295Y substitutions. In some embodiments, the substitutions at position Y296 comprises Y296A, Y296D, Y296E, Y296G, Y296H, Y296I, Y296K, Y296L, Y296M, Y296N, Y296Q, Y296R, Y296S, Y296T, and Y296V substitutions. In some embodiments, the substitutions at position N297 comprises N297A, N297D, N297E, N297F, N297G, N297H, N297I, N297K, N297L, N297M, N297P, N297Q, N297R, N297S, N297T, N297V, N297W, and N297Y substitutions. In some embodiments, the substitutions at position S298 comprises S298D, S298E, S298F, S298H, S298I, S298K, S298M, S298N, S298Q, S298R, S298T, S298W, and S298Y substitutions. In some embodiments, the substitutions at position T299 comprises T299A, T299D, T299E, T299F, T299G, T299H, T299I, T299K, T299L, T299M, T299N, T299P, T299Q, T299R, T299S, T299V, T299W, and T299Y substitutions. In some embodiments, the substitutions at position Y300 comprises Y300A, Y300D, Y300E, Y300G, Y300H, Y300K, Y300L, Y300M, Y300N, Y300P, Y300Q, Y300R, Y300S, Y300T, Y300V, and Y300W substitutions. In some embodiments, the substitutions at position R301 comprises R301D, R301E, R301H, and R301Y substitutions. In some embodiments, the substitutions at position V302 comprises V302I substitution. In some embodiments, the substitutions at position V303 comprises V303D, V303E, and V303Y substitutions. In some embodiments, the substitutions at position S304 comprises S304D, S304H, S304L, S304N, and S304T substitutions. In some embodiments, the substitutions at position V305 comprises V305E, V305T, and V305Y substitutions. In some embodiments, the substitutions at position W313 comprises W313F substitution. In some embodiments, the substitutions at position K317 comprises K317E, and K317Q substitutions. In some embodiments, the substitutions at position E318 comprises E318H, E318L, E318Q, E318R, and E318Y substitutions. In some embodiments, the substitutions at position K320 comprises K320D, K320F, K320G, K320H, K320I, K320L, K320N, K320P, K320S, K320T, K320V, K320W, and K320Y substitutions. In some embodiments, the substitutions at position K322 comprises K322A, K322D, K322F, K322G, K322H, K322I, K322P, K322Q, K322S, K322T, K322V, K322W, and K322Y substitutions. In some embodiments, the substitutions at position V323 comprises V323I substitution. In some embodiments, the substitutions at position S324 comprises S324D, S324F, S324G, S324H, S324I, S324L, S324M, S324P, S324R, S324T, S324V, S324W, and S324Y substitutions. In some embodiments, the substitutions at position N325 comprises N325A, N325D, N325E, N325F, N325G, N325H, N325I, N325K, N325L, N325M, N325P, N325Q, N325R, N325S, N325T, N325V, N325W, and N325Y substitutions. In some embodiments, the substitutions at position K326 comprises K326I, K326L, K326P, and K326T substitutions. In some embodiments, the substitutions at position A327 comprises A327D, A327E, A327Q, A327G, A327H, A327I, A327K, A327L, A327M, A327N, A327P, A327R, A327S, A327T, A327V, A327W, and A327Y substitutions. In some embodiments, the substitutions at position L328 comprises L328A, L328D, L328E, L328F, L328G, L328H, L328I, L328K, L328M, L328N, L328P, L328Q, L328R, L328S, L328T, L328V, L328W, and L328Y substitutions. In some embodiments, the substitutions at position P329 comprises P329A, P329D, P329E, P329F, P329G, P329H, P329I, P329K, P329L, P329M, P329N, P329Q, P329R, P329S, P329T, P329V, P329W, and P329Y substitutions. In some embodiments, the substitutions at position A330 comprises A330E, A330F, A330G, A330H, A330I, A330L, A330M, A330N, A330P, A330R, A330S, A330T, A330V, A330W, and A330Y substitutions. In some embodiments, the substitutions at position P331 comprises P331D, P331F, P331H, P331I, P331L, P331M, P331Q, P331R, P331S, P331T, P331V, P331W, and P331Y substitutions. In some embodiments, the substitutions at position I332 comprises I332A, I332D, I332E, I332F, I332H, I332K, I332L, I332M, I332N, I332P, I332Q, I332R, I332S, I332T, I332V, I332W, and I332Y substitutions. In some embodiments, the substitutions at position E333 comprises E333F, E333H, E333I, E333L, E333M, E333P, E333T, and E333Y substitutions. In some embodiments, the substitutions at position K334 comprises K334F, K334I, K334L, K334P, and K334T substitutions. In some embodiments, the substitutions at position T335 comprises T335D, T335F, T335G, T335H, T335I, T335L, T335M, T335N, T335P, T335R, T335S, T335V, T335W, and T335Y substitutions. In some embodiments, the substitutions at position I336 comprises I336E, I336K, and I336Y substitutions. In some embodiments, the substitutions at position S337 comprises S337E, S337H, and S337N substitutions. In some embodiments, the substitutions at position P396 comprises P396L substitution.

In some embodiments, a human IgG1 heavy chain constant domain described herein comprises two substitutions. In some embodiments, the two substitutions are located at positions L234 and L235, per EU numbering. In some embodiments, the two substitutions are L234A and L235A substitutions.

In some embodiments, a human IgG1 heavy chain constant domain described herein comprises three substitutions. In some embodiments, the three substitutions are located at positions L234, L235 and P329, per EU numbering. In some embodiments, the three substitutions are L234A, L235A and P329A substitutions, per EU numbering. In some embodiments, the three substitutions are L234A, L235A and P329G substitutions, per EU numbering. In some embodiments, the three substitutions are located at positions M252, S254 and T256, per EU numbering. In some embodiments, the three substitutions are M252Y, S254T and T256E substitutions, per EU numbering.

In some embodiments, a human IgG1 heavy chain constant domain described herein comprises one or more substitutions according to at least one group selected from: (1) L234A, L235A and P329A; (2) M252Y, S254T and T256E; (3) N287A; and (4) D265A, per EU numbering. Accordingly, in some embodiments, an engineered BsAb described herein can comprise: (1) one or modifications of amino acids that can result in pH-dependent target binding activity; and (2) one or more substitutions in a human IgG1 heavy chain constant domain of the engineered BsAb according to at least one group selected from: (a) L234A, L235A and P329G; (b) M252Y, S254T and T256E; (c) N287A; and (d) D265A, per EU numbering.

In some embodiments, a human IgG1 heavy chain constant domain described herein comprises one or more substitutions according to at least one group selected from: (1) L234A, L235A and P329G; (2) M252Y, S254T and T256E; (3) N287A; and (4) D265A, per EU numbering. Accordingly, in some embodiments, an engineered BsAb described herein can comprise: (1) one or modifications of amino acids that can result in pH-dependent target binding activity; and (2) one or more substitutions in a human IgG1 heavy chain constant domain of the engineered BsAb according to at least one group selected from: (a) L234A, L235A and P329G; (b) M252Y, S254T and T256E; (c) N287A; and (d) D265A, per EU numbering.

In some embodiments, a human IgG1 heavy chain constant domain described herein comprises one or more substitutions according to at least one group selected from: (1) S239D, A330L, I332E, S239D, A330Y, I332E, L234I, S239D, A330Y, I332E, and V266I; (2) S239D, D265F, N297D, and I332E; (3) S239D, D265H, N297D, and I332E; (4) S239D, D265I, N297D, and I332E; (5) S239D, D265L, N297D, and I332E; (6) S239D, D265T, N297D, and I332E; (7) S239D, D265Y, N297D, and I332E; (8) S239D, E272I, A330L, and I332E; (9) S239D, E272I, and I332E; (10) S239D, E272K, A330L, and I332E; (11) S239D, E272K, and I332E; (12) S239D, E272S, A330L, and I332E; (13) S239D, E272S, and I332E; (14) S239D, E272Y, A330L, and I332E; (15) S239D, E272Y, and I332E; (16) S239D, F241S, F243H, V262T, V264T, N297D, A330Y, and I332E; (17) S239D, and H268D; (18) S239D, and H268E; (19) S239D, and I332D; (20) S239D, and I332E; (21) S239D, I332E, and A327D; (22) S239D, I332E, and A330I; (23) S239D, I332E, and A330Y; (24) S239D, I332E, and E272H; (25) S239D, I332E, and E272R; (26) S239D, I332E, and E283H; (27) S239D, I332E, and E283L; (28) S239D, I332E, and G236A; (29) S239D, I332E, and G236S; (30) S239D, I332E, and H268D; (31) S239D, I332E, and H268E; (32) S239D, I332E, and K246H; (33) S239D, I332E, and R255Y; (34) S239D, I332E, and S267E; (35) S239D, I332E, and V264I; (36) S239D, I332E, V264I, and A330L; (37) S239D, I332E, V264I, and S298A; (38) S239D, I332E, and V284D; (39) S239D, I332E, and V284E; (40) S239D, I332E, and V284E; (41) S239D, and I332N; (42) S239D, and I332Q; (43) S239D, K274E, A330L, and I332E; (44) S239D, K274E, and I332E; (45) S239D, K326E, A330L, and I332E; (46) S239D, K326E, A330Y, and I332E; (47) S239D, K326E, and I332E; (48) S239D, K326T, A330Y, and I332E; (49) S239D, K326T, and I332E; (50) S239D, N297D, A330Y, and I332E; (51) S239D, N297D, and I332E; (52) S239D, N297D, K326E, and I332E; (53) S239D, S267E, A330L, and I332E; (54) S239D, S267E, and I332E; (55) S239D, S298A, K326E, and I332E; (56) S239D, S298A, K326T, and I332E; (57) S239D, V240I, A330Y, and I332E; (58) S239D, V264T, A330Y, and I332E; (59) S239D, Y278T, A330L, and I332E; (60) S239D, Y278T, and I332E; (61) S239E, and D265G; (62) S239E, and D265N; (63) S239E, and D265Q; (64) S239E, and I332E; (65) S239E, and I332N; (66) S239E, and I332Q; (67) S239E, N297D, and I332E; (68) S239E, V264I, A330Y, and I332E; (69) S239E, V264I, and I332E; (70) S239E, V264I, S298A, A330Y, and I332E; (71) S239N, and I332D; (72) S239N, and I332E; (73) S239N, I332E, and A330L; (74) S239N, I332E, and A330Y; (75) S239N, and I332N; (76) S239N, and I332Q; (77) S239Q, and I332D; (78) S239Q, and I332E; (79) S239Q, and I332N; (80) S239Q, and I332Q; (81) S239Q, V264I, and I332E; (82) F241E, F243Q, V262T, V264E, and I332E; (83) F241E, F243Q, V262T, and V264E; (84) F241E, F243R, V262E, V264R, and I332E; (85) F241E, F243R, V262E, and V264R; (86) F241E, F243Y, V262T, V264R, and I332E; (87) F241E, F243Y, V262T, and V264R; (88) F241L, F243L, V262I, and V264I; (89) F241L, and V262I; (90) F241R, F243Q, V262T, V264R, and I332E; (91) F241R, F243Q, V262T, and V264R; (92) F241W, and F243W; (93) F241W, F243W, V262A, and V264A; (94) F241Y, F243Y, V262T, V264T, N297D, and I332E; (95) F241Y, F243Y, V262T, and V264T; (96) F243L, V262I, and V264W; (97) F243L, and V264I; (98) P244H, P245A, and P247V; (99) V264E, N297D, and I332E; (100) V264I, A330L, and I332E; (101) V264I, A330Y, and I332E; (102) V264I, and I332E; (103) D265Y, N297D, and I332E; (104) D265Y, N297D, T299L, and I332E; (105) S267E, and A327D; (106) S267E, and P331D; (107) S267E, and S324I; (108) S267E, and V282G; (109) S267L, and A327S; (110) S267Q, and A327S; (111) Y278W, E283R, and V302I; (112) G281D, and V282G; (113) V282G, and P331D; (114) E283R, V302I, Y278W, and E283R; (115) N297D, and I332E; (116) N297D, I332E, S239D, and D265V; (117) N297D, I332E, and T299E; (118) N297D, I332E, and T299F; (119) N297D, I332E, and T299H; (120) N297D, I332E, and T299I; (121) N297D, I332E, and T299L; (122) N297D, I332E, and T299V; (123) N297D, I332E, and Y296D; (124) N297D, I332E, and Y296E; (125) N297D, I332E, and Y296H; (126) N297D, I332E, and Y296N; (127) N297D, I332E, and Y296Q; (128) N297D, I332E, and Y296T; (129) N297E, and I332E; (130) N297S, and I332E; (131) S298A, and I332E; (132) S298A, and K326E; (133) S298A, K326E, and K334L; (134) S298A, and K334L; (135) S324I, and A327D; (136) L328D, and I332E; (137) L328E, and I332E; (138) L328H, and I332E; (139) L328I, and I332E; (140) L328I, and I332E; (141) L328M, and I332E; (142) L328N, and I332E; (143) L328Q, and I332E; (144) L328Q, and I332E; (145) L328T, and I332E; (146) L328V, and I332E; (147) A330L, and I332E; (148) A330Y, and I332E; (149) I332E, and G281D; (150) I332E, and H268D; (151) I332E, and H268E; (152) I332E, S239D, and S298A; (153) I332E, S239N, and S298A; (154) I332E, V264I, and S298A; (155) I332E, and V284E; (156) S239E, and I332D; (157) Y278W, and V302I; (158) N297D, I332E, and A330Y; (159) N297D, I332E, S239D, and A330L; and (160) N297D, I332E, S298A, and A330Y, per EU numbering.

In some embodiments, the Fc region comprises an amino acid sequence that is at least 60%, at least 65%, at least 70%, at least 75%, at least 80%, at least 85%, at least 90%, at least 95% or 100% identical to a corresponding parent sequence of the Fc region of IgG2 (SEQ ID NO: 482).

In some embodiments, the Fc region described herein comprises one or more modifications relative to a corresponding parent sequence of the Fc region of IgG2 (SEQ ID NO: 482). In some embodiments, the Fc region of IgG2 comprises at least one, at least two, at least three, at least four, at least five, at least six, at least seven, at least eight, at least nine, at least ten, at least eleven, at least twelve, at least thirteen, at least fourteen or at least fifteen modifications relative to a corresponding parent sequence of SEQ ID NO: 482. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen deletions, substitutions, additions or combinations thereof relative to a corresponding parent sequence of SEQ ID NO: 482. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen substitutions relative to a corresponding parent sequence of SEQ ID NO: 482.

In some embodiments, a Fc region described herein is derived from a human IgG2 heavy chain constant domain. In some embodiments, the Fc region comprises at least one substitution, at least two substitutions, at least three substitutions, at least four substitutions, at least five substitutions, at least six substitutions or at least seven substitutions relative to the human IgG2 heavy chain constant domain. In some embodiments, the at least one substitution is selected from positions C232, C233, V234, G237, P238, M252, 5254, T256, H268, N297, V309, A330, and P331, per EU numbering. In some embodiments, the at least two substitutions are selected from positions C232, C233, V234, G237, P238, M252, S254, T256, H268, N297, V309, A330, and P331, per EU numbering. In some embodiments, the at least three substitutions are selected from positions C232, C233, V234, G237, P238, M252, S254, T256, H268, N297, V309, A330, and P331, per EU numbering. In some embodiments, the at least four substitutions are selected from positions C232, C233, V234, G237, P238, M252, S254, T256, H268, N297, V309, A330, and P331, per EU numbering. In some embodiments, the at least five substitutions are selected from positions C232, C233, V234, G237, P238, M252, S254, T256, H268, N297, V309, A330, and P331, per EU numbering. In some embodiments, the at least six substitutions are selected from positions C232, C233, V234, G237, P238, M252, S254, T256, H268, N297, V309, A330, and P331, per EU numbering. In some embodiments, the at least seven substitutions are selected from positions C232, C233, V234, G237, P238, M252, S254, T256, H268, N297, V309, A330, and P331, per EU numbering. In some embodiments, the substitutions at position C232 comprises C232S substitution. In some embodiments, the substitutions at position C233 comprises C233S substitution. In some embodiments, the substitutions at position V234 comprises V234A substitution. In some embodiments, the substitutions at position G237 comprises G237A substitution. In some embodiments, the substitutions at position P238 comprises P238S substitution. In some embodiments, the substitutions at position M252 comprises M252Y substitution. In some embodiments, the substitutions at position S254 comprises S254T substitution. In some embodiments, the substitutions at position T256 comprises T256E substitution. In some embodiments, the substitutions at position H268 comprises H268A, H268E and H268Q substitutions. In some embodiments, the substitutions at position N297 comprises N297A and N297Q substitutions. In some embodiments, the substitutions at position N297 comprises N297A substitution. In some embodiments, the substitutions at position V309 comprises V309L substitution. In some embodiments, the substitutions at position A330 comprises A330S substitution. In some embodiments, the substitutions at position P331 comprises P331S substitution.

In some embodiments, the Fc region comprises an amino acid sequence that is at least 60%, at least 65%, at least 70%, at least 75%, at least 80%, at least 85%, at least 90%, at least 95% or 100% identical to a corresponding parent sequence of the Fc region of IgG4 (SEQ ID NO: 483).

In some embodiments, the Fc region described herein comprises one or more modifications relative to a corresponding parent sequence of the Fc region of IgG4 (SEQ ID NO: 483). In some embodiments, the Fc region of IgG4 comprises at least one, at least two, at least three, at least four, at least five, at least six, at least seven, at least eight, at least nine, at least ten, at least eleven, at least twelve, at least thirteen, at least fourteen or at least fifteen modifications relative to a corresponding parent sequence of SEQ ID NO: 483. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen deletions, substitutions, additions or combinations thereof relative to a corresponding parent sequence of SEQ ID NO: 483. In some embodiments, the Fc region described herein comprises at least one, at least two, at least three, at least four, at least five, at least six, at least seven, at least eight, at least nine, at least ten, at least eleven, at least twelve, at least thirteen, at least fourteen or at least fifteen substitutions relative to a corresponding parent sequence of SEQ ID NO: 483.

In some embodiments, a Fc region described herein is derived from a human IgG4 heavy chain constant domain. In some embodiments, the Fc region comprises at least one substitution, at least two substitutions, at least three substitutions, at least four substitutions, at least five substitutions, at least six substitutions or at least seven substitutions relative to the human IgG4 heavy chain constant domain. In some embodiments, the at least one substitution is selected from positions S228, E233, F234, L235, L236, G237, S241, L248, M252, 5254, T256, N297, E318, and T394, per EU numbering. In some embodiments, the at least two substitutions are selected from positions S228, E233, F234, L235, L236, G237, S241, L248, M252, S254, T256, N297, E318, and T394, per EU numbering. In some embodiments, the at least three substitutions are selected from positions S228, E233, F234, L235, L236, G237, S241, L248, M252, S254, T256, N297, E318, and T394, per EU numbering. In some embodiments, the at least four substitutions are selected from positions S228, E233, F234, L235, L236, G237, S241, L248, M252, S254, T256, N297, E318, and T394, per EU numbering. In some embodiments, the at least five substitutions are selected from positions S228, E233, F234, L235, L236, G237, S241, L248, M252, S254, T256, N297, E318, and T394, per EU numbering. In some embodiments, the at least six substitutions are selected from positions S228, E233, F234, L235, L236, G237, S241, L248, M252, S254, T256, N297, E318, and T394, per EU numbering. In some embodiments, the at least seven substitutions are selected from positions S228, E233, F234, L235, L236, G237, S241, L248, M252, S254, T256, N297, E318, and T394, per EU numbering. In some embodiments, the substitutions at position S228 comprises S228P substitution. In some embodiments, the substitutions at position E233 comprises E233P substitution. In some embodiments, the substitutions at position F234 comprises F234V substitution. In some embodiments, the substitutions at position L235 comprises L235A substitution. In some embodiments, the substitutions at position G237 comprises G237A substitution. In some embodiments, the substitutions at position S241 comprises S241P substitution. In some embodiments, the substitutions at position L248 comprises L248E substitution. In some embodiments, the substitutions at position M252 comprises M252Y substitution. In some embodiments, the substitutions at position S254 comprises S254T substitution. In some embodiments, the substitutions at position T256 comprises T256E substitution. In some embodiments, the substitutions at position N297 comprises N297A and N297Q substitutions. In some embodiments, the substitutions at position N297 comprises N297A substitution. In some embodiments, the substitutions at position E318 comprises E318A substitution. In some embodiments, the substitutions at position T394 comprises T394D substitution.

### Single Domain Antibodies

The immune systems of camelids (lamas and camels) and cartilaginous fish (nurse sharks) use single V-domains fused to a Fc demonstrating that a single domain can confer high affinity binding to a target. Camelid, shark and even human V domains represent alternatives to antibodies, but they also be used for BsAbs generation. They can be reformatted into a classical IgG in which each arm has the potential to bind two targets either via its VH or VL domain.

The bispecific antibodies of the present disclosure can be made by any process disclosed in the application or otherwise known in the art.

### Dual Variable Domain Immunoglobulin

Exemplary bispecific antibodies can comprise individually encoded peptides or “segments” which, in a single continuous chain, would comprise a compact tertiary structure. The component peptides are chosen so as to be asymmetric in their assumed structure, so as not to self-associate to form homo-multimers, but rather to associate in a complementary fashion, adopting a stable complex which resembles the parent tertiary structure. On the genetic level, these segments may be encoded by interchangeable cassettes with suitable restriction sites. These standardized cassettes may be fused C- or N-terminally to different recombinant proteins via a linker or hinge in a suitable expression vector system. Polypeptide segments which do not have the ability to assemble as homodimers are derived by cutting a parental polypeptide which has a compact tertiary structure. These polypeptide segments can then be fused to one or more different functional domains at the genetic level. These distinct polypeptide segments which are now fused to one or more functional domains can be, for example, co-expressed resulting in the formation of a native like parental structure attached to functional domains. This parental structure is formed by the dimerization of the polypeptide segments which were derived from the original parental polypeptide. The resulting multifunctional construct would appear as a compact tertiary structure attached to the one or more functional domains. Once structural sub-domains are identified, the protein is dissected in such a way these sub-domains remain intact. As part of this disclosure, DNA sequences, vectors, preferably bicistronic vectors, vector cassettes, can be made and characterized in that they comprise a DNA sequence encoding an amino acid sequence and optionally at least one further (poly)peptide comprised in the multifunctional polypeptide of the invention, and additionally at least one, preferably singular cloning sites for inserting the DNA encoding at least one further functional domain or that they comprise DNA sequences encoding the amino acid sequences, and optionally the further (poly)peptide(s) comprised in the multifunctional polypeptide of the invention and suitable restriction sites for the cloning of DNA sequences encoding the functional domains, such that upon expression of the DNA sequences after the insertion of the DNA sequences encoding the functional domains into said restriction sites, in a suitable host the multifunctional polypeptide of the invention may be formed. Said vector cassette is characterized in that it comprises the inserted DNA sequence(s) encoding said functional domain(s) and host cells transformed with at least one vector or vector cassette of the invention which can be used for the preparation of said bispecific or multifunctional polypeptides. The host cell may be a mammalian, preferably human, yeast, insect, plant or bacterial, preferably E. coli cell. The bispecific antibodies can be prepared by a method which comprises culturing at least two host cells of the invention in a suitable medium, said host cells each producing only one of said first and said second amino acid sequences attached to at least one further functional domain, recovering the amino acid sequences, mixing thereof under mildly denaturing conditions and allowing in vitro folding of the multifunctional polypeptide of the invention from said amino acid sequences. The method may be characterized in that the further amino acid sequences attached to at least one further functional domain are/is produced by at least one further host cell not producing said first or second amino acid sequence. Additionally, the method may be characterized in that at least one further amino acid sequence attached to at least one further functional domain is produced by the host cell of the invention producing said first or second amino acid sequence.

When either the second or the first portion of an antibody construct described herein comprises two antibody variable domains, these two antibody variable domains can be a VH- and VL-domain which are associated with one another. However, it is also contemplated that the two antibody variable domains comprised in either the second or the first portion may be two VH domains or two VL regions which are associated with one another. In some embodiments, where the two antibody variable domains of the first or second portion are covalently associated with one another, the two antibody variable domains may be designed as an scFv fragment, meaning that the two domains are separated from one another by a peptide linker long enough to allow intermolecular association between these two domains. In other words, a bispecific antibody may be a construct with a total of three antibody variable domains. One antibody variable domain specifically binds alone, i.e., without being paired with another antibody variable domain (a) either to a human immune effector cell by specifically binding to an effector on the human immune effector cell or to a target cell, while the remaining two antibody variable domains together specifically bind (b) either to the target on the target cell or to a human immune effector cell by specifically binding to an effector on the human immune effector cell, respectively. In this case, the presence of three antibody variable domains in the bispecific antibody entails unique advantages. Often, an scFv exhibiting the desired binding specificity for a target is already known and optimized, and omitting one of its two antibody variable domains would abolish or at least attenuate its binding characteristics. Such an scFv may make up part of an antibody construct described herein. Specifically, such a three-domain antibody may advantageously comprise an entire scFv as either its effector or target conferring portion. Effectively, then, this allows a bispecific antibody to be formed starting from a desired scFv by simple incorporation of only one additional antibody variable domain into the same polypeptide chain as the scFv, wherein the one additional antibody variable domain incorporated has a binding specificity different than that of the scFv. The first and second portions of the bispecific antibody may be separated from one another by a synthetic polypeptide spacer moiety, which covalently (i.e., peptidically) links either the C-terminus of the first portion with the N-terminus of the second portion, or the C-terminus of the second portion with the N-terminus of the first portion. As such, the portions of these bispecific antibodies may be arranged, as either N-(first portion)-(second portion)-C or N-(second portion)-(first portion)-C. In some embodiments, binding sites of a second specificity are fused to the N- or C-terminus of the heavy or light chain, e.g., in the form of an scFv fragment or a variable single domain, resulting in bispecific, tetravalent molecules. Bispecific molecules generated through fusion of an scFv fragment to a mAb offer great flexibility. ScFv molecules can be linked to the N-terminus but also the C-terminus of the heavy or light chain variable domain of a mAb, generally without compromising productivity or target-binding activity. This group of bispecific molecules also includes DVD-Igs, where a second VH and VL domain is fused to the heavy and light chain, respectively, of a mAb, two-in-one antibodies, where a second specificity is introduced into the natural binding site of an IgG molecule, and mAb2 molecules, where a second specificity is built into the CH3 domain of the Fc region. A characteristic feature of all these molecules is a symmetry caused by dimeric assembly of two identical heavy chains, an intrinsic property of these chains.

Heavy chain heterodimerization can be achieved by engineering a charged CH3 interface to introduce an electrostatic steering effect or using the strand-exchange engineered domain technology (SEEDbody) with CH3 sequences composed of alternating segments from human IgA and IgG. In contrast to the bispecific IgG-like molecules, these bispecific antibodies are bivalent with a size basically identical to that of IgG. Fc heterodimerization was recently applied to generate a trivalent, bispecific molecule fusing a VH and a VL domain to the C-termini of the engineered heavy chains (HA-TF Fc variant.) Bispecific antibodies with a molecular mass in the range of 50-100 kDa can be generated by combining the variable domains of two antibodies. For example, two scFv have been connected by a more or less flexible peptide linker in a tandem orientation (tandem scFv, taFv, tascFv), which can be extended further by additional scFv, e.g., generating bispecific or trispecific triple bodies (sctb). Diabodies are heterodimeric molecules composed of the variable domains of two antibodies arranged either in the order VHA-VLB and VHB-VLA (VH-VL orientation) or in the order VLA-VHB and VLB-VHA (VL-VH orientation). The linker connecting the two domains within one chain is approximately 5 residues leading, after co-expression of the two chains within one cell, to a head-to-tail assembly and hence formation of a compact molecule with two functional binding sites. The diabody (Db) format was further stabilized by introducing interchain disulfide bonds (dsDb, DART molecules) or by generating a single-chain derivative (scDb). ScDbs can be converted into tetravalent molecules by reducing the middle linker, resulting in homodimerization of two chains. Small bispecific molecules have also been produced by fusing a scFv to the heavy or light chain of a Fab fragment. Furthermore, tandem scFv, diabodies and scDb have been fused to the Fc or a CH3 domain to generate tetravalent derivatives. Also, scFv can be combined with Fc or CH3 domains to generate tetravalent molecules, e.g., fusing scFvs to the N- and C-terminus of an Fc fragment, or using the knobs-into-holes approach to generate bivalent scFv-Fc or scFv-CH3 molecules. A different approach for the generation of bispecific antibodies of the present invention is the dock-and-lock method (DNL). Many of the established bispecific antibody formats can also be combined with additional proteins and components, e.g., drugs, toxins, enzymes and cytokines, enabling dual targeting and delivery of a fusion partner. In addition, fusion to plasma proteins such as serum albumin or albumin-binding moieties can be applied to extend the plasma half-life of bispecific antibodies.

### Structure of Bispecific Antibodies

In one example the bispecific antibody may be a binding protein comprising a first polypeptide chain, wherein the polypeptide chain comprises VH1-(X1)n-VH2-C--(X2)n, wherein VH1 is a first heavy chain variable domain, VH2 is a second heavy chain variable domain, C is a constant domain, X1 represents a polypeptide linker, X2 represents an Fc region and n is 0 or 1. In some embodiments, the VH1 and VH2 in the binding protein may be heavy chain variable domains selected from the group consisting of a murine heavy chain variable domain, a human heavy chain variable domain, a CDR grafted heavy chain variable domain, and a humanized heavy chain variable domain. VH1 and VH2 may be capable of binding different targets. C may be a heavy chain constant domain. For example, X1 is a linker peptide. For example, X1 is a linker listed herein. In an embodiment, X2 is an Fc region. In another embodiment, X2 is a variant Fc region. In some embodiments, VH1 is capable of binding a first target and VH2 is capable of binding a second target. In some embodiments, VH1 is capable of binding a second target and VH2 is capable of binding a first target.

In one example the bispecific antibody may be a binding protein comprising a second polypeptide chain, wherein the polypeptide chain comprises VL1-(X1)n-VL2-C--(X2)n, wherein VL1 is a first light chain variable domain, VL2 is a second light chain variable domain, C is a constant domain, X1 represents a polypeptide linker, X2 represents an Fc region and n is 0 or 1. In some embodiments The VL1 and VL2 in the binding protein may be light chain variable domains selected from the group consisting of a murine light chain variable domain, a human light chain variable domain, a CDR grafted light chain variable domain, and a humanized light chain variable domain. VL1 and VL2 may be capable of binding different targets. C may be a heavy chain constant domain. For example, X1 is a linker peptide. For example, X1 is a linker listed herein. In an embodiment, X2 is an Fc region. In another embodiment, X2 is a variant Fc region. In some embodiments, VL1 is capable of binding a first target and VL2 is capable of binding a second target. In some embodiments, VL1 is capable of binding a first target and VL2 is capable of binding a second target. In some embodiments, the bispecific antibody construct comprises both the first polypeptide chain and the second polypeptide chain. The bispecific antibodies of the present disclosure can be a dual-variable domain immunoglobulin (DVD-Ig™) as described in Jakob 2013 which combines the target binding domains of two monoclonal antibodies via flexible naturally occurring linkers, which yields a tetravalent IgG-like molecule.

The present disclosure additionally provides a method of making a DVD-Ig binding protein by preselecting the parent antibodies against a first target and a second target. A method of making a Dual Variable Domain Immunoglobulin that binds two targets comprises the steps of a) obtaining a first parent antibody, or functional fragment thereof, that binds a first target; b) obtaining a second parent antibody or functional fragment thereof, that binds a second target; c) constructing two copies of a first polypeptide chains, each of which comprises VH1-(X1)n-VH2-C-(X2)n, wherein, VH1 is a first heavy chain variable domain obtained from said first parent antibody, or functional fragment thereof; VH2 is a second heavy chain variable domain obtained from said second parent antibody or functional fragment thereof, which can be the same as or different from the first parent antibody; C is a heavy chain constant domain; (X1)n is a linker wherein said (X1)n is either present or absent; and (X2)n is an Fc region, d) constructing two copies of a second polypeptide chains each of which comprises VL1-(X1)n-VL2-C-(X2)n, wherein, VL1 is a first light chain variable domain obtained from said first parent antibody, or functional fragment thereof; VL2 is a second light chain variable domain obtained from said second parent antibody, or functional fragment thereof, which can be the same as or different from the first parent antibody; C is a light chain constant domain; (X1)n is a linker, wherein said (X1)n is either present or absent; and (X2)n does not comprise an Fc region, wherein said (X2)n is either present or absent; and e) expressing two copies of said first and second polypeptide chains; such that a DVD-Ig binds said first target and said second target is generated.

Generation of First Binding Domain and/or Second Binding Domain

The variable domains of the DVD binding protein can be obtained from parent antibodies, including polyclonal and mAbs that bind targets of interest. These antibodies may be naturally occurring or may be generated by recombinant technology, or can be designed de novo. MAbs can be prepared using a wide variety of techniques known in the art including the use of hybridoma, recombinant, and phage display technologies, or a combination thereof. Monoclonal antibodies can be prepared by methods disclosed herein.

The dual variable domain immunoglobulin (DVD-Ig) molecule is designed such that two different light chain variable domains (VL) from the two different parent monoclonal antibodies are linked in tandem directly or via a short linker by recombinant DNA techniques, followed by the light chain constant domain, and optionally, an Fc region. Similarly, the heavy chain comprises two different heavy chain variable domains (VH) linked in tandem, followed by the constant domain CH1 and Fc region. The variable domains can be obtained using recombinant DNA techniques from a parent antibody generated by any one of the methods described herein. The variable domain may be a murine heavy or light chain variable domain, a CDR a human heavy or light chain variable domain. The first and second variable domains may be linked directly to each other using recombinant DNA techniques, linked via a linker sequence, or the two variable domains are linked. The variable domains may bind the same target or may bind different targets. The constant domain may be linked to the two linked variable domains using recombinant DNA techniques. Sequence comprising linked heavy chain variable domains may be linked to a heavy chain constant domain and sequence comprising linked light chain variable domains is linked to a light chain constant domain. The constant domains may also be human heavy chain constant domain and human light chain constant domain respectively. The DVD heavy chain may be further linked to an Fc region. The Fc region may be a native sequence Fc region, or a variant Fc region, or a human Fc region. Two heavy chain DVD polypeptides and two light chain DVD polypeptides may be combined to form a DVD-Ig molecule.

The design of the “dual-specific multivalent full length binding proteins” of the present disclosure leads to a dual variable domain light chain and a dual variable domain heavy chain which assemble primarily to the desired “dual-specific multivalent full length binding proteins”.

### Construction of DVD Molecules

The dual variable domain immunoglobulin (DVD-Ig) molecule is designed such that two different light chain variable domains (VL) from the two parent monoclonal antibodies, which can be the same or different, are linked in tandem directly or via a short linker by recombinant DNA techniques, followed by the light chain constant domain, and optionally, an Fc region. Similarly, the heavy chain comprises two different heavy chain variable domains (VH) linked in tandem, followed by the constant domain CH1 and Fc region

The variable domains can be obtained using recombinant DNA techniques from a parent antibody generated by any one of the methods described herein. In an embodiment, the variable domain is a murine heavy or light chain variable domain. In another embodiment, the variable domain is a CDR grafted or a humanized variable heavy or light chain domain. In an embodiment, the variable domain is a human heavy or light chain variable domain.

In one embodiment the first and second variable domains are linked directly to each other using recombinant DNA techniques. In another embodiment the variable domains are linked via a linker sequence. In an embodiment, two variable domains are linked. Three or more variable domains may also be linked directly or via a linker sequence. The variable domains may bind the same target or may bind different targets. DVD-Ig molecules of the invention may include one immunoglobulin variable domain and one non-immunoglobulin variable domain, such as ligand binding domain of a receptor, or an active domain of an enzyme. DVD-Ig molecules may also comprise two or more non-Ig domains.

In an embodiment a constant domain is linked to the two linked variable domains using recombinant DNA techniques. In an embodiment, sequence comprising linked heavy chain variable domains is linked to a heavy chain constant domain and sequence comprising linked light chain variable domains is linked to a light chain constant domain. In an embodiment, the constant domains are human heavy chain constant domain and human light chain constant domain respectively. In an embodiment, the DVD heavy chain is further linked to an Fc region. The Fc region may be a native sequence Fc region, or a variant Fc region. In another embodiment, the Fc region is a human.

In another embodiment two heavy chain DVD polypeptides and two light chain DVD polypeptides are combined to form a DVD-Ig molecule.

Binding proteins of the present invention may be produced by any of a number of techniques known in the art. For example, expression from host cells, wherein expression vector(s) encoding the DVD heavy and DVD light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the DVD proteins of the invention in either prokaryotic or eukaryotic host cells, DVD proteins are expressed in eukaryotic cells, for example, mammalian host cells, because such eukaryotic cells (and in particular mammalian cells) are more likely than prokaryotic cells to assemble and secrete a properly folded and immunologically active DVD protein.

Exemplary mammalian host cells for expressing the recombinant antibodies of the invention include Chinese Hamster Ovary (CHO cells including dhfr-CHO cells), NS0 myeloma cells, COS cells, SP2 and PER.C6 cells. When recombinant expression vectors encoding DVD proteins are introduced into mammalian host cells, the DVD proteins are produced by culturing the host cells for a period of time sufficient to allow for expression of the DVD proteins in the host cells or secretion of the DVD proteins into the culture medium in which the host cells are grown. DVD proteins can be recovered from the culture medium using standard protein purification methods.

In an exemplary system for recombinant expression of DVD proteins in constructs described herein, a recombinant expression vector encoding both the DVD heavy chain and the DVD light chain is introduced into dhfr-CHO cells by calcium phosphate-mediated transfection. Within the recombinant expression vector, the DVD heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the DVD heavy and light chains and intact DVD protein is recovered from the culture medium. Standard molecular biology techniques are used to prepare the recombinant expression vector, transfect the host cells, select for transformants, culture the host cells and recover the DVD protein from the culture medium. Still further the invention provides a method of synthesizing a DVD protein of the invention by culturing a host cell of the invention in a suitable culture medium until a DVD protein of the invention is synthesized. The method can further comprise isolating the DVD protein from the culture medium.

An important feature of DVD-Ig is that it can be produced and purified in a similar way as a conventional antibody. The production of DVD-Ig results in a homogeneous, single major product with desired dual-specific activity, without any sequence modification of the constant region or chemical modifications of any kind. Other previously described methods to generate “bi-specific”, “multi-specific”, and “multi-specific multivalent” full length binding proteins do not lead to a single primary product but instead lead to the intracellular or secreted production of a mixture of assembled inactive, mono-specific, multi-specific, multivalent, full length binding proteins, and multivalent full length binding proteins with combination of different binding sites.

The design of the “dual-specific multivalent full length binding proteins” for use in constructs described herein leads to a dual variable domain light chain and a dual variable domain heavy chain which assemble primarily to the desired “dual-specific multivalent full length binding proteins”.

In some embodiments, at least 50%, at least 75% and at least 90% of the assembled, and expressed dual variable domain immunoglobulin molecules are the desired dual-specific tetravalent protein. This aspect of the invention particularly enhances the commercial utility of the invention. Therefore, the present invention includes a method to express a dual variable domain light chain and a dual variable domain heavy chain in a single cell leading to a single primary product of a “dual-specific tetravalent full length binding protein”.

Provided herein are methods of expressing a dual variable domain light chain and a dual variable domain heavy chain in a single cell leading to a “primary product” of a “dual-specific tetravalent full length binding protein,” where the “primary product” is more than 50% of all assembled protein, comprising a dual variable domain light chain and a dual variable domain heavy chain.

Provided herein are methods of expressing a dual variable domain light chain and a dual variable domain heavy chain in a single cell leading to a single “primary product” of a “dual-specific tetravalent full length binding protein,” where the “primary product” is more than 75% of all assembled protein, comprising a dual variable domain light chain and a dual variable domain heavy chain.

Provided herein are methods of expressing a dual variable domain light chain and a dual variable domain heavy chain in a single cell leading to a single “primary product” of a “dual-specific tetravalent full length binding protein,” where the “primary product” is more than 90% of all assembled protein, comprising a dual variable domain light chain and a dual variable domain heavy chain.

### Kappa-Lambda Antibodies

In some embodiments, provided herein are multispecific (e.g., bispecific, trispecific) antibodies in the kappa-lambda antibody format. The bispecific antibodies provided herein have a common heavy chain, two light chains—one Kappa (K), one Lambda (λ)—that each has a different specificity (i.e., two light chains, two specificities). The methods provided herein produce molecules having specific binding where diversity is restricted to the VL region. These methods produce the bispecific antibodies through controlled co-expression of the three chains (one VH chains, two VL chains), and purification of the bispecific antibody.

This type of molecule is composed of two copies of a unique heavy chain polypeptide, a first light chain variable region fused to a constant Kappa domain and second light chain variable region fused to a constant Lambda domain. Each combining site displays a different target specificity to which both the heavy and light chain contribute. The light chain variable regions can be of the Lambda or Kappa family and are preferably fused to a Lambda and Kappa constant domains, respectively. This is preferred in order to avoid the generation of non-natural polypeptide junctions. However, it is also possible to obtain bispecific antibodies of the invention by fusing a Kappa light chain variable domain to a constant Lambda domain for a first specificity and fusing a Lambda light chain variable domain to a constant Kappa domain for the second specificity.

An essential step of exemplary methods is the identification of two antibody Fv regions (each composed by a variable light chain and variable heavy chain domain) having different target specificities that share the same heavy chain variable domain. Numerous methods have been described for the generation of monoclonal antibodies and functional fragments thereof. Fully human antibodies are antibody molecules in which the sequence of both the light chain and the heavy chain, including the CDRs 1 and 2, arise from human genes. The CDR3 region can be of human origin or designed by synthetic means. Such antibodies are termed “human antibodies”, or “fully human antibodies” herein. Human monoclonal antibodies can be prepared by using the trioma technique; the human B-cell hybridoma technique; and the EBV hybridoma technique to produce human monoclonal antibodies. Human monoclonal antibodies may be utilized and may be produced by using human hybridomas or by transforming human B-cells with Epstein Barr Virus in vitro.

Monoclonal antibodies may be generated, e.g., by immunizing an animal with a target or an immunogenic functional fragment, derivative or variant thereof. Alternatively, the animal is immunized with cells transfected with a vector containing a nucleic acid molecule encoding the target, such that the target is expressed and associated with the surface of the transfected cells. A variety of techniques are well-known in the art for producing xenogenic non-human animals. For example, see U.S. Pat. Nos. 6,075,181 and 6,150,584, which is hereby incorporated by reference in its entirety.

Alternatively, antibodies may be obtained by screening a library that contains antibody or binding domain sequences for binding to the target peptide. This library may be prepared, e.g., in bacteriophage as protein or peptide fusions to a bacteriophage coat protein that is expressed on the surface of assembled phage particles and the encoding DNA sequences contained within the phage particles (i.e., “phage displayed library”).

Hybridomas resulting from myeloma/B cell fusions are then screened for reactivity to the target. Monoclonal antibodies may be prepared, for example, using hybridoma methods. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

Kappa-lambda antibodies having the same heavy chain variable domain can be generated by the use of antibody libraries in which the heavy chain variable domain is the same for all the library members and thus the diversity is confined to the light chain variable domain. However, as the light chain variable domain is expressed in conjunction with the heavy variable domain, both domains can contribute to binding. To further facilitate the process, antibody libraries containing the same heavy chain variable domain and either a diversity of Lambda variable light chains or Kappa variable light chains can be used in parallel for in vitro selection of antibodies against different targets. This approach enables the identification of two antibodies having a common heavy chain but one carrying a Lambda light chain variable domain and the other a Kappa light chain variable domain that can be used as building blocks for the generation of a bispecific antibody in the full immunoglobulin format of the invention. Numerous methods for the modification of the Fc portion have been described and are applicable to antibodies of the invention.

Another step of exemplary embodiments is the optimization of co-expression of the common heavy chain and two different light chains into a single cell to allow for the assembly of a bispecific antibody of the invention. If all the polypeptides get expressed at the same level and get assembled equally well to form an immunoglobulin molecule then the ratio of monospecific (same light chains) and bispecific (two different light chains) should be 50%.

The co-expression of the heavy chain and two light chains generates a mixture of three different antibodies into the cell culture supernatant: two monospecific bivalent antibodies and one bispecific bivalent antibody. The latter has to be purified from the mixture to obtain the molecule of interest. The method described herein greatly facilitates this purification procedure by the use of affinity chromatography media that specifically interact with the Kappa or Lambda light chain constant domains such as the CaptureSelect Fab Kappa and CaptureSelect Fab Lambda affinity matrices (BAC BV, Holland). This multi-step affinity chromatography purification approach is efficient and generally applicable to antibodies of the invention. This is in sharp contrast to specific purification methods that have to be developed and optimized for each bispecific antibodies derived from quadromas or other cell lines expressing antibody mixtures. Indeed, if the biochemical characteristics of the different antibodies in the mixtures are similar, their separation using standard chromatography technique such as ion exchange chromatography can be challenging or not possible at all.

The purified bispecific antibodies were characterized as follows. The flow-through and elution from each affinity purification step was analyzed by SDS-PAGE. The specificity and affinity of Kk-bodies was determined by ELISA and surface plasmon resonance. The methods of the invention allow for the identification of antibodies with affinities in the sub-nanomolar to nanomolar range without optimization. This is not obvious as the diversity in antibody libraries described herein is restricted to the light chain which contributes less to the binding energy in standard antibodies.

To avoid the requirement of having access to two antibodies having light chain variable domains of the Kappa and Lambda type being perceived as a limitation to the instant invention, the methods described herein allow for the generation of hybrid light chain in which a Lambda variable domain can be fused to a Kappa constant domain and conversely a Kappa variable domain can be fused to a Lambda constant domain. In some embodiments, the methods of generating bispecific and/or multi-specific antibodies use a complete serum-free chemically defined process. These methods incorporate the most widely used mammalian cell line in pharmaceutical industry, the Chinese Hamster Ovary (CHO) cell line. The methods described therein are used to generate both semi-stable and stable cell lines. The methods can be used to manufacture the bispecific and/or multi-specific antibodies of the invention at small scale (e.g., in an Erlenmeyer flask) and at mid-scale (e.g., in 25 L Wave bag). The methods are also readily adaptable for larger scale production of the bispecific and/or multi-specific antibodies, as well as antibody mixtures of the invention.

### Binding Affinity

Binding affinity is generally represented by the dissociation constant (K_{D}). K_{D} values for antibodies can be determined by any of the methods known in the art. Exemplary methods for determining K_{D} includes by using surface plasmon resonance (SPR), isothermal titration calorimetry (ITC), spectroscopic assays, biolayer interferometry (BLI), and grating-coupled interferometry (GCI).

In some embodiments, multispecific antibody described herein comprises a binding affinity in a range of from 1 pM to 1 μM, from 10 pM to 1 μM, from 100 pM to 1 μM, from 1 nM to 1 μM, from 10 nM to 1 μM, from 100 nM to 1 μM, from 500 nM to 1 μM, from 1 pM to 500 nM, from 10 pM to 500 nM, from 100 pM to 500 nM, from 1 nM to 500 nM, from 10 nM to 500 nM, from 100 nM to 500 nM, from 1 pM to 100 nM, from 10 pM to 100 nM, from 100 pM to 100 nM, from 1 nM to 100 nM, from 10 nM to 100 nM, from 1 pM to 10 nM, from 10 pM to 10 nM, from 100 pM to 10 nM, from 1 nM to 10 nM, from 1 pM to 1 nM, from 10 pM to 1 nM, from 100 pM to 1 nM, from 1 pM to 100 pM, or from 10 pM to 100 pM.

In some embodiments, BsAb antibody described herein comprises an average binding affinity for target antigen is in a range of from 1 pM to 1 μM, from 10 pM to 1 μM, from 100 pM to 1 μM, from 1 nM to 1 μM, from 10 nM to 1 μM, from 100 nM to 1 μM, from 500 nM to 1 μM, from 1 pM to 500 nM, from 10 pM to 500 nM, from 100 pM to 500 nM, from 1 nM to 500 nM, from 10 nM to 500 nM, from 100 nM to 500 nM, from 1 pM to 100 nM, from 10 pM to 100 nM, from 100 pM to 100 nM, from 1 nM to 100 nM, from 10 nM to 100 nM, from 1 pM to 10 nM, from 10 pM to 10 nM, from 100 pM to 10 nM, from 1 nM to 10 nM, from 1 pM to 1 nM, from 10 pM to 1 nM, from 100 pM to 1 nM, from 1 pM to 100 pM, or from 10 pM to 100 pM.

In some embodiments, multispecific antibody described herein comprises at least two binding domains. In some embodiments, the first binding domain is TL1A binding domain. In some embodiments, the second binding domain targets IL-6, IL-12, IL-23, IL-23p19, IL-12p40, IL-12p35, IL-6R, an IL-17 family cytokine, or IL-17R. In some embodiments, the binding affinity of multispecific antibody is measured with only one target molecule (e.g., TL1A, IL-6, IL-12, IL-23, IL-23p19, IL-12p40, IL-12p35, IL-6R, an IL-17 family cytokine, or IL-17R). In some embodiments, a binding affinity of TL1A binding domain for TL1A is greater than a binding affinity of second binding domain for IL IL-6, IL-12, IL-23, IL-23p19, IL-12p40, IL-12p35, IL-6R, an IL-17 family cytokine, or IL-17R. In some embodiments, the multispecific antibody comprises the binding affinity of TL1A binding domain for TL1A that is at least two times, at least three time, at least four times, at least five times, at least ten times, at least fifteen times, at least twenty times, at least forty times, at least sixty times, at least eight times, or at least hundred times higher than the binding affinity of second binding domain for IL-6, IL-12, IL-23, IL-23p19, IL-12p40, IL-12p35, IL-6R, an IL-17 family cytokine, or IL-17R. Alternatively, in some embodiments, a binding affinity of second binding domain for IL-6, IL-12, IL-23, IL-23p19, IL-12p40, IL-12p35, IL-6R, an IL-17 family cytokine, or IL-17R is greater than a binding affinity of TL1A binding domain for TL1A. Accordingly, in some embodiments, the multispecific antibody comprises the binding affinity of second binding domain for IL-6, IL-12, IL-23, IL-23p19, IL-12p40, IL-12p35, IL-6R, an IL-17 family cytokine, or IL-17R that is at least two times, at least three time, at least four times, at least five times, at least ten times, at least fifteen times, at least twenty times, at least forty times, at least sixty times, at least eight times, or at least hundred times higher than the binding affinity of TL1A binding domain for TL1A.

Alternatively, in some embodiments, multispecific antibodies described herein undergo cooperative binding event. In some embodiments, the multispecific antibody undergo positive cooperative binding event, wherein binding of multispecific antibody to the first target molecule results in increase in binding affinity for the second target molecule. For example, in some embodiments, a binding affinity of TL1A bound multispecific antibody for IL-6, IL-12, IL-23, IL-23p19, IL-12p40, IL-12p35, IL-6R, an IL-17 family cytokine, or IL-17R is greater than a binding affinity of TL1A unbound multispecific antibody for IL-6, IL-12, IL-23, IL-23p19, IL-12p40, IL-12p35, IL-6R, an IL-17 family cytokine, or IL-17R. In some embodiments, a binding affinity of IL-6, IL-12, IL-23, IL-23p19, IL-12p40, IL-12p35, IL-6R, an IL-17 family cytokine, or IL-17R bound multispecific antibody for TL1A is greater than a binding affinity of IL-6, IL-12, IL-23, IL-23p19, IL-12p40, IL-12p35, IL-6R, an IL-17 family cytokine, or IL-17R unbound multispecific antibody for TL1A. Alternatively, in some embodiments, the multispecific antibody undergo negative cooperative binding event, wherein binding of multispecific antibody to the first target molecule results in decrease in binding affinity for the second target molecule. For example, in some embodiments, a binding affinity of TL1A bound multispecific antibody for IL-6, IL-12, IL-23, IL-23p19, IL-12p40, IL-12p35, IL-6R, an IL-17 family cytokine, or IL-17R that is lower than a binding affinity of TL1A unbound multispecific antibody for IL-6, IL-12, IL-23, IL-23p19, IL-12p40, IL-12p35, IL-6R, an IL-17 family cytokine, or IL-17R. In some embodiments, a binding affinity of IL-6, IL-12, IL-23, IL-23p19, IL-12p40, IL-12p35, IL-6R, an IL-17 family cytokine, or IL-17R bound multispecific antibody for TL1A is lower than a binding affinity of IL-6, IL-12, IL-23, IL-23p19, IL-12p40, IL-12p35, IL-6R, an IL-17 family cytokine, or IL-17R unbound multispecific antibody for TL1A.

For example, in some embodiments, BsAb described herein comprises two binding domains. In some embodiments, the first binding domain and the second binding domain are TL1A binding domain and IL-12 binding domain, respectively. In some embodiments, the binding affinity of BsAb is measured with only one target molecule (e.g., TL1A or IL-12 (includes one or more subunits of IL-12 (e.g., IL-12p35 and/or IL-12p40)). Accordingly, in some embodiments, the BsAb comprises a binding affinity for TL1A that is at least two times, at least three time, at least four times, at least five times, at least ten times, at least fifteen times, at least twenty times, at least forty times, at least sixty times, at least eight times, or at least hundred times higher than the binding affinity for the IL-12. Alternatively, in some embodiments, the BsAb comprises a binding affinity for IL-12 that is at least two times, at least three time, at least four times, at least five times, at least ten times, at least fifteen times, at least twenty times, at least forty times, at least sixty times, at least eight times, or at least hundred times higher than the binding affinity for the TL1A. In some embodiments, a binding affinity of TL1A bound bispecific antibody for IL-12 is greater than a binding affinity of TL1A unbound bispecific antibody for IL-12. In some embodiments, a binding affinity of IL-12 bound bispecific antibody for TL1A is greater than a binding affinity of IL-12 unbound bispecific antibody for TL1A. In some embodiments, a binding affinity of TL1A bound bispecific antibody for IL-12 is lower than a binding affinity of TL1A unbound bispecific antibody for IL-12. In some embodiments, a binding affinity of IL-12 bound bispecific antibody for TL1A is lower than a binding affinity of IL-12 unbound bispecific antibody for TL1A.

Similarly, in some embodiments, the first binding domain and the second binding domain are TL1A binding domain and IL-23 binding domain, respectively. In some embodiments, the binding affinity of BsAb is measured with only one target molecule (e.g., TL1A or IL-23 (includes one or more subunits of IL-23 (e.g., IL-23p19 and/or IL-12p40)). Accordingly, in some embodiments, the BsAb comprises a binding affinity for TL1A that is at least two times, at least three time, at least four times, at least five times, at least ten times, at least fifteen times, at least twenty times, at least forty times, at least sixty times, at least eight times, or at least hundred times higher than the binding affinity for the IL-23, when neither binding domains are bound to their respective binding partner. Alternatively, in some embodiments, the BsAb comprises a binding affinity for IL-23 that is at least two times, at least three time, at least four times, at least five times, at least ten times, at least fifteen times, at least twenty times, at least forty times, at least sixty times, at least eight times, or at least hundred times higher than the binding affinity for the TL1A, when neither binding domains are bound to their respective binding partner. In some embodiments, a binding affinity of TL1A bound bispecific antibody for IL-23 is greater than a binding affinity of TL1A unbound bispecific antibody for IL-23. In some embodiments, a binding affinity of IL-23 bound bispecific antibody for TL1A is greater than a binding affinity of IL-23 unbound bispecific antibody for TL1A. In some embodiments, a binding affinity of TL1A bound bispecific antibody for IL-23 is lower than a binding affinity of TL1A unbound bispecific antibody for IL-23. In some embodiments, a binding affinity of IL-23 bound bispecific antibody for TL1A is lower than a binding affinity of IL-23 unbound bispecific antibody for TL1A.

### Methods of Introducing pH Dependent Binding Activity

Disclosed herein, in some embodiments, are methods of making multispecific antibodies with a binding affinity for a target peptide selected from TL1A, IL-6R, IL-6, IL-12, IL-23, IL-23p19, IL-12p40, IL-12p35, an IL-17 family cytokine, IL-17R, a variant thereof and a fragment thereof, wherein the multispecific antibodies comprise a pH dependent target peptide binding activity. In some embodiments, the methods comprise identifying the target epitope comprising a histidine rich binding pocket; constructing the TL1A binding antibody that comprises at least one heavy chain variable region and at least one light chain variable region that bind the target epitope; and modifying at least one amino acid of the at least one heavy chain variable region, the at least one light chain variable region, or a combination thereof. In some embodiments, a binding affinity of the multispecific antibodies comprising the at least one modification for the target peptide in neutral pH is increased by at least 2%, at least 5%, at least 10%, at least 15%, at least 20%, at least 30%, at least 40%, at least 50% or more relative to a corresponding antibody prior to the at least one modification. In some embodiments, a binding affinity of the multispecific antibodies comprising the at least one modification for the target peptide in acidic pH is not more than 25% higher relative to a corresponding antibody prior to the at least one modification. In some embodiments, a binding affinity of the multispecific antibodies comprising the at least one modification for the target peptide in acidic pH is reduced by at least 2%, at least 5%, at least 10%, at least 15%, at least 20%, at least 30%, at least 40%, at least 50% or more relative to a corresponding antibody prior to the at least one modification. In some embodiments, the methods further comprise modifying constant regions of the multispecific antibodies. In some embodiments, a modified constant region of a multispecific antibody decreases plasma clearance (CL), increases plasma retention time, or increases plasma half-life (t½) of, compared to a corresponding antibody prior to modification of the constant regions. In some embodiments, a modified constant region of a multispecific antibody increases multispecific molecules mediated antigen clearance (CL), compared to a corresponding antibody prior to modification of the constant regions.

### Methods of Treatment

Disclosed herein, in some embodiments, are methods of treating a subject in need thereof, comprising administering to the subject, a dose in an effective amount of a multispecific (e.g., bispecific, trispecific) antibody or multispecific (e.g., bispecific, trispecific) molecule described herein or a pharmaceutical composition comprising said multispecific (e.g., bispecific, trispecific) antibody or multispecific (e.g., bispecific, trispecific) molecule described herein. In some embodiments, the subject has a disease or condition that is related to impaired mitochondrial dysfunction. In some embodiments, the subject has a disease or condition selected from the group consisting of: an infectious disease, or an autoimmune disease. In some embodiments, the subject has: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

In some embodiments, the multispecific (e.g., bispecific, trispecific) antibody or multispecific (e.g., bispecific, trispecific) molecule is administered with one or more additional therapeutic agents. In some embodiments, the multispecific (e.g., bispecific, trispecific) antibody or multispecific (e.g., bispecific, trispecific) molecule and one or more additional therapeutic agents are co-administered. In some embodiments, the multispecific (e.g., bispecific, trispecific) antibody or multispecific (e.g., bispecific, trispecific) molecule and one or more additional therapeutic agents are sequentially administered.

### Methods of Diagnosis

Disclosed herein, in some embodiments, are methods of diagnosing a condition of a subject, comprising incubating a sample with an effective amount of a composition comprising a multispecific (e.g., bispecific, trispecific) antibody or multispecific (e.g., bispecific, trispecific) molecule described herein. In some embodiments, the diagnosis is based on the expression level of TL1A, wherein an elevated level of TL1A in the subject relative to that in a healthy individual indicates that the individual suffers from an autoimmune condition. In some embodiments, the autoimmune condition comprises rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

### Dosages

Provided herein are compositions comprising a multispecific (e.g., bispecific, trispecific) antibody or multispecific (e.g., bispecific, trispecific) molecule or functional fragment thereof for treatment (including prevention) of a disease (e.g., an infectious condition, disorder or disease, an autoimmune condition, disorder or disease, a dermatological condition, disorder or disease). In some embodiments, the compositions are pharmaceutical compositions comprising a pharmaceutically acceptable carrier. The compositions are administered in an amount effective for treatment (including prophylaxis) of an infectious condition, disorder or disease, an autoimmune condition, disorder or disease, a dermatological condition, disorder or disease. In some embodiments, the compositions (e.g., the antibodies or the functional fragment thereof or the nucleic acid molecules encoding said antibody or functional fragment thereof) are administered in an amount effective for enhancing an immune response and/or increasing T cell activation in a subject. The compositions are to be used for in vivo administration to a subject by any available means, such as parenteral administration. For administration to a subject, a composition or medicament comprising the antibodies or functional fragment thereof described herein can be sterile, which can readily be accomplished by filtration through sterile filtration membranes, or other methods known to those of skill in the art. In one embodiment, a composition or medicament has been treated to be free of pyrogens or endotoxins. Testing pharmaceutical compositions or medicaments for pyrogens or endotoxins and preparing pharmaceutical compositions or medicaments free of pyrogens or endotoxins or preparing pharmaceutical compositions or medicaments that have endotoxins at a clinically acceptable level, are well understood to one of ordinary skill in the art. Commercial kits are available to test pharmaceutical compositions or medicaments for pyrogens or endotoxins.

The compositions to be used for in vivo administration, such as parenteral administration, in the methods described herein can be sterile, which is readily accomplished by filtration through sterile filtration membranes, or other methods known to those of skill in the art.

### Pharmaceutical Compositions and Dosage Forms

Disclosed herein, in certain embodiments, are pharmaceutical compositions comprising a multispecific (e.g., bispecific, trispecific) antibody or multispecific (e.g., bispecific, trispecific) molecule or a functional fragment thereof disclosed herein for administration in a subject.

In some embodiments, pharmaceutical compositions comprising a multispecific (e.g., bispecific, trispecific) molecule or multispecific (e.g., bispecific, trispecific) antibody described herein are formulated in a conventional manner using one or more physiologically acceptable carriers including excipients and auxiliaries which facilitate processing of the active compounds into preparations which can be used pharmaceutically. Proper formulation is dependent upon the route of administration chosen.

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions may also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some embodiments, the pharmaceutical composition is formulated for parenteral (e.g., intravenous, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

In some embodiments, the pharmaceutical compositions are formulated into capsules. In some embodiments, the pharmaceutical compositions are formulated into solutions (for example, for IV administration). In some embodiments, the pharmaceutical composition is formulated as an infusion. In some embodiments, the pharmaceutical composition is formulated as an injection.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

The compositions disclosed herein, comprising an antibody or functional fragment, described herein, can also contain more than one active compound as necessary for the particular indication being treated, preferably those with complementary activities that do not adversely affect each other. For example, the composition can further comprise retinoids, such as Acitretin (e.g., Soriatane®) and isotretinoin, immune system suppressants (e.g., rapamycin, Tcell blockers [e.g., Amevive® (alefacept) and Raptiva® [efalizumab]), cyclosporine, methotrexate, mycophenolate mofetil, mycophenolic acid, leflunomide, tacrolimus, etc.), hydroxyurea (e.g., Hydrea®), sulfasalazine, 6-thioguanine, fumarates (e.g., dimethylfumarate and fumaric acid esters), azathioprine, colchicine, alitretinoin, steroids, corticosteroids, certolizumab, aprelimast, mometasone, rosiglitazone, pioglitazone, botulinium toxin, triamcinolone, IFN-1 (InflaRx), bimekizumab (UCB), MaBpl (XBiotech), LY-3041658 (Eli Lilly), TE-2232 (Immunwork), NSAIDs, prescription narcotics, ketoprofen, codeine, gabapentin, pregabalin gentanyl, antibiotics (topical, oral, IV) (e.g., clindamycin, rifampin, tetracycline, sarecycline, doxycycline, minocycline, lymecycline, trimethoprim-sulfamethoxazole, erythromycin, ceftriaxone, moxifloxacin, metronidazole, separately or as combinations), corticosteroid (injectable or oral), antiandrogen/hormonal therapy (oral contraceptives, spironolactone, finasteride, dutasteride, progesterone IUD, cyproterone acetate, ethinyloestradiol, gestodene, norgestimate, desogestrel, drospirenone, spironolactone), Triamcinolone Acetonide, MEDI8968, hydroxychloroquine, dapsone, metformin, adapalene, azelaic acid and zinc. Such molecules are suitably present in combination in amounts that are effective for the purpose intended. The active ingredients of the compositions comprising an antibody or functional fragment thereof described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microparticle, microemulsions, nano-particles and nanocapsules) or in macroemulsions. The pharmaceutical composition can be also delivered in a vesicle, in particular a liposome. Liposomes include emulsions, foams, micelles, insoluble monolayers, phospholipid dispersions, lamellar layers and the like, and can serve as vehicles to target the M-CSF antibodies to a particular tissue as well as to increase the half-life of the composition. A variety of methods are available for preparing liposomes, as described in, e.g., U.S. Pat. Nos. 4,837,028 and 5,019,369, which patents are incorporated herein by reference.

For oral, buccal, and sublingual administration, powders, suspensions, granules, tablets, pills, capsules, gelcaps, and caplets are acceptable as solid dosage forms. These can be prepared, for example, by mixing one or more compounds of the instant invention, or pharmaceutically acceptable salts or tautomers thereof, with at least one additive such as a starch or other additive. Suitable additives are sucrose, lactose, cellulose sugar, mannitol, maltitol, dextran, starch, agar, alginates, chitins, chitosans, pectins, tragacanth gum, gum arabic, gelatins, collagens, casein, albumin, synthetic or semi-synthetic polymers or glycerides. Optionally, oral dosage forms can contain other ingredients to aid in administration, such as an inactive diluent, or lubricants such as magnesium stearate, or preservatives such as paraben or sorbic acid, or anti-oxidants such as ascorbic acid, tocopherol or cysteine, a disintegrating agent, binders, thickeners, buffers, sweeteners, flavoring agents or perfuming agents. Tablets and pills may be further treated with suitable coating materials known in the art.

Liquid dosage forms for oral administration may be in the form of pharmaceutically acceptable emulsions, syrups, elixirs, suspensions, and solutions, which may contain an inactive diluent, such as water. In some embodiments, pharmaceutical formulations and medicaments may be prepared as liquid suspensions or aqueous solutions, for example, using a sterile liquid, such as, but not limited to, an oil, water, an alcohol, and combinations of these. In some embodiments, pharmaceutical compositions can be prepared in a lyophilized form. The lyophilized preparations can comprise a cryoprotectant known in the art. The term “cryoprotectants” as used herein generally includes agents, which provide stability to the protein from freezing-induced stresses. Examples of cryoprotectants include polyols such as, for example, mannitol, and include saccharides such as, for example, sucrose, as well as including surfactants such as, for example, polysorbate, poloxamer or polyethylene glycol, and the like. Cryoprotectants also contribute to the tonicity of the formulations. Pharmaceutically suitable surfactants, suspending agents, emulsifying agents, may be added for oral or par-enteral administration.

Injectable dosage forms generally include aqueous suspensions or oil suspensions which may be prepared using a suitable dispersant or wetting agent and a suspending agent. Injectable forms may be in solution phase or in the form of a suspension, which is prepared with a solvent or diluent. Acceptable solvents or vehicles include sterilized water, Ringer's solution, or an isotonic aqueous saline solution. Alternatively, sterile oils may be employed as solvents or suspending agents. Preferably, the oil or fatty acid is non-volatile, including natural or synthetic oils, fatty acids, mono-, di- or tri-glycerides.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

### Kits

Provided herein are also kits, medicines, compositions, and unit dosage forms for use in any of the methods described herein. Provided herein is a kit comprising a therapeutically effective amount of at least one of the multispecific (e.g., bispecific, trispecific) antibody or multispecific (e.g., bispecific, trispecific) molecule or functional fragment thereof disclosed herein. In some embodiments, the kit further comprises a second therapeutic agent (e.g., an immune system suppressant, immunomodulating agent, or other agent including but not limited to an agent disclosed herein). In some embodiments, the antibody or functional fragment thereof is an aqueous form or a lyophilized form. The kit further comprises a diluent or a reconstitution solution.

Kits can include one or more containers comprising an antibody (or unit dosage forms and/or articles of manufacture). In some embodiments, a unit dosage is provided wherein the unit dosage contains a predetermined amount of a composition comprising an antibody (e.g., a therapeutically effective amount), with or without one or more additional agents. In some embodiments, such a unit dosage is supplied in single-use prefilled syringe for injection. In some embodiments, the composition comprising the antibody or functional fragment thereof can comprise saline, sucrose, or the like; a buffer, such as phosphate, or the like; and/or be formulated within a stable and effective pH range. In some embodiments, the antibody or functional fragment thereof can be provided as a lyophilized powder that may be reconstituted upon addition of an appropriate liquid, for example, sterile water. In some embodiments, the antibody or functional fragment thereof further comprises one or more substances that inhibit protein aggregation, including, but not limited to, sucrose and arginine. In some embodiments, the antibody or functional fragment thereof further comprises heparin and/or a proteoglycan.

In some embodiments, kits further comprise instructions for use in the treatment of a disease or condition in accordance with any of the methods described herein. The kit may further comprise a description of selection an individual suitable or treatment. Instructions supplied in the kits are typically written instructions on a label or package insert (for example, a paper sheet included in the kit), but machine-readable instructions (for example, instructions carried on a magnetic or optical storage disk) are also acceptable. In some embodiments, the kit further comprises another therapeutic agent.

The kits are in suitable packaging. Suitable packaging includes, but is not limited to, vials, bottles, jars, flexible packaging (for example, sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

### Exemplary Embodiments

Disclosed herein are bispecific molecules comprising: a first binding domain and a second binding domain, wherein the first binding domain is a TL1A binding domain, a variant thereof or a functional fragment thereof, and wherein the second binding domain is any one of IL-6R binding domain, IL-6 binding domain, IL-12 binding domain, IL-23 binding domain, IL-23p19 binding domain, IL-12p40 binding domain, IL-12p35 binding domain, IL-17 family cytokine binding domain, IL-17R binding domain, a variant thereof or a functional fragment thereof. In some embodiments, the bispecific molecule is an antibody, a variant thereof, or a functional fragment thereof. In some embodiments, the TL1A binding domain comprises an IgV domain or a stalk region. In some embodiments, the bispecific molecule is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the bispecific molecule comprises a heterodimeric antibody or a functional fragment thereof. In some embodiments, the bispecific molecule comprises a constant region. In some embodiments, the molecule comprises a sequence knock out in a constant region. In some embodiments, the first binding domain comprises a TL1A-binding heavy chain variable domain. In some embodiments, the first binding domain comprises a TL A-binding light chain variable domain. In some embodiments, the second binding domain is selected from IL-6R binding domain, IL-6 binding domain, IL-12 binding domain, IL-23 binding domain, IL-23p19 binding domain, IL-12p40 binding domain, IL-12p35 binding domain, IL-17 family cytokine binding domain or IL-17R binding domain. In some embodiments, the second domain comprising IL-6R binding heavy chain variable domain, IL-6 binding heavy chain variable domain, IL-12 binding heavy chain variable domain, IL-23 binding heavy chain variable domain, IL-23p19 binding heavy chain variable domain, IL-12p40 binding heavy chain variable domain, IL-12p35 binding heavy chain variable domain, IL-17 family cytokine binding heavy chain variable domain, IL-17R binding heavy chain variable domain, IL-6R binding light chain variable domain, IL-6 binding light chain variable domain, IL-12 binding light chain variable domain, IL-23 binding light chain variable domain, IL-23p19 binding light chain variable domain, IL-12p40 binding light chain variable domain, IL-12p35 binding light chain variable domain, IL-17 family cytokine binding light chain variable domain, IL-17R binding light chain variable domain, or a combination thereof. In some embodiments, at least one of the first binding domain and the second binding domain comprises a light chain constant domain and/or heavy chain constant domain. In some embodiments, the bispecific molecules comprise a pH-dependent target binding activity.

Also disclosed herein are bispecific molecules for use in the treatment of a disease or condition. In some embodiments, the disease or condition is selected from the group consisting of: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof. In some embodiments, the bispecific molecule comprises at least one of a Fc region and/or a Fab region. In some embodiments, the bispecific molecule comprises at least one scFv region.

Also disclosed herein are pharmaceutical compositions comprising a bispecific molecule described herein, and a pharmaceutically acceptable carrier. Also disclosed herein are methods of treating a disease or condition in a subject, the method comprising administering to the subject an effective amount of the bispecific molecule or the pharmaceutical composition of any one of the preceding claims, thereby treating the disease or condition.

Also disclosed herein are methods of treating an infectious disease, autoimmune disease and/or dermatological disease in a subject, the method comprising administering to the subject an effective amount of a bispecific molecule that comprises: a TL1A binding domain that comprises at least one of the heavy chain complementarity-determining regions (CDR-Hs) recited in TABLE 38, at least one of the light chain CDR-Ls recited in TABLE 40, variants thereof, or combinations thereof; and a second binding domain. In some embodiments, the second binding domain comprises at least one of the heavy chain complementarity-determining regions (CDR-Hs) recited in TABLE 2, variants thereof or combinations thereof, and at least one of the light chain CDR-Ls recited in TABLE 4, variants thereof or combinations thereof. In some embodiments, the second binding domain comprises at least one of the heavy chain complementarity-determining regions (CDR-Hs) recited in TABLE 9, variants thereof or combinations thereof, and at least one of the light chain CDR-Ls recited in TABLE 11, variants thereof or combinations thereof. In some embodiments, the second binding domain comprises at least one of the heavy chain complementarity-determining regions (CDR-Hs) recited in TABLE 16, variants thereof or combinations thereof, and at least one of the light chain CDR-Ls recited in TABLE 18, variants thereof or combinations thereof. In some embodiments, the second binding domain comprises at least one of the heavy chain complementarity-determining regions (CDR-Hs) recited in TABLE 23, variants thereof or combinations thereof, and at least one of the light chain CDR-Ls recited in TABLE 25, variants thereof or combinations thereof. In some embodiments, the second binding domain comprises at least one of the heavy chain complementarity-determining regions (CDR-Hs) recited in TABLE 30, variants thereof or combinations thereof, and at least one of the light chain CDR-Ls recited in TABLE 32, variants thereof or combinations thereof.

Also disclosed herein are compositions comprising a bispecific molecule comprising a first binding domain and a second binding domain, wherein the first binding domain is a TL1A binding domain, a variant thereof or a functional fragment thereof, wherein the second binding domain binds any one of IL-6R, IL-6, IL-12, IL-23, IL-23p19, IL-12p40, IL-12p35, an IL-17 family cytokine, IL-17R, a variant thereof or a functional fragment thereof, and wherein administration of an effective amount of the composition to a subject in need thereof results in treatment of a disease or condition. In some embodiments, the bispecific molecule is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the bispecific molecule comprises a heterodimeric antibody or a functional fragment thereof. In some embodiments, the bispecific molecule comprises a constant region. In some embodiments, the bispecific molecule comprises a sequence knock out in a constant region. Also disclosed herein are nucleic acids encoding at least a portion of any one of the bispecific molecules disclosed herein.

Provided are multispecific molecule and other exemplary compositions that bind both TL1A, and a protein selected from IL-6R, IL-6, an IL-17 family cytokine, IL-17R. For example, in some embodiments, a multispecific molecule, such as a bispecific molecule, comprises a first binding domain and a second binding domain. In some embodiments, the first binding domain binds TL1A, a variant thereof or a functional fragment thereof. In some embodiments, the second binding domain binds any one of IL-6R, an IL-17 family cytokine, IL-17R, a variant thereof, and a functional fragment thereof. In some embodiments, IL-17 family cytokine comprises any one of IL-17A, IL-17B, IL-17C, IL17-D, IL-17E and IL-17F. In some embodiments, IL-17 family cytokine comprises IL-17A and IL-17A/F. In some embodiments, the bispecific molecule is an antibody, a variant thereof, or a functional fragment thereof. In some embodiments, the bispecific molecule is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the bispecific molecule comprises a heterodimeric antibody or a functional fragment thereof. In some embodiments, the bispecific molecule comprises a constant region. In some embodiments, the first binding domain comprises a TL1A-binding heavy chain variable domain. In some embodiments, the first binding domain comprises a TL1A-binding light chain variable domain. In some embodiments, the second binding domain comprises any one of IL-6R binding heavy chain variable domain, IL-6 binding heavy chain variable domain, IL-17 family cytokine binding heavy chain variable domain and IL-17R binding heavy chain variable domain. In some embodiments, the second binding domain comprises any one of IL-6R binding light chain variable domain, IL-6 binding light chain variable domain, IL-17 family cytokine binding light chain variable domain and IL-17R binding light chain variable domain. In some embodiments, at least one of the first binding domain and the second binding domain comprises a light chain constant domain and/or heavy chain constant domain. In some embodiments, the bispecific molecule comprises at least one of a Fc region, a Fab region, and/or an scFv region. In some embodiments, the multispecific molecule comprises a pH-dependent target binding activity.

Provided are multispecific molecule and other exemplary compositions that bind both TL1A, and IL-12. For example, in some embodiments, a multispecific molecule, such as a bispecific molecule, comprises a first binding domain and a second binding domain. In some embodiments, the first binding domain binds TL1A, a variant thereof or a functional fragment thereof. In some embodiments, the second binding domain binds IL-12, a subunit thereof, a variant thereof, and a functional fragment thereof. In some embodiments, wherein a binding affinity of the first binding domain for TL1A is at least five times higher than a binding affinity of the second domain for IL-12. In some embodiments, the bispecific molecule is an antibody, a variant thereof, or a functional fragment thereof. In some embodiments, the bispecific molecule is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the bispecific molecule comprises a heterodimeric antibody or a functional fragment thereof. In some embodiments, the bispecific molecule comprises a constant region. In some embodiments, the first binding domain comprises a TL1A-binding heavy chain variable domain. In some embodiments, the first binding domain comprises a TL1A-binding light chain variable domain. In some embodiments, the second binding domain comprises an IL-12 binding heavy chain variable domain. In some embodiments, the second binding domain comprises an IL-12 binding light chain variable domain. In some embodiments, at least one of the first binding domain and the second binding domain comprises a light chain constant domain and/or heavy chain constant domain. In some embodiments, the bispecific molecule comprises at least one of a Fc region, a Fab region, and/or an scFv region. In some embodiments, the multispecific molecule comprises a pH-dependent target binding activity.

Provided are multispecific molecule and other exemplary compositions that bind both TL1A, and IL-12. For example, in some embodiments, a multispecific molecule, such as a bispecific molecule, comprises a first binding domain and a second binding domain. In some embodiments, the first binding domain binds TL1A, a variant thereof or a functional fragment thereof. In some embodiments, the second binding domain binds IL-12, a subunit thereof, a variant thereof, and a functional fragment thereof. In some embodiments, wherein a binding affinity of the first binding domain for TL1A is lower than a binding affinity of the second domain for IL-12. In some embodiments, the bispecific molecule is an antibody, a variant thereof, or a functional fragment thereof. In some embodiments, the bispecific molecule is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the bispecific molecule comprises a heterodimeric antibody or a functional fragment thereof. In some embodiments, the bispecific molecule comprises a constant region. In some embodiments, the first binding domain comprises a TL1A-binding heavy chain variable domain. In some embodiments, the first binding domain comprises a TL1A-binding light chain variable domain. In some embodiments, the second binding domain comprises an IL-12 binding heavy chain variable domain. In some embodiments, the second binding domain comprises an IL-12 binding light chain variable domain. In some embodiments, at least one of the first binding domain and the second binding domain comprises a light chain constant domain and/or heavy chain constant domain. In some embodiments, the bispecific molecule comprises at least one of a Fc region, a Fab region, and/or an scFv region. In some embodiments, the multispecific molecule comprises a pH-dependent target binding activity.

Provided are multispecific molecule and other exemplary compositions that bind both TL1A, and IL-23. For example, in some embodiments, a multispecific molecule, such as a bispecific molecule, comprises a first binding domain and a second binding domain. In some embodiments, the first binding domain binds TL1A, a variant thereof or a functional fragment thereof. In some embodiments, the second binding domain binds IL-23, a subunit thereof, a variant thereof, and a functional fragment thereof. In some embodiments, wherein a binding affinity of the first binding domain for TL1A is at least five times higher than a binding affinity of the second domain for IL-23. In some embodiments, the bispecific molecule is an antibody, a variant thereof, or a functional fragment thereof. In some embodiments, the bispecific molecule is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the bispecific molecule comprises a heterodimeric antibody or a functional fragment thereof. In some embodiments, the bispecific molecule comprises a constant region. In some embodiments, the first binding domain comprises a TL1A-binding heavy chain variable domain. In some embodiments, the first binding domain comprises a TL1A-binding light chain variable domain. In some embodiments, the second binding domain comprises an IL-23 binding heavy chain variable domain. In some embodiments, the second binding domain comprises an IL-23p19 binding heavy chain variable domain. In some embodiments, the second binding domain comprises an IL-23 binding light chain variable domain. In some embodiments, the second binding domain comprises an IL-23p19 binding light chain variable domain. In some embodiments, at least one of the first binding domain and the second binding domain comprises a light chain constant domain and/or heavy chain constant domain. In some embodiments, the bispecific molecule comprises at least one of a Fc region, a Fab region, and/or an scFv region. In some embodiments, the multispecific molecule comprises a pH-dependent target binding activity.

Provided are multispecific molecule and other exemplary compositions that bind both TL1A, and IL-23. For example, in some embodiments, a multispecific molecule, such as a bispecific molecule, comprises a first binding domain and a second binding domain. In some embodiments, the first binding domain binds TL1A, a variant thereof or a functional fragment thereof. In some embodiments, the second binding domain binds IL-23, a subunit thereof, a variant thereof, and a functional fragment thereof. In some embodiments, wherein a binding affinity of the first binding domain for TL1A is lower than a binding affinity of the second domain for IL-23. In some embodiments, the bispecific molecule is an antibody, a variant thereof, or a functional fragment thereof. In some embodiments, the bispecific molecule is a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof. In some embodiments, the bispecific molecule comprises a heterodimeric antibody or a functional fragment thereof. In some embodiments, the bispecific molecule comprises a constant region. In some embodiments, the first binding domain comprises a TL1A-binding heavy chain variable domain. In some embodiments, the first binding domain comprises a TL1A-binding light chain variable domain. In some embodiments, the second binding domain comprises an IL-23 binding heavy chain variable domain. In some embodiments, the second binding domain comprises an IL-23p19 binding heavy chain variable domain. In some embodiments, the second binding domain comprises an IL-23 binding light chain variable domain. In some embodiments, the second binding domain comprises an IL-23p19 binding light chain variable domain. In some embodiments, at least one of the first binding domain and the second binding domain comprises a light chain constant domain and/or heavy chain constant domain. In some embodiments, the bispecific molecule comprises at least one of a Fc region, a Fab region, and/or an scFv region. In some embodiments, the multispecific molecule comprises a pH-dependent target binding activity.

Also described herein are multispecific molecules for use for use in the treatment of a disease or condition, wherein the multispecific molecules are any one of the multispecific molecules described herein. In some embodiments, the disease or condition is related to impaired mitochondrial dysfunction. In some embodiments, the disease or condition is selected from the group consisting of: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

Also described herein are pharmaceutical compositions, wherein the pharmaceutical compositions comprise any one of the multispecific molecules described herein and a pharmaceutically acceptable carrier.

Also described herein are methods of treating a disease or condition in a subject. In some embodiments, the methods comprise administering to the subject an effective amount of the bispecific molecule or the pharmaceutical composition of any one of the preceding claims, thereby treating the disease or condition.

Also described herein are compositions. In some embodiments, the compositions comprise a bispecific molecule comprising a first binding domain and a second binding domain, wherein the first binding domain binds TL1A, a variant thereof or a functional fragment thereof, wherein the second binding domain binds any one of IL-6R, IL-6, an IL-17 family cytokine, IL-17R, a variant thereof, and a functional fragment thereof, and wherein administration of an effective amount of the composition to a subject in need thereof results in treatment of a disease or condition. In some embodiments, the disease or condition is selected from the group consisting of: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

Also described herein are compositions comprising a bispecific molecule, wherein the bispecific molecule comprises a first binding domain and a second binding domain, wherein the first binding domain binds TL1A, a variant thereof, or a functional fragment thereof, wherein the second binding domain binds IL-12, a subunit thereof, a variant thereof, or a functional fragment thereof, wherein a binding affinity of the first binding domain for TL1A is at least five times higher than a binding affinity of the second domain for IL-12, and wherein administration of an effective amount of the composition to a subject in need thereof results in treatment of a disease or condition. In some embodiments, the disease or condition is selected from the group consisting of: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

Also described herein are compositions comprising a bispecific molecule, wherein the bispecific molecule comprises a first binding domain and a second binding domain, wherein the first binding domain binds TL1A, a variant thereof, or a functional fragment thereof, wherein the second binding domain binds IL-12, a subunit thereof, a variant thereof, or a functional fragment thereof, wherein a binding affinity of the first binding domain for TL1A is lower than a binding affinity of the second domain for IL-12, and wherein administration of an effective amount of the composition to a subject in need thereof results in treatment of a disease or condition. In some embodiments, the disease or condition is selected from the group consisting of: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

Also described herein are compositions comprising a bispecific molecule, wherein the bispecific molecule comprises a first binding domain and a second binding domain, wherein the first binding domain binds TL1A, a variant thereof, or a functional fragment thereof, wherein the second binding domain binds IL-23, a subunit thereof, a variant thereof, or a functional fragment thereof, wherein a binding affinity of the first binding domain for TL1A is at least two times higher than a binding affinity of the second domain for IL-23, and wherein administration of an effective amount of the composition to a subject in need thereof results in treatment of a disease or condition. In some embodiments, the disease or condition is selected from the group consisting of: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

Also described herein are compositions comprising a bispecific molecule, wherein the bispecific molecule comprises a first binding domain and a second binding domain, wherein the first binding domain binds TL1A, a variant thereof or a functional fragment thereof, wherein the second binding domain binds IL-23, a subunit thereof, a variant thereof, or a functional fragment thereof, wherein a binding affinity of the first binding domain for TL1A is lower than a binding affinity of the second domain for IL-23, and wherein administration of an effective amount of the composition to a subject in need thereof results in treatment of a disease or condition. In some embodiments, the disease or condition is selected from the group consisting of: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

Also described herein are compositions comprising a bispecific molecule, wherein the bispecific molecule comprises a first binding domain and a second binding domain, wherein the first binding domain binds TL1A, a variant thereof, or a functional fragment thereof, wherein the second domain binds any one of IL-6R, IL-6, IL-12, IL-23, IL-12p35, IL-12p40, IL-23p19, an IL-17 family cytokine, IL-17R, a variant thereof, and a functional fragment thereof, wherein the bispecific molecule further comprises a mutant Fc domain, wherein the mutant Fc domain comprises a deletion of a c-terminal lysine relative to corresponding wild-type Fc domain, and wherein subcutaneous administration of an effective amount of the composition to a subject in need thereof results in treatment of a disease or condition. In some embodiments, IL-17 family cytokine comprises any one of IL-17A, IL-17B, IL-17C, IL17-D, IL-17E and IL-17F.

Also described herein are bispecific molecule comprising: a first binding domain and a second binding domain, wherein the second domain binds to IL-12p35, a variant thereof, or a functional fragment thereof. In some embodiments, the first binding domain binds to TL1A.

Also described herein are nucleic acids. In some embodiments, the nucleic acids encode at least a portion of any one of the multispecific molecules described herein.

Also described herein are compositions comprising: a bispecific antibody comprising a first binding domain and a second binding domain, wherein the first binding domain binds a first target of TL1A, a variant thereof, or a functional fragment thereof, wherein the second binding domain binds a second target selected from IL-6R, IL-6, an IL-17 family cytokine, IL-17R, a variant thereof, and a functional fragment thereof, wherein the first binding domain comprises a first heavy chain variable region, and a first light chain variable region, wherein the first binding domain comprises at least one amino acid modification in the first heavy chain variable region and/or the first light chain variable region, wherein the at least one amino acid modification changes an isoelectric point (pI) of the bispecific antibody relative to a pI of a corresponding antibody prior to the at least one modification, thereby resulting in pH-dependent binding activity to the bispecific antibody, and wherein a binding affinity of the first binding domain to the first target is at least 75% of a corresponding binding domain prior to the at least one modification as measured by surface plasmon resonance spectroscopy. In some embodiments, the at least one modification increases a binding affinity of the first binding domain for the first target in neutral pH condition relative to the corresponding binding domain prior to the at least one modification. In some embodiments, the at least one modification increases the binding affinity of the first binding domain by at least 2%, at least 5%, at least 10%, at least 15%, at least 20%, at least 30%, at least 40%, at least 50% or more relative to the corresponding binding domain prior to the at least one modification. In some embodiments, the at least one modification reduces a binding affinity of the first binding domain for the first target in acidic pH condition by at least 10% relative to the corresponding binding domain prior to the at least one modification. In some embodiments, the at least one modification is within the first heavy chain variable region that comprises a CDR-H1, a CDR-H2 and a CDR-H3. In some embodiments, the at least one modification is within any one of the CDR-H1, the CDR-H2 and the CDR-H3. In some embodiments, the at least one modification is within at least one framework region of the first heavy chain variable region. In some embodiments, the at least one modification is within the first light chain variable region that comprises a CDR-L1, a CDR-L2 and a CDR-L3. In some embodiments, the at least one modification is within any one of the CDR-L1, the CDR-L2 and the CDR-L3. In some embodiments, the at least one modification is within at least one framework region of the first light chain variable region. In some embodiments, the first binding domain comprises a first constant region comprising at least one modification relative to any one of the amino acid sequences recited in TABLE 44. In some embodiments, a binding affinity of the first constant region for a neonatal fragment crystallizable receptor (FcRn) is increased in acidic pH condition relative to corresponding constant region prior to the at least one modification, and wherein a binding affinity of the constant region for the FcRn in neutral pH condition remains within 20% of corresponding binding affinity of the corresponding constant region prior to the at least one modification. In some embodiments, the binding affinity of the constant region for the FcRn is increased by at least 5%, at least 10%, at least 15%, at least 20%, at least 25%, at least 30%, at least 50%, at least 70% or more in acidic pH condition relative to the corresponding constant region prior to the at least one modification. In some embodiments, the bispecific antibody comprises decreased plasma clearance (CL), increased plasma retention time, or increased plasma half-life (t½) relative to a corresponding bispecific antibody prior to the at least one modification in the constant region. In some embodiments, a binding affinity of the constant region for a FcRn is increased in neutral pH condition relative to a corresponding constant region prior to the at least one modification, and wherein a binding affinity of the constant region for the FcRn in acidic pH condition remains within 20% of the corresponding constant region prior to the at least one modification. In some embodiments, the binding affinity of the constant region for the FcRn is increased by at least 5%, at least 10%, at least 15%, at least 20%, at least 25%, at least 30%, at least 50%, at least 70% or more in neutral pH condition relative to the corresponding constant region prior to the at least one modification. In some embodiments, the at least one modification increases plasma clearance of the first target by the bispecific antibody, compared to a corresponding plasma clearance by a bispecific molecule prior to the at least one modification in the constant region.

### Additional Embodiments

Embodiment 1: An IgG antibody construct comprising: a TL1A binding region that binds an epitope on TL1A, a variant thereof or a functional fragment thereof, and an IL-23 binding region that binds an epitope on IL-23, a variant thereof or a functional fragment thereof, wherein:
said TL1A binding region comprises a TL1A binding heavy chain variable domain and a TL1A binding light chain variable domain,
at least one amino acid modification in said TL1A binding heavy chain variable domain and/or said TL1A binding light chain variable domain, and
said amino acid modification changes an isoelectric point (pI) of said IgG antibody construct relative to a pI of a corresponding IgG antibody construct prior to said amino acid modification, thereby resulting in pH-dependent binding activity for said TL1A in said IgG antibody construct, as measured by surface plasmon resonance spectroscopy.

Embodiment 2: The IgG antibody construct of embodiment 1, wherein said IL-23 binding region comprises: an IL-23 binding heavy chain variable domain and an IL-23 binding light chain variable domain, and at least one amino acid modification in said IL-23 binding heavy chain variable domain and/or said IL-23 binding light chain variable domain, wherein said amino acid modification changes an isoelectric point (pI) of said IgG antibody construct relative to a pI of a corresponding IgG antibody construct prior to said amino acid modification, thereby resulting in pH-dependent binding activity for said IL-23 in said IgG antibody construct, as measured by surface plasmon resonance spectroscopy.

Embodiment 3: An IgG antibody construct comprising: a TL1A binding region that binds an epitope on TL1A, a variant thereof or a functional fragment thereof, and an IL-23 binding region that binds an epitope on IL-23, a variant thereof or a functional fragment thereof, wherein:
said TL1A binding region comprises a TL1A binding heavy chain variable domain,
said IL-23 binding region comprises an IL-23 binding heavy chain variable domain, and
a binding affinity of said TL1A binding region for said TL1A is at least 10% higher at pH 7.4 relative to said binding affinity at pH 5.8, as measured by surface plasmon resonance spectroscopy.

Embodiment 4: The IgG antibody construct of embodiment 3, wherein a binding affinity of said IL-23 binding region for said IL-23 is at least 10% higher at pH 7.4 relative to said binding affinity at pH 5.8, as measured by surface plasmon resonance spectroscopy.

Embodiment 5: An IgG antibody construct comprising: a TL1A binding region that binds an epitope on TL1A, a variant thereof or a functional fragment thereof, and an IL-23 binding region that binds an epitope on IL-23, a variant thereof or a functional fragment thereof, wherein:
said TL1A binding region comprises a TL1A binding heavy chain variable domain, and
said IL-23 binding region comprises an IL-23 binding heavy chain variable domain.

Embodiment 6: The IgG antibody construct of embodiment 5, wherein a binding affinity of said TL1A binding region for said TL1A is higher than a binding affinity of said IL-23 binding region for said IL-23 under neutral pH condition, as measured by surface plasmon resonance spectroscopy.

Embodiment 7: The IgG antibody construct of embodiment 6, wherein said binding affinity of said TL1A binding region for said TL1A is at least two times higher than said binding affinity of said IL-23 binding region for said IL-23.

Embodiment 8: The IgG antibody construct of embodiment 5, wherein a binding affinity of said IL-23 binding region for said IL-23 is higher than a binding affinity of said TL1A binding region for said TL1A under neutral pH condition, as measured by surface plasmon resonance spectroscopy.

Embodiment 9: The IgG antibody construct of embodiment 8, wherein a binding affinity of said IL-23 binding region for said IL-23 is at least two times higher than a binding affinity of said TL A binding region for said TL1A.

Embodiment 10: The IgG antibody construct of any one of embodiments 3-9, wherein
said IgG antibody construct comprises a TL1A binding light chain variable domain that interact with said TL1A binding heavy chain variable domain, thereby forming said TL1A binding region,
said TL1A binding heavy chain variable domain and/or said TL1A binding light chain variable domain comprises at least one amino acid modification that
(a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or
(b) decreases binding affinity of said TL1A binding region for said TL1A relative to said binding affinity of corresponding TL1A binding region prior to said at least one amino acid modification under acidic pH condition, as measured by surface plasmon resonance spectroscopy.

Embodiment 11: The IgG antibody construct of any one of embodiments 3-10, wherein:
said IgG antibody construct comprises an IL-23 binding light chain variable domain that interact with said IL-23 binding heavy chain variable domain, thereby forming said IL-23 binding region,
said IL-23 binding heavy chain variable domain and/or said IL-23 binding light chain variable domain comprises at least one amino acid modification that
(a) increases binding affinity of said IL-23 binding region for said IL-23 relative to said binding affinity of corresponding IL-23 binding region prior to said at least one amino acid modification under neutral pH condition, as measured by surface plasmon resonance spectroscopy, and/or
(b) decreases binding affinity of said IL-23 binding region for said IL-23 relative to said binding affinity of corresponding IL-23 binding region prior to said at least one amino acid modification under acidic pH condition, as measured by surface plasmon resonance spectroscopy.

Embodiment 12: The IgG antibody construct of any one of embodiments 1-2 and 10-11, wherein said at least one amino acid modification is within a CDR-H1, a CDR-H2, and/or a CDR-H3 of said TL1A binding heavy chain variable domain.

Embodiment 13: The IgG antibody construct of embodiment 12, wherein said at least one amino acid modification is within a CDR-H3 of said TL1A binding heavy chain variable domain.

Embodiment 14: The IgG antibody construct of embodiment 13, wherein said at least one amino acid modification comprises a substitution of at least one uncharged amino acid with a charged amino acid.

Embodiment 15: The IgG antibody construct of any one of embodiments 1-2 and 10-14, wherein said at least one amino acid modification is within at least one framework region of said TL1A binding heavy chain variable domain.

Embodiment 16: The IgG antibody construct of any one of embodiments 1-2 and 10-15, wherein said at least one amino acid modification is within a CDR-L1, a CDR-L2, and/or a CDR-L3 of said TL1A binding light chain variable domain.

Embodiment 17: The IgG antibody construct of any one of embodiments 1-2 and 10-16, wherein said at least one amino acid modification is within at least one framework region of said TL1A binding light chain variable domain.

Embodiment 18: The IgG antibody construct of any one of embodiments 1-2 and 10-17, wherein said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to said binding affinity of corresponding TL1A binding region prior to said at least one amino acid modification, as measured by surface plasmon resonance spectroscopy.

Embodiment 19: The IgG antibody construct of any one of embodiments 1-2 and 10-18, wherein said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy.

Embodiment 20: The IgG antibody construct of any one of embodiments 1-2 and 10-19, wherein said at least one amino acid modification is within a CDR-H1, a CDR-H2, and/or a CDR-H3 of said IL-23 binding heavy chain variable domain.

Embodiment 21: The IgG antibody construct of any one of embodiments 1-2 and 10-20, wherein said at least one amino acid modification is within at least one framework region of said IL-23 binding heavy chain variable domain.

Embodiment 22: The IgG antibody construct of any one of embodiments 1-2 and 11-21, wherein said at least one amino acid modification is within a CDR-L1, a CDR-L2, and/or a CDR-L3 of said IL-23 binding light chain variable domain.

Embodiment 23: The IgG antibody construct of any one of embodiments 1-2 and 10-22, wherein said at least one amino acid modification is within at least one framework region of said TL1A binding light chain variable domain.

Embodiment 24: The IgG antibody construct of any one of embodiments 1-2 and 10-23, wherein said at least one amino acid modification increases binding affinity of said IL-23 binding region for said IL-23 in neutral pH condition by at least 2%, at least 5%, at least 10%, at least 15%, at least 20%, at least 30%, at least 40%, at least 50% or more relative to said binding affinity of corresponding IL-23 binding region prior to said at least one amino acid modification, as measured by surface plasmon resonance spectroscopy.

Embodiment 25: The IgG antibody construct of any one of embodiments 1-2 and 10-24, wherein said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy.

Embodiment 26: The IgG antibody construct of any one of embodiments 1-4 and 6-25, wherein said IL-23 binding heavy chain variable region further binds an epitope that is present on IL-12p40, a variant thereof or a functional fragment thereof.

Embodiment 27: The IgG antibody construct of any one of embodiments 1-26, wherein said IgG antibody construct has higher binding affinity for trimeric TL1A relative to monomeric TL1A under neutral pH condition.

Embodiment 28: The IgG antibody construct of any one of embodiments 1-26, wherein said IgG antibody construct has higher binding affinity for monomeric TL1A relative to trimeric TL1A under neutral pH condition.

Embodiment 29: The IgG antibody construct of any one of embodiments 1-28, wherein said IgG antibody construct has higher binding affinity for monomeric IL-23 relative to heterodimeric IL-23 under neutral pH condition.

Embodiment 30: The IgG antibody construct of any one of embodiments 1-28, wherein said IgG antibody construct has higher binding affinity for heterodimeric IL-23 relative to monomeric IL-23 under neutral pH condition.

Embodiment 31: The IgG antibody construct of any one of embodiments 11-30, wherein said TL1A binding light chain variable domain and said IL-23 binding light chain variable domain are at least 90%, at least 95%, at least 98% or 100% identical relative to each other.

Embodiment 32: The IgG antibody construct of any one of embodiments 1-2 and 10-31, wherein said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification.

Embodiment 33: An IgG antibody construct comprising: a TL1A binding region that binds an epitope on TL1A, a variant thereof or a functional fragment thereof, and an IL-12 binding region that binds an epitope on IL-12p40, IL-12p35, a variant thereof or a functional fragment thereof, wherein:
said TL1A binding region comprises a TL1A binding heavy chain variable domain and a TL1A binding light chain variable domain,
at least one amino acid modification in said TL1A binding heavy chain variable domain and/or said TL1A binding light chain variable domain, and
said amino acid modification changes an isoelectric point (pI) of said IgG antibody construct relative to a pI of a corresponding IgG antibody construct prior to said amino acid modification, thereby resulting in pH-dependent binding activity for said TL1A in said IgG antibody construct, as measured by surface plasmon resonance spectroscopy.

Embodiment 34: The IgG antibody construct of embodiment 33, wherein said IL-12 binding region comprises: an IL-12 binding heavy chain variable domain and an IL-12 binding light chain variable domain, and at least one amino acid modification in said IL-12 binding heavy chain variable domain and/or said IL-12 binding light chain variable domain, wherein said amino acid modification changes an isoelectric point (pI) of said IgG antibody construct relative to a pI of a corresponding IgG antibody construct prior to said amino acid modification, thereby resulting in pH-dependent binding activity for said IL-12 in said IgG antibody construct, as measured by surface plasmon resonance spectroscopy.

Embodiment 35: An IgG antibody construct comprising: a TL1A binding region that binds an epitope on TL1A, a variant thereof or a functional fragment thereof, and an IL-12 binding region that binds an epitope on IL-12p40, IL-12p35, a variant thereof or a functional fragment thereof, wherein:
said TL1A binding region comprises a TL1A binding heavy chain variable domain,
said IL-12 binding region comprises an IL-12 binding heavy chain variable domain, and a binding affinity of said TL1A binding region for said TL1A is at least 10% higher at pH 7.4 relative to said binding affinity at pH 5.8, as measured by surface plasmon resonance spectroscopy.

Embodiment 36: The IgG antibody construct of embodiment 35, wherein a binding affinity of said IL-12 binding region for said IL-12 is at least 10% higher at pH 7.4 relative to said binding affinity at pH 5.8, as measured by surface plasmon resonance spectroscopy.

Embodiment 37: An IgG antibody construct comprising a TL1A binding region that binds an epitope on TL1A, a variant thereof or a functional fragment thereof, and an IL-12 binding region that binds an epitope on IL-12p40, IL-12p35, a variant thereof or a functional fragment thereof, wherein:
said TL1A binding region comprises a TL1A binding heavy chain variable domain,
said IL-12 binding region comprises an IL-12 binding heavy chain variable domain, and
a binding affinity of the TL1A binding region for said TL1A is more than four times a binding affinity of the IL-12 binding region for said IL-12 under neutral pH condition, as measured by surface plasmon resonance spectroscopy.

Embodiment 38: An IgG antibody construct comprising a TL1A binding region that binds an epitope on TL1A, a variant thereof or a functional fragment thereof, and an IL-12 binding region that binds an epitope on IL-12p40, IL-12p35, a variant thereof or a functional fragment thereof, wherein:
said TL1A binding region comprises a TL1A binding heavy chain variable domain,
said IL-12 binding region comprises an IL-12 binding heavy chain variable domain, and
a binding affinity of the IL-12 binding region for said IL-12 is higher than a binding affinity of the TL1A binding region for said TL1A under neutral pH condition, as measured by surface plasmon resonance spectroscopy.

Embodiment 39: The IgG antibody construct of embodiment 38, wherein a binding affinity of the IL-12 binding region for said IL-12 is at least two times higher than a binding affinity of the TL1A binding region for said TL1A.

Embodiment 40: The IgG antibody construct of any one of embodiments 35-39, wherein
said IgG antibody construct comprises a TL1A binding light chain variable domain that interact with said TL1A binding heavy chain variable domain, thereby forming said TL1A binding region,
said TL1A binding heavy chain variable domain and/or said TL1A binding light chain variable domain comprises at least one amino acid modification that
(a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or
(b) decreases binding affinity of said TL1A binding region for said TL1A relative to said binding affinity of corresponding TL1A binding region prior to said at least one amino acid modification under acidic pH condition, as measured by surface plasmon resonance spectroscopy.

Embodiment 41: The IgG antibody construct of any one of embodiments 35-40, wherein:
said IgG antibody construct comprises an IL-12 binding light chain variable domain that interact with said IL-12 binding heavy chain variable domain, thereby forming said IL-12 binding region,
said IL-12 binding heavy chain variable domain and/or said IL-12 binding light chain variable domain comprises at least one amino acid modification that
(a) increases binding affinity of said IL-12 binding region for said IL-12 relative to said binding affinity of corresponding IL-12 binding region prior to said at least one amino acid modification under neutral pH condition, as measured by surface plasmon resonance spectroscopy, and/or
(b) decreases binding affinity of said IL-12 binding region for said IL-12 relative to said binding affinity of corresponding IL-12 binding region prior to said at least one amino acid modification under acidic pH condition, as measured by surface plasmon resonance spectroscopy.

Embodiment 42: The IgG antibody construct of any one of embodiments 33-34 and 40-41, wherein said at least one amino acid modification is within a CDR-H1, a CDR-H2, and/or a CDR-H3 of said TL1A binding heavy chain variable domain.

Embodiment 43: The IgG antibody construct of embodiment 42, wherein said at least one amino acid modification is within a CDR-H3 of said TL1A binding heavy chain variable domain.

Embodiment 44: The IgG antibody construct of embodiment 43, wherein said at least one amino acid modification comprises a substitution of at least one uncharged amino acid with a charged amino acid.

Embodiment 45: The IgG antibody construct of any one of embodiments 33-34 and 40-44, wherein said at least one amino acid modification is within at least one framework region of said TL1A binding heavy chain variable domain.

Embodiment 46: The IgG antibody construct of any one of embodiments 33-34 and 40-45, wherein said at least one amino acid modification is within a CDR-L1, a CDR-L2, and/or a CDR-L3 of said TL1A binding light chain variable domain.

Embodiment 47: The IgG antibody construct of any one of embodiments 33-34 and 40-46, wherein said at least one amino acid modification is within at least one framework region of said TL1A binding light chain variable domain.

Embodiment 48: The IgG antibody construct of any one of embodiments 33-34 and 40-47, wherein said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy.

Embodiment 49: The IgG antibody construct of any one of embodiments 33-34 and 40-48, wherein said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy.

Embodiment 50: The IgG antibody construct of any one of embodiments 34 and 40-49, wherein said at least one amino acid modification is within a CDR-H1, a CDR-H2, and/or a CDR-H3 of said IL-12 binding heavy chain variable domain.

Embodiment 51: The IgG antibody construct of any one of embodiments 34 and 40-50, wherein said at least one amino acid modification is within at least one framework region of said IL-12 binding heavy chain variable domain.

Embodiment 52: The IgG antibody construct of any one of embodiments 34 and 41-51, wherein said at least one amino acid modification is within a CDR-L1, a CDR-L2, and/or a CDR-L3 of said IL-12 binding light chain variable domain.

Embodiment 53: The IgG antibody construct of any one of embodiments 33-34 and 40-52, wherein said at least one amino acid modification increases binding affinity of said IL-12 binding region for said IL-12 in neutral pH condition by at least 2%, at least 5%, at least 10%, at least 15%, at least 20%, at least 30%, at least 40%, at least 50% or more relative to binding affinity of corresponding IL-12 binding region prior to said at least one amino acid modification, as measured by surface plasmon resonance spectroscopy.

Embodiment 54: The IgG antibody construct of any one of embodiments 33-36 and 40-53, wherein said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy.

Embodiment 55: The IgG antibody construct of any one of embodiments 33-54, wherein said IL-12 binding heavy chain variable domain further binds an epitope that is present on IL-23, a variant thereof or a functional fragment thereof.

Embodiment 56: The IgG antibody construct of any one of embodiments 33-55, wherein said IgG antibody construct has higher binding affinity for trimeric TL1A relative to monomeric TL1A under neutral pH condition.

Embodiment 57: The IgG antibody construct of any one of embodiments 33-55, wherein said IgG antibody construct has higher binding affinity for monomeric TL1A relative to trimeric TL1A under neutral pH condition.

Embodiment 58: The IgG antibody construction of any one of embodiments 41-57, wherein said TL1A binding light chain variable domain and said IL-12 binding light chain variable domain are at least 90%, at least 95%, at least 98% or 100% identical relative to each other.

Embodiment 59: The IgG antibody construct of any one of embodiments 33-34 and 40-58, wherein said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification.

Embodiment 60: An IgG antibody construct comprising a TL1A binding region that binds an epitope on TL1A, a variant thereof or a functional fragment thereof, and an IL-6R binding region that binds an epitope on IL-6R, a variant thereof or a functional fragment thereof, wherein:
said TL1A binding region comprises a TL1A binding heavy chain variable domain and a TL1A binding light chain variable domain,
at least one amino acid modification in said TL1A binding heavy chain variable domain and/or said TL1A binding light chain variable domain, and
said amino acid modification changes an isoelectric point (pI) of said IgG antibody construct relative to a pI of a corresponding IgG antibody construct prior to said amino acid modification, thereby resulting in pH-dependent binding activity for said TL1A in said IgG antibody construct, as measured by surface plasmon resonance spectroscopy.

Embodiment 61: The IgG antibody construct of embodiment 60, wherein said IL-6R binding region comprises an IL-6R binding heavy chain variable domain and an IL-6R binding light chain variable domain, and at least one amino acid modification in said IL-6R binding heavy chain variable domain and/or said IL-6R binding light chain variable domain, wherein said amino acid modification changes an isoelectric point (pI) of said IgG antibody construct relative to a pI of a corresponding IgG antibody construct prior to said amino acid modification, thereby resulting in pH-dependent binding activity for said IL-6R in said IgG antibody construct, as measured by surface plasmon resonance spectroscopy.

Embodiment 62: An IgG antibody construct comprising a TL1A binding region that binds an epitope on TL1A, a variant thereof or a functional fragment thereof, and an IL-6R binding region that binds an epitope on IL-6R, a variant thereof or a functional fragment thereof, wherein:
said TL1A binding region comprises a TL1A binding heavy chain variable domain,
said IL-6R binding region comprises an IL-6R binding heavy chain variable domain, and
a binding affinity of said TL1A binding region for said TL1A is at least 10% higher at pH 7.4 relative to said binding affinity at pH 5.8, as measured by surface plasmon resonance spectroscopy.

Embodiment 63: The IgG antibody construct of embodiment 62, wherein a binding affinity of said IL-6R binding region for said IL-6R is at least 10% higher at pH 7.4 relative to said binding affinity at pH 5.8, as measured by surface plasmon resonance spectroscopy.

Embodiment 64: An IgG antibody construct comprising a TL1A binding region that binds an epitope on TL1A, a variant thereof or a functional fragment thereof, and an IL-6R binding region that binds an epitope on IL-6R, a variant thereof or a functional fragment thereof, wherein:
said TL1A binding region comprises a TL1A binding heavy chain variable domain, and
said IL-6R binding region comprises an IL-6R binding heavy chain variable domain.

Embodiment 65: The IgG antibody construct of embodiment 64, wherein a binding affinity of said TL1A binding region for said TL1A is higher than a binding affinity of said IL-6R binding region for said IL-6R under neutral pH condition, as measured by surface plasmon resonance spectroscopy.

Embodiment 66: The IgG antibody construct of embodiment 65, wherein said binding affinity of said TL1A binding region for said TL1A is at least two times higher than said binding affinity of said IL-6R binding region for said IL-6R.

Embodiment 67: The IgG antibody construct of embodiment 64, wherein a binding affinity of said IL-6R binding region for said IL-6R is higher than a binding affinity of said TL1A binding region for said TL1A under neutral pH condition, as measured by surface plasmon resonance spectroscopy.

Embodiment 68: The IgG antibody construct of embodiment 67, wherein a binding affinity of said IL-6R binding region for said IL-6R is at least two times higher than a binding affinity of said TL1A binding region for said TL1A.

Embodiment 69: The IgG antibody construct of any one of embodiments 62-68, wherein
said IgG antibody construct comprises a TL1A binding light chain variable domain that interact with said TL1A binding heavy chain variable domain, thereby forming said TL1A binding region, and
said TL1A binding heavy chain variable domain and/or said TL1A binding light chain variable domain comprises at least one amino acid modification that
(a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or
(b) decreases binding affinity of said TL1A binding region for said TL1A relative to said binding affinity of corresponding TL1A binding region prior to said at least one amino acid modification under acidic pH condition, as measured by surface plasmon resonance spectroscopy.

Embodiment 70: The IgG antibody construct of any one of embodiments 62-69, wherein:
said IgG antibody construct comprises an IL-6R binding light chain variable domain that interact with said IL-6R binding heavy chain variable domain, thereby forming said IL-6R binding region, and
said IL-6R binding heavy chain variable domain and/or said IL-6R binding light chain variable domain comprises at least one amino acid modification that
(a) increases binding affinity of said IL-6R binding region for said IL-6R relative to said binding affinity of corresponding IL-6R binding region prior to said at least one amino acid modification under neutral pH condition, as measured by surface plasmon resonance spectroscopy, and/or
(b) decreases binding affinity of said IL-6R binding region for said IL-6R relative to said binding affinity of corresponding IL-6R binding region prior to said at least one amino acid modification under acidic pH condition, as measured by surface plasmon resonance spectroscopy.

Embodiment 71: The IgG antibody construct of any one of embodiments 60-61 and 69-70, wherein said at least one amino acid modification is within a CDR-H1, a CDR-H2, and/or a CDR-H3 of said TL1A binding heavy chain variable domain.

Embodiment 72: The IgG antibody construct of embodiment 71, wherein said at least one amino acid modification is within a CDR-H3 of said TL1A binding heavy chain variable domain.

Embodiment 73: The IgG antibody construct of embodiment 72, wherein said at least one amino acid modification comprises a substitution of at least one uncharged amino acid with a charged amino acid.

Embodiment 74: The IgG antibody construct of any one of embodiments 60-61 and 69-73, wherein said at least one amino acid modification is within at least one framework region of said TL1A binding heavy chain variable domain.

Embodiment 75: The IgG antibody construct of any one of embodiments 60-61 and 69-74, wherein said at least one amino acid modification is within a CDR-L1, a CDR-L2, and/or a CDR-L3 of said TL1A binding light chain variable domain.

Embodiment 76: The IgG antibody construct of any one of embodiments 60-61 and 69-75, wherein said at least one amino acid modification is within at least one framework region of said TL1A binding light chain variable domain.

Embodiment 77: The IgG antibody construct of any one of embodiments 60-61 and 69-76, wherein said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy.

Embodiment 78: The IgG antibody construct of any one of embodiments 60-61 and 69-77, wherein said at least one amino acid modification reduces binding affinity of said TL A binding region for said TL1A in acidic pH condition by at least 10% relative to binding affinity of corresponding TL1A binding region prior to said at least one amino acid modification, as measured by surface plasmon resonance spectroscopy.

Embodiment 79: The IgG antibody construct of any one of embodiments 61 and 70-78, wherein said at least one amino acid modification is within a CDR-H1, a CDR-H2, and/or a CDR-H3 of said IL-6R binding heavy chain variable domain and/or a CDR-L1, a CDR-L2, and/or a CDR-L3 of said IL-6R binding light chain variable domain.

Embodiment 80: The IgG antibody construct of any one of embodiments 61 and 70-79, wherein said at least one amino acid modification is within at least one framework region of said IL-6R binding heavy chain variable domain and/or at least one framework region of said IL-6R binding light chain variable domain.

Embodiment 81: The IgG antibody construct of any one of embodiments 60-61 and 60-80, wherein said IgG antibody construct has higher binding affinity for trimeric TL1A relative to monomeric TL1A under neutral pH condition.

Embodiment 82: The IgG antibody construct of any one of embodiments 60-81, wherein said IgG antibody construct has higher binding affinity for monomeric TL1A relative to trimeric TL1A under neutral pH condition.

Embodiment 83: The IgG antibody construct of any one of embodiments 61 and 70-86, wherein said TL1A binding light chain variable domain and said IL-6R binding light chain variable domain are at least 90%, at least 95%, at least 98% or 100% identical relative to each other.

Embodiment 84: The IgG antibody construct of any one of embodiments 60-61 and 69-83, wherein said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification.

Embodiment 85: An IgG antibody construct comprising a TL1A binding region that binds an epitope on TL1A, a variant thereof or a functional fragment thereof, and an IL-6 binding region that binds an epitope on IL-6, a variant thereof or a functional fragment thereof, wherein:
said TL1A binding region comprises a TL1A binding heavy chain variable domain and a TL1A binding light chain variable domain,
at least one amino acid modification in said TL1A binding heavy chain variable domain and/or said TL1A binding light chain variable domain, and
said amino acid modification changes an isoelectric point (pI) of said IgG antibody construct relative to a pI of a corresponding IgG antibody construct prior to said amino acid modification, thereby resulting in pH-dependent binding activity for said TL1A in said IgG antibody construct, as measured by surface plasmon resonance spectroscopy.

Embodiment 86: The IgG antibody construct of embodiment 85, wherein said IL-6 binding region comprises an IL-6 binding heavy chain variable domain and an IL-6 binding light chain variable domain, and at least one amino acid modification in said IL-6 binding heavy chain variable domain and/or said IL-6 binding light chain variable domain, wherein said amino acid modification changes an isoelectric point (pI) of said IgG antibody construct relative to a pI of a corresponding IgG antibody construct prior to said amino acid modification, thereby resulting in pH-dependent binding activity for said IL-6 in said IgG antibody construct, as measured by surface plasmon resonance spectroscopy.

Embodiment 87: An IgG antibody construct comprising a TL1A binding region that binds an epitope on TL A, a variant thereof or a functional fragment thereof, and an IL-6 binding region that binds an epitope on IL-6, a variant thereof or a functional fragment thereof, wherein:
said TL1A binding region comprises a TL1A binding heavy chain variable domain,
said IL-6 binding region comprises an IL-6 binding heavy chain variable domain, and
a binding affinity of said TL1A binding region for said TL1A is at least 10% higher at pH 7.4 relative to said binding affinity at pH 5.8, as measured by surface plasmon resonance spectroscopy.

Embodiment 88: The IgG antibody construct of embodiment 87, wherein a binding affinity of said IL-6 binding region for said IL-6 is at least 10% higher at pH 7.4 relative to said binding affinity at pH 5.8, as measured by surface plasmon resonance spectroscopy.

Embodiment 89: An IgG antibody construct comprising a TL1A binding region that binds an epitope on TL1A, a variant thereof or a functional fragment thereof, and an IL-6 binding region that binds an epitope on IL-6, a variant thereof or a functional fragment thereof, wherein:
said TL1A binding region comprises a TL1A binding heavy chain variable domain, and
said IL-6 binding region comprises an IL-6 binding heavy chain variable domain.

Embodiment 90: The IgG antibody construct of embodiment 89, wherein a binding affinity of said TL1A binding region for said TL1A is higher than a binding affinity of said IL-6 binding region for said IL-6 under neutral pH condition, as measured by surface plasmon resonance spectroscopy.

Embodiment 91: The IgG antibody construct of embodiment 90, wherein said binding affinity of said TL1A binding region for said TL1A is at least two times higher than said binding affinity of said IL-6 binding region for said IL-6.

Embodiment 92: The IgG antibody construct of embodiment 89, wherein a binding affinity of said IL-6 binding region for said IL-6 is higher than a binding affinity of said TL1A binding region for said TL1A under neutral pH condition, as measured by surface plasmon resonance spectroscopy.

Embodiment 93: The IgG antibody construct of embodiment 92, wherein a binding affinity of said IL-6 binding region for said IL-6 is at least two times higher than a binding affinity of said TL1A binding region for said TL1A.

Embodiment 94: The IgG antibody construct of any one of embodiments 87-93, wherein
said IgG antibody construct comprises a TL1A binding light chain variable domain that interact with said TL1A binding heavy chain variable domain, thereby forming said TL1A binding region,
said TL1A binding heavy chain variable domain and/or said TL1A binding light chain variable domain comprises at least one amino acid modification that
(a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or
(b) decreases binding affinity of said TL1A binding region for said TL1A relative to said binding affinity of corresponding TL1A binding region prior to said at least one amino acid modification under acidic pH condition, as measured by surface plasmon resonance spectroscopy.

Embodiment 95: The IgG antibody construct of any one of embodiments 87-94, wherein:
said IgG antibody construct comprises an IL-6 binding light chain variable domain that interact with said IL-6 binding heavy chain variable domain, thereby forming said IL-6 binding region,
said IL-6 binding heavy chain variable domain and/or said IL-6 binding light chain variable domain comprises at least one amino acid modification that
(a) increases binding affinity of said IL-6 binding region for said IL-6 relative to said binding affinity of corresponding IL-6 binding region prior to said at least one amino acid modification under neutral pH condition, as measured by surface plasmon resonance spectroscopy, and/or
(b) decreases binding affinity of said IL-6 binding region for said IL-6 relative to said binding affinity of corresponding IL-6 binding region prior to said at least one amino acid modification under acidic pH condition, as measured by surface plasmon resonance spectroscopy.

Embodiment 96: The IgG antibody construct of embodiment 85-86 and 94-95, wherein said at least one amino acid modification is within a CDR-H1, a CDR-H2, and/or a CDR-H3 of said TL1A binding heavy chain variable domain.

Embodiment 97: The IgG antibody construct of embodiment 96, wherein said at least one amino acid modification is within a CDR-H3 of said TL1A binding heavy chain variable domain.

Embodiment 98: The IgG antibody construct of embodiment 97, wherein said at least one amino acid modification comprises a substitution of at least one uncharged amino acid with a charged amino acid.

Embodiment 99: The IgG antibody construct of any one of embodiments 85-86 and 94-98, wherein said at least one amino acid modification is within at least one framework region of said TL1A binding heavy chain variable domain.

Embodiment 100: The IgG antibody construct of any one of embodiments 85-86 and 94-99, wherein said at least one amino acid modification is within a CDR-L1, a CDR-L2, and/or a CDR-L3 of said TL1A binding light chain variable domain.

Embodiment 101: The IgG antibody construct of any one of embodiments 85-86 and 94-100, wherein said at least one amino acid modification is within at least one framework region of said TL1A binding light chain variable domain.

Embodiment 102: The IgG antibody construct of any one of embodiments 85-86 and 94-101, wherein said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy.

Embodiment 103: The IgG antibody construct of any one of embodiments 85-86 and 94-102, wherein said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy.

Embodiment 104: The IgG antibody construct of any one of embodiments 86 and 95-103, wherein said at least one amino acid modification is within a CDR-H1, a CDR-H2, and/or a CDR-H3 of said IL-6 binding heavy chain variable domain.

Embodiment 105: The IgG antibody construct of any one of embodiments 86 and 95-104, wherein said at least one amino acid modification is within at least one framework region of said IL-6 binding heavy chain variable domain.

Embodiment 106: The IgG antibody construct of any one of embodiments 86 and 95-105, wherein said at least one amino acid modification is within a CDR-L1, a CDR-L2, and/or a CDR-L3 of said IL-6 binding light chain variable domain.

Embodiment 107: The IgG antibody construct of any one of embodiments 86 and 95-106, wherein said at least one amino acid modification is within at least one framework region of said TL1A binding light chain variable domain.

Embodiment 108: The IgG antibody construct of any one of embodiments 85-86 and 94-107, wherein said at least one amino acid modification increases binding affinity of said IL-6 binding region for said IL-6 in neutral pH condition by at least 2%, at least 5%, at least 10%, at least 15%, at least 20%, at least 30%, at least 40%, at least 50% or more relative to binding affinity of corresponding IL-6 binding region prior to said at least one amino acid modification, as measured by surface plasmon resonance spectroscopy.

Embodiment 109: The IgG antibody construct of any one of embodiments 85-86 and 94-108, wherein said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy.

Embodiment 110: The IgG antibody construct of any one of embodiments 85-109, wherein said IgG antibody construct has higher binding affinity for trimeric TL1A relative to monomeric TL1A under neutral pH condition.

Embodiment 111: The IgG antibody construct of any one of embodiments 85-109, wherein said IgG antibody construct has higher binding affinity for monomeric TL1A relative to trimeric TL1A under neutral pH condition.

Embodiment 112: The IgG antibody construct of any one of embodiments 86 and 94-111, wherein said TL1A binding light chain variable domain and said IL-6 binding light chain variable domain are at least 90%, at least 95%, at least 98% or 100% identical relative to each other.

Embodiment 113: The IgG antibody construct of any one of embodiments 85-86 and 94-112, wherein said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification.

Embodiment 114: An IgG antibody construct comprising: a TL1A binding region that binds an epitope on TL1A, a variant thereof or a functional fragment thereof, and an IL-17 binding region that binds an epitope on IL-17 family cytokine, IL-17R, a variant thereof or a functional fragment thereof, wherein:
said TL1A binding region comprises a TL1A binding heavy chain variable domain and a TL1A binding light chain variable domain,
at least one amino acid modification in said TL1A binding heavy chain variable domain and/or said TL1A binding light chain variable domain, and
said amino acid modification changes an isoelectric point (pI) of said IgG antibody construct relative to a pI of a corresponding IgG antibody construct prior to said amino acid modification, thereby resulting in pH-dependent binding activity for said TL1A in said IgG antibody construct, as measured by surface plasmon resonance spectroscopy.

Embodiment 115: The IgG antibody construct of embodiment 114, wherein said IL-17 binding region comprises an IL-17 binding heavy chain variable domain and an IL-17 binding light chain variable domain, and at least one amino acid modification in said IL-17 binding heavy chain variable domain and/or said IL-17 binding light chain variable domain, wherein said amino acid modification changes an isoelectric point (pI) of said IgG antibody construct relative to a pI of a corresponding IgG antibody construct prior to said amino acid modification, thereby resulting in pH-dependent binding activity for said IL-17 in said IgG antibody construct, as measured by surface plasmon resonance spectroscopy.

Embodiment 116: An IgG antibody construct comprising a TL1A binding region that binds an epitope on TL1A, a variant thereof or a functional fragment thereof, and an IL-17 binding region that binds an epitope on IL-17 family cytokine, IL-17R, a variant thereof or a functional fragment thereof, wherein:
said TL1A binding region comprises a TL1A binding heavy chain variable domain,
said IL-17 binding region comprises an IL-17 binding heavy chain variable domain, and
a binding affinity of said TL1A binding region for said TL1A is at least 10% higher at pH 7.4 relative to said binding affinity at pH 5.8, as measured by surface plasmon resonance spectroscopy.

Embodiment 117: The IgG antibody construct of embodiment 116, wherein a binding affinity of said IL-17 binding region for said IL-17 is at least 10% higher at pH 7.4 relative to said binding affinity at pH 5.8, as measured by surface plasmon resonance spectroscopy.

Embodiment 118: An IgG antibody construct comprising a TL1A binding region that binds an epitope on TL1A, a variant thereof or a functional fragment thereof, and an IL-17 binding region that binds an epitope on IL-17 family cytokine, IL-17R, a variant thereof or a functional fragment thereof, wherein:
said TL1A binding region comprises a TL1A binding heavy chain variable domain, and
said IL-17 binding region comprises an IL-17 binding heavy chain variable domain.

Embodiment 119: The IgG antibody construct of embodiment 118, wherein a binding affinity of said TL1A binding region for said TL1A is higher than a binding affinity of said IL-17 binding region for said IL-17 under neutral pH condition, as measured by surface plasmon resonance spectroscopy.

Embodiment 120: The IgG antibody construct of embodiment 119, wherein said binding affinity of said TL1A binding region for said TL1A is at least two times higher than said binding affinity of said IL-17 binding region for said IL-17.

Embodiment 121: The IgG antibody construct of embodiment 118, wherein a binding affinity of said IL-17 binding region for said IL-17 is higher than a binding affinity of said TL1A binding region for said TL1A under neutral pH condition, as measured by surface plasmon resonance spectroscopy.

Embodiment 122: The IgG antibody construct of embodiment 121, wherein a binding affinity of said IL-17 binding region for said IL-17 is at least two times higher than a binding affinity of said TL1A binding region for said TL1A.

Embodiment 123: The IgG antibody construct of any one of embodiments 116-122, wherein
said IgG antibody construct comprises a TL1A binding light chain variable domain that interact with said TL1A binding heavy chain variable domain, thereby forming said TL1A binding region,
said TL1A binding heavy chain variable domain and/or said TL1A binding light chain variable domain comprises at least one amino acid modification that
(a) increases binding affinity of said TL1A binding region for said TL1A relative to said binding affinity of corresponding TL1A binding region prior to said at least one amino acid modification under neutral pH condition, as measured by surface plasmon resonance spectroscopy, and/or
(b) decreases binding affinity of said TL1A binding region for said TL1A relative to said binding affinity of corresponding TL1A binding region prior to said at least one amino acid modification under acidic pH condition, as measured by surface plasmon resonance spectroscopy.

Embodiment 124: The IgG antibody construct of any one of embodiments 116-123, wherein:
said IgG antibody construct comprises an IL-17 binding light chain variable domain that interact with said IL-17 binding heavy chain variable domain, thereby forming said IL-17 binding region,
said IL-17 binding heavy chain variable domain and/or said IL-17 binding light chain variable domain comprises at least one amino acid modification that
(a) increases binding affinity of said IL-17 binding region for said IL-17 relative to said binding affinity of corresponding IL-17 binding region prior to said at least one amino acid modification under neutral pH condition, as measured by surface plasmon resonance spectroscopy, and/or
(b) decreases binding affinity of said IL-17 binding region for said IL-17 relative to said binding affinity of corresponding IL-17 binding region prior to said at least one amino acid modification under acidic pH condition, as measured by surface plasmon resonance spectroscopy.

Embodiment 125: The IgG antibody construct of any one of embodiments 114-115 and 123-124, wherein said at least one amino acid modification is within a CDR-H1, a CDR-H2, and/or a CDR-H3 of said TL1A binding heavy chain variable domain.

Embodiment 126: The IgG antibody construct of embodiment 125, wherein said at least one amino acid modification is within a CDR-H3 of said TL1A binding heavy chain variable domain.

Embodiment 127: The IgG antibody construct of embodiment 126, wherein said at least one amino acid modification comprises a substitution of at least one uncharged amino acid with a charged amino acid.

Embodiment 128: The IgG antibody construct of any one of embodiments 114-115 and 123-127, wherein said at least one amino acid modification is within at least one framework region of said TL1A binding heavy chain variable domain.

Embodiment 129: The IgG antibody construct of any one of embodiments 114-115 and 123-128, wherein said at least one amino acid modification is within a CDR-L1, a CDR-L2, and/or a CDR-L3 of said TL1A binding light chain variable domain.

Embodiment 130: The IgG antibody construct of any one of embodiments 114-115 and 123-129, wherein said at least one amino acid modification is within at least one framework region of said TL1A binding light chain variable domain.

Embodiment 131: The IgG antibody construct of any one of embodiments 114-115 and 123-130, wherein said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to binding affinity of corresponding TL1A binding region prior to said at least one amino acid modification, as measured by surface plasmon resonance spectroscopy.

Embodiment 132: The IgG antibody construct of any one of embodiments 114-115 and 123-131, wherein said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy.

Embodiment 133: The IgG antibody construct of any one of embodiments 115 and 124-132, wherein said at least one amino acid modification is within a CDR-H1, a CDR-H2, and/or a CDR-H3 of said IL-17 binding heavy chain variable domain.

Embodiment 134: The IgG antibody construct of any one of embodiments 115 and 124-133, wherein said at least one amino acid modification is within at least one framework region of said IL-17 binding heavy chain variable domain.

Embodiment 135: The IgG antibody construct of any one of embodiments 115 and 124-134, wherein said at least one amino acid modification is within a CDR-L1, a CDR-L2, and/or a CDR-L3 of said IL-17 binding light chain variable domain.

Embodiment 136: The IgG antibody construct of any one of embodiments 115 and 124-135, wherein said at least one amino acid modification is within at least one framework region of said TL1A binding light chain variable domain.

Embodiment 137: The IgG antibody construct of any one of embodiments 114-115 and 124-135, wherein said at least one amino acid modification increases said binding affinity of said IL-17 binding region for said IL-17 in neutral pH condition by at least 2%, at least 5%, at least 10%, at least 15%, at least 20%, at least 30%, at least 40%, at least 50% or more relative to said binding affinity of corresponding IL-17 binding region prior to said at least one amino acid modification, as measured by surface plasmon resonance spectroscopy.

Embodiment 138: The IgG antibody construct of any one of embodiments 114-115 and 124-137, wherein said at least one amino acid modification reduces a binding affinity of said TL1A binding region for said TL1A in acidic pH condition by at least 10% relative to said binding affinity of corresponding TL1A binding region prior to said at least one amino acid modification, as measured by surface plasmon resonance spectroscopy.

Embodiment 139: The IgG antibody construct of any one of embodiments 114-138, wherein said IgG antibody construct has higher binding affinity for trimeric TL1A relative to monomeric TL1A under neutral pH condition.

Embodiment 140: The IgG antibody construct of any one of embodiments 114-138, wherein said IgG antibody construct has higher binding affinity for monomeric TL1A relative to trimeric TL1A under neutral pH condition.

Embodiment 141: The IgG antibody construct of any one of embodiments 115 and 124-140, wherein said TL1A binding light chain variable domain and said IL-17 binding light chain variable domain are at least 90%, at least 95%, at least 98% or 100% identical relative to each other.

Embodiment 142: The IgG antibody construct of any one of embodiments 114-115 and 123-141, wherein said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification.

Embodiment 143: The IgG antibody construct of any one of embodiments 1-142, wherein the IgG antibody construct is an antibody, a Fab2 antibody, a bis-scFv antibody, a diabody, a DVD-Ig, a TandAb, a tandem scFv-Fc, a one-armed tandem scFv-Fc, a DART, a DART-Fc, or a functional fragment thereof.

Embodiment 144: The IgG antibody construct of any one of embodiments 1-142, wherein the IgG antibody construct is an antibody, a variant thereof, or a functional fragment thereof.

Embodiment 145: The IgG antibody construct of any one of embodiments 1-144 further comprising a constant region, wherein:
said constant region comprises a first Fc domain and/or a second Fc domain.

Embodiment 146: The IgG antibody construct of embodiment 128, wherein said first Fc domain and said second Fc domain are derived from IgG1, IgG2, IgG3 or IgG4.

Embodiment 147: The IgG antibody construct of embodiment 146, wherein said first Fc domain and said second Fc domain are derived from IgG1 constant domain.

Embodiment 148: The IgG antibody construct of embodiment 147, wherein said first Fc domain and said second Fc domain independently comprise M252Y, S254T, and T256E substitutions.

Embodiment 149: The IgG antibody construct of embodiment 147 or 148, wherein said first Fc domain and said second Fc domain independently comprise L234A and L235A substitutions.

Embodiment 150: The IgG antibody construct of any one of embodiments 147-149, wherein said first Fc domain and said second Fc domain independently comprise P329G substitution.

Embodiment 151: The IgG antibody construct of any one of embodiments 147-150, wherein said first Fc domain comprises a knob modification and said second Fc domain comprises a hole modification, wherein:
said knob modification comprises replacing an original amino acid residue from an interface of the Fc region with an amino acid residue selected from tyrosine and tryptophan, and
said hole modification comprises replacing an original amino acid residue from the interface of the Fc region with an amino acid residue selected from serine, threonine, valine and alanine.

Embodiment 152: The IgG antibody construct of any one of embodiments 147-151, wherein the first Fc domain comprises at least two modifications at positions selected from L351, F405 and Y407, per EU numbering, and the second Fc domain comprises at least one modification at positions selected from T366, K392 and T394, per EU numbering.

Embodiment 153: The IgG antibody construct of any one of embodiments 147-152, wherein the first Fc domain comprises at least two modifications at positions selected from Q347, Y349, T350, K370, G371, D399 and S400, per EU numbering, and the second Fc domain comprises at least one modification at positions selected from T350, S354, E357, K360, Q362E, S364, N390, K409 and T411, per EU numbering.

Embodiment 154: The IgG antibody construct of any one of embodiments 147-153, wherein the first Fc domain comprises at least one substitution at positions selected from S354 and T366, per EU numbering, and the second Fc domain comprises at least one modification at positions selected from T366, L368 and Y407, per EU numbering.

Embodiment 155: The IgG antibody construct of any one of embodiments 147-154, wherein the first Fc domain comprises at least one substitution selected from S354 and T366W, per EU numbering, and the second Fc domain comprises at least one substitution selected from T366S. L368A, Y407T, and Y407V, per EU numbering.

Embodiment 156: The IgG antibody construct of any one of embodiments 147-155, wherein said constant region comprises at least one amino acid modifications, wherein said at least one amino acid modification increases binding affinity of said constant region for a neonatal fragment crystallizable receptor (FcRn) in acidic pH condition relative to said binding affinity of corresponding constant region prior to the at least one amino acid modification, and wherein a binding affinity of said constant region for said FcRn in neutral pH condition remains within 20% of corresponding binding affinity of said corresponding constant region prior to the at least one modification.

Embodiment 157: The IgG antibody construct of embodiment 156, wherein said binding affinity of said constant region for said FcRn is increased by at least 5%, at least 10%, at least 15%, at least 20%, at least 25%, at least 30%, at least 50%, at least 70% or more in acidic pH condition relative to the corresponding constant region prior to the at least one modification.

Embodiment 158: The IgG antibody construct of embodiment 156 or 157, wherein said at least one modification decreases plasma clearance (CL), increases plasma retention time, or increases plasma half-life (t½) relative to a corresponding IgG antibody construct prior to said at least one modification in said constant region.

Embodiment 159: The IgG antibody construct of any one of embodiments 156-158, wherein a binding affinity of said constant region for a FcRn is increased in neutral pH condition relative to said binding affinity of a corresponding constant region prior to said at least one modification, and wherein a binding affinity of said constant region for said FcRn in acidic pH condition remains within 20% of said binding affinity of corresponding constant region prior to said at least one modification.

Embodiment 160: The IgG antibody construct of embodiment 159, wherein said binding affinity of said constant region for said FcRn is increased by at least 5%, at least 10%, at least 15%, at least 20%, at least 25%, at least 30%, at least 50%, at least 70% or more in neutral pH condition relative to said binding affinity of corresponding constant region prior to the at least one modification.

Embodiment 161: The IgG antibody construct of embodiment 159 or 160, wherein the at least one modification increases plasma clearance of TL1A by the IgG antibody construct, compared to a corresponding plasma clearance by an IgG antibody construct prior to the at least one modification in the constant region.

Embodiment 162: An antibody construct comprising:
a TL1A binding region that binds an epitope on TL1A, a variant thereof or a functional fragment thereof, wherein the TL1A binding region comprises a heavy chain variable region, wherein the heavy chain variable region comprises at least one modification at positions selected from S30, T69, L83, N84, T104 and F107, relative to the amino acid position numbering of SEQ ID NO: 923.

Embodiment 163: The antibody construct of embodiment 162, wherein the VH sequence comprises at least one modification comprises a substitution selected from the group consisting of S30T, T69I, L83V, N84K, T104D, F107N and F107D, relative to the amino acid position numbering of SEQ ID NO: 923.

Embodiment 164: The antibody construct of embodiment 162 or 163, wherein the at least one modification improves binding interaction with H109, H118 and/or H121 of TL1A comprising an amino acid sequencer of SEQ ID NO: 139.

Embodiment 165: An antibody construct comprising:
an IL-23 binding region that binds an epitope on IL-23, a variant thereof or a functional fragment thereof, wherein the IL-23 binding region comprises a heavy chain variable region, wherein the heavy chain variable region comprises at least one modification at positions selected from T28, T30, 131, A33, 134, G56, G58, H59, Q62, Q65, R98, E101, N102 and L108 relative to the amino acid position numbering of SEQ ID NO: 1544.

Embodiment 166: The antibody construct of embodiment 165, the VH sequence comprises at least one modification comprises a substitution selected from the group consisting of T28V, T28A, T28L, T28P, T30S, 131Q, A33T, I34M, I34V, G56K, G56A, G56N, G58A, H59Y, H59V, Q62S, Q62K, Q65R, Q65K, Q65A, R981, E101S, E101Y, E101T, N102F, N102Y, N102S, N102D, N102K, N102R, L108T and L108M relative to the amino acid position numbering of SEQ ID NO: 1544.

Embodiment 167: A pharmaceutical composition comprising the IgG antibody construct of any one of embodiments 1-161, or the antibody construct of any one of embodiments 162-166, and a pharmaceutically acceptable carrier.

Embodiment 168: A method of treating a disease or a condition, wherein the method comprises administering an effective amount of the IgG antibody construct of any one of embodiments 1-161, the antibody construct of any one of embodiments 162-166, or the pharmaceutical composition of embodiment 167 to a subject in need thereof results in treatment of a disease or condition.

Embodiment 169: The method of embodiment 168, wherein the disease or condition is related to impaired mitochondrial dysfunction.

Embodiment 170: The method of embodiment 168 or 169, wherein the disease or condition is selected from the group consisting of: rheumatoid arthritis, Crohn's Disease (CD), ulcerative colitis (UC), psoriatic arthritis, ankylosing spondylitis, psoriasis, primary biliary cirrhosis, systemic lupus erythematosus or combinations thereof.

### EXAMPLES

### Example 1. Bispecific Antibody Constructs

Bispecific antibody capable of binding two different targets are generated. The two different targets are (a) TL1A, a variant thereof or a functional fragment thereof, and (b) any one of IL-6R, IL-6, IL-12, IL-23, IL-23p19, IL-12p40, IL-12p35, an IL-17 family cytokine, IL-17R, a variant thereof or a functional fragment thereof. The bispecific antibody comprises a first binding domain and a second binding domain. The first binding domain comprises a first VH sequence and a first VL sequence, wherein the first binding domain is capable of binding TL1A, a variant thereof or a functional fragment thereof. The second binding domain comprises a second VH sequence and a second VL sequence, wherein the second binding domain is capable of binding to any one of IL-6R, IL-6, IL-12, IL-23, IL-23p19, IL-12p40, IL-12p35, an IL-17 family cytokine, IL-17R, a variant thereof or a functional fragment thereof. The first VH sequence comprises any one of amino acid sequences of TABLE 39. The first VL sequence comprises any one of amino acid sequences of TABLE 42. The second VH sequence and the second VL sequence comprises any one of amino acid sequences of TABLE 3, TABLE 10, TABLE 17, TABLE 24 and TABLE 31, and TABLE 6, TABLE 13, TABLE 20, TABLE 27 and TABLE 34, respectively.

### Example 2. Proof of Concept Phase 1 Trial for a Bispecific Antibody

Bispecific antibody capable of binding two different targets are generated as described in Example 1. Briefly, the two different targets are (a) TL1A, a variant thereof or a functional fragment thereof, and (b) a protein from any one of IL-6R, IL-6, IL-12, IL-23, IL-23p19, IL-12p40, IL-12p35, an IL-17 family cytokine, IL-7R, a variant thereof or a functional fragment thereof. The bispecific antibody is administered to a subject in need thereof. The subject is selected according to criteria described in TABLE 45.

TABLE 45
Eligibility Criteria:
Ages Eligible for Study: 18 Years to 80 Years (Adult, Older Adult)
Sexes Eligible for Study: All
Accepts Healthy Volunteers: No
Inclusion Criteria:
18-80 years age
Recent onset of autoimmune condition and have known doctor diagnosis ≤1 years and
symptoms for ≤2 years.
Clinically stable with no significant changes in health status within 2 weeks prior to
randomization
Exclusion Criteria:
Presence of active infection
History of chronic viral infections including Hepatitis B or C or HIV. Treated Hepatitis
C is allowed if the viral in non-detectable
Known chronic liver disease
Pregnant, breastfeeding, or desire to become pregnant or unwilling to practice birth
control during participation in the study and for twelve months after completing the
study infusion, unless surgically sterilized or postmenopausal during the study.
Active tuberculosis (TB) requiring treatment within 3 years prior to baseline
Latent TB diagnosed during screening that has not been appropriately treated
Chronic obstructive pulmonary disease or known lung disease except for mild asthma
treated with bronchodilators.
Use of an investigational agent within the 4-week period prior to screen
If Dimethyl sulfoxide (DMSO) is used in the preparation of MSCs then subjects with
known sensitivity to DMSO will be excluded
History of Transient Ischemic Attack
History of Cerebrovascular Accident (stroke)
Clinically significant heart disease (New York Heart Association, class III and class
IV).

### Example 3. Screening for TL1A Targeting Lead Candidates for Generating Bispecific Antibody

Initial screening and assessment for identifying lead TL1A binding antibody candidates having pH dependent binding activity was performed on Octet BLI platform. Briefly, trimeric TL1A was diluted to 100 nM in kinetics buffer (1×PBS pH 7.4, 0.1% BSA, 0.05% Tween20, 0.05% sodium azide) and loaded onto biosensors for a period of 5 minutes. Sensors were then baselined in kinetics buffer before being dipped into antibodies diluted to 100 nM for a 5-minute association step, followed by a 10-minute dissociation step. Baseline, association, and dissociation steps were carried out in pH 6 kinetics buffer or pH 7.4 kinetics buffer. Sensors were then regenerated with 20 mM glycine pH 2 and neutralized with pH 7.4 kinetics buffer. Referencing was performed using a loaded sensor that was dipped into buffer as opposed to antibody. Then, data was aligned and fit with a 1:2 (bivalent analyte) model. Binding affinities calculated for the lead candidates at different pH are described in TABLE 46.

TABLE 46
Binding affinities of TL1A binding antibody candidate

| | VH | VL | K_{D} at pH 7.4 | K_{D} at pH 5.8 |
|---|---|---|---|---|
| Candidate | (SEQ | (SEQ | for trimeric | for trimeric |
| No. | ID NO:) | ID NO:) | TL1A | TL1A |
| 1 | 923 | 1768 | 4.69E−09 | 4.21E−07 |
| 2 | 1048 | 1768 | 8.09E−09 | 2.00E−08 |

### Example 4. Potency Assay of TL1A Binding Candidate for Generating Bispecific Antibody

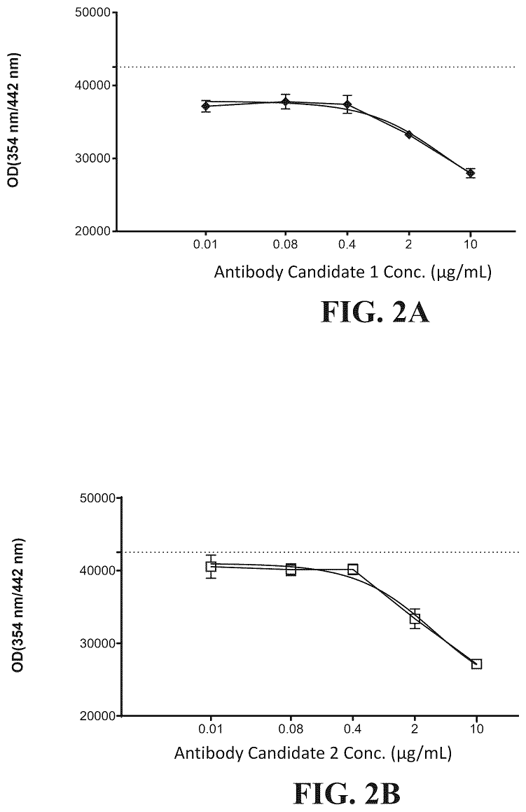

TL1A potency assay was performed for the lead TL1A binding antibody candidates described in Example 3. Briefly, TF1 cells were cultured in black 96-well flat bottom plates at 1×10⁵ cells/well. In a separate 96-well plate, TL1A was combined with test antibodies and allowed to pre-incubate for 30 minutes. Following the pre-incubation, TF1 cells were treated where indicated with cycloheximide (2.5 μg/ml) along TL1A binding antibody candidates in combination with TL1A. Cells were incubated at 37° C. and 5% CO₂ for 5 hours. Following incubation, cells were assayed for the detection of activated Caspase 3/7, the results of which are provided in FIGS. 2A-2B. An analysis of FIGS. 2A-2B indicates that the lead TL1A binding antibody candidates as identified in Example 4 have TL1A antagonistic activity.

### Example 5. Screening for TL1A Binding Candidates Having pH Dependent Binding Activity for Bispecific Antibody

Binding kinetic assays were performed for determining pH dependent binding activity of a lead TL1A binding antibody candidate, as identified in Example 3, and 7 different variants thereof using surface plasma resonance (SPR) spectroscopy. Each TL1A binding candidate comprised a VH sequence that comprised at least one modification in CDR or framework region of variable region of the VH sequence. Briefly, an amine-coupled anti-human IgG-Fc specific polyclonal antibody was immobilized on a SPR sensor chip. All TL1A binding antibody candidates were expressed at 1 mL scale in chinese hamster ovary (CHO) cells for 6 days, and supernatant was cleared of cells by centrifugation. To form a captured antibody array, TL1A binding antibody candidates were batch diluted to approximately 1.7-13.3 nM (either after Protein A purification or directly from CHO supernatant) and injected onto the chip. Non-regenerative kinetics were measured by injecting TL1A monomer starting at a concentration of 2.57E-10 M, with 6-fold increase in concentration up to 3.33E-07 M at pH 7.4 and 5.8, respectively. Likewise, non-regenerative kinetics were also measured for TL1A trimer starting at a concentration of 2.57E-10 M, with 6-fold increase in concentration up to 3.33E-07 M at pH 7.4. Each analyte injection flowed over the array at 2 mL/min for a 5-minute association phase, followed by a 10-minute buffer dissociation phase. Binding was detected and relative capture response units (RU) were collected for each antibody on the surface. Kinetic values were calculated, which are summarized in TABLE 47 below.

TABLE 47
Summary of results of binding kinetic assays

| | | | K_{D} at pH | K_{D} at pH | K_{D} at pH |
|---|---|---|---|---|---|
| | VH | VL | 7.4 for | 7.4 for | 5.8 for |
| Candidate | (SEQ | (SEQ | trimeric | monomeric | monomeric |
| No. | ID NO:) | ID NO:) | TL1A | TL1A | TL1A |
| 1 | 902 | 1768 | 3.34E−10 | 6.46E−09 | N/A |
| 2 | 903 | 1768 | 2.65E−10 | 5.53E−09 | N/A |
| 3 | 904 | 1768 | 2.83E−10 | 4.78E−09 | 3.43E−08 |
| 4 | 905 | 1768 | 3.02E−10 | 6.15E−09 | 1.43E−08 |
| 5 | 907 | 1768 | 3.21E−10 | 5.19E−09 | N/A |
| 6 | 908 | 1768 | 3.22E−10 | 3.05E−09 | N/A |
| 7 | 909 | 1768 | 3.29E−10 | 4.57E−09 | N/A |
| 8 | 933 | 1768 | 4.31E−10 | 9.66E−09 | N/A |

*N/A = binding not detected

An analysis of TABLE 47 indicates that all tested TL1A binding candidates have shown substantial reduction in binding affinity for monomeric TL1A under acidic condition. Moreover, the analysis also indicates that the TL1A binding antibody candidates having higher binding affinity for trimeric TL1A relative to monomeric TL1A.

### Example 6. Screening for IL-23 Binding Candidates Having pH Dependent Binding Activity for Bispecific Antibody

Binding kinetic assays were performed for determining pH dependent binding activity of two lead IL-23 binding antibody candidates using surface plasma resonance (SPR) spectroscopy. Briefly, an amine-coupled anti-human IgG-Fc specific polyclonal antibody was immobilized on a SPR sensor chip. Both IL-23 binding antibody candidates were expressed at 1 mL scale in chinese hamster ovary (CHO) cells for 6 days, and supernatant was cleared of cells by centrifugation. To form a captured antibody array, IL-23 binding antibody candidates were batch diluted to approximately 1.7-13.3 nM (either after Protein A purification or directly from CHO supernatant) and injected onto the chip. Non-regenerative kinetics were measured by injecting IL-23 heterodimer (IL-23α & IL-12β) starting at a concentration of 2.57E-10 M, with 6-fold increase in concentration up to 3.33E-07 M. Each analyte injection flowed over the array at 2 mL/min for a 5-minute association phase, followed by a 10-minute buffer dissociation phase. Binding was detected and relative capture response units (RU) were collected for each antibody on the surface. Kinetic values were calculated, which are summarized in TABLE 48 below.

TABLE 48
Summary of results of binding kinetic assays

| Candidate | VH | VL | K_{D} for |
|---|---|---|---|
| No. | (SEQ ID NO:) | (SEQ ID NO:) | heteromeric IL-23 |
| 1 | 1544 | 1768 | 7.60E−10 |
| 2 | 1621 | 1768 | 4.75E+04 |
| 3 | 1466 | 1768 | 1.32E−10 |

### Example 7. Potency Assay of IL-23 Binding Candidate for Generating Bispecific Antibody

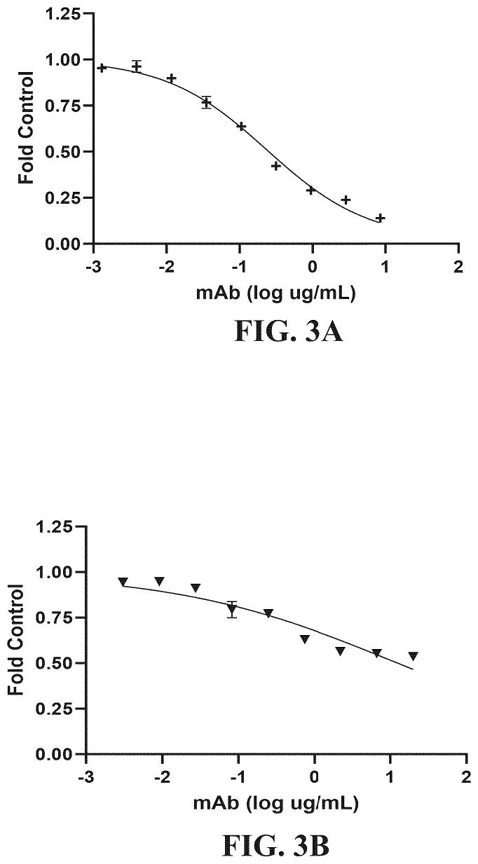

IL-23 potency assay was performed for the two IL-23 binding antibody candidates described in Example 6. For the assay, a genetically engineered cell line that measures IL-23 receptor activation of the STAT3 signal transduction pathway was used. The assay was carried out using the manufacturer recommended protocol. Briefly, the two IL-23 binding antibody candidates (Candidate No. 1 and 2) were quantified (A280) and normalized for concentration. Each antibody was diluted to 3.75× the final test concentration and titrated at 3-fold dilutions in duplicate for 9 points (133 nM to 0.02 nM). In an opaque 96-well tissue culture plate, 5 μL of IL-23 heterodimer (IL-23α & IL-12β) was added to the plate, for a final concentration of 10 ng/mL. Next, 20 μL of the diluted antibody titrations were added to the plate containing the IL-23 and incubated at ambient temperature for 15 minutes. The IL-23 reporter cells were thawed, diluted in assay buffer, and 50 μL of cells were added to each well, for a final volume of 75 μL per well. The plate was then incubated at 37° C., 5% CO₂ for 6 hours. Finally, the plate was removed from the incubator, equilibrated to ambient temperature for 15 minutes, and 75 μL of luciferase detection reagent was added to all wells. The plate was incubated for 10 minutes at ambient temperature, and luminescent signal was measured on a plate reader. Neutralization of IL-23 signaling was reported by comparing the average signal of each treatment to the IL-23 only (non-antibody treated) average. FIGS. 3A and 3B provides results of potency assay for IL-23 binding candidate-1 and IL-23 binding candidate-2, respectively. An analysis of FIGS. 3A-3B indicates that the IL-23 binding antibody candidate as identified in Example 6 have Il-23 antagonistic activity.

### Example 8. Use of Fab-Arm Exchange Method for Generating Bispecific/Trispecific Antibody Targeting TL1A and IL-23

Bispecific antibody constructs were made using fab-arm exchange method. Briefly, TL1A binding antibody comprising an affinity tag (e.g., HIS tag) was mixed with either IL-23 binding antibody or IL-23/IL-12 binding antibody in the presence of one or more reducing agents. The TL1A binding antibody comprises a VH sequence comprising an amino acid sequence of SEQ ID NO: 487 and a VL sequence comprising an amino acid sequence of SEQ ID NO: 491. The IL-23 binding antibody comprises a VH sequence comprising an amino acid sequence of SEQ ID NO: 375 and a VL sequence comprising an amino acid sequence of SEQ ID NO: 463. The IL-23/IL-12 binding antibody comprises a VH sequence comprising an amino acid sequence of SEQ ID NO: 283 and a VL sequence comprising an amino acid sequence of SEQ ID NO: 303. All three, the TL1A binding antibody, the IL-23 binding antibody and the IL-23/IL-12 binding antibody have same human IgG1 Fc region that comprises L234A, L235A, M252Y, S254T, T256E and P329G modifications. Gradual removal of reducing agent results reoxidation of interchain disulfide bonds, which resulted in formation of a mixture of the bispecific antibody, the TL1A binding antibody, the half-TL1A binding antibody, the IL-23 binding antibody and the half-IL-23 binding antibody. Followingly, the bispecific antibody, the TL1A binding antibody and the half-TL1A binding antibody were separated from the mixture. Then, the bispecific antibody was separated from the TL1A binding antibody and the half-TL1A binding antibody.

### Example 9. Blocking Effect of TL1A Binding Region of Multispecific Antibodies Prepared by Fab-Arm Exchange Method

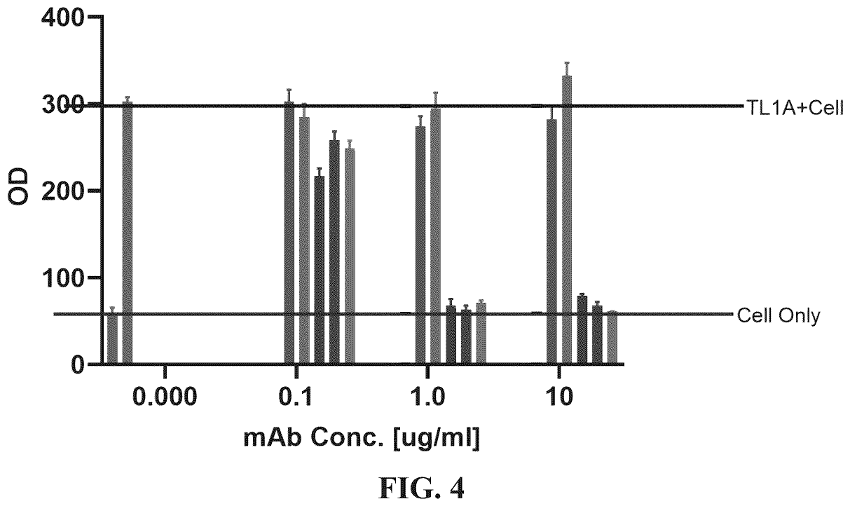

Blocking effect of TL1A binding region of multispecific antibodies was determined using luciferase assay. Briefly, five antibodies, TL1A binding antibody, IL-23 binding antibody, IL-23/IL-12 binding antibody, TL1A & IL-23 binding bispecific antibody and TL1A and IL-23/IL-12 binding trispecific antibody, as described in Example 8, were tested for their antagonistic effect on TL1A-stimulated Jurkat cells. For luciferase assay, about 30,000 cells/well were taken in 50 μL of thaw medium according to manufacturer's instructions. Then, TL1A agonist at a concentration of 130.0 ng/mL was mixed with each antibody at a concentration ranging from 0.1-10 μg/mL. The mixtures were incubated for 1 hour at room temperature before loading into the well containing Jurkat cells. The mixture was incubated with Jurkat cells for about 5 hours at 37° C. in 5% CO₂. Followingly, 100 μL of luciferase reagent was added to each well, and subsequently luminescence was measured. Result of the luciferase assay are provided in FIG. 4. An analysis of FIG. 4 indicates that proteins that contain TL1A binding region have blocking effect on TL1A activity.

### Example 10. Knob-in-Hole Bispecific Antibody Targeting TL1A and IL-23

### Parental Bispecific Matrix Generation: Sequence Selection & Cloning

To initiate the Bispecific matrix generation two sets of parental bispecific controls were established. Set I included favored functional monoclonals to be paired as Knob-in-Hole Bispecifics (1:1 format) with the IL-23 sequence on the Hole-Fc domain and the TL1a sequence on the Knob-Fc domain. Due to the Isoelectric point differential of these lead parental monoclonals being too similar a 6× Histidine tag was added to the Hole-Fc domain of Set I bispecifics for ease of purification. Set II included an alternative set of parental monoclonals from lead optimization. These clones were selected as a back-up parental set and built the same as Set I Knob-in-Hole bispecifics. This set did not need a Histidine tag on the Hole-Fc domain, since Isoelectric point differentials for this set were greater to aid in the purification process.

Sequences were compiled for unique variable heavy and light chain pairs. The light chain for all clones comprises a common amino acid sequence of SEQ ID NO: 1768. Sequences were codon optimized. Synthetic oligonucleotide was engineered for the Knob-in-Hole bispecifics (1:1 format). KIH (Knob-in-Hole) mutations plus disulfide stabilizers used in the constant region designs were as follows: Knob: T366W (S354C) & Hole: T366. L368A. Y407V (Y349C). For all clones, the constant region isotype, hIgG1, comprised L234A, L235A, M252Y, L254T, T256E, P329G and Y407T modifications. TABLE 49 summarizes engineered knob-in-hole antibody candidates that were prepared.

TABLE 49

| | Knob-IL-23 | | | | Hole-TL1A | | | |
|---|---|---|---|---|---|---|---|---|
| | VH | VL | | | VH | VL | | |
| | (SEQ | (SEQ | | | (SEQ | (SEQ | | |
| Candidate | ID | ID | Fc region | | ID | ID | Fc region | |
| No | NO:) | NO:) | mutations | PI | NO:) | NO:) | mutations | PI |
| I-1 | 1544 | 1768 | L234A, | 7.82 | 923 | 1768 | L234A, | 7.82 |
| | | | L235A, | | | | L235A, | |
| | | | M252Y, | | | | M252Y, | |
| | | | S254T, | | | | S254T, | |
| | | | T256E, | | | | T256E, | |
| | | | P329G and | | | | P329G, | |
| | | | T366W | | | | T366S, | |
| | | | (S354C) | | | | L368A, and | |
| | | | | | | | Y407V | |
| | | | | | | | (Y349C) | |
| I-2 | 1621 | 1768 | L234A, | 8.03 | 923 | 1768 | L234A, | 7.82 |
| | | | L235A, | | | | L235A, | |
| | | | M252Y, | | | | M252Y, | |
| | | | S254T, | | | | S254T, | |
| | | | T256E, | | | | T256E, | |
| | | | P329G and | | | | P329G, | |
| | | | T366W | | | | T366S, | |
| | | | (S354C) | | | | L368A, and | |
| | | | | | | | Y407V | |
| | | | | | | | (Y349C) | |
| II-1 | 1544 | 1768 | L234A, | 7.82 | 933 | 1768 | L234A, | 7.22 |
| | | | L235A, | | | | L235A, | |
| | | | M252Y, | | | | M252Y, | |
| | | | S254T, | | | | S254T, | |
| | | | T256E, | | | | T256E, | |
| | | | P329G and | | | | P329G, | |
| | | | T366W | | | | T366S, | |
| | | | (S354C) | | | | L368A, and | |
| | | | | | | | Y407V | |
| | | | | | | | (Y349C) | |
| II-2 | 1621 | 1768 | L234A, | 8.03 | 933 | 1768 | L234A, | 7.22 |
| | | | L235A, | | | | L235A, | |
| | | | M252Y, | | | | M252Y, | |
| | | | S254T, | | | | S254T, | |
| | | | T256E, | | | | T256E, | |
| | | | P329G and | | | | P329G, | |
| | | | T366W | | | | T366S, | |
| | | | (S354C) | | | | L368A, and | |
| | | | | | | | Y407V | |
| | | | | | | | (Y349C) | |
| II-3 | 1466 | 1768 | L234A, | 7.55 | 923 | 1768 | L234A, | 8.02 |
| | | | L235A, | | | | L235A, | |
| | | | M252Y, | | | | M252Y, | |
| | | | S254T, | | | | S254T, | |
| | | | T256E, | | | | T256E, | |
| | | | P329G and | | | | P329G, | |
| | | | T366W | | | | T366S, | |
| | | | (S354C) | | | | L368A, and | |
| | | | | | | | Y407V | |
| | | | | | | | (Y349C) | |
| II-4 | 1621 | 1768 | L234A, | | 933 | 1768 | L234A, | |
| | | | L235A, | | | | L235A, | |
| | | | M252Y, | | | | M252Y, | |
| | | | S254T, | | | | S254T, | |
| | | | T256E, | | | | T256E, | |
| | | | P329G and | | | | P329G and | |
| | | | Y407T | | | | T366Y | |

All constructs were cloned and transformed in DH5a competent cells. The transformed cells were then plated and grown in shaker at 37° C. Single colonies were then picked from each construct and grown at 37° C. shaking in 15 mL falcon tubes, seeded at 5 mL LB-CARB media.

The cultured cells tubes were pelleted, discarding the media and plasmid was purified. Plasmid DNA is then checked for concentration and sequences were confirmed.

### Bispecific Expression & Purification

Unique variable heavy chains and a common light chain (SEQ ID NO: 1768) were cloned into vectors designed to express bispecifics and relevant controls in HEK293 cells under the control of a CMV promoter. Antibody expression vectors were complexed with polyethylenimine and transfected into HEK293 cultures. Knob and Hole technology was used to enrich for heterodimerization, and disfavor homodimerization, during expression. After 5 days of shaking at 37° C. in 293 cell culture media, cultures were harvested and the supernatant containing the secreted antibodies was clarified and filtered. Antibodies were captured out of the supernatant via an agarose-based protein A resin on a fast protein liquid chromatography (FPLC). After several washes with phosphate buffer solution (PBS), antibodies were eluted in a phosphoric acid and saline solution (pH 3) and neutralized with a basic phosphate saline solution (pH 11) to pH ~5.5. Analytical size exclusion chromatography and SDS PAGE analysis assisted in determining the optimal fractions from each protein A elution to move forward into secondary/polishing purification.

Automated cation exchange chromatography (CEX), again on the FPLC, Bind and elute methodology was employed to further enrich the samples for heterodimer bispecifics or controls. After dilution, to lower the salt concentration, a strong cation exchange column was used for capturing the material of interest in the protein A elute, followed by washing and elution with increasing levels of sodium chloride at a constant pH of 6.5. Again, the combination of analytic SEC and SDS PAGE led to the determination of which fractions from CEX were suitable for pooling. If the presence of contaminating products was still observed, for those constructs with a HIS tag on one chain, immobilized metal affinity chromatography (IMAC) was employed to further enrich the heterodimer via negative or positive selection. If IMAC was used, subsequent analytical SEC and SDS PAGE occurred, followed by pooling, samples were sterilized, assayed for purity via analytical SEC and endotoxin were performed.

### Example 11. Blocking Effect of TL1A Binding Region of Multispecific Antibodies Prepared by Knob-In-Hole Method

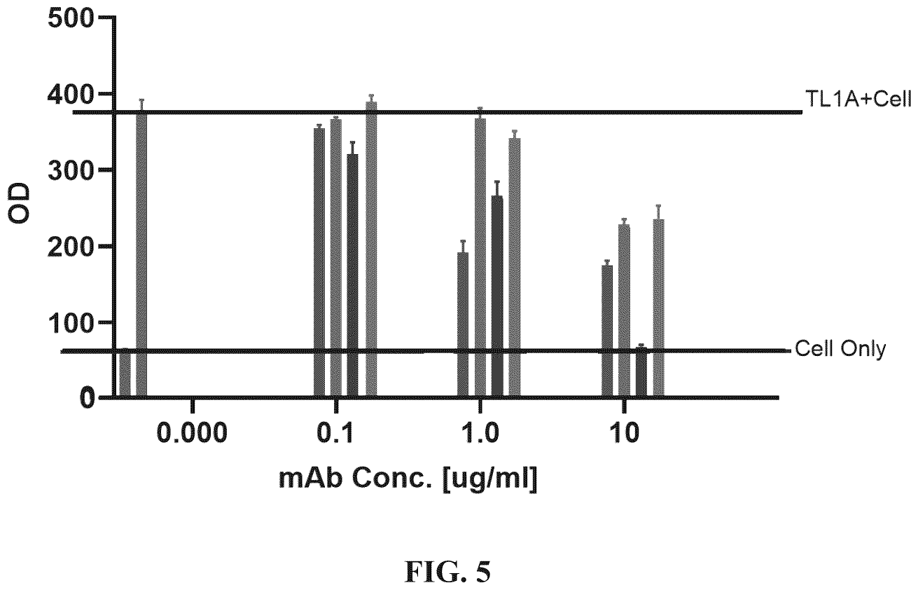

Blocking effect of TL1A binding region of multispecific antibodies was determined using luciferase assay. Briefly, four knob-in-hole bispecific antibodies (II-1 to II-4) were prepared, as explained in Example 10, and used for this assay. For testing antagonistic effect of the knob-in-hole antibodies in TL1A-stimulated Jurkat cells, a luciferase assay was performed as provided in Example 6. For luciferase assay, about 30,000 cells/well were taken in 50 μL of thaw medium according to manufacturer's instructions. Then, TL1A agonist at a concentration of 130.0 ng/mL was mixed with each antibody at a concentration ranging from 0.1-10 μg/mL. The mixtures were incubated for 1 hour at room temperature before loading into the well containing Jurkat cells. The mixture was incubated with Jurkat cells for about 5 hours at 37° C. in 5% CO₂. Followingly, 100 μL of luciferase reagent was added to each well, and subsequently luminescence was measured. Result of the luciferase assay are provided in FIG. 5. An analysis of FIG. 5 indicates that proteins that contain TL1A binding region have blocking effect on TL1A activity.

### Example 12. TL1A Binding Kinetics for Multispecific Antibodies Prepared by Knob-In-Hole Method

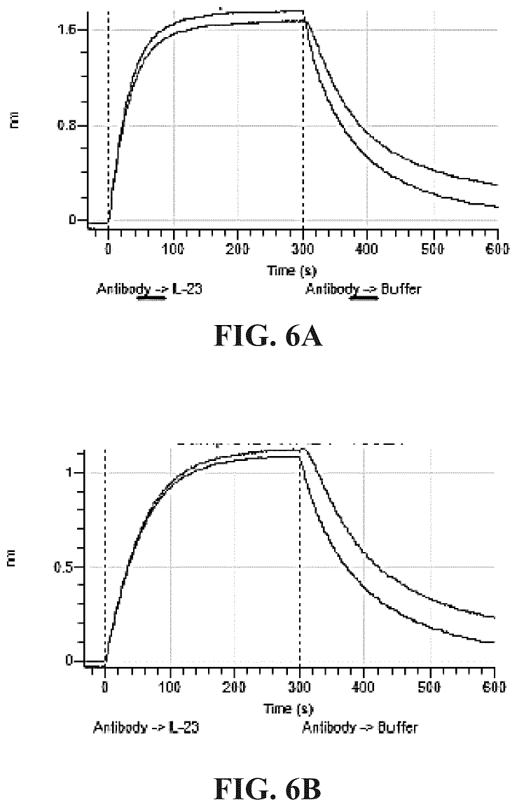

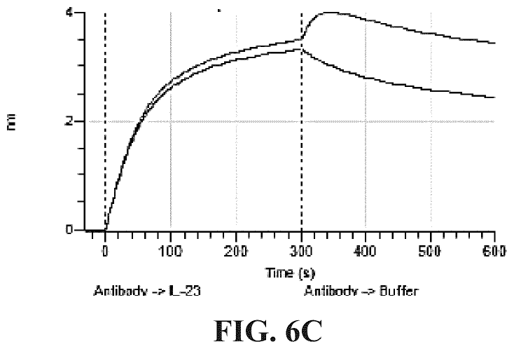

Kinetic determination and assessment of pH dependency was performed on Octet BLI platform. Briefly, knob-in-hole bispecific antibodies that were prepared according to method described in Example 10 were used for this assay. Briefly, biotinylated recombinant human TL1A was diluted to 7.5 nM in kinetics buffer (1×PBS pH 7.4, 0.1% BSA, 0.05% Tween20, 0.05% sodium azide) and loaded onto streptavidin sensors for a period of 5 minutes. Sensors were then baselined in kinetics buffer before being dipped into bispecific antibodies diluted to varying concentrations, with two-fold serial dilutions for a 5-minute association step, followed by a 15-minute dissociation step. Top concentrations were determined based on approximate K_{D} determined during a scouting experiment. Referencing was performed using a loaded sensor that was dipped into buffer as opposed to antibody, as well as unloaded sensors dipped into antibody. Then, data was aligned and fitted. FIGS. 6A-6C shows representative binding curves for three of the knob-in-hole antibody constructions prepare in Example 10. An analysis of FIGS. 6A-6C shows binding of antibody construct to IL-23 and TL1A.

### Example 13. Screening for IL-6, IL-12 and IL-17 Binding Candidates Having pH Dependent Binding Activity for Bispecific Antibody

Binding kinetic assays can be performed for determining pH dependent binding activity of lead IL-6R, IL-6, IL-12 and IL-17 binding antibody candidates using surface plasma resonance (SPR) spectroscopy. TABLE 50 summarizes VH and VL sequences of lead antibody candidates. Briefly, an amine-coupled anti-human IgG-Fc specific polyclonal antibody is immobilized on a SPR sensor chip. All antibody candidates are expressed at 1 mL scale in chinese hamster ovary (CHO) cells for 6 days, and supernatant is cleared of cells by centrifugation. To form a captured antibody array, antibody candidates are batch diluted to approximately 1.7-13.3 nM (either after Protein A purification or directly from CHO supernatant) and injected onto the chip. Non-regenerative kinetics are measured by injecting appropriate antigen (e.g., IL-6R, IL-6, IL-12 or IL-17). Each analyte injection is flown over the array at 2 mL/min for a 5-minute association phase, followed by a 10-minute buffer dissociation phase. Binding is detected and relative capture response units (RU) are collected for each antibody on the surface. Kinetic values are calculated.

TABLE 50
Lead antibody candidates

| Target | Candidate | VH | VL |
|---|---|---|---|
| Antigen | No. | (SEQ ID NO:) | (SEQ ID NO:) |
| IL-6R | 1 | 11 | 24 |
| IL-6 | 2 | 241 | 265 |
| IL-6 | 3 | 242 | 266 |
| IL-6 | 4 | 244 | 268 |
| IL-12 | 5 | 283 | 303 |
| IL-17 | 6 | 87 | 129 |
| IL-17 | 7 | 88 | 130 |

### Example 14. Use of Fab-Arm Exchange Method for Generating Bispecific Antibodies

Bispecific antibody constructs can be made using fab-arm exchange method. Briefly, TL1A binding antibody comprising an affinity tag (e.g., HIS tag) is mixed with either IL-6R binding antibody candidate, IL-6 binding antibody candidate, IL-12 binding antibody candidate or IL-17 binding antibody candidate in the presence of one or more reducing agents. Sequences of bispecific antibodies that can be used according to the method described herein are summarized in TABLE 51.

TABLE 50
Lead antibody candidates

| | | First Binding Region | | Second Binding Region | |
|---|---|---|---|---|---|
| | | VH | VL | VH | VL |
| Target | Candidate | (SEQ | (SEQ | (SEQ | (SEQ |
| Antigen | No. | ID NO:) | ID NO:) | ID NO:) | ID NO:) |
| IL-6R X | 1 | 11 | 24 | 487 | 491 |
| TL1A | | | | | |
| IL-6 X | 2 | 241 | 265 | 487 | 491 |
| TL1A | | | | | |
| IL-6 X | 3 | 242 | 266 | 487 | 491 |
| TL1A | | | | | |
| IL-6 X | 4 | 244 | 268 | 487 | 491 |
| TL1A | | | | | |
| IL-12 X | 5 | 283 | 303 | 487 | 491 |
| TL1A | | | | | |
| IL-17 X | 6 | 87 | 129 | 487 | 491 |
| TL1A | | | | | |
| IL-17 X | 7 | 88 | 130 | 487 | 491 |
| TL1A | | | | | |

All antibody candidates can have same human IgG1 Fc region that comprises L234A, L235A, M252Y, S254T, T256E and P329G modifications. Gradual removal of reducing agent can result in reoxidation of interchain disulfide bonds, which can form a mixture of the bispecific antibody, which can be further processed to purify each antibody candidate.

### Example 15. Blocking Effect of IL-23 Binding Region of Multispecific Antibodies Prepared by Fab-Arm Exchange Method

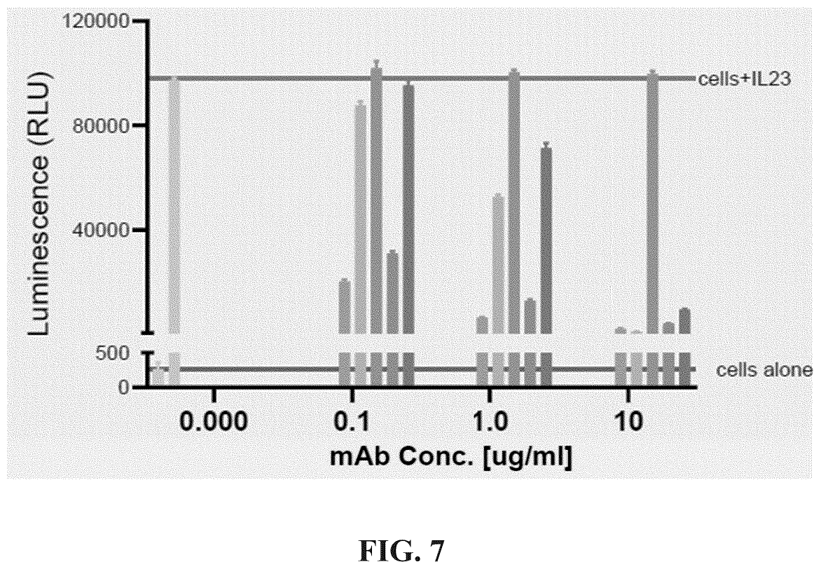

Blocking effect of IL-23 binding region of multispecific antibodies was determined using luciferase assay. Briefly, five antibodies, TL1A binding antibody, IL-23 binding antibody, IL-23/IL-12 binding antibody, TL1A & IL-23 binding bispecific antibody and TL1A and IL-23/IL-12 binding trispecific antibody, as described in Example 8, were tested for their antagonistic effect on IL-23-stimulated bioassay cells. For the luciferase assay, bioassay cells that are engineered to express IL-23 receptor and luciferase reporter gene were used. Briefly, the bioassay cells were thawed and plated in 96-well plate. IL-23 was added to each well and the plate was incubated at 37° C. for 6 hours. Appropriate antibody was then added to each well and the mixtures were incubated for 1 hour at room temperature. Followingly, luciferase reagent was added to each well, and subsequently luminescence was measured. Result of the luciferase assay are provided in FIG. 7. An analysis of FIG. 7 indicates that proteins that contain IL-23 binding region have blocking effect on IL-23 activity.

### Example 16. Blocking Effect of IL-23 Binding Region of Multispecific Antibodies Prepared by Knob-In-Hole Method

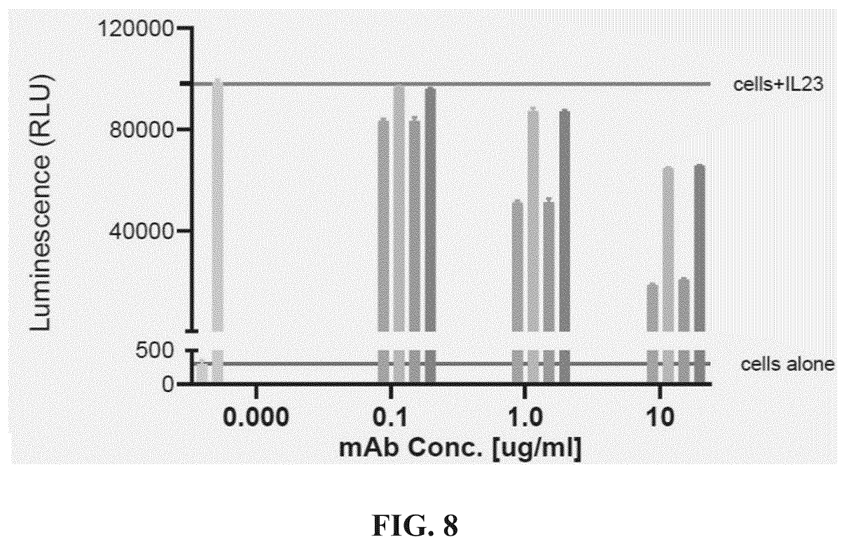

Blocking effect of IL-23 binding region of multispecific antibodies was determined using luciferase assay. Briefly, four knob-in-hole bispecific antibodies (II-1 to II-4) were prepared, as explained in Example 10, and used for this assay. For the luciferase assay, bioassay cells that are engineered to express IL-23 receptor and luciferase reporter gene were used. Briefly, the bioassay cells were thawed and plated in 96-well plate. IL-23 was added to each well and the plate was incubated at 37° C. for 6 hours. Appropriate antibody was then added to each well and the mixtures were incubated for 1 hour at room temperature. Followingly, luciferase reagent was added to each well, and subsequently luminescence was measured. Result of the luciferase assay are provided in FIG. 8. An analysis of FIG. 8 indicates that antibodies that contain IL-23 binding region have blocking effect on IL-23 activity.

What is claimed is:

1. An IgG antibody construct comprising:
a TL1A binding region that binds an epitope on TL1A, a variant thereof or a functional fragment thereof, and
an IL-23 binding region that binds an epitope on IL-23, a variant thereof or a functional fragment thereof,
wherein:
said TL1A binding region comprises a TL1A binding heavy chain variable domain and a TL1A binding light chain variable domain,
at least one amino acid modification in said TL1A binding heavy chain variable region and/or said TL1A binding light chain variable region, and
said amino acid modification changes an isoelectric point (pI) of said IgG antibody construct relative to a pI of a corresponding IgG antibody construct prior to said amino acid modification, thereby resulting in pH-dependent binding activity for said TL1A in said IgG antibody construct, as measured by surface plasmon resonance spectroscopy.

2. The IgG antibody construct of claim 1, wherein said IL-23 binding region comprises:
an IL-23 binding heavy chain variable domain and an IL-23 binding light chain variable domain, and
at least one amino acid modification in said IL-23 binding heavy chain variable domain and/or said IL-23 binding light chain variable domain,
wherein said amino acid modification changes an isoelectric point (pI) of said IgG antibody construct relative to a pI of a corresponding IgG antibody construct prior to said amino acid modification, thereby resulting in pH-dependent binding activity for said IL-23 in said IgG antibody construct, as measured by surface plasmon resonance spectroscopy.

3. The IgG antibody construct of claim 2: wherein:
a binding affinity of said TL1A binding region for said TL1A is at least 10% higher at pH 7.4 relative to said binding affinity at pH 5.8, as measured by surface plasmon resonance spectroscopy; or
a binding affinity of said IL-23 binding region for said IL-23 is at least 10% higher at pH 7.4 relative to said binding affinity at pH 5.8, as measured by surface plasmon resonance spectroscopy.

4. The IgG antibody construct of claim 2, wherein a binding affinity of said TL1A binding region for said TL1A is different from a binding affinity of said IL-23 binding region for said IL-23 under neutral pH condition, as measured by surface plasmon resonance spectroscopy.

5. The IgG antibody construct of claim 4, wherein said binding affinity of said TL1A binding region for said TL1A is at least two times higher than said binding affinity of said IL-23 binding region for said IL-23.

6. The IgG antibody construct of claim 4, wherein a binding affinity of said IL-23 binding region for said IL-23 is at least two times higher than a binding affinity of said TL1A binding region for said TL1A.

7. The IgG antibody construct of claim 2, wherein
(I) said IgG antibody construct comprises a TL1A binding light chain variable domain that interact with said TL1A binding heavy chain variable domain, thereby forming said TL1A binding region, and
said TL1A binding heavy chain variable domain and/or said TL1A binding light chain variable domain comprises at least one amino acid modification that
(a) increases binding affinity of said TL1A binding region for said TL1A relative to said binding affinity of corresponding TL1A binding region prior to said at least one amino acid modification under neutral pH condition, as measured by surface plasmon resonance spectroscopy, or
(b) decreases binding affinity of said TL1A binding region for said TL1A relative to said binding affinity of corresponding TL1A binding region prior to said at least one amino acid modification under acidic pH condition, as measured by surface plasmon resonance spectroscopy or
(II) said IgG antibody construct comprises an IL-23 binding light chain variable domain that interact with said IL-23 binding heavy chain variable domain, thereby forming said IL-23 binding region, and
said IL-23 binding heavy chain variable domain and/or said IL-23 binding light chain variable domain comprises at least one amino acid modification that
(c) increases binding affinity of said IL-23 binding region for said IL-23 relative to said binding affinity of corresponding IL-23 binding region prior to said at least one amino acid modification under neutral pH condition, as measured by surface plasmon resonance spectroscopy, or
(d) decreases binding affinity of said IL-23 binding region for said IL-23 relative to said binding affinity of corresponding IL-23 binding region prior to said at least one amino acid modification under acidic pH condition, as measured by surface plasmon resonance spectroscopy.

8. The IgG antibody construct of claim 1, wherein said at least one amino acid modification is
(a) within a CDR-H1, a CDR-H2, and/or a CDR-H3 of said TL1A binding heavy chain variable domain,
(b) within at least one framework region of said TL1A binding heavy chain variable domain,
(c) within a CDR-L1, a CDR-L2, and/or a CDR-L3 of said TL1A binding light chain variable domain, or
(d) within at least one framework region of said TL1A binding light chain variable domain.

9. The IgG antibody construct of claim 8, wherein said at least one amino acid modification is within a CDR-H3 of said TL1A binding heavy chain variable domain and the at least one amino acid modification comprises a substitution of at least one uncharged amino acid with a charged amino acid.

10. The IgG antibody construct of claim 8, wherein said at least one amino acid modification increases binding affinity of said TL1A binding region for said TL1A in neutral pH condition by at least 2%, at least 5%, at least 10%, at least 15%, at least 20%, at least 30%, at least 40%, at least 50% or more relative to said binding affinity of corresponding TL1A binding region prior to said at least one amino acid modification, as measured by surface plasmon resonance spectroscopy.

11. The IgG antibody construct of claim 2, wherein said at least one amino acid modification is
(a) within a CDR-H1, a CDR-H2, and/or a CDR-H3 of said IL-23 binding heavy chain variable domain,
(b) within at least one framework region of said IL-23 binding heavy chain variable domain,
(c) within a CDR-L1, a CDR-L2, and/or a CDR-L3 of said IL-23 binding light chain variable domain, or
(b) (d) within at least one framework region of said IL-23 binding light chain variable domain.

12. The IgG antibody construct of claim 11, wherein said at least one amino acid modification increases binding affinity of said IL-23 binding region for said IL-23 in neutral pH condition by at least 2%, at least 5%, at least 10%, at least 15%, at least 20%, at least 30%, at least 40%, at least 50% or more relative to said binding affinity of corresponding IL-23 binding region prior to said at least one amino acid modification, as measured by surface plasmon resonance spectroscopy.

13. The IgG antibody construct of claim 11, wherein said at least one amino acid modification reduces binding affinity of said TL1A binding region for said TL1A in acidic pH condition by at least 10% relative to said binding affinity of corresponding TL1A binding region prior to said at least one amino acid modification, as measured by surface plasmon resonance spectroscopy.

14. The IgG antibody construct of claim 1, wherein said IgG antibody construct has higher binding affinity for trimeric TL1A relative to monomeric TL1A under neutral pH condition or wherein said IgG antibody construct has higher binding affinity for monomeric TL1A relative to trimeric TL1A under neutral pH condition.

15. The IgG antibody construct of claim 2, wherein said IgG antibody construct has higher binding affinity for monomeric IL-23 relative to heterodimeric IL-23 under neutral pH condition or wherein said IgG antibody construct has higher binding affinity for heterodimeric IL-23 relative to monomeric IL-23 under neutral pH condition.

16. The IgG antibody construct of claim 2, wherein said TL1A binding light chain variable domain and said IL-23 binding light chain variable domain are at least 90%, at least 95%, at least 98% or 100% identical relative to each other.

17. The IgG antibody construct of claim 1, wherein said at least one amino acid modification in said TL1A binding heavy chain variable domain increases half-life of said IgG antibody construct in vivo by at least 2%, at least 5%, at least 10%, at least 15%, at least 20%, at least 30%, at least 40%, at least 50% or more relative to half-life of corresponding IgG antibody construct prior to said at least one amino acid modification.

18. The IgG antibody construct of claim 2, wherein TL1A binding heavy chain variable domain comprises a heavy chain variable region, wherein the heavy chain variable region comprises at least one modification at positions selected from S30, T69, L83, N84, T104 and F107, relative to SEQ ID NO: 923; and wherein IL-23 binding heavy chain variable domain comprises at least one modification at positions selected from T28, T30, I31, A33, I34, G56, G58, H59, Q62, Q65, R98, E101, N102 and L108 relative to SEQ ID NO: 1544.

19. An IgG antibody construct comprising:
a TL1A binding region that binds an epitope on TL1A, a variant thereof or a functional fragment thereof, and
a second binding region that binds an epitope on IL-12p40, IL-12p35, IL-6R, IL-6, IL-17 family cytokine, IL-17R, a variant thereof or a functional fragment thereof,
wherein:
said TL1A binding region comprises a TL1A binding heavy chain variable domain and a TL1A binding light chain variable domain, and
at least one amino acid modification in said TL1A binding heavy chain variable domain and/or said TL1A binding light chain variable domain, and
said amino acid modification changes an isoelectric point (pI) of said IgG antibody construct relative to a pI of a corresponding IgG antibody construct prior to said amino acid modification, thereby resulting in pH-dependent binding activity for said TL1A in said IgG antibody construct, as measured by surface plasmon resonance spectroscopy.

20. A method of treating a disease or a condition, wherein the method comprises administering an effective amount of the IgG antibody construct of claim 1.
